(19) United States
(12) Patent Application Publication
(10) Pub. No.: US 2024/0307592 A1
(43) Pub. Date: Sep. 19, 2024

(54) PLANT-DERIVED AEROGELS, HYDROGELS, AND FOAMS, AND METHODS AND USES THEREOF

(71) Applicant: Spiderwort Inc., Ottawa (CA)

(72) Inventors: Ryan Hickey, Ottawa (CA); Kama Szereszewski, Ottawa (CA); Andrew E. Pelling, Ottawa (CA); Paula Cristina de Sousa Faria Tischer, Parana (BR); Anna CANTO, Carleton Place (CA); Joshua SALAMUN, Ottawa (CA); Matthew LOURENCO, Mississauga (CA)

(21) Appl. No.: 18/034,455

(22) PCT Filed: Oct. 29, 2021
(86) PCT No.: PCT/CA2021/051537

§ 371 Date: Apr. 28, 2023

Related U.S. Application Data
(60) Provisional application No. 63/107,226, filed on Oct. 29, 2020.

(51) Int. Cl. A61L 27/52; A23L 29/262; A23L 31/00; A23P 30/40; A61L 27/20; A61L 27/38; A61L 27/54; A61L 27/56; C08L 5/14

(52) U.S. Cl. CPC A61L 27/52; A23L 29/262; A23L 31/00; A23P 30/40; A61L 27/20; A61L 27/383; A61L 27/54; A61L 27/56; C08L 5/14; A61L 2300/402; A61L 2400/06; A61L 2430/02; A61L 2430/34; A61L 2430/38

(57) ABSTRACT

Provided herein are aerogels and foams including: single structural cells and/or groups of structural cells derived from a plant or fungal tissue, the single structural cells having a decellularized 3D structure lacking cellular materials and nucleic acids of plant or fungal tissue; the single structural cells and/or groups of structural cells being distributed within a carrier derived from a dehydrated, lyophilized, or freeze-dried hydrogel. Also provided herein are methods for preparing aerogels or foams, including steps of: providing a decellularized plant or fungal tissue; obtaining single structural cells and/or groups of structural cells from the decellularized plant or fungal tissue by performing mercerization; mixing or distributing the single structural cells and/or groups of structural cells in a hydrogel, to provide a mixture; and dehydrating, lyophilizing, or freeze-drying the mixture to provide the aerogel or foam. Related methods and uses are also provided.

### FIELD OF INVENTION

The present invention relates generally to aerogels, hydrogels, and foams. More specifically, the present invention relates to aerogels, hydrogels, and foams derived from and/or comprising decellularized plant or fungal tissue or structural cells thereof.

### BACKGROUND

Scaffold materials are highly sought after in a number of different fields, especially those providing homogenous and/or reproducible 3-dimensional structures. In the pharmaceutical, medical device, therapy, and food spaces, biocompatible and/or edible scaffold materials are particularly sought after, and those capable of supporting cell growth are highly desirable.

Various scaffold materials have been developed, many of which are based on synthetic polymers or other such materials. Some of which are known to be biocompatible and/or bio-inert, but additional scaffold materials are still of significant interest for a variety of applications.

Scaffold biomaterials comprising decellularized plant or fungal tissue have been developed and described in PCT patent publication WO2017/136950, entitled “Decellularised Cell Wall Structures From Plants and Fungus and Use Thereof as Scaffold Materials”. Remarkable biocompatibility, and uses in a variety of therapeutic applications, are described. These scaffold biomaterials are of significant interest for a variety of different applications.

Nevertheless, additional scaffold materials, and particularly those providing aerogels, hydrogels, and/or foams, are desirable in a variety of fields. Aerogels, hydrogels, and/or foams providing tunable or customizable physical/mechanical properties and/or micro/macro-scale architectures are especially sought after.

Alternative, additional, and/or improved aerogels, hydrogels, and/or foams, as well as methods and/or uses thereof, are desirable.

### SUMMARY OF INVENTION

Provided herein are aerogels, hydrogels, and foams derived from and/or comprising decellularized plant or fungal tissue or structural cells thereof. As described in detail hereinbelow, aerogels, hydrogels, and foams have now been developed which may be derived from and/or may comprise decellularized plant or fungal tissue or structural cells thereof, and which: may comprise plant or fungal microstructures and/or architectures of interest; may be produced by readily scalable production methods; may provide for a wide range of scaffold microstructures and/or macrostructures and/or biochemistry; may provide tunable mechanical properties; may provide tunable porosity; may be biocompatible in vitro and/or in vivo; may be stable to a variety of conditions (such as cooking conditions in the case of food products); or any combinations thereof. By using single structural cells, groups of structural cells, or both, derived from a plant or fungal tissue (the single structural cells or groups of structural cells having a decellularized 3-dimensional structure lacking cellular materials and nucleic acids of plant or fungal tissue), distributed within a carrier derived from one or more dehydrated, lyophilized, or freeze-dried hydrogels, a variety of aerogels, hydrogels, and foams have now been developed and prepared having desirable properties. In certain embodiments, the single structural cells, groups of structural cells, or both, may be derived from plant or fungal tissue (typically decellularized plant or fungal tissue) using mercerization treatment as described herein, which allows for reproducible and scalable production. Related methods and uses, as well as productions methods, are also described in detail herein.

In an embodiment, there is provided herein an aerogel or foam comprising:
single structural cells, groups of structural cells, or both, derived from a plant or fungal tissue, the single structural cells or groups of structural cells having a decellularized 3-dimensional structure lacking cellular materials and nucleic acids of plant or fungal tissue;
the single structural cells, groups of structural cells, or both, being distributed within a carrier, the carrier derived from a dehydrated, lyophilized, or freeze-dried hydrogel.

In another embodiment of the above aerogel or foam, the aerogel or foam has been rehydrated.

In still another embodiment of any of the above aerogel or aerogels or foam or foams, the plant or fungal tissue from which the single structural cells or groups of structural cells are derived may comprise decellularized plant or fungal tissue.

In yet another embodiment of any of the above aerogel or aerogels or foam or foams, the plant or fungal tissue may be decellularized using SDS and optionally CaCl₂).

In another embodiment of any of the above aerogel or aerogels or foam or foams, the single structural cells, groups of structural cells, or both, may be derived from the plant or fungal tissue by mercerization. In certain embodiments, the plant or fungal tissue may be decellularized plant or fungal tissue.

In still another embodiment of any of the above aerogel or aerogels or foam or foams, the mercerization may comprise treatment of the plant or fungal tissue using sodium hydroxide and hydrogen peroxide with heating.

In yet another embodiment of any of the above aerogel or aerogels or foam or foams, the aerogel or foam may comprise a particle size distribution of the single structural cells with an average feret diameter within a range of about 1 μm to about 1000 μm, such as about 100 to about 500 μm, for example about 100 to about 300 μm.

In another embodiment of any of the above aerogel or aerogels or foam or foams, the hydrogel may comprise alginate, pectin, gelatin, methylcellulose, carboxymethylcellulose, hydroxypropylcellulose, hydroxypropyl methyl cellulose, hydroxyethyl methyl cellulose, agar, pluronic acid, triblock PEO-PPO-PEO copolymers of poly(ethylene oxide) (PEO) and poly(propylene oxide) (PPO), dissolved or regenerated plant cellulose, dissolved cellulose-based hydrogel, hyaluronic acid, extracellular matrix proteins (e.g. collagen, gelatin, or fibronectin, or any combinations thereof), monoacrylated poly(ethylene glycol), poly(ethylene glycol) diacrylate, poly(ethylene glycol) diacrylate (PEGDA)-co-PEGMA, poly(vinyl alcohol), poly(vinylpyrrolidone), poly(lactic-co-glycolic acid), chitosan, chitin, xanthan gum, elastin, fibrin, fibrinogen, cellulose derivatives, carrageenan, or microcrystalline cellulose, or any combinations thereof; wherein the hydrogel is optionally cross-linked.

In still another embodiment of any of the above aerogel or aerogels or foam or foams, the aerogel or foam may comprise templated or aligned microchannels created by directional freezing; or by molding using that possess microscale features; by punching, pressing, stamping, or otherwise forming geometric patterns, depressions, holes, channels, grooves, ridges, wells, or other structural features within and/or onto at least one surface; or any combinations thereof.

In yet another embodiment of any of the above aerogel or aerogels or foam or foams, the plant tissue may comprise apple tissue or a pear tissue.

In another embodiment of any of the above aerogel or aerogels or foam or foams, the aerogel or foam may comprise about 5% to about 95% m/m single structural cells, groups of structural cells, or both, when the aerogel or foam is in hydrated form.

In still another embodiment of any of the above aerogel or aerogels or foam or foams, the hydrogel may comprise alginate, pectin, or both, and the aerogel or foam may be rehydrated with a CaCl₂) solution providing cross-linking.

In yet another embodiment of any of the above aerogel or aerogels or foam or foams, the aerogel or foam may have bulk modulus within a range of about 0.1 to about 500 kPa, such as about 1 to about 200 kPa.

In another embodiment of any of the above aerogel or aerogels or foam or foams, the aerogel or foam may be rehydrated and may further comprise one or more animal cells.

In still another embodiment of any of the above aerogel or aerogels or foam or foams, at least some cellulose and/or cellulose derivative(s) of the aerogel or foam may be cross-linked by physical cross-linking (e.g. using glycine) and/or chemical cross-linking (e.g. using citric acid in the presence of heat); wherein at least some cellulose and/or cellulose derivative(s) of the aerogel or foam is functionalized with a linker (e.g. succinic acid) to which one or more functional moieties are optionally attached (e.g. amine-containing groups, wherein cross-linking may further optionally be achieved with one or more protein cross-linkers such as formalin, formaldehyde, glutaraldehyde, and/or transglutaminase); or any combinations thereof.

In yet another embodiment, there is provided herein single structural cells, groups of structural cells, or both, derived from a decellularized plant or fungal tissue by mercerization of the decellularized plant or fungal tissue, the single structural cells or groups of structural cells having a decellularized 3-dimensional structure lacking cellular materials and nucleic acids of plant or fungal tissue, and lacking one or more base-soluble lignin components of the plant or fungal tissue.

In yet another embodiment, there is provided herein a method for preparing an aerogel or foam, comprising:
providing a decellularized plant or fungal tissue;
obtaining single structural cells, groups of structural cells, or both, from the decellularized plant or fungal tissue, by performing mercerization of the decellularized plant or fungal tissue and collecting the resultant single structural cells or groups of structural cells having a decellularized 3-dimensional structure;
mixing or distributing the single structural cells, groups of structural cells, or both, in a hydrogel, to provide a mixture; and
dehydrating, lyophilizing, or freeze-drying the mixture to provide the aerogel or foam.

In yet another embodiment of the above method, the mercerization may comprise treatment of the decellularized plant or fungal tissue with a base and a peroxide, preferably sodium hydroxide or another hydroxide base as base, and hydrogen peroxide as peroxide.

In still another embodiment of any of the above method or methods, the mercerization may comprise treatment of the decellularized plant or fungal tissue with aqueous sodium hydroxide solution and hydrogen peroxide while heating.

In yet another embodiment of any of the above method or methods, the decellularized plant or fungal tissue may be treated with the aqueous sodium hydroxide solution for a first period of time before the hydrogen peroxide is added to the reaction.

In another embodiment of any of the above method or methods, the hydrogen peroxide may be added as a 30% aqueous hydrogen peroxide solution.

In still another embodiment of any of the above method or methods, the hydrogen peroxide for mercerization may be used in a ratio of:
about 20 mL to about 5 mL of 30% hydrogen peroxide solution:about 100 g decellularized plant or fungal tissue:about 500 mL of 1M NaOH solution;
such as:
about 20 mL of 30% hydrogen peroxide solution:about 100 g decellularized plant or fungal tissue:about 500 mL of 1M NaOH solution;
about 10 mL of 30% hydrogen peroxide solution:about 100 g decellularized plant or fungal tissue:about 500 mL of 1M NaOH solution; or
about 5 mL of 30% hydrogen peroxide solution:about 100 g decellularized plant or fungal tissue:about 500 mL of 1M NaOH solution.

In yet another embodiment of any of the above method or methods, the method may further comprise a step of neutralizing the pH with one or more neutralization treatments.

In another embodiment of any of the above method or methods, the neutralization treatment may comprise treatment with an acid solution, preferably an aqueous HCl solution.

In still another embodiment of any of the above method or methods, the mercerization may be performed with heating to about 80° C.

In yet another embodiment of any of the above method or methods, the mercerization may be performed using a ratio of decellularized plant or fungal tissue:aqueous sodium hydroxide solution (m:v, in g:L) of about 1:5 for a 1M aqueous sodium hydroxide solution, or an equivalent ratio for another aqueous sodium hydroxide solution concentration.

In another embodiment of any of the above method or methods, the mercerization may be performed for at least about 30 minutes, preferably for about 1 hour.

In still another embodiment of any of the above method or methods, the resultant single structural cells or groups of structural cells having a decellularized 3-dimensional structure may be collected by centrifugation.

In yet another embodiment of any of the above method or methods, the single structural cells, groups of structural cells, or both, may be mixed or distributed in a hydrogel comprising alginate, pectin, gelatin, methylcellulose, carboxymethylcellulose, hydroxypropylcellulose, hydroxypropyl methyl cellulose, hydroxyethyl methyl cellulose, agar, pluronic acid, triblock PEO-PPO-PEO copolymers of poly(ethylene oxide) (PEO) and poly(propylene oxide) (PPO), dissolved or regenerated plant cellulose, dissolved cellulose-based hydrogel, hyaluronic acid, extracellular matrix proteins (e.g. collagen, gelatin, or fibronectin, or any combinations thereof), monoacrylated poly(ethylene glycol), poly(ethylene glycol) diacrylate, poly(ethylene glycol) diacrylate (PEGDA)-co-PEGMA, poly(vinyl alcohol), poly(vinylpyrrolidone), poly(lactic-co-glycolic acid), chitosan, chitin, xanthan gum, elastin, fibrin, fibrinogen, cellulose derivatives, carrageenan, or microcrystalline cellulose, or any combinations thereof; wherein the hydrogel is optionally cross-linked.

In another embodiment of any of the above method or methods, the method may further comprise a step of performing directional freezing of the mixture to introduce templated or aligned microchannels on a surface of the mixture, within the mixture, or both; a step of molding the mixture using molds having microscale features contacting one or more surfaces of the mixture and/or the aerogel or foam resulting from dehydrating, lyophilizing, or freeze-drying of the mixture, so as to introduce templated or aligned microchannels; a step of punching, pressing, stamping, or otherwise forming geometric patterns, depressions, holes, channels, grooves, ridges, wells, or other structural features within and/or onto at least one surface of the mixture and/or the aerogel or foam before, during, or after dehydrating, lyophilizing, or freeze-drying of the mixture; or any combinations thereof.

In still another embodiment of any of the above method or methods, the directional freezing may be performed by creating a thermal gradient across the mixture from one or more directions so as to form aligned ice crystals beginning from the cold side(s) of the thermal gradient.

In still another embodiment of any of the above method or methods, a microarchitecture of the microchannels produced from directional freezing may be controlled by creating the mixture including a solvent containing varying amounts of one or more other dissolved compounds such as Sucrose, Dextrose, Trehalose, Corn Starch, Glycerol, Ethanol, Mannitol, Sodium chloride, CaCl2, Gelatine, Citric acid, PVA, PEG, Dextran, NaF, NaBr, NaI, phosphate buffer, or another such agent, which alters the structural properties of aligned ice crystals which grow from the cold side of the thermal gradient.

In yet another embodiment of any of the above method or methods, the mixture may be directionally frozen over a period of at least about 30 minutes, preferably over a period of about 2 hours.

In another embodiment of any of the above method or methods, the mixture may be directionally frozen by cooling to a temperature between about −190° C. and about 0° C., such as at least about −15° C., preferably about −25° C.

In still another embodiment of any of the above method or methods, the step of dehydrating, lyophilizing, or freeze-drying the mixture to provide the aerogel or foam may comprise freezing the mixture followed by lyophilizing or freeze-drying the mixture.

In yet another embodiment of any of the above method or methods, the method may comprise a further step of cross-linking the hydrogel (such as cross-linking the hydrogel before or after freezing/lyophilisation, for example), rehydrating the aerogel or foam, or both; optionally using CaCl₂) solution to provide cross-linking where alginate or pectin or agar hydrogel is present.

In another embodiment of any of the above method or methods, the method may comprise a further step of culturing animal cells on or in the aerogel or foam.

In another embodiment, there is provided herein an aerogel or foam produced by any of the method or methods as described herein.

In still another embodiment, there is provided herein a use of any of the aerogel or aerogels or foam or foams as described herein, for bone tissue engineering.

In yet another embodiment, there is provided herein a use of any of the aerogel or aerogels or foam or foams as described herein, for templating or aligning growth of cells.

In another embodiment of the above use, the cells may comprise muscle cells.

In still another embodiment of the above use, the cells may comprise nerve cells.

In yet another embodiment, there is provided herein a use of any of the aerogel or aerogels or foam or foams as described herein for repair of spinal cord injury.

In another embodiment, there is provided herein a use of any of the aerogel or aerogels or foam or foams as described herein as insulation or packaging foam.

In still another embodiment, there is provided herein a method for bone tissue engineering or repair in a subject in need thereof, comprising:
implanting any of the aerogel or aerogels or foam or foams as described herein at an affected site of the subject in need thereof;
such that the aerogel or foam promotes bone tissue generation or repair.

In yet another embodiment, there is provided herein a method for templating or aligning growth of cells, comprising:
culturing cells on any of the aerogel or aerogels or foam or foams as described herein, wherein the aerogel or foam comprises templated or aligned microchannels on at least one surface of the aerogel or foam, within the aerogel or foam, or both, optionally formed by directional freezing; by molding using molds having microscale features; by punching, pressing, stamping, or otherwise forming geometric patterns, depressions, holes, channels, grooves, ridges, wells, or other structural features within and/or onto at least one surface; or any combinations thereof;
such that the cultured cells align along the microchannels.

In another embodiment of the above method, the cells may comprise muscle cells or nerve cells.

In still another embodiment, there is provided herein a method for repairing spinal cord injury in a subject in need thereof, comprising:
implanting any of the aerogel or aerogels or foam or foams as defined herein at an affected site of the subject in need thereof, wherein the aerogel or foam comprises templated or aligned microchannels optionally formed directional freezing;
such that the aerogel or foam promotes spinal cord repair by aligning growth of nerve cells along the templated or aligned microchannels.

In another embodiment, there is provided herein a food product comprising any of the aerogel or aerogels or foam of foams as described herein.

In still another embodiment of the above food product, the food product may be created for a cell-based or plant-based meat industry, and may utilize cellular agriculture techniques to create cultured meat products or plant-based meat products comprising or using aerogels and/or foams as described herein such as those including materials derived from decellularized plant or fungal tissues. The Examples included hereinbelow support that aerogels and/or foams as described herein may be cooked, may support mammalian cell growth, may be coloured and formed into plant-based and/or cell-based meat products. The Examples set out hereinbelow include a detailed example of a plant-based tuna fish mimic as an illustrative example.

In still another embodiment of the above food product, the food product may comprise a dye or coloring agent.

In yet another embodiment of any of the above food product or food products, the food product may comprise two or more aerogel or foam subunits glued together.

In another embodiment of any of the above food product or food products, the glue may comprise agar.

In still another embodiment of any of the above food product or food products, the aerogel or foam may comprise templated or aligned microchannels optionally formed by directional freezing, and wherein the aerogel or foam comprises muscle cells, fibroblasts, myofibroblasts, neurons, dorsal root ganglion cells, neuronal structures such as axons, neural precursor cells, neural stem cells, glial cells, endogenous stem cells, neutrophils, mesenchymal stem cells, satellite cells, myoblasts, myotubes, muscle progenitor cells, adipocytes, preadipocytes, chondrocytes, osteoblasts, osteoclasts, pre-osteoblasts, tendon progenitor cells, tenocytes, periodontal ligament stem cells, or endothelial cells, or any combinations thereof, aligned along the templated or aligned microchannels; preferably wherein the aerogel or foam comprises templated or aligned microchannels optionally formed by directional freezing, and wherein the aerogel or foam comprises muscle cells, fat cells, connective tissue cells (e.g. fibroblasts), cartilage, bone, epithelial, or endothelial cells, or any combinations thereof, aligned along the templated or aligned microchannels.

In another embodiment, there is provided herein a use of any of the aerogel or aerogels or foam or foams as described herein in a food product.

In still another embodiment, there is provided herein a method for preparing single structural cells, groups of structural cells, or both, from decellularized plant or fungal tissue, comprising:
providing a decellularized plant or fungal tissue;
obtaining single structural cells, groups of structural cells, or both, from the decellularized plant or fungal tissue, by performing mercerization of the decellularized plant or fungal tissue and collecting the resultant single structural cells or groups of structural cells having a decellularized 3-dimensional structure.

In yet another embodiment of the above method, the mercerization may comprise treatment of the decellularized plant or fungal tissue with a base and a peroxide, preferably sodium hydroxide or another hydroxide base as base, and hydrogen peroxide as peroxide.

In another embodiment of any of the above method or methods, the mercerization may comprise treatment of the decellularized plant or fungal tissue with aqueous sodium hydroxide solution and hydrogen peroxide while heating.

In still another embodiment of any of the above method or methods, the decellularized plant or fungal tissue may be treated with the aqueous sodium hydroxide solution for a first period of time before the hydrogen peroxide is added to the reaction.

In yet another embodiment of any of the above method or methods, the hydrogen peroxide may be added as a 30% aqueous hydrogen peroxide solution.

In still another embodiment of any of the above method or methods, the hydrogen peroxide for mercerization may be used in a ratio of:
about 20 mL to about 5 mL of 30% hydrogen peroxide solution:about 100 g decellularized plant or fungal tissue:about 500 mL of 1M NaOH solution;
such as:
about 20 mL of 30% hydrogen peroxide solution:about 100 g decellularized plant or fungal tissue:about 500 mL of 1M NaOH solution;
about 10 mL of 30% hydrogen peroxide solution:about 100 g decellularized plant or fungal tissue:about 500 mL of 1M NaOH solution; or
about 5 mL of 30% hydrogen peroxide solution:about 100 g decellularized plant or fungal tissue:about 500 mL of 1M NaOH solution.

In yet another embodiment of any of the above method or methods, the method may further comprise a step of neutralizing the pH with one or more neutralization treatments.

In another embodiment of any of the above method or methods, the neutralization treatment may comprise treatment with an acid solution, preferably an aqueous HCl solution.

In still another embodiment of any of the above method or methods, the mercerization may be performed with heating to about 80° C.

In yet another embodiment of any of the above method or methods, the mercerization may be performed using a ratio of decellularized plant or fungal tissue: aqueous sodium hydroxide solution (m:v, in g:L) of about 1:5 for a 1M aqueous sodium hydroxide solution, or an equivalent ratio for another aqueous sodium hydroxide solution concentration.

In still another embodiment of any of the above method or methods, the mercerization may be performed for at least about 30 minutes, preferably for about 1 hour.

In yet another embodiment of any of the above method or methods, the resultant single structural cells or groups of structural cells having a decellularized 3-dimensional structure may be collected by centrifugation.

In still another embodiment, there is provided herein single structural cells, groups of structural cells, or both, prepared by any of the method or methods as described herein.

In yet another embodiment, there is provided herein a cellulose-based hydrogel comprising cellulose derived from decellularized plant or fungal tissue via dissolution with dimethylacetamide and lithium chloride followed by regeneration with ethanol.

In still another embodiment, there is provided herein a method for preparing a cellulose-based hydrogel comprising:
providing a decellularized plant or fungal tissue;
dissolving cellulose of the decellularized plant or fungal tissue by treatment with dimethylacetamide (DMAc) and lithium chloride (LiCl); and
regenerating a cellulose-based hydrogel from the dissolved cellulose by solvent exchange with ethanol,
thereby providing the cellulose-based hydrogel.

In another embodiment of the above method, the solvent exchange with ethanol may be performed using a dialysis membrane, or by adding ethanol on top of the dissolved cellulose to promote solvent exchange.

In still another embodiment of any of the above method or methods, the method may further comprise bleaching the cellulose-based hydrogel with hydrogen peroxide.

In yet another embodiment, there is provided herein a cellulose-based hydrogel comprising cellulose derived from decellularized plant or fungal tissue via dissolution with: dimethylacetamide and lithium chloride, LiClO₄, xanthate, EDA/KSCN, H₃PO₄, NaOH/urea, ZnCl₂, TBAF/DMSO, NMMO, an ionic liquid (IL) (such as 1-butylpyridinium chloride and aluminum chloride, alkyl imidazolium in association with nitrate, preferably a room temperature ionic liquid), or any combinations thereof.

In still another embodiment, there is provided herein a method for preparing a cellulose-based hydrogel comprising:
providing a decellularized plant or fungal tissue;
dissolving cellulose of the decellularized plant or fungal tissue by treatment with dimethylacetamide and lithium chloride, LiClO₄, xanthate, EDA/KSCN, H₃PO₄, NaOH/urea, ZnCl₂, TBAF/DMSO, NMMO, an ionic liquid (IL) (such as 1-butylpyridinium chloride and aluminum chloride, alkyl imidazolium in association with nitrate, preferably a room temperature ionic liquid), or any combinations thereof;
obtaining the dissolved cellulose and preparing the cellulose-based hydrogel using the dissolved cellulose.

In another embodiment, there is provided herein a cellulose-based hydrogel prepared by any of the method or methods as described herein.

In another embodiment of any of the aerogel or aerogels or foam or foams as described herein, the hydrogel may comprise any of the cellulose-based hydrogel or cellulose-based hydrogels as described herein.

In still another embodiment, there is provided herein a food product comprising any of the aerogel or aerogels or foam or foams or structural cell or structural cells as described herein, wherein the food product is a meat mimic and comprises a plurality of lines providing the appearance of fatty white lines found in tuna, salmon, or another fish-type meat.

In yet another embodiment of the above food product, the food product may be a mimic of tuna, salmon, or another fish meat.

In another embodiment of any of the above food product or food products, the food product may contain one or more dyes or colorants providing the color of tuna, salmon, or another fish meat.

In still another embodiment of any of the above food product or food products, the plurality of lines may be formed in cuts or channels formed in the aerogel or foam.

In yet another embodiment of any of the above food product or food products, the plurality of lines may comprise titanium dioxide, optionally combined with agar binding agent or another such binding agent.

In another embodiment of any of the above food product or food products, the titanium dioxide, optionally combined with agar binding agent, may be applied into cuts or channels formed in the aerogel or foam to provide the appearance of the fatty white lines found in tuna, salmon, or another fish-type meat.

In another embodiment, there is provided herein a method for preparing a food product, the food product being a tuna, salmon, or other fish meat mimic, said method comprising:
providing any of the aerogel or aerogels or foam or foams as described herein;
optionally, dying or coloring the aerogel a color of tuna, salmon, or other fish meat;
cutting or otherwise processing the aerogel in order to form cuts or channels along the surface of the aerogel; and
applying a dye or coloring agent to the cuts or channels to provide an appearance of fatty white lines characteristic of tuna, salmon, or other fish meat.

In another embodiment of the above method, the dye or coloring agent applied to the cuts or channels may comprise titanium dioxide.

In still another embodiment of any of the above method or methods, the dye or coloring agent applied to the cuts or channels may be combined with a binding agent.

In yet another embodiment of any of the above method or methods, the binding agent may comprise agar.

In another embodiment, there is provided herein a food product prepared by any of the method or methods described herein.

In another embodiment, there is provided herein a non-resorbable dermal filler comprising an aerogel, foam, structural cell, or cellulose-based hydrogel as described herein; or any combinations thereof.

In yet another embodiment, there is provided herein a dermal filler comprising single structural cells, groups of structural cells, or both, derived from a plant or fungal tissue, the single structural cells or groups of structural cells having a decellularized 3-dimensional structure lacking cellular materials and nucleic acids of plant or fungal tissue, the single structural cells, groups of structural cells, or both, being derived from the plant or fungal tissue by mercerization.

In another embodiment of any of the above dermal filler or dermal fillers, the dermal filler may further comprise a carrier fluid or gel.

In still another embodiment of any of the above dermal filler or dermal fillers, the carrier fluid or gel may comprise water, an aqueous solution, or a hydrogel.

In yet another embodiment of any of the above dermal filler or dermal fillers, the carrier fluid or gel may comprise a saline solution, or a collagen, hyaluronic acid, methylcellulose, and/or dissolved plant-derived decellularized cellulose-based hydrogel.

In another embodiment of any of the above dermal filler or dermal fillers, the dermal filler may further comprise an anesthetic agent.

In still another embodiment of any of the above dermal filler or dermal fillers, the anesthetic agent may comprise lidocaine, benzocaine, tetracaine, polocaine, epinephrine, or any combinations thereof.

In another embodiment of any of the above dermal filler or dermal fillers, the dermal filler may comprise PBS (saline), hyaluronic acid (cross-linked or non-crosslinked), alginate, collagen, pluronic acid (e.g. pluronic F 127), agar, agarose, or fibrin, calcium hydroxylapatite, Poly-L-lactic acid, autologous fat, silicone, dextran, methylcellulose, or any combinations thereof.

In another embodiment of any of the above dermal filler or dermal fillers, the dermal filler may comprise at least one of: 2% lidocaine gel; a triple anesthetic gel comprising 20% benzocaine, 6% lidocaine, and 4% tetracaine (BLTgel); 3% Polocaine; or a mixture of 2% lidocaine with epinephrine.

In another embodiment of any of the above dermal filler or dermal fillers, the structural cells may have a size, diameter, or minimum feret diameter of at least about 20 μm.

In another embodiment of any of the above dermal filler or dermal fillers, the structural cells may have a size, diameter, or maximum feret diameter of less than about 1000 μm.

In still another embodiment of any of the above dermal filler or dermal fillers, the structural cells may have a size, diameter, or feret diameter distribution within a range of about 20 μm to about 1000 μm.

In yet another embodiment of any of the above dermal filler or dermal fillers, the structural cells may have a particle size, diameter, or feret diameter distribution having a peak about 200-300 μm.

In another embodiment of any of the above dermal filler or dermal fillers, the structural cells may have a mean particle size, diameter, or feret diameter within a range of about 200 μm to about 300 μm.

In another embodiment of any of the above dermal filler or dermal fillers, the structural cells may have an average projected particle area within a range of about 30,000 to about 75,000 μm².

In still another embodiment of any of the above dermal filler or dermal fillers, the dermal filler may be sterilized.

In yet another embodiment of any of the above dermal filler or dermal fillers, the sterilization may be by gamma sterilization.

In still another embodiment of any of the above dermal filler or dermal fillers, the dermal filler may be formulated for subdermal injection, deep dermal injection, subcutaneous injection (e.g. subcutaneous fat injection), or any combinations thereof.

In another embodiment of any of the above dermal filler or dermal fillers, the dermal filler may be provided in a syringe or injection device.

In another embodiment, there is provided herein a use of any of the dermal filler or dermal fillers as described herein as a soft tissue filler, for reconstructive surgery, or both.

In another embodiment, there is provided herein a use of any of the dermal filler or dermal fillers as described herein for improving cosmetic appearance of a subject in need thereof.

In another embodiment, there is provided herein a use of any of the dermal filler or dermal fillers as described herein for increasing tissue volume, smoothing wrinkles, or both, in a subject in need thereof.

In another embodiment, there is provided herein a method for improving cosmetic appearance, increasing tissue volume, smoothing wrinkles, or any combinations thereof, in a subject in need thereof, said method comprising:
administering or injecting any of the dermal filler or dermal fillers as described herein to a region in need thereof;
thereby improving cosmetic appearance, increasing tissue volume, smoothing wrinkles, or any combinations thereof, of the subject.

In another embodiment of the above use or uses or method or methods, native cells of the subject may infiltrate the dermal filler.

In yet another embodiment of the above use or uses or method or methods, the dermal filler may be non-resorbable such that the decellularized plant or fungal tissue remains substantially intact within the subject.

### BRIEF DESCRIPTION OF DRAWINGS

These and other features will become further understood having regard to the following Description and accompanying Drawings, wherein:

FIG. 1 shows results of AA (apple) mercerization and discolouring in a smaller sample of AA (100 g in the images), as described in Example 1. 100 g of decellularized AA (apple) material was mercerized in 500 mL of 1M NaOH at 80° C. for one hour. A total of 75 mL of H₂O₂ was added throughout the mercerization process to discolour the samples (reaction formed Na₂O₂ (sodium peroxide) which is a strong oxidizer). FIG. 1(A) shows AA samples in NaOH (T=0 min). FIG. 1(B) shows AA samples in NaOH right after the addition of 25 mL of H₂O₂. The discolouration process started right after the addition. (T=2 min). In FIG. 1(C) the samples appear yellowish (T=10 min). In FIG. 1(D) AA samples appear off-white after 60 minutes of mercerization in NaOH and the H₂O₂ additions;

FIG. 2(A) shows the decellularized AA tissue used as the starting material for the mercerization process, and FIG. 2(B) shows the product obtained after the mercerization, as described in Example 1. The product is shown after follow-up neutralization and centrifugation. The obtained product material shown in FIG. 2(B) is very thick and viscous, resembling a sort of apple “paste”;

FIG. 3 shows images of the apple-derived decellularized single structural cells (and some groups of structural cells comprising a small plurality of single structural cells linked together) obtained/isolated following mercerization as described in Example 1. In FIG. 3, dilution and fluorescent staining of the structural cells with congo red dye revealed the microarchitecture of the cells is intact;

FIG. 4 shows the particle size distribution of mercerated (1M NaOH) decellularized AA with the addition of H₂O₂ (N=10 images analyzed) as described in Example 1. Average size confirms the presence of intact single structural cells which have maintained their microarchitectural characteristics;

FIG. 5 shows colour change of AA-NaOH solution throughout the 60-minute mercerization of all three ratio conditions (i.e., 20 g, 50 g, and 100 g of AA in 100 mL 1M NaOH) as described in Example 1;

FIG. 6 shows that after mercerization in the various solutions, the isolated single AA cells were imaged and their ferret diameters were measured as described in Example 1. The results show that there was no significant difference in the average size, number and distribution of isolated mercerized cells under each condition;

FIG. 7 shows a 5% Alginate aerogel as described in Example 1. The scaffold is 6 cm in diameter and 0.7 cm thick;

FIG. 8 shows a microscope image of a 50% Alginate aerogel as described in Example 1 (scale bar=500 um);

FIG. 9 shows a cross-linked 50% Alginate aerogel that has been rehydrated as described in Example 1 (aerogel is about 1 cm diameter, 4 mm thick);

FIG. 10 shows an example of a hydrated aerogel (being alginate-based in this example) on a frying pan with butter at the start of cooking, as described in Example 1;

FIG. 11 shows the same aerogel depicted in FIG. 10 but after several minutes of cooking, where it is observed that the aerogel maintained its shape and integrity, and a crust was formed;

FIG. 12 shows a comparison of “raw” (left) and cooked (right) aerogels, as described in Example 1;

FIG. 13 shows the custom-built directional freezing apparatus used in Example 1;

FIG. 14 shows a schematic diagram of the directional freezing apparatus depicted in FIG. 13;

FIG. 15 shows a syringe mixing apparatus used to mix an alginate hydrogel with a gel comprising structural cells obtained from mercerization of decellularized apple tissue, as described in Example 1;

FIG. 16 shows a top down view of the aerogel still in the falcon tube as described in Example 1, in which porous structures are observable;

FIG. 17 shows aerogels after removal from the falcon tubes as described in Example 1;

FIG. 18 shows aerogel foam prepared without additional freezing time in the −20° C. freezer, which collapsed during lyophilisation (left); and aerogel foam which was left overnight in the freezer (−20° C.) prior to lyophilisation (right); as described in Example 1. Each scaffold is ˜3 cm tall;

FIG. 19 shows a reflected light image of an entire aerogel cross section (1× condenser, 0.75× magnification) as described in Example 1;

FIG. 20 shows brightfield cross-section perpendicular to the axis of the aerogel cylinder (Stereomicroscope 2× Condenser, 1.25 Zoom) as described in Example 1;

FIG. 21 shows brightfield cross-section parallel to the axis of the aerogel cylinder (Stereomicroscope 2× Condenser, 1.25 Zoom) as described in Example 1;

FIG. 22 shows darkfield cross-section perpendicular to the axis of the aerogel cylinder (Stereomicroscope 2× Condenser, 1.25 Zoom) as described in Example 1;

FIG. 23 shows darkfield cross-section parallel to the axis of the aerogel cylinder (Stereomicroscope 2× Condenser, 1.25 Zoom) as described in Example 1;

FIG. 24 shows SEM cross-section perpendicular to the axis of the aerogel cylinder, revealing microchannels as described in Example 1;

FIG. 25 shows SEM cross-section perpendicular to the axis of the aerogel cylinder, revealing microchannels as described in Example 1;

FIG. 26 shows SEM cross-section perpendicular to the axis of the cylinder as described in Example 1;

FIG. 27 shows SEM cross-section perpendicular to the axis of the aerogel cylinder;

FIG. 28 shows SEM cross-section parallel to the axis of the aerogel cylinder, revealing long range alignment as described in Example 1;

FIG. 29 shows SEM cross-section parallel to the axis of the aerogel cylinder as described in Example 1;

FIG. 30 shows SEM cross-section parallel to the axis of the aerogel cylinder as described in Example 1;

FIG. 31 shows SEM cross-section parallel to the axis of the aerogel cylinder as described in Example 1;

FIG. 32 shows images of a dry aerogel section (left) and 0.1M CaCl₂-treated rehydrated aerogel section (right) as described in Example 1. Images were acquired at approximately the same height and magnification. The aerogel sections remained intact, maintained their microstructure, and could be picked up and manipulated. In this case, rehydration in CaCl₂ solution crosslinked and stabilized the alginate of the rehydrated aerogel (right);

FIG. 33 depicts a freezing apparatus in a styrofoam box, in which LN₂ had just been added immediately before the photo was taken, and can be seen boiling in the bottom, as described in Example 1;

FIG. 34 shows three formulations of hydrogel mixture that were prepared in which the solvent was either A) PBS, B) 0.9% saline, or C) water in order to assess if salts would alter ice crystal formation and channel alignment/architecture during directional freezing (scale bar=2 mm and applies to all). In all cases, the material froze so quickly and without significant ice crystal formation that aligned channels were not observed. A very dense and soft foam resulted from the process, as described in Example 1;

FIG. 35 shows (A) water-based alginate mixture which was directionally frozen on the LN₂ system of FIG. 33 (scale bar=2 mm). The scaffold was very dense and soft, and appeared homogeneous to the eye. This was in stark contrast to the scaffolds created on the peltier-based directional freezing platform in which the channeled architecture was clearly visible to the eye. As shown in (B), however, at high resolution (scale bar=200 um), the small pore size of the scaffold becomes visible which creates the opportunity for cell invasion and several potential other applications in tissue engineering and food science, for example, as described in Example 1;

FIG. 36 shows 5% alginate and pectin stock solutions as described in Example 2;

FIG. 37 shows preparation of pluronic stock solution as described in Example 2;

FIG. 38 shows preparation of a gelatin-AA aerogel as described in Example 2;

FIG. 39 shows syringe-based mixing apparatus for mixing hydrogel with mercerized structural cells as described in Example 2;

FIG. 40 depicts representations of the different aerogel formulations prepared as described in Example 2, before and after the freeze-drying of the samples;

FIG. 41 shows results in which GFP 3T3 cells (green) were seeded onto certain aerogel (as shown) stained with Congo Red (red) as described in Example 2. Agar, alginate, pectin, and gelatin hydrogels were used in combination with 1.5 g of decellularized, mercerized apple (10%) or 7.5 g of decellularized, mercerized apple (50%) (Scale=200 μm). Images were acquired on the BX53 upright microscope at 10× with the GFP filter for the cells and the TXRED filter for the scaffold;

FIG. 42 shows stress-strain curves for the dry agar based gels with 1.5 g of mercerized AA as described in Example 2;

FIG. 43 shows stress-strain curves for the dry agar based gels with 7.5 g of mercerized AA as described in Example 2;

FIG. 44 shows stress-strain curves for the dry alginate based gels with 1.5 g of mercerized AA as described in Example 2;

FIG. 45 shows stress-strain curves for the dry alginate based gels with 7.5 g of mercerized AA as described in Example 2;

FIG. 46 shows stress-strain curves for the dry pectin based gels with 1.5 g of mercerized AA as described in Example 2;

FIG. 47 shows stress-strain curves for the dry pectin based gels with 7.5 g of mercerized AA as described in Example 2;

FIG. 48 shows stress-strain curves for the dry gelatin based gels with 1.5 g of mercerized AA as described in Example 2;

FIG. 49 shows stress-strain curves for the dry gelatin based gels with 7.5 g of mercerized AA as described in Example 2;

FIG. 50 shows stress-strain curves for the dry methylcellulose based gels with 1.5 g of mercerized AA as described in Example 2;

FIG. 51 shows stress-strain curves for the dry methylcellulose based gels with 7.5 g of mercerized AA as described in Example 2;

FIG. 52 shows stress-strain curves for the dry pluronic based gels with 1.5 g of mercerized AA as described in Example 2;

FIG. 53 shows stress-strain curves for the dry pluronic and alginate based gels with 7.5 g of mercerized AA as described in Example 2;

FIG. 54 shows Young's moduli for the dry samples that have a hydrate counterpart. The volumes of the mercerized AA are indicated by 1.5 and 7.5; both are in grams and correspond to a 10% and 50% solution respectively. The base hydrogels of 1% agar, alginate and pectin were used. Gelatin was a 5% final solution, as described in Example 2;

FIG. 55 shows stress-strain curves for the hydrated agar based gels with 1.5 g of mercerized AA as described in Example 2;

FIG. 56 shows stress-strain curves for the hydrated agar based gels with 7.5 g of mercerized AA as described in Example 2;

FIG. 57 shows stress-strain curves for the hydrated alginate based gels with 1.5 g of mercerized AA as described in Example 2;

FIG. 58 shows stress-strain curves for the hydrated alginate based gels with 7.5 g of mercerized AA as described in Example 2;

FIG. 59 shows stress-strain curves for the hydrated pectin based gels with 1.5 g of mercerized AA as described in Example 2;

FIG. 60 shows stress-strain curves for the hydrated pectin based gels with 7.5 g of mercerized AA as described in Example 2;

FIG. 61 shows stress-strain curves for the hydrated gelatin based gels with 1.5 g of mercerized AA as described in Example 2;

FIG. 62 shows stress-strain curves for the hydrated gelatin based gels with 7.5 g of mercerized AA as described in Example 2;

FIG. 63 shows stress-strain curves for the hydrated pluronic and alginate based gels with 7.5 g of mercerized AA as described in Example 2;

FIG. 64 shows Young's moduli for the hydrated samples. The volumes of the mercerized AA are indicated by 1.5 and 7.5; both are in grams and correspond to a 10% and 50% solution respectively. The base hydrogels of 1% agar, alginate and pectin were used. Gelatin was a 5% final solution, as described in Example 2;

FIG. 65 shows SEM of alginate based aerogels with 1.5 g (10%) and 7.5 g (50%) of decellularized and mercerated AA as described in Example 2;

FIG. 66 shows SEM of pectin based aerogels with 1.5 g (10%) and 7.5 g (50%) of decellularized and mercerated AA as described in Example 2;

FIG. 67 shows maximum intensity z-projections of confocal images of alginate foams with 7.5 g of mercerized AA (50%) as described in Example 2. The red is the scaffold stained with Congo Red. The green is the GFP of the stably transfected 3T3 cells, and blue is the nucleus of the GFP 3T3 cells;

FIG. 68 shows dissolution solution of DMAc and LiCl with decellularized apple after the 72 h reaction as described in Example 3;

FIG. 69 shows dissolution solution of DMAc and LiCl with decellularized apple after centrifugation to remove undissolved material as described in Example 3;

FIG. 70 shows cellulose film regeneration. Dissolved cellulose was poured into a 60 mm Petri dish to cover the bottom surface. 95% ethanol was poured on top of the dissolution solution to promote solution exchange and regenerate the cellulose. Wrinkles are observed as the film forms, as described in Example 3;

FIG. 71 shows that within 5 minutes of the ethanol addition, the film could be pushed and bundled with a spatula, as described in Example 3;

FIG. 72 shows regenerated cellulose gel that was collected, as described in Example 3;

FIG. 73 shows regenerated cellulose film, when left undisturbed, as described in Example 3;

FIG. 74 shows regenerated cellulose file, titled to show the wafer slide in the petri dish, as described in Example 3;

FIG. 75 shows regenerated cellulose with a density gradient. Solvent exchange was accomplished with a dialysis membrane. The regeneration occurred in a 50 mL falcon tube. The cylindrical end was in contact with the membrane and had the greatest amount of solution exchange. It was stiffer and held its shape compared to the less stiff and less dense tail region, as described in Example 3;

FIG. 76 shows regenerated cellulose film set-up with the dialysis membrane secured by the lid with a hole cut out of the centre, as described in Example 3;

FIG. 77 shows a lyophilized section of the dense region from FIG. 76. The lyophilization led to scaffold collapse, as described in Example 3;

FIG. 78 shows a regenerated cellulose 5 mm punch from a regenerated film bleached with H₂O₂ (30%). The materials were light brown before treatment, and after treatment with peroxide they were clear. In fact, they were difficult to see because of their clarity, as described in Example 3:

FIG. 79 shows a regenerated cellulose 5 mm punch from a regenerated film bleached with H₂O₂ (30%) imaged with dark-field imaging, as described in Example 3;

FIG. 80 shows a regenerated cellulose 5 mm punch from a regenerated film bleached with H₂O₂ (30%) stained with Congo Red to visualize the micro-structure. The surface was very flat with small pores. This is a fluorescence image with TRITC, as described in Example 3;

FIG. 81 shows DMAc LiCl dissolved cellulose mixed with mercerated AA (the colour comes from the DMAc LiCl dissolved cellulose solution; the mercerized material was white), as described in Example 3;

FIG. 82 shows dissolved cellulose with mercerized AA mixed into it. The membrane was regenerated by coating with a layer of 95% ethanol overnight. A composite film is obtained, as described in Example 3;

FIG. 83 shows a fluorescence microscopy image of the regenerated cellulose with the mercerized material mixed into it. The apple structural cells from the mercerized material can be seen tightly packed together. This topography is distinct from the smooth material obtained from pure regenerated cellulose, as described in Example 3;

FIG. 84 shows the reaction arrangement as described in Example 3. The reaction was carried out in small beakers with a magnetic stir bar. These beakers were covered with parafilm and put in a larger beaker which contained an ice bath;

FIG. 85 shows methylcellulose and mercerized AA. The methylcellulose mixed with glycine (upper in the weigh boats) and the mercerized AA (lower in the Petri dishes). The 1 g of methylcellulose was more viscous (right two images) compared to the 0.5 g (left two images), as described in Example 3;

FIG. 86 shows methylcellulose gels with mercerized AA (apple) and glycine (AA introduced after glycine addition) after incubation at room temperature overnight to crosslink. The gels could be removed from the Petri dishes and maintain their shape. The 1 g methylcellulose gels were more stiff, as described in Example 3;

FIG. 87 shows methylcellulose and mercerized AA gel. 1 g of methylcellulose, 1 g of AA mixed in 10 mL of 2 M NaOH for 1 h mixed by magnetic stirring in an ice bath, then 5 mL of 30% glycine in 2 M NaOH was added for an additional hour of stirring on ice. Crosslinking at room temperature overnight in a 60 mm Petri dish. The gels can be handled and maintain their shape, as described in Example 3;

FIG. 88 shows the same gel from FIG. 87 cut with a scalpel blade into two halves. One was kept, and the other was used to test the neutralization as described in Example 3. The neutralization was 5% acetic acid for 1 h followed by 10 water washed. It was also tested whether after doing this there would be a slow release of NaOH which would result in the pH increasing. This did occur. As a result, the half-aerogel was washed 70 times and was also neutralized with 30% acetic acid;

FIG. 89 shows the excessively washed “half-aerogel” from FIG. 88 was frozen at −20° C. and then lyophilized at −46° C. and 0.050 mbar (upper). The dried material appears fragile, but was actually fairly stiff to the touch. Directional freezing was also observed. A section was then torn off and immersed in dH2O (lower image). It remained intact and had a soft, sticky texture, as described in Example 3;

FIG. 90 shows the second half of the aerogel cut from FIG. 88 was neutralized. The neutralization was performed with 30% acetic acid right away. This had a similar, but opposite consequence: the pH would drift to acidic values and the slow release of the acetic acid made the pH drift to lower values over time. This was corrected with a slow titration with 1 M NaOH. Nevertheless this indicates an optimal neutralization step somewhere between 5% and 30% acetic acid will likely be a faster, more efficient approach. The neutral sample was kept for future dye testing, as described in Example 3;

FIG. 91 shows methyl cellulose with mercerized AA (1:1) half-aerogel neutralized with 15% acetic acid. It was also found that the methyl cellulose gels (with and without the AA) swelled greatly. This can occur while freezing and freeze drying as well, as described in Example 3;

FIG. 92 shows Methyl cellulose with mercerized AA (1:1) half-aerogel neutralized with 15% acetic acid. The aerogels shown in FIG. 92 were neutralized as half-aerogels (FIG. 91). During the freezing, they expanded to fill the 60 mm petri dish. Once freeze-dried, they produce a white foam that is easily handled and relatively stiff. Once hydrated, they expand and if they keep expanding, they turn into a loose material with a sticky consistency, as described in Example 3;

FIG. 93 shows Methyl cellulose with mercerized AA (1:1) expansion. The half-aerogel was placed on it's original 60 mm dish for comparison, as described in Example 3;

FIG. 94 shows Methyl cellulose with mercerized AA (1:1) continued expansion into a loose material, as described in Example 3;

FIG. 95 shows crystallization of glycine at reduced temperatures (˜4° C.) from a 40% solution, as described in Example 3;

FIG. 96 shows carboxymethyl cellulose gel in the absence of glycine gives a similar physically crosslinked material;

FIG. 97 shows alginate (left) and pectin (right) aerogel scaffolds prior to implantation into trephinated defects as described in Example 4;

FIG. 98 shows alginate (left) and pectin (right) aerogel biomaterials implanted in the trephinated defects of the parietal bone as described in Example 4;

FIG. 99 shows alginate aerogel implants in the rat calvarium prior to resection as described in Example 4;

FIG. 100 shows resected rat calvarium as described in Example 4;

FIG. 101 shows rat calvariums with trephinated defects resected after 8 weeks and scanned with Computational Tomography (CT). Alginate biomaterials (left) and Pectin biomaterials (right). The results reveal the aerogel biomaterials support cellular infiltration and regeneration in vivo, as described in Example 4;

FIG. 102 shows bleaching during mercerization with 20 mL of hydrogen peroxide over the course of 1 h, as described in Example 5;

FIG. 103 shows bleaching during mercerization with 10 mL of hydrogen peroxide over the course of 1 h, as described in Example 5;

FIG. 104 shows bleaching during mercerization with 5 mL of hydrogen peroxide over the course of 1 h, as described in Example 5;

FIG. 105 shows that (A) after the 1 h mercerization with different amounts of peroxide, the colour is slightly more clear for the higher peroxide concentrations; (B) after neutralization, the slight colour variations disappear and all three have a clear/off-white colour; and (C) the final concentrated product was comparable for the three hydrogen peroxide ratios, as described in Example 5;

FIG. 106 shows fluorescent microscopy images of the three different AA:NaOH ratio conditions (i.e. mercerization conditions) as described in Example 6. (A)—1:5, (B)—1:2, and (C)—1:1. Images were captured with the Olympus SZX16 microscope at 2.5× magnification using the BV filter and Congo red stain;

FIG. 107 shows a histogram of the particle size distributions from the mercerization of decellularized AA in different ratios with 1 M NaOH, as described in Example 6;

FIG. 108 shows an example of an alginate aerogel biomaterial excised from a 60 mm dish following freeze drying as described in Example 7;

FIG. 109 shows a 10 mm Biopsy punch of dry (left) and crosslinked/wet (right) alginate biomaterial being compressed-axial measurement, as described in Example 7;

FIG. 110 shows results in which CMC cross-linked with citric acid is depicted. The CMC control was a clear gel, whereas the CMC with mercerized material (structural cells) was a translucent white gel, as described in Example 8;

FIG. 111 shows results for CMC crosslinked with citric acid membranes. The CMC control (left) was a clear membrane, whereas the CMC with mercerized material (structural cells) was a translucent white membrane that was more stiff—it had the texture of shrimp shells, as described in Example 8;

FIG. 112 shows cellulose after the reaction is complete, as described in Example 8;

FIG. 113 shows cellulose after intensely washing with water is completed, as described in Example 8;

FIG. 114 shows FTIR spectra, showing FTIR spectra of decellularized scaffolds (2 AP-DECEL) and the chemically bonded composite of succinylated plant-derived cellulose (5AP-AS), as described in Example 8;

FIG. 115 shows lyophilized aerogels produced with the formulations as described in Example 3 (samples P1, P2, P3, P4, P5, P6), about 1 cm in diameter;

FIG. 116 shows larger scale lyophilized (3 cm diameter) aerogels produced with the formulations as described in Example 3 (P2 (Left), P7 (Middle), P3 (Right) images);

FIG. 117 shows a “Tuna” sushi mimic (red coloured) which was created by layering and gluing pieces of aerogel (cross-linked 50% Alginate) scaffolds with more alginate. The construct was then cut into a 3×2 cm piece (approx) and coloured with red food dye to mimic real tuna. Small diagonal slices were cut along its length to mimic the interface between muscle layers, as described in Example 9;

FIG. 118 shows a “Tuna” sushi mimic (red coloured) which was created by layering and gluing pieces of previously dyed aerogel (cross-linked 50% Alginate) with an agar “glue” that had been coloured with titanium dioxide (TiO₂), a common white food colorant. This construct allowed to more convincingly mimic the fascia that exists between distinct layers of muscle tissue in real tuna, as described in Example 9;

FIG. 119 shows a “Tuna” (red coloured) which was created by layering and gluing pieces of previously dyed aerogel (cross-linked 50% Alginate) with an agar “glue” that had been coloured with titanium dioxide (TiO₂), a common white food colorant. The agar glue may be placed between layers, or into thin grooves cut along the surface of the aerogel to produce the linear pattern of fascia which exists between muscle layers, as described in Example 9;

FIG. 120 shows the needle occlusion test with mercerized AA as described in Example 10. In (A), a 27 G needle and syringe is shown. (B) shows extrusion of mercerized AA. (C) shows an example for 3D printing or controlled injection/extrusion, for example;

FIG. 121 shows force-displacement curves for N=10 extrusions of water from a 1 cc syringe as described in Example 10;

FIG. 122 shows force-displacement curves for N=10 extrusions of a 20% mercerized AA in saline mixture from a 1 cc syringe as described in Example 10;

FIG. 123 shows force-displacement curves for N=10 extrusions of undiluted mercerized AA from a 1 cc syringe as described in Example 10;

FIG. 124 shows maximum extrusion force for water only, a 20% mercerized AA solution diluted in 0.9% saline, and undiluted mercerized material as described in Example 10;

FIG. 125 shows generation II dermal fillers. (A) shows MER, (B) shows MER20SAL80, (C) shows MER20COL80, and (D) shows MER20REG80. The injections contained 0.3% lidocaine and were prepared as 600 μL injections in 1 cc syringes, as described in Example 10;

FIG. 126 shows results for generation II dermal fillers used as dermal filler in a rat model. (A) shows Pre-injection, and (B) shows Post-injection, as described in Example 10. The black outline was used to track the implant sites from week to week. The bumps under the skin were measured. The bump sizes were measured using Vernier calipers. The ellipsoid estimate was used to estimate the area and volume of the injections;

FIG. 127 shows dermal filler size measurements for the rat model injections as described in Example 10. (A) shows the normalized height, (B) shows the normalized ellipse area, and (C) shows the normalized ellipsoid volume;

FIG. 128 shows a flow chart depicting illustrative examples of aerogel/foam preparation using cross-linking before or after lyophilization;

FIG. 129 shows aerogel scaffolds cut using a 5 mm biopsy punch (A), then removed using a thin wire (B) resulting in the final scaffolds (C and D);

FIG. 130 shows an aerogel produced with crosslinked regenerated cellulose (D1) and succinylated cellulose;

FIG. 131 shows an aerogel produced with crosslinked mercerized cellulose (AS4) and succinylated cellulose;

FIG. 132 shows a brightfield microscopic image of the circled bottom surface of the bottom layer of an aerogel prepared from crosslinked regenerated cellulose (AD1CLS);

FIG. 133 shows a brightfield microscopic image of the circled top surface of the bottom layer of the aerogel of FIG. 132;

FIG. 134 shows a brightfield microscopic image of the circled bottom surface of the top layer of an aerogel prepared from crosslinked mercerized cellulose (AS4);

FIG. 135 shows a brightfield microscopic image of the circled top surface of the bottom layer of the aerogel of FIG. 134;

FIG. 136 shows aerogels AS6, AS9 and AS10 prepared from crosslinked mercerized cellulose (samples S6, S9 and S10) mixed with succinylated mercerized cellulose;

FIG. 137 shows microscope images of the bottom surface of the bottom layer of each aerogel AS6, AS9 and AS10;

FIG. 138 shows stability of each aerogel AS6, AS9 and AS10 after 45 minutes in PBS compared to the aerogel at t=0;

FIG. 139 shows the hydrogels mixed in two 50 mL syringes connected with an f/f luer lock connector (A) and inserted into steel tubes before directional freezing (B and C);

FIG. 140 shows the aerogels Merc.AA, D1A and Merc.AA+D1A after directional freezing, before crosslinking;

FIG. 141 shows the aerogels Merc.AA, D1A and Merc.AA+D1A after crosslinking;

FIG. 142 shows microscope images of the Merc.AA aerogel of FIG. 141;

FIG. 143 shows microscope images of the D1A aerogel of FIG. 141;

FIG. 144 shows microscope images of the Merc.AA+D1A aerogel of FIG. 141;

FIG. 145 shows microscope images of the Merc.AA+succinylated cellulose aerogel of FIG. 141;

FIG. 146 shows aerogels prepared with Merc.AA crosslinked after lyophilisation, after 5 minutes (A and B) and 6 h (C) incubation in PBS;

FIG. 147 shows aerogels prepared with D1A crosslinked after lyophilisation, after 5 minutes (A and B) and 6 h (C) incubation in PBS;

FIG. 148 shows aerogels prepared with Merc.AA+D1A crosslinked after lyophilisation, after 5 minutes (A and B) and 6 h (C) incubation in PBS;

FIG. 149 shows aerogels prepared with Merc.AA+succinylated cellulose crosslinked after lyophilisation, after 24 h incubation in PBS;

FIG. 150 shows microscopy images of aerogel prepared with Merc. AA crosslinked with citric acid for 2 h;

FIG. 151 shows microscopy images of aerogel prepared with regenerated cellulose (D1A) crosslinked with citric acid for 2 h;

FIG. 152 shows microscopy images of aerogel prepared with Merc. AA+regenerated cellulose (D1A) crosslinked with citric acid for 2 h;

FIG. 153 shows the silicone molds and needles (30G) used to optimize pore formation in the aerogels, which were prepared as described above;

FIG. 154 shows an aerogel prepared from Merc. AA using silicone mold needles before crosslinking (A, B) and after crosslinking with citric acid (C, D);

FIG. 155 shows an aerogel prepared from Merc. AA+regenerated cellulose using silicone mold needles before crosslinking (A, B, C) and after crosslinking with citric acid (D);

FIG. 156 shows an aerogel prepared from Merc.AA+succinylated cellulose using silicone mold needles after lyophilization (left) and after removal from the needle mold (right);

FIG. 157 shows the crosslinked aerogel of FIG. 156 (left) cut into thin slices (right) for subsequent imaging;

FIG. 158 shows microscopy images of the aerogel prepared from Merc. AA crosslinked with citric acid with scale bar=2000 μm (A), 1000 μm (B) and 500 μm (C, D, E);

FIG. 159 shows scanning electron microscopy (SEM) images of a top view (A) of the aerogel of FIG. 158 showing a cross-section perpendicular (B) and parallel (C) to the axis of the aerogel;

FIG. 160 shows microscopy images of the aerogel prepared from Merc. AA+regenerated cellulose (D1A) crosslinked with citric acid with the scale bar=2000 μm (A), 1000 μm (B) and 500 μm (C);

FIG. 161 shows scanning electron microscopy (SEM) images of a top view (A) of the aerogel of FIG. 160 showing a cross-section perpendicular (B) and parallel (C) to the axis of the aerogel;

FIG. 162 shows microscopy images of the aerogel prepared from Merc. AA+succinylated cellulose crosslinked with citric acid with the scale bar=1000 μm (A) and 500 μm (B);

FIG. 163 shows scanning electron microscopy (SEM) images of a top view (A) of the aerogel of FIG. 162 showing a cross-section perpendicular (B) and parallel (C) to the axis of the aerogel;

FIG. 164 shows Fourier-transformed infrared spectra (FTIR) of mercerized succinylated cellulose crosslinked with different concentrations of citric acid;

FIG. 165 shows Fourier-transformed infrared spectra (FTIR) of aerogels prepared from Merc.AA, Merc.AA+regenerated cellulose, and Merc.AA+succinylated cellulose crosslinked with 10% citric acid and compared to mercerized cellulose (Merc.AA 151);

FIG. 166 shows the aerogels prepared from Merc.AA, Merc.AA+Succinylated cellulose and Merc.AA+regenerated cellulose in a 60 mm TC dish, then crosslinked for 1.5 hrs at 110° C.;

FIG. 167 shows the 5 mm wet aerogel samples of FIG. 166 soaked in saline for 30 min prior to mechanical testing;

FIG. 168 shows the dry Merc.AA+regenerated cellulose (A) and wet Merc.AA+regenerated cellulose (B) scaffolds before (left) and after (right) compression testing;

FIG. 169 shows the mechanical properties of dried aerogels which were calculated using the slope of the linear portion of the strain-stress curves obtained with uniaxial compression tests;

FIG. 170 shows the mechanical properties of wet aerogels which were calculated using the slope of the linear portion of the strain-stress curves obtained with uniaxial compression tests;

FIG. 171 shows each aerogel formulation plated along one row (n=6) of a 24-well TC dish;

FIG. 172 shows the lyophilized aerogel before crosslinking;

FIG. 173 shows the lyophilized aerogel after crosslinking;

FIG. 174 shows the change in the colour of the growth media from red to yellow within 10 min of incubation with the aerogels;

FIG. 175 shows the absence of colour change when the aerogels were incubated in MEM alpha (left) for 24 hrs after neutralization and subsequent water washes. No colour change was observed relative to the tube of stock media (right);

FIG. 176 shows the resulting aerogels prepared from Merc.AA, Merc.AA+Succinylated cellulose and Merc.AA+regenerated cellulose;

FIG. 177 shows the aerogels of FIG. 176 on which 100 μL of the final cell suspension was plated and incubated for 2.5 hrs, then topped up with 1.5 mL of growth media per well;

FIG. 178 shows GFP-NIH3T3 cells stained with Hoechst on (A) MercAA aerogel, (B) MercAA+Succinylated cellulose aerogel, and (C) MercAA+regenerated cellulose aerogel with scale bar=100 μm. Purple=scaffold, Yellow Dots=cell nuclei;

FIG. 179 shows the one hour mercerization using 10% bicarbonate solution at 80° C.;

FIG. 180 shows bicarbonate mercerized apple (bottom) compared to NaOH mercerized apple (top);

FIG. 181 shows the five days mercerization reaction using 10% bicarbonate solution at room temperature;

FIG. 182 shows the bicarbonate mercerized apple mercerized apple (mer AA) product;

FIG. 183 shows mercerized AA for 5 days at room temperature using bicarbonate (A), for 1 h at 80° C. using bicarbonate (B) and 1 h at 80° C. using NaOH (control);

FIG. 184 shows 1% alginate pucks of mercerized AA for 5 days at room temperature using bicarbonate (A), for 1 h at 80° C. using bicarbonate (B) and 1 h at 80° C. using NaOH (control);

FIG. 185 shows dark field microscopy images of mercerized AA for 5 days at room temperature using bicarbonate (A), for 1 h at 80° C. using bicarbonate (B) and 1 h at 80° C. using NaOH (control) after lyophilization (6.3×);

FIG. 186 shows FTIR of mercerized AA for 5 days at room temperature using bicarbonate (red), for 1 h at 80° C. using bicarbonate (yellow) and 1 h at 80° C. using NaOH (blue);

FIG. 187 shows fluorescent microscopy images of single particles of mercerized AA for 5 days at room temperature using bicarbonate (A), for 1 h at 80° C. using bicarbonate (B), and 1 h at 80° C. using NaOH (C);

FIG. 188 shows an histogram of the particle size distribution of mercerized AA for 5 days at room temperature using bicarbonate;

FIG. 189 shows an histogram of the particle size distribution of mercerized AA for 1 h at 80° C. using bicarbonate;

FIG. 191 shows MacIntosh apples processed using a food processor in the kitchen prior to the decellularization;

FIG. 192 shows the mercerization of AA 136 at 15 minutes interval for 60 minutes using 10% bicarbonate at 80° C. and 15% H₂O₂ stock solution;

FIG. 193 shows an histogram of the particle size distribution of mercerized AA using bicarbonate and bleached with 30% H₂O₂ stock solution;

FIG. 194 shows an histogram of the particle size distribution of mercerized AA using NaOH and bleached with 30% H₂O₂ stock solution;

FIG. 195 shows an histogram of the particle size distribution of mercerized AA using bicarbonate and bleached with 15% H₂O₂ stock solution;

FIG. 196 shows fluorescent microscopy images of single cells of mercerized AA with bicarbonate bleached with 30% H₂O₂ (A) and 15% H₂O₂ (B) stock solutions stained with Congo red under 10× magnification;

FIG. 197 shows FTIR of mercerized AA using bicarbonate bleached with either 15% H₂O₂ or 30% H₂O₂ compared to mercerized AA using NaOH and bleached with 30% H₂O₂;

FIG. 198 shows FTIR of mercerized AA using bicarbonate bleached with either 15% H₂O₂ or mercerized AA using NaOH using decellularized or raw apples;

FIG. 199 shows raw apple processing in a large Hobart stand mixer bowl;

FIG. 200 shows processed apple in 0.1% SDS during the decellularization process;

FIG. 201 shows processed apple in 0.1M CaCl₂) solution;

FIG. 202 mercerization of the decellularized apple on stovetop;

FIG. 203 shows sieving of decellularized apple, using a 25 μl stainless steel sieve;

FIG. 204 shows 2% alginate solution being prepared on the stovetop;

FIG. 205 shows mixture of mercerized apple and 2% alginate via standmixer;

FIG. 206 shows depositing of biomaterial into silicone molds;

FIG. 207 shows silicone molds with frozen biomaterial in lyophilizer;

FIG. 208 shows cooked biomaterial;

FIG. 209 shows cooked 60 mm alginate/merAA pucks via sous vide (A), pan frying (b), and baking (C);

FIG. 210 shows apple (AA138) processing;

FIG. 211 shows decellularization and mercerization of the processed apples (Mer 138);

FIG. 212 shows scaffold fabrication;

FIG. 213 shows deep fried biomaterial (A) and calamari (B);

FIG. 214 shows sous vide, seared biomaterial (A) and cod (B);

FIG. 215 shows colour test of raw biomaterial (RB), cooked biomaterial (CB), raw cod (RC), cooked cod (CC), raw calamari squid (RS) cooked calamari squid (CS);

FIG. 216 shows odour station of 6 samples and ground coffee;

FIG. 217 shows texture comparison station of raw and cooked biomaterial compared to cod and squid;

FIG. 218 shows apple chopping and decellularization of AA 139;

FIG. 219 shows mercerization of decell AA 139;

FIG. 220 shows scaffold fabrication;

FIG. 221 shows bleached MerAA139 (left) and unbleached (right) 1% Alginate/AA139 biomaterial before freezing;

FIG. 222 shows sensory results for flavour-frequency of words;

FIG. 223 shows sensory results for texture/mouthfeel-frequency of words;

FIG. 224 shows unidirectional freezing of 1% Alginate treatment;

FIG. 225 shows microscopy images of the top side of the 1% Alginate biomaterial after unidirectional freezing in 0.7× (left), and 1.6× (right) magnifications;

FIG. 226 shows microscopy images of the bottom side of the 1% Alginate biomaterial after unidirectional freezing in 0.7× (left), and 1.25× (right) magnifications;

FIG. 227 shows unidirectional freezing of Mer AA:2% Sodium Alginate (1:1) in a petri dish;

FIG. 228 shows microscopy images of the edge (left) and center (right) of the “Mer AA:2% Sodium Alginate (1:1) in a petri dish” biomaterial after unidirectional freezing;

FIG. 229 shows microscopy images of the of the edge (left) and center (right) of the “Mer AA:2% Sodium Alginate (1:1)-petri dish” biomaterial after unidirectional freezing in 0.7× magnification;

FIG. 230 shows biomaterial preparation of Treatment A (left), UF treatment (middle), and Lyophilized biomaterial (right);

FIG. 231 shows microscopy images of a longitudinal cut from Treatment A using the 1×;

FIG. 232 shows biomaterial preparation of Treatment B;

FIG. 233 shows unidirectional freezing of Treatment B;

FIG. 234 shows lyophilized biomaterial of Treatment B;

FIG. 235 shows microscopy images of Lyophilized Treatment B in 1.6× (left) and 0.7× (right) magnifications;

FIG. 236 shows microscopy images of cross-linked Treatment B in 0.7× (left) and 1.6× (right) magnifications;

FIG. 237 shows Mercerized/decellularized palm heart blend in metal moulds;

FIG. 238 shows Lyophilized biomaterial of decellularized and mercerized palm heart before crosslinking;

FIG. 239 shows raw, crosslinked biomaterial “fishstick” (left) and “scallop” (right) of decellularized and mercerized palm heart;

FIG. 240 shows cooked, crosslinked biomaterial “fishstick” (left) and “scallop” (right) of decellularized and mercerized palm heart;

FIG. 241 shows peeled back layer of cooked palm heart biomaterial;

FIG. 242 shows preparation of the biomaterial and layers of Treatment C;

FIG. 243 shows gluing process and two different pieces fabrication from the treatment B;

FIG. 244 shows gluing process and two different pieces fabrication from the treatment C;

FIG. 245 shows cross-link step with 1% CaCl₂) for 1 h at room temperature or in the fridge for 24 h;

FIG. 246 shows Treatment B cross-linked for 1 h at room temperature;

FIG. 247 shows cross-linked (left) and pan-cooked treatment C;

FIG. 248 shows pan-cooking process and pan-cooked treatment B;

FIG. 249 shows Treatment B cross-linked in the fridge for 24 h;

FIG. 250 shows boiling process and boiled Treatment B;

FIG. 251 shows Ingredient mixing and product fabrication-Fish A and Fish B;

FIG. 252 shows Fish A after Sous Vide treatment;

FIG. 253 shows pan-cooking and cooked Fish A;

FIG. 254 shows pan-cooked Fish A-Cross-section;

FIG. 255 shows Fish B placed in the inox mold;

FIG. 256 shows lyophilized Fish B;

FIG. 257 shows cross-linked Fish B;

FIG. 258 shows Fish B Vacuum sealed before the Sous Vide (left) and during the Sous Vide (right);

FIG. 259 shows pan-cooking and cross-section of pan-cooked Fish B;

FIG. 260 shows high throughput continuous crosslinking from injectable composite materials. A: injectable pectin and MerAA mixture. B: hydrogel material loaded into a platen extruded with a perforated plate. C: extrusion into the crosslinking bath. D: the resultant crosslinked hydrogels with predefined shapes. E: The physical properties can be tuned; here the material can be handled easily. F: collection and preparation for lyophilization if desired;

FIG. 261 shows schematic of representation of continuous feed crosslinking;

FIG. 262 shows directionally frozen scaffolds—HE (A,B) and MT (C, D) 4× and 10× excised after 4 weeks of subcutaneous implantation;

FIG. 263 shows directionally frozen scaffolds—HE (A,B) and MT (C, D) 4× and 10× excised after 12 weeks of subcutaneous implantation;

FIG. 264 shows aerogel material prior to surgical subcutaneous implantation in 0.9% sterile saline solution;

FIG. 265 shows Sprague Dawley Rat with aerogel materials implanted subcutaneously each into their own site prior to suturing;

FIG. 266 shows non-directionally frozen aerogel scaffolds—HE (A,B) and MT (C, D) 4× and 10× excised after 4 weeks of subcutaneous implantation;

FIG. 267 shows non-directionally frozen aerogel scaffolds—HE (A,B) and MT (C, D) 4× and 10× excised after 12 weeks of subcutaneous implantation;

FIG. 268 shows directionally frozen scaffolds prior to implantation in sterile 0.9% Saline solution;

FIG. 269 shows directionally frozen scaffold implanted into spinal cord of Sprague Dawley Rat;

FIG. 270 shows aerogel biomaterials prior to surgical implantation into calvarial defect;

FIG. 271 shows Sprague Dawley Rat with implanted aerogel materials crosslinked with alginate and calcium chloride; and

FIG. 272 shows CT scan of resected cranium with calvarial defects in a Sprague Dawley Rat resected after implantation of aerogel material 8 weeks prior.

### DETAILED DESCRIPTION

Described herein are aerogels, hydrogels, and foams derived from and/or comprising decellularized plant or fungal tissue or structural cells thereof. It will be appreciated that embodiments and examples are provided for illustrative purposes intended for those skilled in the art, and are not meant to be limiting in any way.

Provided herein are aerogels, hydrogels, and foams derived from and/or comprising decellularized plant or fungal tissue or structural cells thereof. As described in detail hereinbelow, aerogels, hydrogels, and foams have now been developed which may be derived from and/or may comprise decellularized plant or fungal tissue or structural cells thereof, and which: may comprise plant or fungal microstructures and/or architectures of interest; may be produced by readily scalable production methods; may provide for a wide range of scaffold microstructures and/or macrostructures and/or biochemistry; may provide tunable mechanical properties; may provide tunable porosity, density, architecture (amorphous, aligned, channeled, etc. . . . ), and/or alignment; may be biocompatible in vitro and/or in vivo; may be stable to a variety of conditions (such as cooking conditions in the case of food products); may be produced at scale with control over micro and/or macro structural properties; may allow for control over density, long range architecture, and/or mass manufacture; may be scalable in terms of quantity of material produced as well as product size and/or shape; may be produced with GRAS components to maintain edibility; may be freeze-dried to provide shelf stability and/or shippability; or any combinations thereof. By using single structural cells, groups of structural cells, or both, derived from a plant or fungal tissue (the single structural cells or groups of structural cells having a decellularized 3-dimensional structure lacking cellular materials and nucleic acids of plant or fungal tissue), distributed within a carrier derived from one or more dehydrated, lyophilized, or freeze-dried hydrogels, a variety of aerogels, hydrogels, and foams have now been developed and prepared having desirable properties. In certain embodiments, the single structural cells, groups of structural cells, or both, may be derived from plant or fungal tissue (typically decellularized plant or fungal tissue) using mercerization treatment as described herein, which allows for reproducible and scalable production. Related methods and uses, as well as productions methods (some or all of which may be automatable), are also described in detail herein.

In an embodiment, there is provided herein an aerogel or foam comprising:
single structural cells, groups of structural cells, or both, derived from a plant or fungal tissue, the single structural cells or groups of structural cells having a decellularized 3-dimensional structure lacking cellular materials and nucleic acids of plant or fungal tissue;
the single structural cells, groups of structural cells, or both, being distributed within a carrier, the carrier derived from a dehydrated, lyophilized, or freeze-dried hydrogel.

As will be understood, an aerogel or foam may comprise generally any 3-dimensional scaffold or matrix. Typically, aerogels and foams as described herein are highly porous and lightweight (low density), although porosity and density may be adjusted as desired as is also described herein. The aerogels and foams are typically hydrophilic, and may be provided as either dry aerogels or foams, or rehydrated or wetted aerogels or foams (sometimes also referred to herein as hydrogels) additionally comprising water, an aqueous solution (such as a cell culture buffer, a salt solution, a buffer, or another aqueous solution), or another liquid (such as an alcohol, for example ethanol, or a non-aqueous liquid).

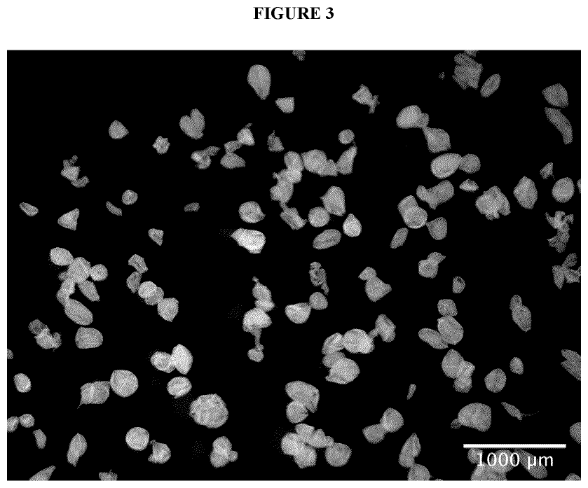

As will be understood, plant or fungal tissue may comprise a plurality of linked plant cells formed as an extended 3D structure. Such plant or fungal tissue may be decellularized (for example, by using the decellularization methods as described in WO2017/136950, entitled “Decellularised Cell Wall Structures From Plants and Fungus and Use Thereof as Scaffold Materials”, which is herein incorporated by reference in its entirety) so as to provide decellularized plant or fungal tissue lacking cellular materials and nucleic acids of plant or fungal cells, but maintaining 3 dimensional structure substantially intact. Such decellularized plant or fungal tissue (such as decellularized hypanthium or pulp tissue, or any other suitable plant or fungal tissues/structures/components of interest) may comprise an extended 3D structure (which may be comprised of any one or more of cellulose, hemicellulose, pectin, lignin, or the like; typically, the extended 3D structure may comprise a lignocellulosic structure/material), which may comprise a plurality of linked structural cells. As described herein, single structural cells, groups of structural cells (comprising a plurality of linked single structural cells), or both, may be derived from the extended 3D structure, the single structural cells or groups of structural cells having a decellularized 3-dimensional structure lacking cellular materials and nucleic acids of plant or fungal tissue. In certain embodiments, the single structural cells or groups of structural cells may comprise isolated structural cells, or small groups of clustered structural cells, the structural cells having a substantially intact 3-dimensional structure typically resembling a hollow cell or pocket as shown in FIG. 3. As will be understood, such structures may typically comprise lignocellulosic materials, such as cellulose and/or lignin-based structures. It will be understood that in certain embodiments, such structures may comprise other building blocks such as chitin and/or pectin, for example.

In certain embodiments of the aerogel or aerogels or foam or foams, the plant or fungal tissue from which the single structural cells or groups of structural cells are derived may comprise decellularized plant or fungal tissue.

As described herein, single structural cells, groups of structural cells, or both, may preferably be derived from a decellularized plant or fungal tissue, and may even more preferably be derived from a decellularized plant or fungal tissue using mercerization treatment as described in detail herein. However, it will be understood that in certain embodiments, single structural cells, groups of structural cells, or both, may instead be derived from plant or fungal tissue and then decellularized afterward, or may be derived from plant or fungal tissue in a manner that concurrently provides decellularization, for example. In certain embodiments, structural cells may comprise decellularized structural cells comprising the cell wall which previously contained one or more plant cells prior to decellularization.

In certain embodiments, the aerogels, foams, hydrogels, and other such materials as described herein may comprise cell wall architectures and/or vascular structures found in the plant and/or fungus kingdoms to create 3D scaffolds which may promote cell infiltration, cell growth, bone tissue repair, bone reconstruction, regenerative therapy, spinal cord repair, etc. As will be understood, biomaterials as described herein may be produced from any suitable part of plant or fungal organisms. Biomaterials may comprise, for example, one or more substances such as cellulose, chitin, lignin, lignan, hemicellulose, pectin, lignocellulose, and/or any other suitable biochemicals/biopolymers which are naturally found in these organisms.

As will be understood, unless otherwise indicated, the meaning/definition of plant and fungi kingdoms used herein is based on the Cavalier-Smith classification (1998).

In certain embodiments, the plant or fungal tissue may comprise generally any suitable plant or fungal tissue or part appropriate for the particular application. In certain embodiments, the plant or fungal tissue may comprise an apple hypanthium (Malus pumila) tissue, a fern (Monilophytes) tissue, a turnip (Brassica rapa) root tissue, a gingko branch tissue, a horsetail (equisetum) tissue, a hermocallis hybrid leaf tissue, a kale (Brassica oleracea) stem tissue, a conifers Douglas Fir (Pseudotsuga menziesii) tissue, a cactus fruit (pitaya) flesh tissue, a Maculata Vinca tissue, an Aquatic Lotus (Nelumbo nucifera) tissue, a Tulip (Tulipa gesneriana) petal tissue, a Plantain (Musa paradisiaca) tissue, a broccoli (Brassica oleracea) stem tissue, a maple leaf (Acer psuedoplatanus) stem tissue, a beet (Beta vulgaris) primary root tissue, a green onion (Allium cepa) tissue, a orchid (Orchidaceae) tissue, turnip (Brassica rapa) stem tissue, a leek (Allium ampeloprasum) tissue, a maple (Acer) tree branch tissue, a celery (Apium graveolens) tissue, a green onion (Allium cepa) stem tissue, a pine tissue, an aloe vera tissue, a watermelon (Citrullus lanatus var. lanatus) tissue, a Creeping Jenny (Lysimachia nummularia) tissue, a cactae tissue, a Lychnis Alpina tissue, rhubarb (Rheum rhabarbarum) tissue, a pumpkin flesh (Cucurbita pepo) tissue, a Dracena (Asparagaceae) stem tissue, a Spiderwort (Tradescantia virginiana) stem tissue, an Asparagus (Asparagus officinalis) stem tissue, a mushroom (Fungi) tissue, a fennel (Foeniculum vulgare) tissue, a rose (Rosa) tissue, a carrot (Daucus carota) tissue, or a pear (Pomaceous) tissue. Additional examples of plant and fungal tissues are described in Example 18 of WO2017/136950, entitled “Decellularised Cell Wall Structures from Plants and Fungus and Use Thereof as Scaffold Materials”, herein incorporated by reference in its entirety.

In certain embodiments, the decellularized plant or fungal tissue may be cellulose-based, chitin-based, chitosan-based, lignin-based, lignan-based, hemicellulose-based, or pectin-based, or any combination thereof. In certain embodiments, the plant or fungal tissue may comprise a tissue from apple hypanthium (Malus pumila) tissue, a fern (Monilophytes) tissue, a turnip (Brassica rapa) root tissue, a gingko branch tissue, a horsetail (equisetum) tissue, a hermocallis hybrid leaf tissue, a kale (Brassica oleracea) stem tissue, a conifers Douglas Fir (Pseudotsuga menziesii) tissue, a cactus fruit (pitaya) flesh tissue, a Maculata Vinca tissue, an Aquatic Lotus (Nelumbo nucifera) tissue, a Tulip (Tulipa gesneriana) petal tissue, a Plantain (Musa paradisiaca) tissue, a broccoli (Brassica oleracea) stem tissue, a maple leaf (Acer psuedoplatanus) stem tissue, a beet (Beta vulgaris) primary root tissue, a green onion (Allium cepa) tissue, a orchid (Orchidaceae) tissue, turnip (Brassica rapa) stem tissue, a leek (Allium ampeloprasum) tissue, a maple (Acer) tree branch tissue, a celery (Apium graveolens) tissue, a green onion (Allium cepa) stem tissue, a pine tissue, an aloe vera tissue, a watermelon (Citrullus lanatus var. lanatus) tissue, a Creeping Jenny (Lysimachia nummularia) tissue, a cactae tissue, a Lychnis Alpina tissue, a rhubarb (Rheum rhabarbarum) tissue, a pumpkin flesh (Cucurbita pepo) tissue, a Dracena (Asparagaceae) stem tissue, a Spiderwort (Tradescantia virginiana) stem tissue, an Asparagus (Asparagus officinalis) stem tissue, a mushroom (Fungi) tissue, a fennel (Foeniculum vulgare) tissue, a rose (Rosa) tissue, a carrot (Daucus carota) tissue, or a pear (Pomaceous) tissue, or a genetically altered tissue produced via direct genome modification or through selective breeding, or any combinations thereof.

As will also be understood, cellular materials and nucleic acids of the plant or fungal tissue may include intracellular contents such as cellular organelles (e.g. chloroplasts, mitochondria), cellular nuclei, cellular nucleic acids, and/or cellular proteins. These may be substantially removed, partially removed, or fully removed from the plant or fungal tissue, and/or from the structural cells. It will recognized that trace amounts of such components may still be present in the decellularised plant or fungal tissues and/or structural cells as described herein. As will also be understood, references to decellularized plant or fungal tissue herein are intended to reflect that such cellular materials found in the plant or fungal source of the tissues have been substantially removed—this does not preclude the possibility that the decellularized plant or fungal tissue or structural cells may in certain embodiments contain or comprise subsequently introduced, or reintroduced, cells, cellular materials, and/or nucleic acids of generally any kind, such as animal or human cells, such as bone or bone progenitor cells/tissues.

Various methods may be used for producing decellularized plant or fungal tissue as described herein. By way of example, in certain embodiments, the decellularised plant or fungal tissue may comprise plant or fungal tissue(s) which have been decellularised by thermal shock, treatment with detergent (e.g. SDS, Triton X, EDA, alkaline treatment, acid, ionic detergent, non-ionic detergents, and zwitterionic detergents), osmotic shock, lyophilisation, physical lysing (e.g. hydrostatic pressure), electrical disruption (e.g. non thermal irreversible electroporation), or enzymatic digestion, or any combination thereof. In certain embodiments, biomaterials as described herein may be obtained from plants and/or fungi by employing decellularization processes which may comprise any of several approaches (either individually or in combination) including, but not limited to, thermal shock (for example, rapid freeze thaw), chemical treatment (for example, detergents), osmotic shock (for example, distilled water), lyophilisation, physical lysing (for example, pressure treatment), electrical disruption and/or enzymatic digestion.

In certain embodiments, the decellularised plant or fungal tissue may comprise plant or fungal tissue which has been decellularised by treatment with a detergent or surfactant. Examples of detergents may include, but are not limited to sodium dodecyl sulphate (SDS), Triton X, EDA, alkyline treatment, acid, ionic detergent, non-ionic detergents, and zwitterionic detergents. In preferred embodiments, the plant or fungal tissue may be decellularized using SDS and CaCl₂).

In still further embodiments, the decellularised plant or fungal tissue may comprise plant or fungal tissue which has been decellularised by treatment with SDS. In still another embodiment, residual SDS may be removed from the plant or fungal tissue by washing with an aqueous divalent salt solution. The aqueous divalent salt solution may be used to precipitate/crash a salt residue containing SDS micelles out of the solution/scaffold, and a dH₂O, acetic acid or dimethylsulfoxide (DMSO) treatment, or sonication, may have been used to remove the salt residue or SDS micelles. In certain embodiments, the divalent salt of the aqueous divalent salt solution may comprise, for example, MgCl₂ or CaCl₂).

In another embodiment, the plant or fungal tissue may be decellularised by treatment with an SDS solution of between 0.01 to 10%, for example about 0.1% to about 1%, or, for example, about 0.1% SDS or about 1% SDS, in a solvent such as water, ethanol, or another suitable organic solvent, and the residual SDS may have been removed using an aqueous CaCl₂) solution at a concentration of about 100 mM followed by incubation in dH₂O. In certain embodiments, the SDS solution may be at a higher concentration than 0.1%, which may facilitate decellularisation, and may be accompanied by increased washing to remove residual SDS. In particular embodiments, the plant or fungal tissue may be decellularised by treatment with an SDS solution of about 0.1% SDS in water, and the residual SDS may have been removed using an aqueous CaCl₂) solution at a concentration of about 100 mM followed by incubation in dH₂O.

Further examples of decellularization protocols which may be adapted for producing decellularized materials as described herein may be found in WO2017/136950, entitled “Decellularised Cell Wall Structures from Plants and Fungus and Use Thereof as Scaffold Materials”, herein incorporated by reference in its entirety.

In certain embodiments, aerogels, foams, and/or hydrogels as described herein may comprise the single structural cells, groups of structural cells, or both, distributed within a carrier. In certain embodiments, the carrier may be derived from a dehydrated, lyophilized, or freeze-dried hydrogel. As will be understood, the carrier may comprise generally any suitable carrier material, structure, or matrix, for providing support and/or structure to the aerogel, foam, and/or hydrogel, and may be used to support, carry, join, or hold the single structural cells, groups of structural cells, or both of the aerogel, foam, and/or hydrogel. The person of skill in the art having regard to the teachings herein will be aware of a variety of different carriers which may be used, and which may be selected to suit the particular application(s) of interest. In certain embodiments, the carrier may comprise a hydrogel into which the single structural cells, groups of structural cells, or both, are mixed, or the carrier may be derived from a dehydrated, lyophilized, or freeze-dried hydrogel, within which the single structural cells, groups of structural cells, or both are distributed/mixed. A variety of different hydrogels may be used for providing the carrier, such as but not limited to hydrogel(s) comprising any one or more of alginate, pectin, gelatin, methylcellulose, carboxymethylcellulose, hydroxypropylcellulose, hydroxypropyl methyl cellulose, hydroxyethyl methyl cellulose, agar, pluronic acid, triblock PEO-PPO-PEO copolymers of poly(ethylene oxide) (PEO) and poly(propylene oxide) (PPO), dissolved or regenerated plant cellulose, dissolved cellulose-based hydrogel, hyaluronic acid, extracellular matrix proteins (e.g. collagen, gelatin, or fibronectin, or any combinations thereof), monoacrylated poly(ethylene glycol), poly(ethylene glycol) diacrylate, poly(ethylene glycol) diacrylate (PEGDA)-co-PEGMA, poly(vinyl alcohol), poly(vinylpyrrolidone), poly(lactic-co-glycolic acid), chitosan, chitin, xanthan gum, elastin, fibrin, fibrinogen, cellulose derivatives, carrageenan, or microcrystalline cellulose, or any combinations thereof. In certain embodiments, the hydrogel/carrier may optionally be cross-linked.

In certain embodiments of any of the aerogel or aerogels or foam or foams described herein, the hydrogel may comprise alginate, pectin, gelatin, methylcellulose, carboxymethylcellulose, hydroxypropylcellulose, hydroxypropyl methyl cellulose, hydroxyethyl methyl cellulose, agar, pluronic acid, triblock PEO-PPO-PEO copolymers of poly(ethylene oxide) (PEO) and poly(propylene oxide) (PPO), dissolved or regenerated plant cellulose, dissolved cellulose-based hydrogel, hyaluronic acid, extracellular matrix proteins (e.g. collagen, gelatin, or fibronectin, or any combinations thereof), monoacrylated poly(ethylene glycol), poly(ethylene glycol) diacrylate, poly(ethylene glycol) diacrylate (PEGDA)-co-PEGMA, poly(vinyl alcohol), poly(vinylpyrrolidone), poly(lactic-co-glycolic acid), chitosan, chitin, xanthan gum, elastin, fibrin, fibrinogen, cellulose derivatives, carrageenan, or microcrystalline cellulose, or any combinations thereof; wherein the hydrogel is optionally cross-linked.

In certain embodiments of the aerogels or foams described herein, the aerogel or foam may be rehydrated, optionally with water, an aqueous solution, a buffer, a cell buffer, an alcohol (such as ethanol), or another aqueous or non-aqueous liquid or solution suitable of the application(s) of interest. Alternatively, the aerogels or foams may be provided in dry form

In preferred embodiments of the aerogel or aerogels or foam or foams described herein, the single structural cells, groups of structural cells, or both, may be derived from the plant or fungal tissue, preferably decellularized plant or fungal tissue, by mercerization. Other approaches for obtaining single structural cells, groups of structural cells, or both, such as maceration or other liquid-based extractions, are also contemplated; however, as described herein mercerization is preferred.

As will be understood, mercerization may comprise any suitable process for treating plant or fungal tissue (preferably, decellularized plant or fungal tissue) to obtain single structural cells, groups of structural cells, or both, typically using a liquid extraction solution employing base and preferably further employing a peroxide. Typically, mercerization of the plant or fungal tissue (preferably decellularized plant or fungal tissue), disassembles the plant or fungal tissue into tissue/cellular components (including single structural cells, groups of structural cells, or both). In certain embodiments, the mercerization may employ an alkaline/base solution and a peroxide. In certain embodiments, more than one treatment or solution may be used, either simultaneously or sequentially.

In certain embodiments, mercerization may comprise at least one treatment with a base solution. As will be understood, the base solution may comprise generally any suitable base, such as any suitable base capable of osmotic shock and/or disruption of hydrogen bonding and/or polymer crystal structure so as to extract intact tissue structures. As will be understood, particularly for food and/or medical applications, the base may be selected to be appropriate for the particular application and may, for example, be selected to be physiologically occurring, easily washed away, non-harmful, and/or selected accordingly to a variety of factors relevant to the particular application, as desired. In certain embodiments, the base may comprise NaOH, KOH, or a combination thereof. In an embodiment, the base may be dissolved/mixed in a suitable solvent, to form the base solution. Typically, the solvent may comprise water, although other solvents, or combinations of solvents (such as, for example, a mixture of water and ethanol), are also contemplated. The base concentration in the base solution may be tailored to suit the particular application of interest. Typically, the base solution may comprise a base concentration of about 0.1 to 10M, or any concentration therebetween (optionally rounded to the nearest 0.1), or any subrange spanning between any two of these concentrations. In certain embodiments, the base concentration may be about 0.5M to 3M, or any value (optionally rounded to the nearest 0.1) therebetween, or any subrange spanning between any two of these concentrations. By way of example, in certain embodiments, the base solution may comprise an aqueous solution of NaOH, having a concentration of about 0.5M-3M. As will be understood, the base solution, as well as the treatment conditions (i.e. heating, stirring) may be tailored to suit the particular application, desired structures to be extracted, plant or fungal tissue being used, etc. . . . , as desired.

In certain embodiments, bases may include a base selected from the group consisting of: Carbonates; Nitrates; Phosphates; Sulfates; Ammonia; Sodium hydroxide; Calcium hydroxide; Magnesium hydroxide; Potassium hydroxide; Lithium hydroxide; Zinc hydroxide; Sodium carbonate; Sodium bicarbonate; Butyl lithium; Sodium azide; Sodium amide; Sodium hydride; Sodium borohydride; or Lithium diisopropylamine. Depending on the base and/or intended use of the products, neutralization and/or washing may be performed to remove residual base and other reagents so as to prevent undesirable contamination, for example.

In preferred embodiments, the mercerization may comprise treatment of the plant or fungal tissue (preferably decellularized plant or fungal tissue) using sodium hydroxide and hydrogen peroxide with heating.

In yet another embodiment of any of the aerogel or aerogels or foam or foams described herein, the aerogel or foam may comprise a particle size distribution of the single structural cells with an average feret diameter within a range of about 1 μm to about 1000 μm, such as about 100 to about 500 μm, for example about 100 to about 300 μm.

In yet another embodiment of any of the aerogel or aerogels or foam or foams described herein, the plant tissue may comprise apple tissue or pear tissue.

In another embodiment of any of the aerogel or aerogels or foam or foams described herein, the aerogel or foam may comprise about 5% to about 95% m/m, such as about 10-50% m/m (or more), single structural cells, groups of structural cells, or both, when the aerogel or foam is in hydrated form.

In still another embodiment of any of the aerogel or aerogels or foam or foams described herein, the hydrogel may comprise alginate, pectin, or both, and the aerogel or foam may be rehydrated with a CaCl₂) solution, providing cross-linking.

In yet another embodiment of any of the aerogel or aerogels or foam or foams described herein, the aerogel or foam may have bulk modulus within a range of about 0.1 to about 500 kPa, such as about 1 to about 200 kPa.

In another embodiment of any of the aerogel or aerogels or foam or foams described herein, the aerogel or foam may be rehydrated and may further comprise one or more animal cells.

In another embodiment of any of the aerogel or aerogels or foam or foams described herein, the aerogel or foam may be rehydrated and may further comprise any one or more cells selected from fibroblasts, myofibroblasts, neurons, dorsal root ganglion cells, neuronal structures such as axons, neural precursor cells, neural stem cells, glial cells, endogenous stem cells, neutrophils, mesenchymal stem cells, satellite cells, myoblasts, myotubes, muscle progenitor cells, adipocytes, preadipocytes, chondrocytes, osteoblasts, osteoclasts, pre-osteoblasts, tendon progenitor cells, tenocytes, periodontal ligament stem cells, endothelial cells, or any combinations thereof. Cells may be selected to suit the particular application(s) of interest. In certain embodiments, where food product applications are of interest, the one or more cells may comprise muscle cells, fat cells, connective tissue cells (i.e. fibroblasts), cartilage, bone, epithelial, or endothelial cells, or any combinations thereof, for example.

In still another embodiment of any of the aerogel or aerogels or foam or foams described herein, at least some cellulose and/or cellulose derivative(s) of the aerogel or foam may be cross-linked by physical cross-linking (e.g. using glycine) and/or chemical cross-linking (e.g. using citric acid in the presence of heat); wherein at least some cellulose and/or cellulose derivative(s) of the aerogel or foam is functionalized with a linker (e.g. succinic acid) to which one or more functional moieties are optionally attached (e.g. amine-containing groups, wherein cross-linking may further optionally be achieved with one or more protein cross-linkers such as formalin, formaldehyde, glutaraldehyde, and/or transglutaminase); or any combinations thereof. In certain embodiments, it is contemplated that cross-linking may impart additional structural integrity to the aerogels, foams, and/or hydrogels as described herein, and the degree of cross-linking may be controlled to adjust physical properties of the resultant products. In certain embodiments, cellulose or cellulose derivatives or other materials of the structural cells of the aerogels or foams may be cross-linked; materials of the carrier (typically derived from a hydrogel) may be cross-linked; or combinations thereof. Example 8 below provides illustrative examples of physical and chemical cross-linking approaches, including those employing linkers. The person of skill in the art having the benefit of the teachings herein, and taking into consideration the structural cells and carrier/hydrogel being used in the particular aerogel/foam, will be aware of suitable approaches for achieving cross-linking.

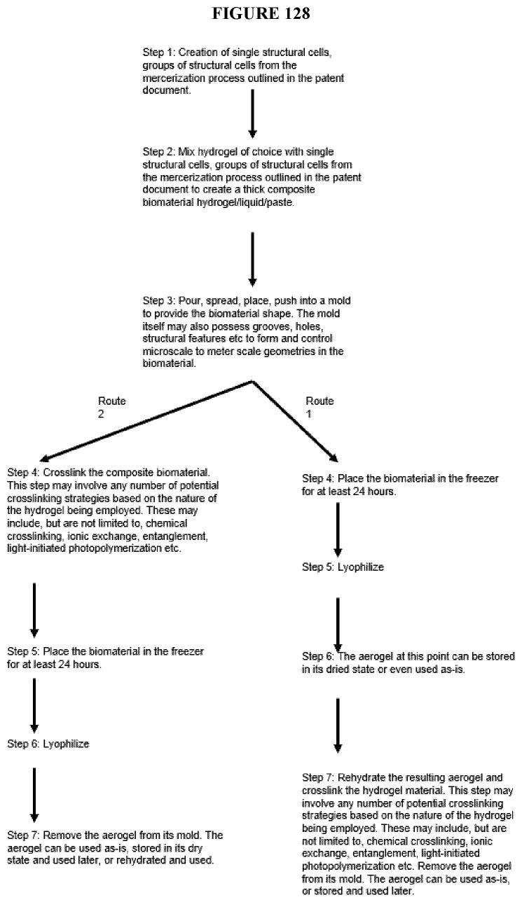

In certain embodiments, the carrier of the aerogel or aerogels or foam or foams as described herein may, or may not, be cross-linked. In certain embodiments, the carrier may be cross-linked before dehydrating, lyophilizing, or freeze-drying; after dehydrating, lyophilizing, or freeze-drying; or both. In embodiments in which the carrier is cross-linked, the carrier may typically be cross-linked after mixing or distribution of the single structural cells, groups of structural cells, or both therein, and before or after dehydrating, lyophilizing, or freeze-drying of the mixture. FIG. 128 shows a flow chart depicting illustrative examples of aerogel/foam preparation using cross-linking before or after freezing and lyophilization.

In still another embodiment of any of the aerogel or aerogels or foam or foams as described herein, the aerogel or foam may comprise templated or aligned microchannels created by directional freezing; by molding using molds having microscale and/or macroscale features; by punching, pressing, stamping, or otherwise forming geometric patterns, depressions, holes, channels, grooves, ridges, wells, or other structural features within and/or onto at least one surface; or any combinations thereof.

Approaches for directional freezing are described in detail in the Examples below. Briefly, by creating a larger thermal gradient on one side of a hydrogel, linear and highly aligned ice crystals may form from the cold side. This may force the surrounding hydrogel polymers to form around the ice crystals, creating aligned microscale channels. After lyophilizing the resulting material, a scaffold may be created with many microchannels. As will be understood, directional freezing may be used to template or form channels and/or other structural features interior to the aerogels and/or foams, on the surface of the aerogels and/or foams, or both.

In still another embodiment of any of the above method or methods, a microarchitecture of the microchannels produced from directional freezing may be controlled by creating the mixture including a solvent containing varying amounts of one or more other dissolved compounds such as Sucrose, Dextrose, Trehalose, Corn Starch, Glycerol, Ethanol, Mannitol, Sodium chloride, CaCl2, Gelatine, Citric acid, PVA, PEG, Dextran, NaF, NaBr, NaI, phosphate buffer, another sugar or salt, or another such agent, which may alter the structural properties of aligned ice crystals which grow from the cold side of the thermal gradient.

In certain embodiments, directional freezing (also referred to as freeze casting, ice-templating) may comprise a process through which well-controlled microscale features (channels, pores, etc) may be created in an aerogel comprising a hydrogel, polymer, biomacromolecules etc. In certain embodiments, the process may typically involve the controlled solidification of an aqueous solution, suspension or sol-gel followed by sublimation in a lyophilizer. The solution which undergoes controlled freezing may typically be placed on a cold plate which creates a non-uniform thermal gradient which typically starts on one side. Ice crystals form from the cold side and grow linearly away from the cold surface. As the ice crystals grow, they displace the solution components (polymer, hydrogel, colloids, single structural cells, etc.) and they collect between the growing ice crystals. After complete freezing, sublimation in a lyophilizer may typically be performed which may remove the ice crystals leaving behind an aerogel or foam with anisotropic templated nano to microscale features, such as aligned channels. The final structural properties of the features may therefore be dependent on the structure of the ice crystals which form in the solution. Therefore, other solutes which will impact ice crystallization may allow for control over the final architecture of the aerogel. In such scenarios it is contemplated that dissolving other salts, lipids, sugars and/or other additives into the aqueous solution may impact ice crystal formation. In certain embodiments, such compounds may include any of the following, alone or in combination: Sucrose, dextrose Trehalose, Corn Starch, Glycerol, Ethanol, Mannitol, Sodium chloride, CaCl2, Gelatine, Citric acid, PVA, PEG, Dextran, NaF, NaBr, NaI, phosphate buffer, etc. In addition to additives in the solution, it is also contemplated that in certain embodiments temperature and freezing rate may also impact ice crystal geometry. Temperatures from −195° C. to 0° C. may be used in certain embodiments, with operating temperatures between −30° C. to −10° C. being more typically employed to create the aligned, directionally frozen scaffolds in certain embodiments.

Molding of the aerogels, foams, and hydrogels as described herein is also described in detail in the Examples below. In certain embodiments, aerogel/foam/hydrogel precursor mixtures may be introduced into containers or molds, and subsequently dehydrated, lyophilized, or freeze-dried. In preferred embodiments, the aerogel/foam/hydrogel precursor mixture may be frozen within the container or mold prior to the dehydrating, lyophilisation, or freeze-drying. In certain embodiments, the mold or container may be designed so as to provide an aerogel/foam/hydrogel having a desired shape and/or size. In certain embodiments, the container or mold may be designed to present microscale and/or macroscale features to the surface of the aerogel/foam/hydrogel contained therein (for example, the mold may have structural features such as projections/depressions on it's interior walls to form structural on the surface and/or internal to the aerogels and/or foams), and/or may be designed to project microscale and/or macroscale features into the aerogel/foam/hydrogel contained therein, so as to impart desired structure to the aerogel/foam/hydrogel by molding. Examples of microscale and/or macroscale features may include geometric patterns, channels, depressions, tunnels, or holes, or any other microscale and/or macroscale features desired or suitable for the particular application(s) of interest.

In certain embodiments, macroscale and/or microscale structural features may be imparted to the aerogels/foams/hydrogels by mechanical processing such as by punching, pressing, stamping, drilling, or otherwise forming geometric patterns, depressions, holes, channels, grooves, ridges, wells, or other structural features within and/or onto at least one surface of the aerogels/foams/hydrogels as desired. In some embodiments, it is contemplated that such mechanical processing may be computer-guided (for example, by numerical control) using automated machinery, for example. Mechanical processing may be performed before, during, and/or after freezing and/or lyophilisation or freeze-drying, for example.

In yet another embodiment, there is provided herein single structural cells, groups of structural cells, or both, derived from a decellularized plant or fungal tissue by mercerization of the decellularized plant or fungal tissue, the single structural cells or groups of structural cells having a decellularized 3-dimensional structure lacking cellular materials and nucleic acids of plant or fungal tissue, and lacking one or more base-soluble lignin components of the plant or fungal tissue. As will be understood, mercization with base (such as NaOH, and optionally additionally in the presence of hydrogen peroxide, for example) may remove one or more base-soluble lignin components, however as shown in the Examples below overall 3-dimensional structure of the resultant single structural cells, groups of structural cells, or both, derived from the decellularized plant or fungal tissue may remain substantially intact. Resultant structural cells from mercerization may differ from structural cells obtained in an alternative manner such as an acid-employing maceration approach, which may remove certain acid-soluble lignin components rather than base-soluble lignin components and therefore provide structural cells having different lignin content, for example. In certain embodiments, the single structural cells, groups of structural cells, or both, may be provided in dried form, or suspended in an aqueous or non-aqueous liquid or solution such as, but not limited to, water, an aqueous buffer, or ethanol.

In certain embodiments, any of the aerogel, aerogels, foam, or foams as described herein may additionally comprise one or more cells cultured or located therein/thereon. In certain embodiments, the one or more cells may comprise any one or more of muscle cells, fat cells, connective tissue cells, fibroblasts, myofibroblasts, neurons, dorsal root ganglion cells, neuronal structures such as axons, neural precursor cells, neural stem cells, glial cells, endogenous stem cells, neutrophils, mesenchymal stem cells, satellite cells, myoblasts, myotubes, muscle progenitor cells, adipocytes, preadipocytes, cartilage cells, bone cells, epithelial cells, endothelial cells, chondrocytes, osteoblasts, osteoclasts, pre-osteoblasts, tendon progenitor cells, tenocytes, periodontal ligament stem cells, or endothelial cells, or any combinations thereof.

In certain embodiments, aerogels and foams as described herein may be generally biocompatible. By way of example, in certain embodiments it is contemplated that aerogels and foams as described herein may be compatible with a variety of different cell types relevant for tissue engineering and/or food applications, and may be biocompatible with cells from many different species and kingdoms including, but not limited to, human, rodent (e.g. mouse, rat, guinea pig), lagomorpha (e.g. rabbit, hare), carpine (goat), ovine (e.g. Sheep, lamb, mutton), porcine (e.g. pig, hog, boar), bovine (e.g. cow, bison, buffalo), feline, canine, fish (e.g. salmon, tuna, snapper, mackerel, cod, trout, carp, catfish and sardines), mullosca, crustacean (e.g. crabs, lobsters, crayfish, shrimps, prawns), avian (e.g. chicken, turkey, duck), reptile, amphibian, insect, and/or plant species. As will be understood, cells may be selected based on the particular application(s) of interest, which may include, but are not limited to, therapeutic (human or veterinary), food, or other such applications.

In yet another embodiment, there is provided herein a method for preparing an aerogel or foam, comprising:
providing a decellularized plant or fungal tissue;
obtaining single structural cells, groups of structural cells, or both, from the decellularized plant or fungal tissue, by performing mercerization of the decellularized plant or fungal tissue and collecting the resultant single structural cells or groups of structural cells having a decellularized 3-dimensional structure;
mixing or distributing the single structural cells, groups of structural cells, or both, in a hydrogel, to provide a mixture; and
dehydrating, lyophilizing, or freeze-drying the mixture to provide the aerogel or foam.

Aerogels, foams, plant or fungal tissue, decellularization, structural cells and groups of structural cells, and hydrogels have already been described in detail hereinabove.

As will be understood, single structural cells, groups of structural cells, or both, may be obtained from plant or fungal tissue (preferably from decellularized plant or fungal tissue) by performing mercerization. Mercerization may comprise any suitable process for treating plant or fungal tissue (preferably, decellularized plant or fungal tissue) to obtain single structural cells, groups of structural cells, or both, typically using a liquid extraction solution employing base and preferably further employing a peroxide. Typically, mercerization of the plant or fungal tissue (preferably decellularized plant or fungal tissue), disassembles the plant or fungal tissue into tissue/cellular components (including single structural cells, groups of structural cells, or both). In certain embodiments, the mercerization may employ an alkaline/base solution and a peroxide. In certain embodiments, more than one treatment or solution may be used, either simultaneously or sequentially. In certain embodiments it is contemplated that mercerization may be performed on plant or fungal tissue and decellularization may be performed afterwards, or mercerization may be performed on plant or fungal tissue and mercerization conditions may be selected so as to simultaneously provide decellularization. However, as described herein, it is preferred that mercerization be performed on plant or fungal tissue that has already previously been decellularized.

In certain embodiments, mercerization may comprise at least one treatment with a base solution.

As will be understood, the base solution may comprise generally any suitable base, such as any suitable base capable of osmotic shock and/or disruption of hydrogen bonding and/or polymer crystal structure so as to extract intact tissue structures. As will be understood, particularly for food and/or medical applications, the base may be selected to be appropriate for the particular application and may, for example, be selected to be physiologically occurring, easily washed away, non-harmful, and/or selected accordingly to a variety of factors relevant to the particular application, as desired. In certain embodiments, the base may comprise NaOH, KOH, or a combination thereof. In an embodiment, the base may be dissolved/mixed in a suitable solvent, to form the base solution. Typically, the solvent may comprise water, although other solvents, or combinations of solvents (such as, for example, a mixture of water and ethanol), are also contemplated. The base concentration in the base solution may be tailored to suit the particular application of interest. Typically, the base solution may comprise a base concentration of about 0.1 to 10M, or any concentration therebetween (optionally rounded to the nearest 0.1), or any subrange spanning between any two of these concentrations. In certain embodiments, the base concentration may be about 0.5M to 3M, or any value (optionally rounded to the nearest 0.1) therebetween, or any subrange spanning between any two of these concentrations. By way of example, in certain embodiments, the base solution may comprise an aqueous solution of NaOH, having a concentration of about 0.5M-3M. As will be understood, the base solution, as well as the treatment conditions (i.e. heating, stirring) may be tailored to suit the particular application, desired structures to be extracted, plant or fungal tissue being used, etc. . . . , as desired.

In certain embodiments, bases may include a base selected from the group consisting of: Carbonates; Nitrates; Phosphates; Sulfates; Ammonia; Sodium hydroxide; Calcium hydroxide; Magnesium hydroxide; Potassium hydroxide; Lithium hydroxide; Zinc hydroxide; Sodium carbonate; Sodium bicarbonate; Butyl lithium; Sodium azide; Sodium amide; Sodium hydride; Sodium borohydride; or Lithium diisopropylamine. Depending on the base and/or intended use of the products, neutralization and/or washing may be performed to remove residual base and other reagents so as to prevent undesirable contamination, for example.

In preferred embodiments, the mercerization may comprise treatment of the plant or fungal tissue (preferably decellularized plant or fungal tissue) using sodium hydroxide and hydrogen peroxide with heating.

The single structural cells or groups of structural cells (having a decellularized 3-dimensional structure) resulting from mercerization may be collected. The resultant single structural cells or groups of structural cells may be provided in dried form, or as a paste or gel, or in another suitable form as desired.

Mercerization processes in other industries, such as in the pulp and paper industry, strip down to cellulose polymers/fibres (i.e. complete destruction of plant structures). In contrast, mercerization processes as described herein (regardless of whether the plant or fungal tissue is decellularized before, during, or after the mercerization) may provide for retention of intact single structural cells or groups of structural cells with 3-dimensional structure. While mercerization may be performed before decellularization of the plant or fungal tissue, this was not preferred as it is expected to take longer, be less efficient, and may result in a less pure resultant material to be decellularized. Accordingly, mercerization of already decellularized plant or fungal tissue is preferred. Mercerization processes as described herein may be used to obtain decellularized but intact single structural cells and/or plant tissue structures of interest (e.g. parenchyma tissue, ground tissue, epidermal tissue, vascular bundles, sieve tubes, petioles, veins, roots, root hairs, etc. . . . ) as desired to suit the particular application(s) of interest.

In embodiments of the methods as described herein, the single structural cells or groups of structural cells (having a decellularized 3-dimensional structure) resulting from mercerization may be mixed or distributed in a hydrogel, to provide a mixture.

In certain embodiments, the hydrogel into which the single structural cells, groups of structural cells, or both, are mixed or distributed may comprise any one or more of alginate, pectin, gelatin, methylcellulose, carboxymethylcellulose, hydroxypropylcellulose, hydroxypropyl methyl cellulose, hydroxyethyl methyl cellulose, agar, pluronic acid, triblock PEO-PPO-PEO copolymers of poly(ethylene oxide) (PEO) and poly(propylene oxide) (PPO), dissolved or regenerated plant cellulose, dissolved cellulose-based hydrogel, hyaluronic acid, extracellular matrix proteins (e.g. collagen, gelatin, or fibronectin, or any combinations thereof), monoacrylated poly(ethylene glycol), poly(ethylene glycol) diacrylate, poly(ethylene glycol) diacrylate (PEGDA)-co-PEGMA, poly(vinyl alcohol), poly(vinylpyrrolidone), poly(lactic-co-glycolic acid), chitosan, chitin, xanthan gum, elastin, fibrin, fibrinogen, cellulose derivatives, carrageenan, or microcrystalline cellulose hydrogel(s), or any combinations thereof. In certain embodiments, the hydrogel/carrier may optionally be cross-linked.

In further embodiments of the methods as described herein, the mixture of the single structural cells, groups of structural cells, or both, and the hydrogel, may be dehydrated, lyophilized, or freeze-dried to provide the aerogel or foam. The person of skill in the art having the benefit of the teachings set out herein, including the Examples provided below, will be aware of a variety of suitable techniques and apparatus for dehydrating, lyophilizing, or freeze-drying, or otherwise drying or removing at least some liquid/solvent from the mixture, to provide aerogels and/or foams as described herein.

In certain embodiments of the above method, the mercerization may comprise treatment of the decellularized plant or fungal tissue with a base and a peroxide, preferably sodium hydroxide or another hydroxide base as base, and hydrogen peroxide as peroxide. Mercerization may chemically disassemble the decellularized plant or fungal tissue into single structural cells, groups of structural cells, or both, without destroying lignocellulose structures contributing to the 3-dimensional structure of the single structural cells.

In still another embodiment of any of the above method or methods, the mercerization may comprise treatment of the decellularized plant or fungal tissue with aqueous sodium hydroxide solution and hydrogen peroxide while heating.

In still another embodiment of any of the above method or methods, the mercerization may be performed with heating to about 80° C. In certain embodiments, such heating may allow for reduced reaction time, particularly when using sodium hydroxide, for example.

In yet another embodiment of any of the above method or methods, the decellularized plant or fungal tissue may be treated with the aqueous sodium hydroxide solution for a first period of time before the hydrogen peroxide is added to the reaction. In certain embodiments, the first period of time may be about 1 minute to about 24 hours, or any time point therebetween, or any subrange spanning between any two such time points.

In certain embodiments, the peroxide may be added to the reaction in intervals, such as about 15 minute intervals. An illustrative non-limiting example of such peroxide interval approaches may proceed as follows:
Protocol on adding peroxide in 15 min intervals:
The volumes presented here are used for a 4 L beaker reaction vessel. As will be understood, the volumes may be adjusted for different setups.
   A. The working station is cleaned with Accel TB solution and then 70% ethanol.
   B. Using a sterile sieve set atop a waste beaker, press out the water from the decellularized material. Separate into 500 g sets using a balance. All processing is done using aseptic technique in the biosafety cabinet with sterile surgical gloves.
   C. Place the 500 g of material into a clean and sterile 4 L beaker.
   D. Add 2.5 L of 1 M NaOH to the beaker. Raise the temperature to 80
   E. Add 125 mL of 30% hydrogen peroxide. Note: this 30% hydrogen peroxide solution is the stock concentration. Add the stock as it is 30%. The addition of the hydrogen peroxide is done in 25 mL aliquots every 15 minutes throughout the 1 h reaction, starting at t=0.
   F. Place a clean, sterile magnetic stir bar in the beaker.
   G. Stir for 1 h at 80° C. Ensure the stirring is adequate to provide movement of the material but does not splash.
   H. Check to make sure the colour is clear or off-white. If it is still yellow, let the reaction proceed until the colour disappears.
   I. Turn off the heat, and remove the beaker from the heat source. Let the solution cool to room temperature.
   J. Neutralize the solution with the stock HCl until the pH is 6.8-7.2.
   K. Centrifuge the material at 8000 rpm for 15 minutes with an Avanti J-26XPI centrifuge. Make sure the centrifuge is properly balanced and the correct rotor is used. Use clean, sterile 1 L centrifuge containers that match the rotor. They are identified with the maximum speed (8000 rpm).
   L. Remove the supernatant by pouring the liquid into a clean waste beaker. Break up the pellet with a sterile spatula. Resuspend the material in 0.75 L of water for each centrifuge vessel (1 L containers). Seal the lid of the centrifuge vessels, and shake to resuspend the material. Pour back into the 4 L beaker for neutralization.
   M. Repeat the neutralization and centrifugation iterations until the pH remains within 6.8-7.2 for back-to-back measurements after centrifugation and resuspension.
   N. Record the final pH and the number of cycles.
   O. Centrifuge the material one final time to concentrate the mercerized material.
   P The samples are stored in the fridge at 4° C.

In another embodiment of any of the above method or methods, the hydrogen peroxide may be added as a 30% aqueous hydrogen peroxide solution.

In still another embodiment of any of the above method or methods, the hydrogen peroxide for mercerization may be used in a ratio of:
about 20 mL to about 5 mL of 30% hydrogen peroxide solution:about 100 g decellularized plant or fungal tissue:about 500 mL of 1M NaOH solution;
such as:
about 20 mL of 30% hydrogen peroxide solution:about 100 g decellularized plant or fungal tissue:about 500 mL of 1M NaOH solution;
about 10 mL of 30% hydrogen peroxide solution:about 100 g decellularized plant or fungal tissue:about 500 mL of 1M NaOH solution; or
about 5 mL of 30% hydrogen peroxide solution:about 100 g decellularized plant or fungal tissue:about 500 mL of 1M NaOH solution.

As will be understood, these ratios may be scaled up or down to suit the particular application as desired—the recited quantities are provided for showing relative ratios, not absolute quantities.

In yet another embodiment of any of the above method or methods, the method may further comprise a step of neutralizing the pH with one or more neutralization treatments. In another embodiment of any of the above method or methods, the neutralization treatment may comprise treatment with an acid solution, preferably an aqueous HCl solution.

In yet another embodiment of any of the above method or methods, the mercerization may be performed using a ratio of decellularized plant or fungal tissue:aqueous sodium hydroxide solution (m:v, in g:L) of about 1:5 for a 1M aqueous sodium hydroxide solution, or an equivalent ratio for another aqueous sodium hydroxide solution concentration. As will be understood, these ratios may be scaled up or down to suit the particular application as desired—the recited quantities are provided for showing relative ratios, not absolute quantities.

In another embodiment of any of the above method or methods, the mercerization may be performed for at least about 30 minutes, preferably for about 1 hour.

In still another embodiment of any of the above method or methods, the resultant single structural cells or groups of structural cells having a decellularized 3-dimensional structure may be collected by centrifugation.

In yet another embodiment of any of the above method or methods, the single structural cells, groups of structural cells, or both, may be mixed or distributed in a hydrogel comprising alginate, pectin, gelatin, methylcellulose, carboxymethylcellulose, hydroxypropylcellulose, hydroxypropyl methyl cellulose, hydroxyethyl methyl cellulose, agar, pluronic acid, triblock PEO-PPO-PEO copolymers of poly(ethylene oxide) (PEO) and poly(propylene oxide) (PPO), dissolved or regenerated plant cellulose, dissolved cellulose-based hydrogel, hyaluronic acid, extracellular matrix proteins (e.g. collagen, gelatin, or fibronectin, or any combinations thereof), monoacrylated poly(ethylene glycol), poly(ethylene glycol) diacrylate, poly(ethylene glycol) diacrylate (PEGDA)-co-PEGMA, poly(vinyl alcohol), poly(vinylpyrrolidone), poly(lactic-co-glycolic acid), chitosan, chitin, xanthan gum, elastin, fibrin, fibrinogen, cellulose derivatives, carrageenan, or microcrystalline cellulose hydrogel, or any combinations thereof; wherein the hydrogel is optionally cross-linked.

In another embodiment of any of the above method or methods, the method may further comprise a step of performing directional freezing of the mixture to introduce templated or aligned microchannels on a surface of the mixture, within the mixture, or both; a step of molding the mixture using molds having microscale features contacting one or more surfaces of the mixture and/or the aerogel or foam resulting from dehydrating, lyophilizing, or freeze-drying of the mixture, so as to introduce templated or aligned microchannels; a step of punching, pressing, stamping, or otherwise forming geometric patterns, depressions, holes, channels, grooves, ridges, wells, or other structural features within and/or onto at least one surface of the mixture and/or the aerogel or foam before, during, or after dehydrating, lyophilizing, or freeze-drying of the mixture; or any combinations thereof.

Directional freezing, molding, and mechanically processing for imparting microscale and/or macroscale structures to the aerogels, foams, and/or hydrogels have already been described in detail hereinabove, and are further described in the Examples set out below.

In still another embodiment of any of the above method or methods, the directional freezing may be performed by creating a thermal gradient across the mixture from one or more directions so as to form aligned ice crystals beginning from the cold side(s) of the thermal gradient.

In yet another embodiment of any of the above method or methods, the mixture may be directionally frozen over a period of at least about 30 minutes, preferably over a period of about 2 hours.

In another embodiment of any of the above method or methods, the mixture may be directionally frozen by cooling to a temperature of between about −190° C. and about 0° C., such as a temperature of at least about −15° C., preferably about −25° C.

In still another embodiment of any of the above method or methods, the step of dehydrating, lyophilizing, or freeze-drying the mixture to provide the aerogel or foam may comprise freezing the mixture followed by lyophilizing or freeze-drying the mixture.

In yet another embodiment of any of the above method or methods, the method may comprise a further step of cross-linking the hydrogel, rehydrating the aerogel or foam, or both; optionally using CaCl₂) solution to provide cross-linking where alginate or pectin or agar hydrogel is present.

As will be understood, a variety of techniques may be used for cross-linking where desired, and may be selected based on the particular aerogel/foam/hydrogel and/or application(s) of interest. In certain embodiments, structural cells may be mixed with a single hydrogel, or a combination of hydrogels, and cross-linking may, or may not, be performed. In certain embodiments, the mixture may then be frozen, followed by lyophilisation or freeze drying to form an aerogel or foam.

In embodiments where the optional cross-linking is desired, an illustrative list of hydrogels, and a corresponding list of potential cross-linkers, is provided below for illustrative and non-limiting purposes for the person of skill in the art. In certain embodiments, such cross-linking approaches may be used if desired, but as will be understood cross-linking may be optional and the decision on whether or not to cross-link may be made to suit the application(s) of interest and specific details or demands thereof. In certain embodiments, combinations of hydrogels may be used, with or without extra crosslinking. Various illustrative examples are provided in the Examples below, particularly in Example 2.

TABLE 1
Examples of Hydrogels and Corresponding Potential Cross-Linkers

| Hydrogel | Potential cross-linker (if desired) |
|---|---|
| Alginate | CaCl₂, MgCl₂ |
| pectin | CaCl₂, MgCl₂ |
| agar | DDI (4,4 diphenyl diisocyanate) and HDI (1,6 |
| | hexamethylene diisocyanate), or no crosslinking. |
| pluronic acid, triblock PEO-PPO- | α-hydroxy or amino acids such as alanine |
| PEO copolymers of poly(ethylene | |
| oxide) (PEO) and poly(propylene | |
| oxide) (PPO) | |
| methylcellulose | citric acid, divinylsulfone, glycine (physical cross-linker), |
| | PVA, EVA, glyoxal |
| carboxymethylcellulose | citric acid, divinylsulfone, glycine (physical cross-linker), |
| | PVA, EVA, glyoxal |
| microcrystalline cellulose | citric acid, divinylsulfone, glycine (physical cross-linker), |
| | PVA, EVA, glyoxal |
| hydroxypropylcellulose | citric acid, divinylsulfone, glycine (physical cross-linker), |
| | PVA, EVA, glyoxal |
| hydroxypropyl methyl cellulose | citric acid, divinylsulfone, glycine (physical cross-linker), |
| | PVA, EVA, glyoxal |
| hydroxyethyl methyl cellulose | citric acid, divinylsulfone, glycine (physical cross-linker), |
| | PVA, EVA, glyoxal |
| dissolved or regenerated plant | citric acid, divinylsulfone, physical entanglement |
| cellulose | |
| extracellular matrix proteins such as | Glutaraldehyde, riboflavin, formalin, formaldehyde, |
| collagen, gelatin and fibronectin | transglutaminase, EDC (1-ethyl-3-(3- |
| | dimethylaminopropyl)carbodiimide hydrochloride), or no |
| | crosslinking at all. |
| hyaluronic acid | BDDE: 1,4-butanediol diglycidyl ether, DVS: divinyl |
| | sulphone, DEO: 2, 7, 8-diepoxyoctane, CPM: |
| | cohesivepolydensified matrix. |
| Monoacrylated Poly(Ethylene | |
| Glycol) | |
| poly(ethylene glycol) diacrylate | UV |
| poly(ethylene glycol) diacrylate | UV |
| (PEGDA)-co-PEGMA | |
| poly(vinyl alcohol) or | UV, gamma, electron beam |
| poly(vinylpyrrolidone) | |
| poly(lactic-co-glycolic acid) | |
| Chitosan, chitin | Methacrylation, UV, ionic, genipine, aldehydes, physical, |
| Xanthan gum | Physical crosslinking, FeCL₃, citric acid |
| Elastin | Glutaraldehyde, riboflavin, formalin, formaldehyde, |
| | transglutaminase, EDC (1-ethyl-3-(3- |
| | dimethylaminopropyl)carbodiimide hydrochloride), or no |
| | crosslinking at all. |
| Fibrin | Glutaraldehyde, riboflavin, formalin, formaldehyde, |
| | transglutaminase, EDC (1-ethyl-3-(3- |
| | dimethylaminopropyl)carbodiimide hydrochloride), or no |
| | crosslinking at all. |
| Fibrinogen | Glutaraldehyde, riboflavin, formalin, formaldehyde, |
| | transglutaminase, EDC (1-ethyl-3-(3- |
| | dimethylaminopropyl)carbodiimide hydrochloride), or no |
| | crosslinking at all. |
| Cellulose derivatives | Methylcellulose, carboxymethylcellulose, the dissolved |
| | and regenerated celluloses described below. Celluloses |
| | above but modified with esters, imides, amides, amines, |
| | ethers, alkyl, phenyl, side groups and active sites. |
| Carrageenan | Temperature (reversible), calcium, potassium |

In another embodiment of any of the above method or methods, the method may comprise a further step of culturing animal cells on or in the aerogel or foam. In certain embodiments, the method may comprise a further step of culturing muscle cells, fibroblasts, myofibroblasts, neurons, dorsal root ganglion cells, neuronal structures such as axons, neural precursor cells, neural stem cells, glial cells, endogenous stem cells, neutrophils, mesenchymal stem cells, satellite cells, myoblasts, myotubes, muscle progenitor cells, adipocytes, preadipocytes, chondrocytes, osteoblasts, osteoclasts, pre-osteoblasts, tendon progenitor cells, tenocytes, periodontal ligament stem cells, or endothelial cells, or any combinations thereof on or in the aerogel or foam.

In another embodiment, there is provided herein an aerogel or foam produced by any of the method or methods as described herein.

As will be understood, aerogels, foams, and/or hydrogels as described here may be configured and/or used for a wide variety of different applications. By way of non-limiting example, it is contemplated that in certain embodiments there is provided herein a use of any of the aerogel or aerogels or foam or foams as described herein for bone tissue engineering; for templating or aligning growth of cells; for regenerative medicine; for repair of spinal cord injury; for preparing a food product; or any combinations thereof.

In another embodiment, there is provided herein a use of any of the aerogel or aerogels or foam or foams as described herein for templating or aligning growth of cells. In certain embodiments, the cells may comprise muscle cells, nerve cells, or both.

In yet another embodiment, there is provided herein a use of any of the aerogel or aerogels or foam or foams as described herein for repair of spinal cord injury.

In another embodiment, there is provided herein a use of any of the aerogel or aerogels or foam or foams as described herein as insulation or packaging foam.

In still another embodiment, there is provided herein a method for bone tissue engineering or repair in a subject in need thereof, comprising:
implanting any of the aerogel or aerogels or foam or foams as described herein at an affected site of the subject in need thereof;
such that the aerogel or foam promotes bone tissue generation or repair.

In yet another embodiment, there is provided herein a method for templating or aligning growth of cells, comprising:
culturing cells on any of the aerogel or aerogels or foam or foams as described herein, wherein the aerogel or foam comprises templated or aligned microchannels on at least one surface of the aerogel or foam, within the aerogel or foam, or both, optionally formed by directional freezing; by molding using molds having microscale features; by punching, pressing, stamping, or otherwise forming geometric patterns, depressions, holes, channels, grooves, ridges, wells, or other structural features within and/or onto at least one surface; or any combinations thereof;
such that the cultured cells align along the microchannels.

In another embodiment of the above method, the cells may comprise muscle cells or nerve cells or both.

In still another embodiment, there is provided herein a method for repairing spinal cord injury in a subject in need thereof, comprising:
implanting any of the aerogel or aerogels or foam or foams as defined herein at an affected site of the subject in need thereof, wherein the aerogel or foam comprises templated or aligned microchannels optionally formed directional freezing;
such that the aerogel or foam promotes spinal cord repair by promoting and/or aligning growth of nerve cells along the templated or aligned microchannels.

In another embodiment, there is provided herein a food product comprising an aerogel or aerogels or foam of foams as described herein, the aerogel(s) or foam(s) being designed/selected so as to be food-safe and edible. In still another embodiment, the food product may additionally comprise a dye or coloring agent; a preservative; a flavoring agent; a salt; a marinade; or other food-related ingredient or agent of interest.

In yet another embodiment of any of the above food product or food products, the food product may comprise two or more aerogel or foam subunits glued together. In certain embodiments, the glue may comprise agar.

In certain embodiments, the food product may be designed or configured to mimic a traditional meat product. By way of example, tuna, salmon and similar fish are characterized by the lines found interspersed between the flakes of meat. These lines are due to the presence of fat (omega-3). Wild salmon typically have fewer and thinner white lines due to the fact that wild salmon typically burn more calories than farmed salmon. As well, their meat is redder from increased blood supply. Therefore, the presence of these white lines and their appearance, thickness, will depend on the desired look of the meat to be achieved. An illustrative and non-limiting example of a protocol to produce these lines in aerogel biomaterials as described herein may proceed as follows:
A. An aerogel biomaterial is produced as described in detail herein with a set concentration of mercerized apple material (structural cells), and a binding agent carrier such as alginate.
B. The material is thoroughly mixed, then poured into a container of desired dimensions and frozen overnight.
C. The material is then lyophilized until dry.
D. It is then crosslinked with calcium chloride (if bound with alginate, or pectin or similar) by adding enough calcium chloride to submerge the material and fully hydrate it for at least 30 minutes-1 hour.
E. The material is then excised from its container and prepared. This entails cutting the material to the desired dimensions.
F. In order to achieve the appearance of tuna or salmon “lines”, the resulting material is cut using a sharp knife, scalpel, or microtome blade.
a. For a salmon sashimi mimic, the biomaterial is cut into a rectangular piece. the rest of the material is cut away.
b. Then, slight diagonal cuts are made into the material at varying interspersed lengths, without cutting all the way through the material (approximately ¾ depth) at (5 mm-1 cm increments down the length of the piece).
G. Once the cuts have been made through the entire material, the white lines are produced by combining another binding agent such as 2% agar with titanium dioxide powder. The proportions may depend on the desired appearance of white colouring. Less than 0.1 g of titanium dioxide for 100 mL of agar is sufficient to achieve a white colour.
H. The premixed agar and titanium dioxide may then be painted, or gently pipetted between the cuts into the wells made by the precut lines made in step (F).
I. The material is allowed to briefly solidify (˜3-5 mins).
J. Once dry or even during drying, the lines may be corrected, shaped or cut if any agar spill over or there is not enough.
a. Care is taken depending on the moisture content of the original scaffold material. A sample that is too wet may absorb much of the agar or may prevent it from drying quickly. The sample is preferably hydrated but not overly wet.

In still another embodiment of any of the food product or food products, the aerogel or foam may comprise templated or aligned microchannels optionally formed by directional freezing.

In certain embodiments, the aerogel or foam may comprise muscle cells, fibroblasts, myofibroblasts, neurons, dorsal root ganglion cells, neuronal structures such as axons, neural precursor cells, neural stem cells, glial cells, endogenous stem cells, neutrophils, mesenchymal stem cells, satellite cells, myoblasts, myotubes, muscle progenitor cells, adipocytes, preadipocytes, chondrocytes, osteoblasts, osteoclasts, pre-osteoblasts, tendon progenitor cells, tenocytes, periodontal ligament stem cells, or endothelial cells, or any combinations thereof, optionally aligned along templated or aligned microchannels; preferably wherein the aerogel or foam comprises templated or aligned microchannels optionally formed by directional freezing, and wherein the aerogel or foam comprises muscle cells, fat cells, connective tissue cells (e.g. fibroblasts), cartilage, bone, epithelial, or endothelial cells, or any combinations thereof, aligned along the templated or aligned microchannels.

In another embodiment, there is provided herein a use of an aerogel or aerogels or foam or foams as described herein in a food product, the aerogel(s) and/or foam(s) being designed/selected so as to be food-safe and edible.

In still another embodiment, there is provided herein a method for preparing single structural cells, groups of structural cells, or both, from decellularized plant or fungal tissue, comprising:
providing a decellularized plant or fungal tissue;
obtaining single structural cells, groups of structural cells, or both, from the decellularized plant or fungal tissue, by performing mercerization of the decellularized plant or fungal tissue and collecting the resultant single structural cells or groups of structural cells having a decellularized 3-dimensional structure.

In yet another embodiment of the above method, the mercerization may comprise treatment of the decellularized plant or fungal tissue with a base and a peroxide, preferably sodium hydroxide or another hydroxide base as base, and hydrogen peroxide as peroxide.

In another embodiment of any of the above method or methods, the mercerization may comprise treatment of the decellularized plant or fungal tissue with aqueous sodium hydroxide solution and hydrogen peroxide while heating.

In still another embodiment of any of the above method or methods, the decellularized plant or fungal tissue may be treated with the aqueous sodium hydroxide solution for a first period of time before the hydrogen peroxide is added to the reaction.

In yet another embodiment of any of the above method or methods, the hydrogen peroxide may be added as a 30% aqueous hydrogen peroxide solution.

In still another embodiment of any of the above method or methods, the hydrogen peroxide for mercerization may be used in a ratio of:
about 20 mL to about 5 mL of 30% hydrogen peroxide solution:about 100 g decellularized plant or fungal tissue:about 500 mL of 1M NaOH solution;
such as:
about 20 mL of 30% hydrogen peroxide solution:about 100 g decellularized plant or fungal tissue:about 500 mL of 1M NaOH solution;
about 10 mL of 30% hydrogen peroxide solution:about 100 g decellularized plant or fungal tissue:about 500 mL of 1M NaOH solution; or
about 5 mL of 30% hydrogen peroxide solution:about 100 g decellularized plant or fungal tissue:about 500 mL of 1M NaOH solution.

In yet another embodiment of any of the above method or methods, the method may further comprise a step of neutralizing the pH with one or more neutralization treatments.

In another embodiment of any of the above method or methods, the neutralization treatment may comprise treatment with an acid solution, preferably an aqueous HCl solution.

In still another embodiment of any of the above method or methods, the mercerization may be performed with heating to about 80° C.

In yet another embodiment of any of the above method or methods, the mercerization may be performed using a ratio of decellularized plant or fungal tissue:aqueous sodium hydroxide solution (m:v, in g:L) of about 1:5 for a 1M aqueous sodium hydroxide solution, or an equivalent ratio for another aqueous sodium hydroxide solution concentration.

In still another embodiment of any of the above method or methods, the mercerization may be performed for at least about 30 minutes, preferably for about 1 hour.

In yet another embodiment of any of the above method or methods, the resultant single structural cells or groups of structural cells having a decellularized 3-dimensional structure may be collected by centrifugation.

In still another embodiment, there is provided herein single structural cells, groups of structural cells, or both, prepared by any of the method or methods as described herein.

Also provided herein are cellulose-based hydrogels that may have a variety of different applications. In certain embodiments, such cellulose-based hydrogels as described herein may be for use as hydrogel for preparing aerogels and/or foams as described herein, by way of non-limiting example.

In an embodiment, there is provided herein a cellulose-based hydrogel comprising cellulose derived from decellularized plant or fungal tissue via dissolution with dimethylacetamide and lithium chloride followed by regeneration with ethanol. Illustrative examples of such dissolution are described in further detail in Example 3 below.

In still another embodiment, there is provided herein a method for preparing a cellulose-based hydrogel comprising:
providing a decellularized plant or fungal tissue;
dissolving cellulose of the decellularized plant or fungal tissue by treatment with dimethylacetamide (DMAc) and lithium chloride (LiCl); and
regenerating a cellulose-based hydrogel from the dissolved cellulose by solvent exchange with ethanol,
thereby providing the cellulose-based hydrogel.

As will be understood, cellulose-based hydrogels may comprise a hydrogel containing one or more cellulose or cellulose derivatives. Typically, the cellulose and/or cellulose derivatives may be obtained by dissolution of the cellulose and/or cellulose derivatives from decellularized plant or fungal tissue. As will be understood, cellulose and/or cellulose derivatives may alternatively be obtained by dissolving plant of fungal tissue which has not been decellularized in certain embodiments, but as described herein dissolution of cellulose and/or cellulose derivatives from decellularized plant or fungal tissue is preferred. Preparation of decellularized plant or fungal tissue has already been described in detail hereinabove, and is further described in the Examples below.

Cellulose and/or cellulose derivatives of the decellularized plant or fungal tissue may be dissolved by treatment with DMAc and LiCl. Illustrative examples of such dissolution treatments are described in further detail in Example 3 below.

In another embodiment of the above methods, the solvent exchange with ethanol may be performed using a dialysis membrane, or by adding ethanol on top of the dissolved cellulose to promote solvent exchange.

In still another embodiment of any of the above method or methods, the method may further comprise bleaching the cellulose-based hydrogel with hydrogen peroxide.

In yet another embodiment, there is provided herein a cellulose-based hydrogel comprising cellulose derived from decellularized plant or fungal tissue via dissolution with: dimethylacetamide and lithium chloride, LiClO₄, xanthate, EDA/KSCN, H₃PO₄, NaOH/urea, ZnCl₂, TBAF/DMSO, NMMO, an ionic liquid (IL) (such as 1-butylpyridinium chloride and aluminum chloride, alkyl imidazolium in association with nitrate, preferably a room temperature ionic liquid), or any combinations thereof.

In still another embodiment, there is provided herein a method for preparing a cellulose-based hydrogel comprising:
providing a decellularized plant or fungal tissue;
dissolving cellulose of the decellularized plant or fungal tissue by treatment with dimethylacetamide and lithium chloride, LiClO₄, xanthate, EDA/KSCN, H₃PO₄, NaOH/urea, ZnCl₂, TBAF/DMSO, NMMO, an ionic liquid (IL) (such as 1-butylpyridinium chloride and aluminum chloride, alkyl imidazolium in association with nitrate, preferably a room temperature ionic liquid; there are an estimated 10¹² ionic liquids), or any combinations thereof;
obtaining the dissolved cellulose and preparing the cellulose-based hydrogel using the dissolved cellulose.

Treatments for dissolving cellulose and/or cellulose derivatives of the decellularized plant or fungal tissue may be designed or selected to suit the particular application(s) of interest. Examples of agents that may be used for dissolving cellulose and/or cellulose derivatives of the decellularized plant or fungal tissue may include, but are not limited to, dimethylacetamide and lithium chloride, LiClO₄, xanthate, EDA/KSCN, H₃PO₄, NaOH/urea, ZnCl₂, TBAF/DMSO, NMMO, an ionic liquid (IL) (such as 1-butylpyridinium chloride and aluminum chloride, alkyl imidazolium in association with nitrate, preferably a room temperature ionic liquid), or any combinations thereof.

In another embodiment, there is provided herein a cellulose-based hydrogel prepared by any of the method or methods as described herein.

In another embodiment of any of the aerogel or aerogels or foam or foams as described herein, the hydrogel may comprise any of the cellulose-based hydrogel or cellulose-based hydrogels as described herein.

In still another embodiment, there is provided herein a food product comprising any of the aerogel or aerogels or foam or foams or structural cell or structural cells as described herein, wherein the food product is a meat mimic and comprises a plurality of lines providing the appearance of fatty white lines found in tuna, salmon, or another fish-type meat.

In yet another embodiment of the above food product, the food product may be a mimic of tuna, salmon, or another fish meat.

In still another embodiment, there is provided herein a food product comprising any of the aerogel or aerogels or foam or foams or structural cell or structural cells as described herein, wherein the food product is a meat mimic, and may optionally comprise a plurality of lines or other patterns providing the appearance of fatty materials or fatty deposits found in a natural meat.

In yet another embodiment of the above food product, the food product may be a mimic of a poultry, bovine, fish, or porcine meat, or any other suitable meat. In certain embodiments, the food product may mimic steak, chicken, pork, or another such meat, for example.

In another embodiment of any of the above food product or food products, the food product may contain one or more dyes or colorants providing the color of tuna, salmon, or another fish meat, or another meat.

In still another embodiment of any of the above food product or food products, the plurality of lines may be formed in cuts or channels formed in the aerogel or foam.

In yet another embodiment of any of the above food product or food products, the plurality of lines may comprise titanium dioxide, optionally combined with agar binding agent or another such binding agent.

In another embodiment of any of the above food product or food products, the titanium dioxide, optionally combined with agar binding agent, may be applied into cuts or channels formed in the aerogel or foam to provide the appearance of the fatty white lines found in tuna, salmon, or another fish-type meat, or another meat.

In another embodiment, there is provided herein a method for preparing a food product, the food product being a tuna, salmon, or other fish meat mimic, or another meat mimic, said method comprising:
providing any of the aerogel or aerogels or foam or foams as described herein;
optionally, dying or coloring the aerogel a color of tuna, salmon, or other fish meat or other meat;
cutting or otherwise processing the aerogel in order to form cuts or channels along the surface of the aerogel; and
applying a dye or coloring agent to the cuts or channels to provide an appearance of fatty white lines characteristic of tuna, salmon, or other fish meat or another meat.

In another embodiment of the above method, the dye or coloring agent applied to the cuts or channels may comprise titanium dioxide.

In still another embodiment of any of the above method or methods, the dye or coloring agent applied to the cuts or channels may be combined with a binding agent.

In yet another embodiment of any of the above method or methods, the binding agent may comprise agar.

In another embodiment, there is provided herein a food product prepared by any of the method or methods described herein.

In another embodiment, there is provided herein a non-resorbable dermal filler comprising an aerogel, foam, structural cell, or cellulose-based hydrogel as described herein; or any combinations thereof.

In yet another embodiment, there is provided herein a dermal filler comprising single structural cells, groups of structural cells, or both, derived from a plant or fungal tissue, the single structural cells or groups of structural cells having a decellularized 3-dimensional structure lacking cellular materials and nucleic acids of plant or fungal tissue, the single structural cells, groups of structural cells, or both, being derived from the plant or fungal tissue by mercerization.

In another embodiment of any of the above dermal filler or dermal fillers, the dermal filler may further comprise a carrier fluid or gel.

In still another embodiment of any of the above dermal filler or dermal fillers, the carrier fluid or gel may comprise water, an aqueous solution, or a hydrogel.

In yet another embodiment of any of the above dermal filler or dermal fillers, the carrier fluid or gel may comprise a saline solution, or a collagen, hyaluronic acid, methylcellulose, and/or dissolved plant-derived decellularized cellulose-based hydrogel.

In another embodiment of any of the above dermal filler or dermal fillers, the dermal filler may further comprise an anesthetic agent.

In still another embodiment of any of the above dermal filler or dermal fillers, the anesthetic agent may comprise lidocaine, benzocaine, tetracaine, polocaine, epinephrine, or any combinations thereof.

In another embodiment of any of the above dermal filler or dermal fillers, the dermal filler may comprise PBS (saline), hyaluronic acid (cross-linked or non-crosslinked), alginate, collagen, pluronic acid (e.g. pluronic F 127), agar, agarose, or fibrin, calcium hydroxylapatite, Poly-L-lactic acid, autologous fat, silicone, dextran, methylcellulose, or any combinations thereof.

In another embodiment of any of the above dermal filler or dermal fillers, the dermal filler may comprise at least one of: 2% lidocaine gel; a triple anesthetic gel comprising 20% benzocaine, 6% lidocaine, and 4% tetracaine (BLTgel); 3% Polocaine; or a mixture of 2% lidocaine with epinephrine.

In another embodiment of any of the above dermal filler or dermal fillers, the structural cells may have a size, diameter, or minimum feret diameter of at least about 20 μm.

In another embodiment of any of the above dermal filler or dermal fillers, the structural cells may have a size, diameter, or maximum feret diameter of less than about 1000 μm.

In still another embodiment of any of the above dermal filler or dermal fillers, the structural cells may have a size, diameter, or feret diameter distribution within a range of about 20 μm to about 1000 μm.

In yet another embodiment of any of the above dermal filler or dermal fillers, the structural cells may have a particle size, diameter, or feret diameter distribution having a peak about 200-300 μm.

In another embodiment of any of the above dermal filler or dermal fillers, the structural cells may have a mean particle size, diameter, or feret diameter within a range of about 200 μm to about 300 μm.

In another embodiment of any of the above dermal filler or dermal fillers, the structural cells may have an average projected particle area within a range of about 30,000 to about 75,000 μm².

In still another embodiment of any of the above dermal filler or dermal fillers, the dermal filler may be sterilized.

In yet another embodiment of any of the above dermal filler or dermal fillers, the sterilization may be by gamma sterilization.

In still another embodiment of any of the above dermal filler or dermal fillers, the dermal filler may be formulated for subdermal injection, deep dermal injection, subcutaneous injection (e.g. subcutaneous fat injection), or any combinations thereof.

In another embodiment of any of the above dermal filler or dermal fillers, the dermal filler may be provided in a syringe or injection device.

In another embodiment, there is provided herein a use of any of the dermal filler or dermal fillers as described herein as a soft tissue filler, for reconstructive surgery, or both.

In another embodiment, there is provided herein a use of any of the dermal filler or dermal fillers as described herein for improving cosmetic appearance of a subject in need thereof.

In another embodiment, there is provided herein a use of any of the dermal filler or dermal fillers as described herein for increasing tissue volume, smoothing wrinkles, or both, in a subject in need thereof.

In another embodiment, there is provided herein a method for improving cosmetic appearance, increasing tissue volume, smoothing wrinkles, or any combinations thereof, in a subject in need thereof, said method comprising:
administering or injecting any of the dermal filler or dermal fillers as described herein to a region in need thereof;
thereby improving cosmetic appearance, increasing tissue volume, smoothing wrinkles, or any combinations thereof, of the subject.

In another embodiment of the above use or uses or method or methods, native cells of the subject may infiltrate the dermal filler.

In yet another embodiment of the above use or uses or method or methods, the dermal filler may be non-resorbable such that the decellularized plant or fungal tissue remains substantially intact within the subject.

Additional details relevant to plant-derived dermal fillers are described in US provisional patent application no. 63/036,126, entitled “Dermal Fillers”, which is herein incorporated by reference in its entirety.

### Example 1-Aerogels Prepared from Decellularized Plant Tissues

Plant-derived scaffolds can provide desirable biological/physical properties (such as in vitro/in vivo biocompatibility), are readily producible, and can provide fixed mechanical/structural properties. However, many plant-derived scaffolds do not provide a significant level of control over parameters such as surface biochemistry, tuneable mechanical properties, tuneable micro/macro-scale architectures, and/or scalable production methods, for example.

This example describes the development of plant-derived scaffolds, with an emphasis toward providing one or more (or all) of the following characteristics: derived from decellularized plant materials; ability to retain desirable plant microarchitectures; amendable to scalable production method(s); ability to provide a wide range of scaffold biochemistry; able to provide tuneable mechanical properties; ability to provide tuneable porosity; in vitro biocompatibility; in vivo biocompatibility; and/or stable during cooking conditions (where food product applications are desired).

The present example describes development of scaffolds that meet all the characteristics above. These scaffold materials are prepared in this example by first decellularizing plant materials, followed by performing a mercerization treatment in which the decellularized materials are treated under basic conditions at high temperatures to separate the plant tissues into single intact decellularized plant structural cells (or groups of structural cells comprising small clusters of linked structural cells). A strong oxidizer is then introduced to make the resulting slurry of cells white in colour. The whitening is performed to produce a final product that provides a blank canvas for various applications in which colouring may be desired (for example, in food products, etc. . . . ). Then, the slurry is neutralized and centrifuged to result in a thick paste comprising a high concentration of decellularized plant structural cells. This resulting product may then be mixed with a wide variety of hydrogels with varying biochemical properties to produce composite hydrogel mixture(s). At this point, the hydrogels can be placed into large scale molds and lyophilized to produce a final product in the form of a lightweight, stable and large format aerogel or foam. A library of these aerogels or foams was created in this example with varying mechanical, structural and biochemical properties which may be useful for a variety of different applications. Moreover, upon (re)hydration, the aerogels and foams (also referred to herein as hydrogels upon rehydration in a liquid, most often water or aqueous solutions) may be further crosslinked and/or further modified for downstream use.

### Materials and Methods

Processes used for producing aerogel formulations of this example are as follows:

### Decellularization:

Decellularization of plant tissue was performed as described in PCT patent publication WO2017/136950, entitled “Decellularised Cell Wall Structures From Plants and Fungus and Use Thereof as Scaffold Materials”, which is herein incorporated by reference in its entirety. A 5-day protocol was used as follows:

Day 1: Apples were peeled and then sliced on a mandolin to 1 mm thick. Slices were added to a 4 L beaker with a 0.1% SDS solution and shaken at 130 RPM. Appropriate Lot #'s were assigned and batch production records maintained.

Day 2: The 0.1% SDS solution was removed from the beakers and replaced with a fresh 0.1% SDS solution. Shaking resumed at 130 RPM.

Day 3: The 0.1% SDS solution was removed from the beakers and replaced with a fresh 0.1% SDS solution. Shaking resumed at 130 RPM.

Day 4: The 0.1% SDS solution was removed from the beakers and replaced with a fresh 0.1M CaCl₂) solution. Shaking resumed at 130 RPM.

Day 5: The 0.1M CaCl₂) solution was removed and the contents were washed 3× with 1 L of sterile water. After removing the water the beaker was filled with 1 L of 70% ethanol incubated for 30 mins. Finally, the ethanol was removed and the contents were washed 3× with 1 L of sterile water.

### Mercerization:

Mercerization was most often performed on Day 5 of the above protocol after the final washes with sterile water. However, the product obtained on the Day 5 step could alternatively be stored in the fridge until needed. Typically decellularized plant materials used in these studies were stored for no more than 2 weeks in the fridge; however, it is expected that such plant materials may be stored much longer if needed or desired. Freezing the decellularized plant materials, or lyophilizing them, are also contemplated steps for preservation of the Day 5 product; however, this was not normally performed in these studies.

In this example, mercerization was performed as follows:

Day 5 (cont'd): Mercerization was performed as follows:
Key Chemicals and Solutions:
   Sterile water (Baxter, catalog #JF7624)
   Sodium Hydroxide (Acros Organics 259860010)
   Hydrochloric acid (Fisher, CAS 7732-18-5)
   Hydrogen peroxide (Aqueous solution 30%, Fisher chemical, CAS 7722-84-1)
Tools and Materials:
   Fume hood
   4 L beaker
   pH meter
   Label stickers
   Centrifuge: 1 L capacity, 8000 rpm.
   Sterile sieve for initial water removal.
Procedure: The following protocol was followed:
   The working station was cleaned with Accel TB solution and then 70% ethanol.
   Using a sterile sieve set atop a waste beaker, manually press out the water from the decellularized material. Separate into 500 g sets using a balance. All processing was done using aseptic technique in the biosafety cabinet with sterile surgical gloves.
   ·Place the 500 g of material into a clean and sterile 4 L beaker.
   Add 2.5 L of 1 M NaOH to the beaker. Raise the temperature to 80° C.
   Add 125 mL of 30% hydrogen peroxide. Note: this 30% hydrogen peroxide solution is the stock concentration. Simply add the stock as it is 30%.
   Place a clean, sterile magnetic stir bar in the beaker.
   Stir for 1 h at 80° C. Ensure the stirring is adequate to provide movement of the material but does not splash.
   Check to make sure the colour is clear or off-white. If it is still yellow, let the reaction proceed until the colour disappears.
   Turn off the heat, and remove the beaker from the heat source. Let the solution cool to room temperature.
   Using a pH meter, neutralize the solution with the stock HCl until the pH is 6.8-7.2. Record the pH value.
   Centrifuge the material at 8000 rpm for 15 minutes. Make sure the centrifuge is properly balanced and the correct rotor is used. Use clean, sterile 1 L centrifuge containers that match the rotor. They are identified with the maximum speed (8000 rpm).
   Remove the supernatant by pouring the liquid into a clean waste beaker. Manually break up the pellet with a sterile spatula. Resuspend the material in 0.75 L of water for each centrifuge vessel (1 L containers). Seal the lid of the centrifuge vessels, and shake to resuspend the material. Pour back into the 4 L beaker for neutralization.
   Repeat the neutralization and centrifugation iterations until the pH remains within 6.8-7.2 for back-to-back measurements after centrifugation and resuspension.
   Record the final pH and the number of cycles.
   Centrifuge the material one final time to concentrate the mercerized material.
   Discard the supernatant, and transfer the pellet of mercerized material to sterile 50 mL Falcon tubes.
   Proper labelling and identification of the container contents is performed.
   The samples are stored in the fridge at 4° C.

### Results:

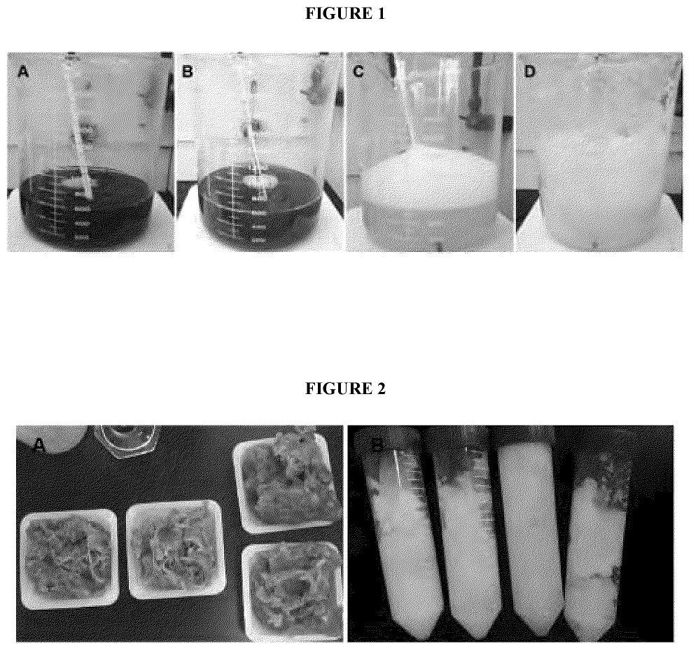

Results are shown in FIGS. 1-4.

FIG. 1 shows results of AA (apple) mercerization and discolouring in a smaller sample of AA (100 g in the images). 100 g of decellularized AA (apple) material was mercerized in 500 mL of 1M NaOH at 80° C. for one hour. A total of 75 mL of H₂O₂ was added throughout the mercerization process to discolour the samples (reaction formed Na₂O₂ (sodium peroxide) which is a strong oxidizer). FIG. 1(A) shows AA samples in NaOH (T=0 min). FIG. 1(B) shows AA samples in NaOH right after the addition of 25 mL of H₂O₂. The discolouration process started right after the addition. (T=2 min). In FIG. 1(C) the samples appear yellowish (T=10 min). In FIG. 1(D) AA samples appear off-white after 60 minutes of mercerization in NaOH and the H₂O₂ additions.

FIG. 2(A) shows the decellularized AA tissue used as the starting material for the mercerization process. FIG. 2(B) shows the product obtained after the mercerization. The product is shown after follow-up neutralization and centrifugation. The obtained product material shown in FIG. 2(B) is very thick and viscous, resembling a sort of apple “paste”.

FIG. 3 shows images of the apple-derived decellularized single structural cells (and some groups of structural cells comprising a small plurality of single structural cells linked together) obtained/isolated following mercerization. In FIG. 3, dilution and fluorescent staining of the structural cells with congo red dye revealed the microarchitecture of the cells is intact.

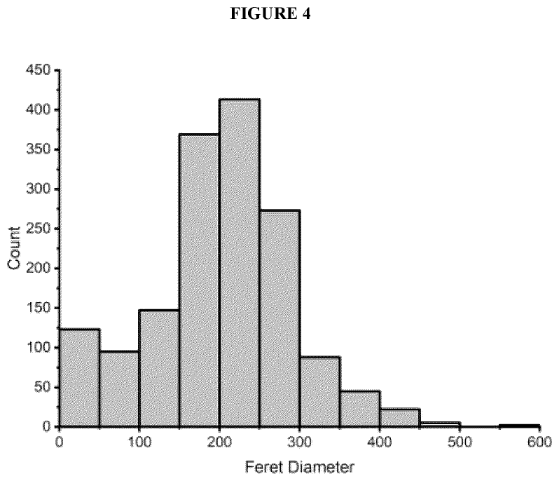

FIG. 4 shows the particle size distribution of mercerated (1M NaOH) decellularized AA with the addition of H₂O₂ (N=10 images analyzed). Average size confirms the presence of intact single structural cells which have maintained their microarchitectural characteristics.

A study examining the yield of material after drying was performed, in which 7 samples were prepared from the mercerized materials prepared as described immediately above, which each had an initial mass of 1 g. After lyophilization, collecting and weighing the dry mass, the average mass was 0.052±0.005 g or ˜5%. This indicates that the mercerized paste was ˜95% water.

### Mercerization: Optimization

Several preparation steps in the procedure described above were determined/optimized based on a number of experiments and observations and analysis of results.

Firstly, in traditional mercerization/maceration protocols used for other applications, H₂O₂ is often added at the start of the process with the base/acid (mercerization/maceration, respectively). In the present studies, it is now found that the early use of peroxide may cause highly exothermic reaction which can make the solutions more difficult to handle and importantly, the reaction produces significantly more foam. By leaving the peroxide step to later in the mercerization process, it has now been found that less foam was produced. Moreover, during subsequent neutralization and centrifugation steps, less foam and gas build up would occur when peroxide was left to the end of the processing steps.

Secondly, studies were performed to investigate optimal amounts of NaOH to be used in the reaction, which is currently set at a 1:5 ratio of AA:NaOH (mass:volume, eg. 500 g:2.5 L). To evaluate these ratios, a series of experiments were performed to determine the minimum ratio of AA material to sodium hydroxide solution (NaOH) that will yield properly mercerized AA. Three different ratios were tested (i.e., 1:1, 1:2, and 1:5) using the mercerization protocol; all samples were mercerized for 60 minutes at 80° C., then neutralized and centrifuged until a stable pH was achieved. The protocol was as follows:
1. Three portions of decellularized AA were weighed: 20 g, 50 g, and 100 g. All AA (apple) material was pressed to remove excess water.
2. Three 1 L beakers were filled with 100 mL of 1M NaOH and placed on magnetic stir plates.
3. All solutions were heated to 80° C.; then, a stir bar and a portion of decellularized AA was added to each and labelled appropriately, corresponding to the weight of AA added.
4. 5 mL of 30% hydrogen peroxide (H₂O₂) was added to each beaker.
5. All three solutions were stirred for 1 hour at 80° C.
6. After the mercerization, all beakers were removed from their respective hot plates and left to cool completely.
7. Once cooled, all three solutions were neutralized and centrifuged separately until a neutral and stable pH was obtained.
8. The three conditions tested (i.e., 20 g of AA in 100 mL NaOH, 50 g of AA in 100 mL NaOH, and 100 g of AA in 100 mL NaOH) were stored separately in their respective 50 mL Falcon tubes and stored in the fridge for microscopy.
9. For particle analysis, each of the three AA to NaOH conditions were resuspended in distilled water and stained with Congo red for 10 minutes, then mounted on a microscope slide.
10. Using the SZX16 Olympus microscope, slides were imaged using fluorescence microscopy (BV light filter) and saved for future analysis with ImageJ software.

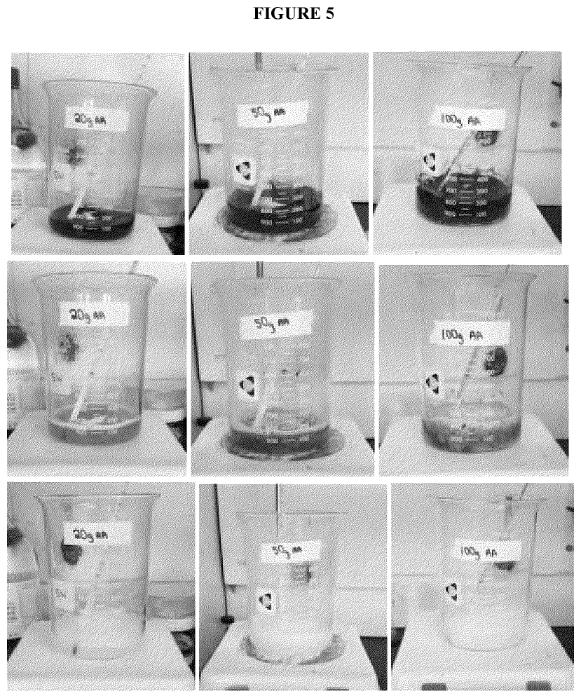

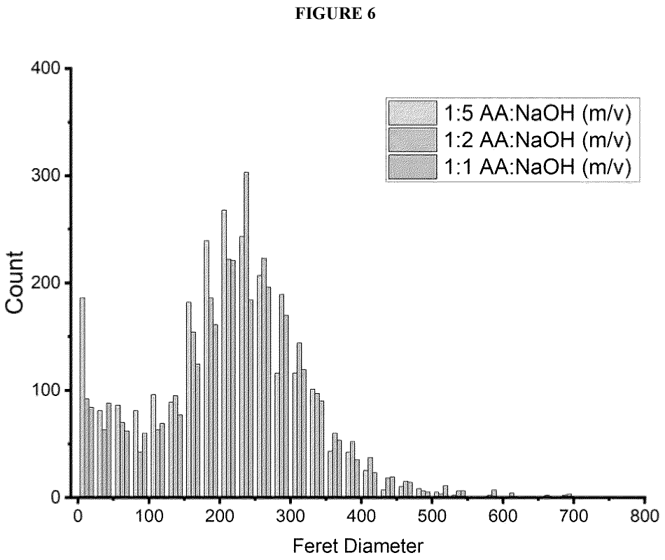

FIG. 5 shows colour change of AA-NaOH solution throughout the 60-minute mercerization of all three ratio conditions (i.e., 20 g, 50 g, and 100 g of AA in 100 mL 1M NaOH). FIG. 6 shows that after mercerization in the various solutions, the isolated single AA cells were imaged and their ferret diameters were measured. The results show that there is no significant difference in the average size, number and distribution of isolated mercerized cells under each condition.

Although the 1:1 solution of AA:NaOH can process the most amount of decellularized AA, and is therefore the most efficient, it was not the optimal approach in the conditions tested. At a 1:1 dilution it was found that the solutions become very thick and demand more peroxide leading to foaming difficulties. Therefore, workable and safely scalable solutions were preferred and it was determined that the 1:5 dilution was the preferred approach for producing raw mercerized materials in these studies.

### Fabrication of Aerogels, Foams, and Hydrogels

(Related Experimental Results for Preparation of Aerogels, Foams, and Hydrogels are also Described in Example 2 Below)

It is contemplated that processes of creating a mercerized apple “paste” as described herein may provide a variety of advantages. Firstly, this raw product may be entirely produced through liquid-based steps from start to finish. With the exception of initial apple peeling and preparation (a process to which automated industrial equipment is available) for decellularization, all further steps may be executed in liquid solutions at large scales if desired. This may provide for generating a large amount of validated raw product of decellularized plant tissue-derived structural cells with intact microarchitectures (single cell units) as opposed to fully dissolved cellulose. Secondly, protocols are developed and described herein to mix the raw product with other hydrogels to create composite biomaterials with controllable structural properties.

Once mixed or distributed into the hydrogel, freezing and lyophilization processes are developed and described herein allowing for control over the architecture of the materials which may be used to produce highly porous and ultralight dry “foams” and “aerogels”. The aerogel and foam formats are convenient and desirable, as they may be highly stable, may be stored under vacuum, may be very light, and may also possess mechanical properties relevant to tissue engineering (for example, 10's-100's of kPa). Moreover, for use in a biological context, it is contemplated that they may then be rehydrated into a hydrogel form, while maintaining their structural integrity.

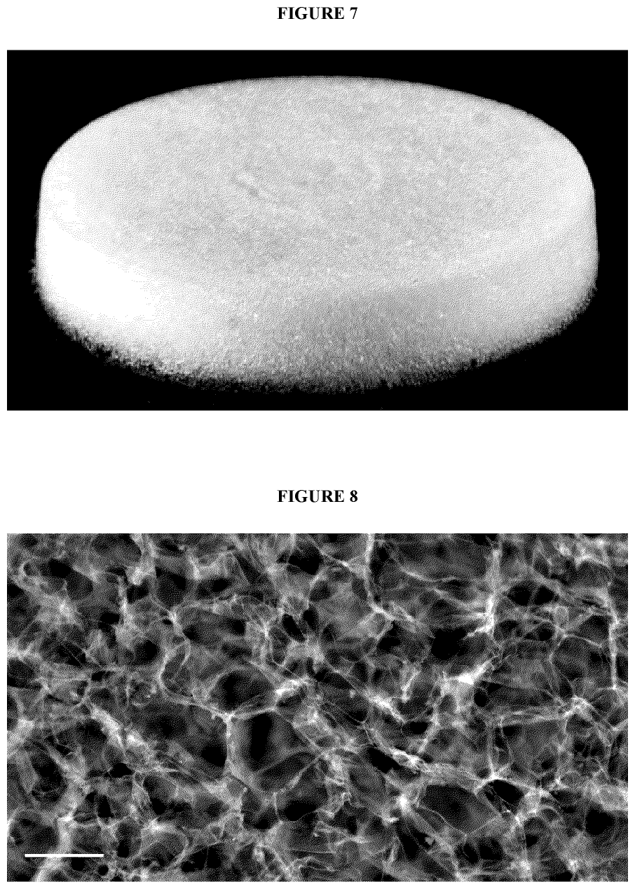

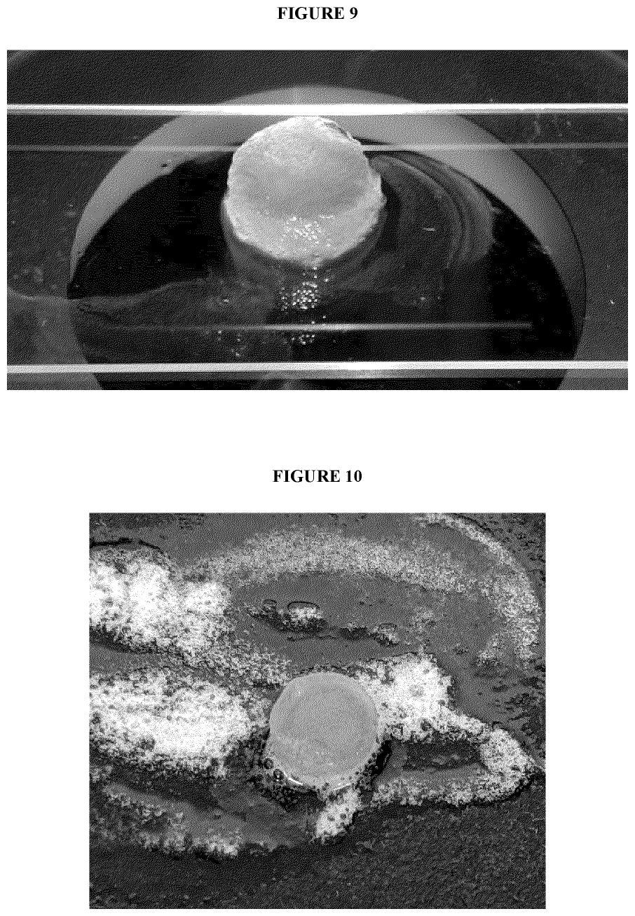

Additional materials on the preparation of aerogels, foams, and hydrogels are provided in Example 2 below. Results of some such aerogel preparations, as prepared in this example, are shown in FIGS. 7-9. FIG. 7 shows an image of an aerogel comprising single structural cells, groups of structural cells, or both, derived from decellularized apple tissue by mercerization thereof, the single structural cells, groups of structural cells, or both, being distributed within a carrier, the carrier derived from a lyophilized hydrogel, in this case a 5% Alginate hydrogel. In this example, the single structural cells, groups of structural cells, or both, were mixed with the 5% alginate hydrogel, and then the mixture was frozen in a mold and subsequently lyophilized to provide the depicted aerogel, which is 6 cm in diameter and 0.7 cm thick. FIG. 8 shows a microscope image of a similarly prepared aerogel, this time using a 50% alginate hydrogel (scale bar=500 μm). FIG. 9 shows a cross-linked and hydrated form of a similar aerogel prepared using 50% alginate hydrogel, the hydrated aerogel (also referred to herein as a hydrogel or hydrogel composite) being about 1 cm in diameter and about 4 mm thick.

### Examples of Aerogel, Foam, and Hydrogel Applications

### Bone Tissue Engineering

Given the highly porous nature of the aerogels, foams, and hydrogels, and their resemblance to bone tissues, it is contemplated that aerogels, foams, and hydrogels as described herein may be used for bone tissue engineering. Ongoing small scale calvarial defect studies assessing the effectiveness of alginate- and pectin-based rehydrated aerogels (comprising the decellularized single structural cells and/or groups of structural cells as described) in bone tissue engineering support such applications. SEM and optical imaging of the aerogel scaffolds are further described in Example 2 below. Production and short-term stability of scaffolds for bone repair are also described in Example 12 below.

### Food Applications and Cooking

Various food formulations have also been developed, and cooking testing has been performed. Results of ongoing studies indicate that rehydrated aerogels may be coloured with beet juice and/or food dyes.

Results also indicate that rehydrated aerogels may be pan fried with butter. Formulations with Alginate have been tested (and additional testing is ongoing), and results obtained so far indicate that shape was stable, a crispy exterior was produced, and what visually appears like a roboust/solid interior was observed.

It is additionally contemplated that composite materials may be produced by “gluing” aerogel scaffolds together to make larger structures. Agar has been tested as a glue, and results have been favourable.

Modification of these materials to add amine groups to cellulose and/or cellulose derivatives is also contemplated. Initial glycine-based modification chemistry is described in Example 3 below. It is contemplated that one purpose of adding this functional group to the materials may be to employ the enzyme transglutaminase (aka “meat glue”), which may provide the possibility of using edible meat-glue (transglutaminase) to glue together aerogel scaffolds with each other or with sections of real meat in large formats, with possibility of controlling long range structure, mechanical properties, and/or other relevant properties.

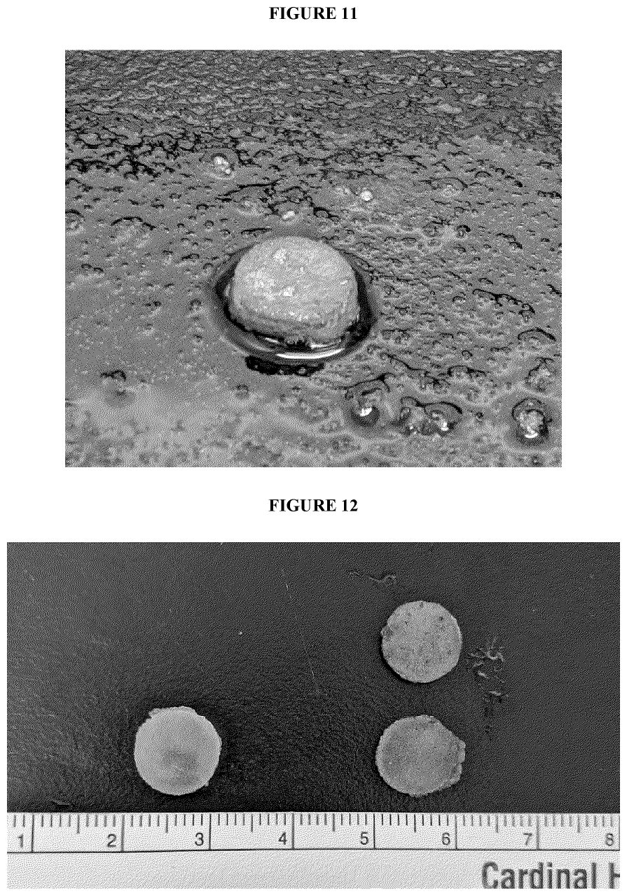

FIG. 10 shows an example of a hydrated aerogel as described herein (being alginate-based in this example) on a frying pan with butter at the start of cooking. FIG. 11 shows the same aerogel after several minutes of cooking, where it is observed that the aerogel maintained its shape and integrity, and a crust was formed. FIG. 12 shows a comparison of “raw” (left) and cooked (right) aerogels.

### Directional Freezing to Produce Structural Features in Aerogels, Foams, and Hydrogels

Directional freezing approaches are described in further detail below. These approaches may provide for, for example, templating of muscle cells to grow into aligned myotubes on the aerogel scaffolds. It is contemplated that directional freezing may be used to produce structural features in aerogels, foams, and hydrogels, which may be useful for a variety of applications including in spinal cord repair, for example. Directional freezing is mainly described below in terms of directional freezing in one direction, however it will be understood that multi-direction directional freezing may also be used as desired to provide various arrangements of structural features. Typically, directional freezing may be achieved by placing a vessel containing the solution to be frozen on a cold plate to ensure that ice crystals form at one edge and grow linearly away from the cold edge. However, it is also contemplated to begin the freeze-casting process in this way and slowly move the vessel so that the location of the cold side changes in time. Conversely, the cold plate itself may be moved to a different location on the vessel to nucleate another group of ice crystals which grow in a different direction from the initial set. In yet another embodiment, the vessel may have two or more cold plates attached to it which can be turned on simultaneously, or at separate points during the freezing process in order to create highly complex, yet controlled, architectures in the resulting aerogel, for example.

Directional freezing approaches have been employed in polymer science applications, and is contemplated herein as a strategy to create aligned biomaterials for tissue engineering applications, for example. By creating a larger thermal gradient on one side of a hydrogel, linear and highly aligned ice crystals may form from the cold side. This may force the surrounding hydrogel polymers to form around the ice crystals, creating aligned microscale channels. After lyophilizing the resulting material, a scaffold may be created with many microchannels.

To achieve directional freezing in this Example, a custom-built apparatus was designed around a peltier module. Briefly, a Phanteks CPU Cooler (PH-TC14PE) with 140 mm fans was used to displace heat and oriented in an upside down configuration (any similar large CPU cooler and fans could be used). A peltier element (TEC-12706) was placed with the hot side down on the CPU block with the cold side facing up. Finally, a 4×4″ copper plate was then mounted on top of the peltier element to become an efficient cooling surface. In between each interface thermal compound (Arctic MX-4) was placed to ensure efficient heat transfer.

The peltier element was sourced from AEP's collection of parts. Based on its power usage (12V/4.2A) it is assumed that the element is a TEC-12706 element; however, there was no code on the element itself. Finally, a k-type thermocouple was embedded in the bottom side of the copper plate as close as possible to the peltier element to track temperatures and freezing rates.

To power the device, 12V was supplied directly to the peltier element and also fed to a voltage buck converter. The buck converter was used to supply 12V to the fans. This allows eventual use of higher voltages to drive the peltier while only supplying 12V to the fans.

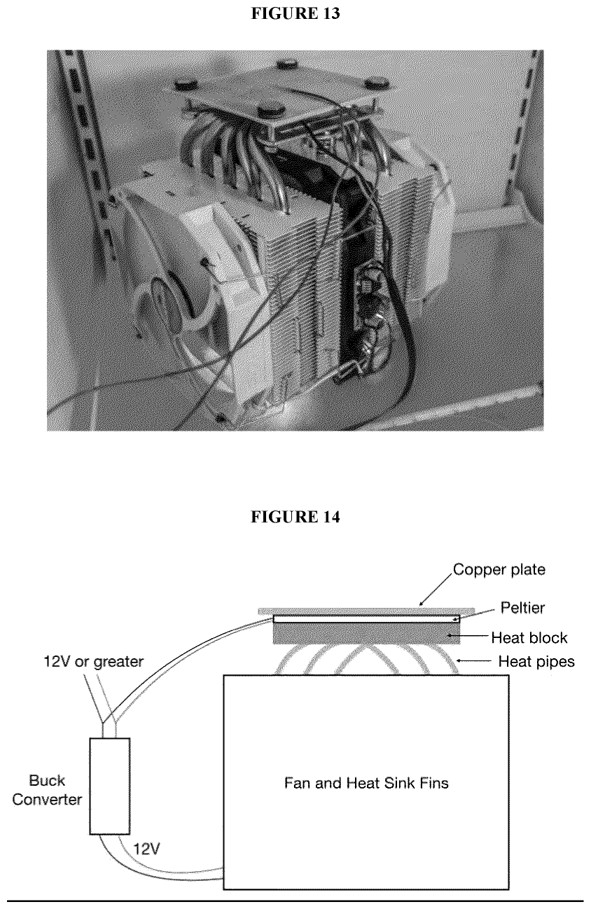

FIG. 13 shows an image of the custom-built directional freezing apparatus, and FIG. 14 shows a schematic view of the directional freezing apparatus. For the purposes of this study, the device itself was operated in the fridge as the peltier element will be able to reach lower temperatures when the ambient temperature is cooler. The device was allowed to cool and equilibrate for several hours. The copper plate reached an initial temperature of ˜5° C. As an initial test (no materials on the copper plate), power was supplied with a 12V/10A power supply. Within ˜15 min the temperature of the copper plate reached approx. −20° C. After one hour the plate equilibrated at approx. −25° C.

Mixing or Distributing of single structural cells, groups of structural cells, or both (obtained from mercerized decellularized plant tissue) with a hydrogel to provide a mixture:

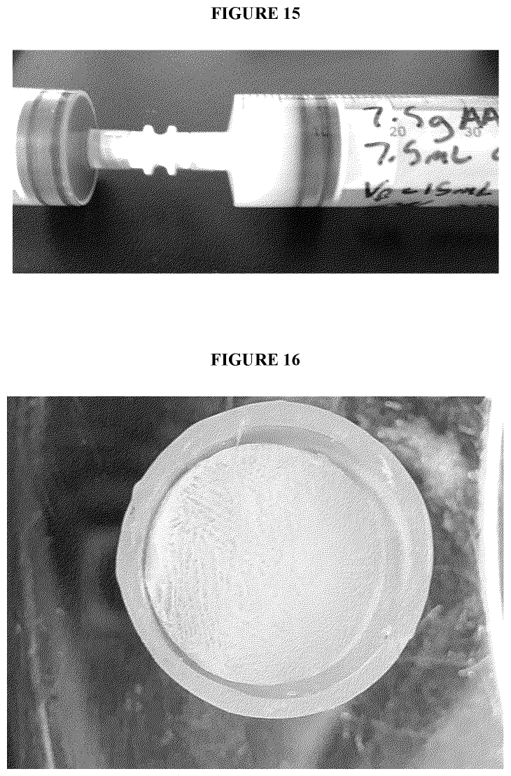

An alginate hydrogel was created by autoclaving alginate powder in dH₂O at a concentration of 5% (w/v). The final concentration of alginate was 1%. A composite biomaterial gel was produced comprising 7.5 g of mercerated apple (i.e. single structural cells, groups of structural cells, or both, obtained from mercerized decellularized apple tissue), 3 mL of 1% alginate, and 4.5 ml of water. The alginate hydrogel and the composite biomaterial gel were mixed using two 50 mL syringes connected with an f/f luer lock connector. The mixture was passed back and forth 30 times. Syringe mixing is shown in FIG. 15.

### Freezing and Dehydrating, Lyophilizing, or Freeze-Drying the Mixture to Provide Aerogels or Foams:

To create a vessel for directional freezing, the tips of 15 mL falcon tubes were cut off and the cap end was tightly sealed with a double layer of parafilm. This allowed the hydrogel mixture prepared above to be delivered from the top open end, while the bottom end is left on top of the copper plate. The parafilm layer ensured a very thin boundary between the cold surface and the gel. In this case ˜3-4 mL of hydrogel mixture was delivered into the tubes (˜3 cm high when standing vertical). The tubes were first allowed to cool in the fridge on the copper plate for at least an hour before the power was turned on. After cooling, the device was powered for two hours to allow time for the entire sample to freeze. Upon freezing, linear features could be seen in the frozen material; however, it was difficult to photograph them. After freezing, the tubes were immediately placed in a lyophilizer which operated for 36 hours. Upon removing the resultant aerogel samples, a porous biomaterial was observed. Multiple images were taken.

It was subsequently determined that an additional freezing was beneficial before lyophilization. Specifically, after freezing on the directional freezer, the samples were placed in a −20° C. freezer overnight to ensure the entire sample had frozen. The next day the samples were then lyophilized. This approach ensured that the resulting aerogels and foams did not collapse.

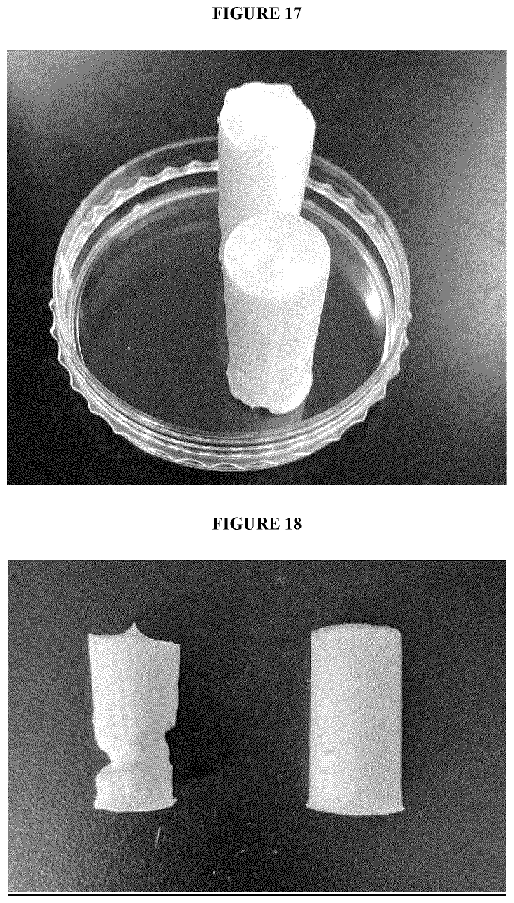

FIGS. 16-18 show images of resultant aerogels produced following lyophilisation. FIG. 16 shows a top-down view of the aerogel still in the falcon tube, and porous structures are observable. FIG. 17 shows an image of two aerogels following removal from falcon tubes. FIG. 18 shows aerogel obtained without performing additional freezing following directional freezing and before lyophilisation (left) in which the aerogel collapsed during lyophilisation, and aerogel which was subjected to additional freezing in a −20° C. freezer overnight after directional freezing and before lyophilisation, where collapse was not observed. The depicted scaffolds are about 3 cm tall.

### Results

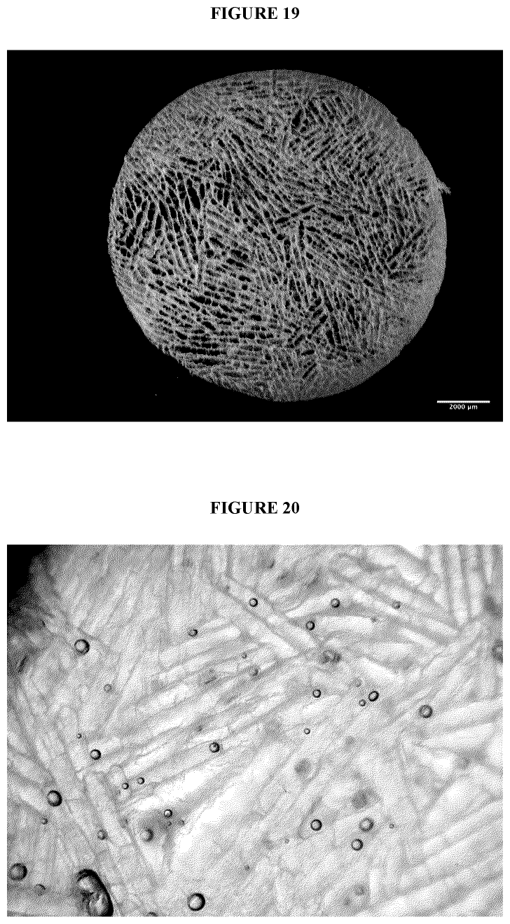

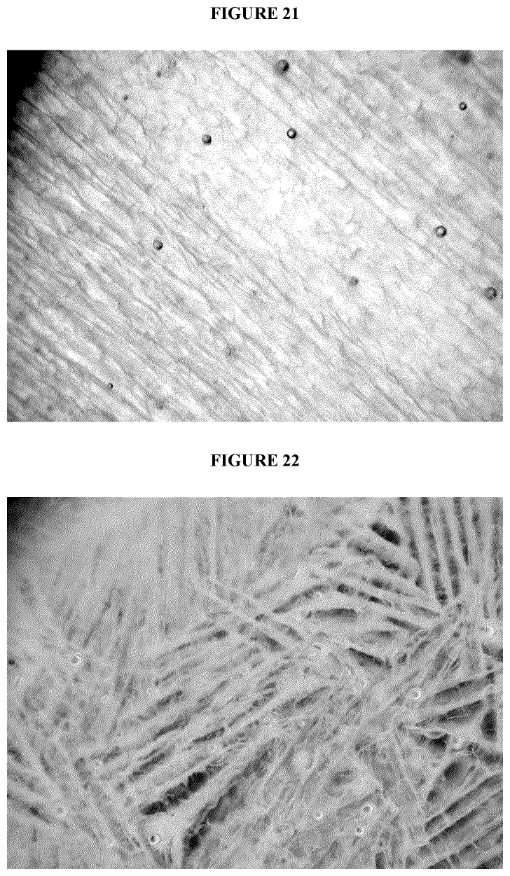

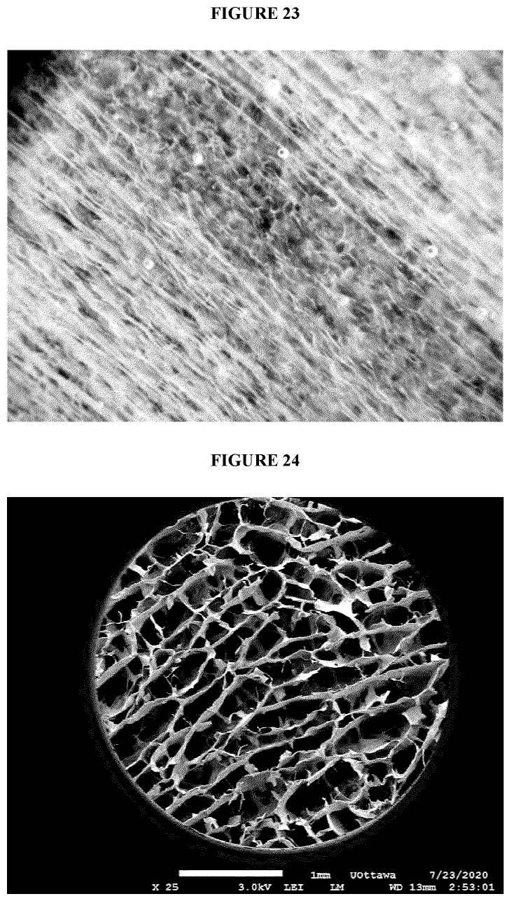

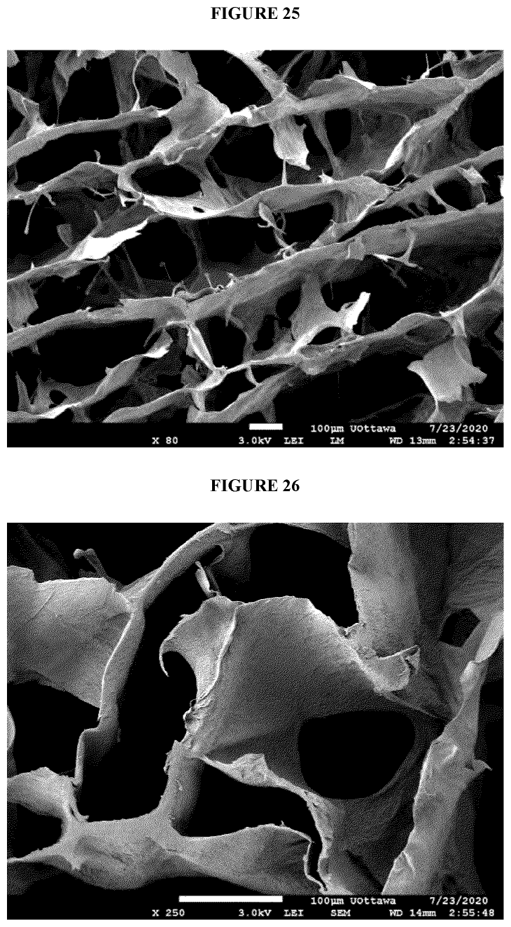

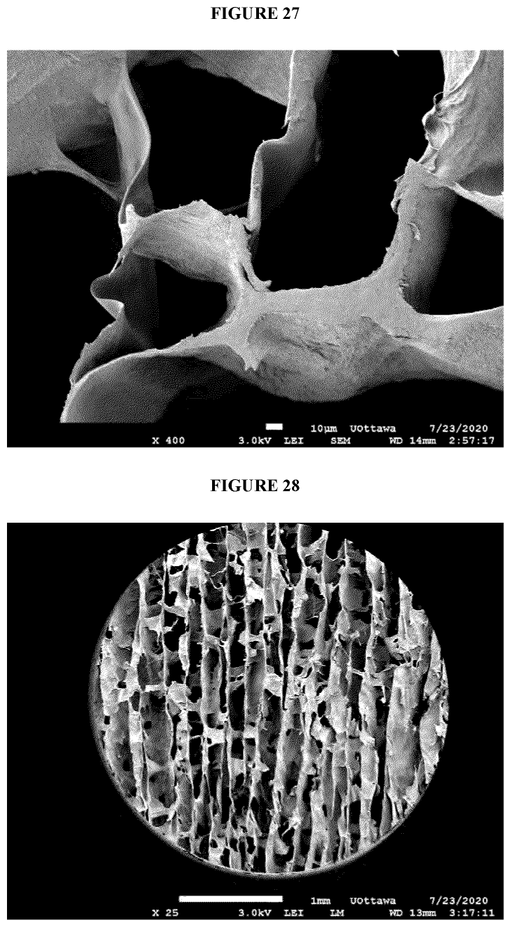

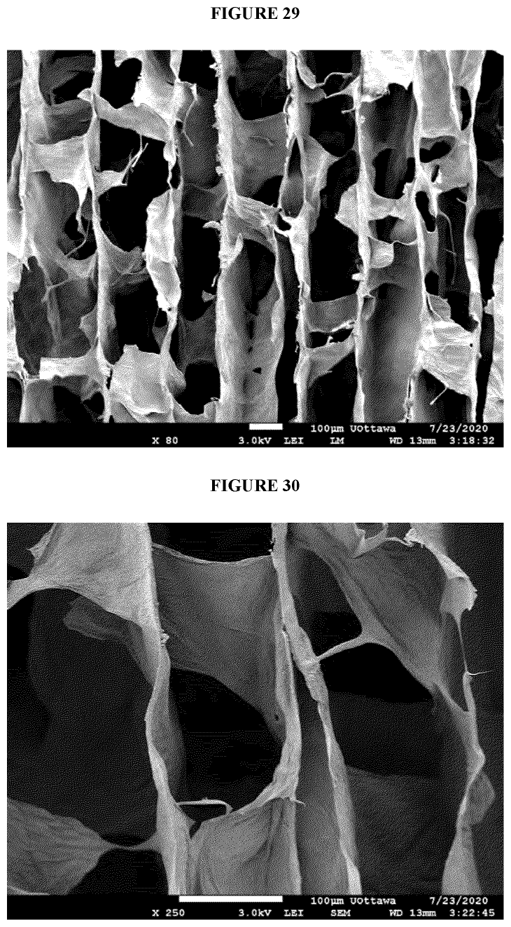

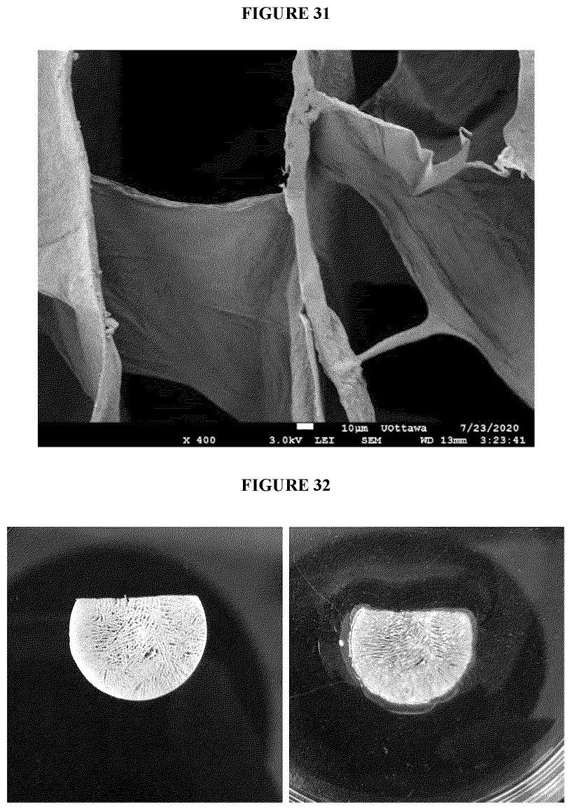

Optical microscopy and SEM was performed. The two scaffolds were sectioned either perpendicular or parallel to their long axis, and subsequently crosslinked/rehydrated in 0.1M CaCl₂), and imaged with optical microscopy or SEM. FIG. 19 shows a reflected light image of an entire aerogel cross section (1× condenser, 0.75× magnification). FIG. 20 shows brightfield cross-section perpendicular to the axis of the cylinder (Stereomicroscope 2× Condenser, 1.25 Zoom). FIG. 21 shows brightfield cross-section parallel to the axis of the cylinder (Stereomicroscope 2× Condenser, 1.25 Zoom). FIG. 22 shows darkfield cross-section perpendicular to the axis of the cylinder (Stereomicroscope 2× Condenser, 1.25 Zoom). FIG. 23 shows darkfield cross-section parallel to the axis of the cylinder (Stereomicroscope 2× Condenser, 1.25 Zoom). FIG. 24 shows SEM cross-section perpendicular to the axis of the cylinder, revealing microchannels. FIG. 25 shows SEM cross-section perpendicular to the axis of the cylinder, revealing microchannels. FIG. 26 shows SEM cross-section perpendicular to the axis of the cylinder. FIG. 27 shows SEM cross-section perpendicular to the axis of the cylinder. FIG. 28 shows SEM cross-section parallel to the axis of the cylinder, revealing long range alignment. FIG. 29 shows SEM cross-section parallel to the axis of the cylinder. FIG. 30 shows SEM cross-section parallel to the axis of the cylinder. FIG. 31 shows SEM cross-section parallel to the axis of the cylinder.

FIG. 32 shows images of a dry aerogel section (left) and 0.1M CaCl₂) treated rehydrated (right) aerogel section. Images were acquired at approximately the same height and magnification. The aerogel sections remained intact, maintained their microstructure, and could be picked up and manipulated. In this case, rehydration in CaCl₂) solution crosslinked and stabilized the alginate of the rehydrated aerogel (right).

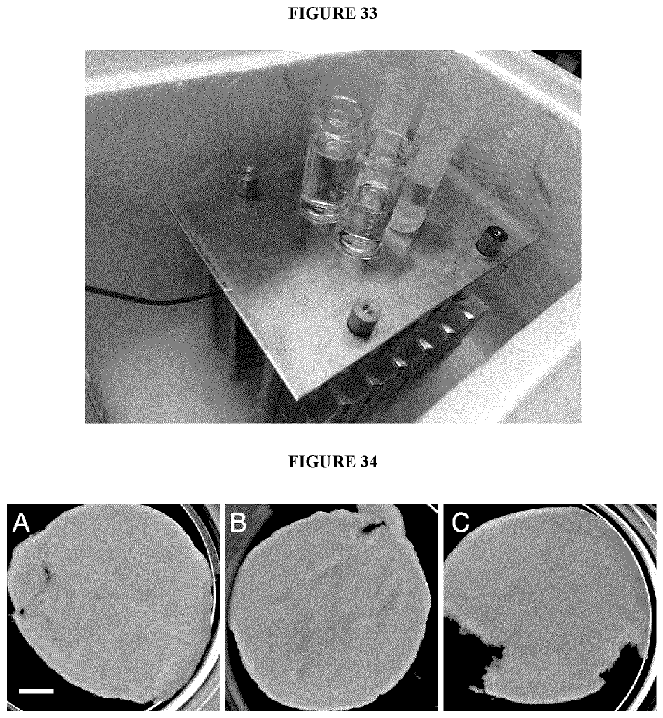

Other approaches to directional freezing were also attempted. Another device was custom-built in a manner almost identical to the device described above. However, the peltier element was removed along with all electronics and the fan. The passive directional freezer could then be placed in a styrofoam box and liquid nitrogen was poured into the box to cover the CPU cooler fins only. In this embodiment, the LN₂ pulls heat away from the copper surface on which the samples are mounted through the heat fins and heat pipes of the CPU cooler. The plate temperature reaches about −130° C. within several minutes and the samples freeze in about 15 mins compared to 2 hours on the original device. The freezing apparatus is depicted in FIG. 33.

FIG. 34 shows three formulations of aerogel that were prepared in which the solvent was either A) PBS, B) 0.9% saline, or C) water in order to assess if salts would alter ice crystal formation and channel alignment/architecture during directional freezing (scale bar=2 mm and applies to all). In all cases, the material froze so quickly and without significant ice crystal formation that aligned channels were not observed. A very dense and soft foam resulted from the process.

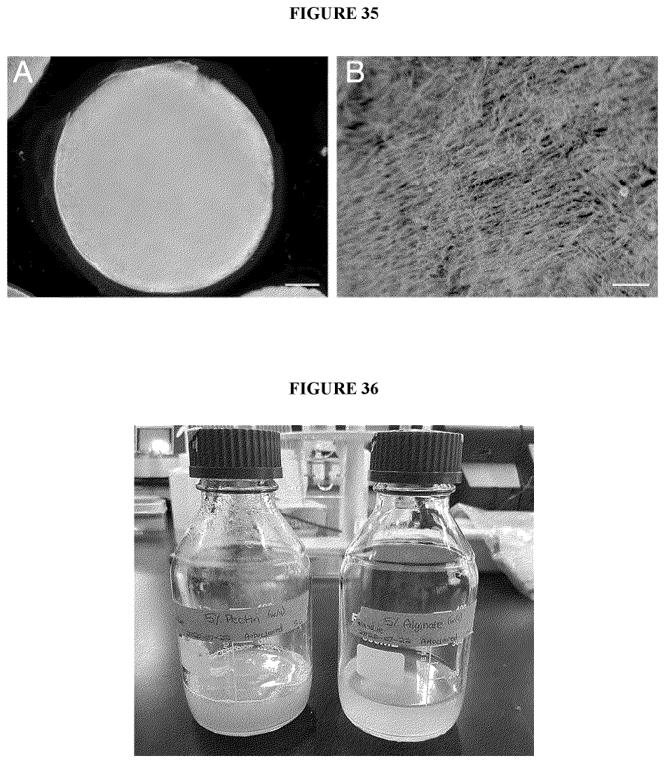

FIG. 35 shows (A) water-based alginate mixture which was directionally frozen on the LN₂ system (scale bar=2 mm). The scaffold was very dense and soft, and appeared homogeneous to the eye. This was in stark contrast to the scaffolds created on the peltier-based directional freezing platform described above in which the channeled architecture is clearly visible to the eye. As shown in (B), however, at high resolution (scale bar=200 um), the small pore size of the scaffold becomes visible which creates the opportunity for cell invasion and several potential other applications in tissue engineering and food science, for example.

Results indicate that the very rapid freezing due to the temperatures reached with LN₂ prevented the creation of large scale, long range, ice crystals and therefore prevented the organization of the hydrogel mixture into channeled, aligned structures. The results are dense, highly uniform aerogel scaffolds. It is contemplated that the amorphous and uniform scaffolds created in this manner may be useful in tissue engineering and food applications, for example. These results further expand the catalog of potential scaffold architectures, and provides additional tunability options, and provides material properties which may be used in a variety of applications.

Based on the results optioned herein, it is contemplated that directional freezing may be used to impart microstructures to aerogels, foams, and hydrogels as described herein which may provide for a variety of beneficial properties for a variety of different applications.

By way of example, directional freezing may be used to provide aerogels, foams, and/or hydrogels for use in spinal cord repair. In certain embodiments, it is contemplated that the aligned structures produced by controlled directional freezing (as in the first method above using the Peltier) may result in a scaffold which may be particularly well-suited for in spinal cord repair by providing biocompatible scaffolds with directional microchannels for aligning/directing spinal cord cells following implantation of the aerogel to promote healing. Such aerogel scaffolds may be produced in a scalable and controllable fashion.

By way of another example, directional freezing may be used to provide aerogels, foams, and/or hydrogels for use in a variety of food applications. It has further been observed that when the same hydrogel mixtures described above are placed into larger format containers (ex. 60 mm diameter petri dishes) which are shallower and wider than the falcon tubes, the long range alignments tend to occur parallel to the surface of the freezing plate. This was an unexpected result, which may be desirable for a number of applications. By way of example, in this case the creation of large, flat “sheets” of material with long range alignment parallel with the plane of the sheet may be desirable for applications in cultured and plant-based meats, for example. In these applications, it is contemplated that cells will align with the structures in the aerogel/hydrogel scaffolds to create cultured muscle tissues that more closely resemble real tissues. In addition, these highly structured scaffolds may also possess structural and mechanical properties similar to real meat, and/or may have value in the plant-only based meats. Furthermore, in certain embodiments it is contemplated that cultured and plant-only based scaffolds may be generated in which they are combined with real meat to provide a new class of alternative meat products which are part plant-based and part animal-based, for example.

By way of yet another example, it is contemplated that fine tuning of formulations used in directional freezing may provide additional control over resultant structural features in the aerogels/foams. By way of example, it is contemplated that the inclusion of various salts in the formulations may be used alter and potentially control the microarchitecture of the aligned structural features by augmenting ice crystal formation. It is also contemplated that channeled molds may be used to form the aligned structures around pillars which may be later removed from the scaffold to impart an array of channels with larger sizes. Alternatively, or in addition, it is contemplated that pressing needle arrays through the scaffolds may be used to create alternative channel sizes which complement the aligned structures from directional freezing, for example.

In this example, decellularized AA (apple) was produced according to a 4-day process and used as starting material. Wet decellularized AA was then broken down into a slurry of single, intact, AA structural cells in a 1-day liquid-based mercerization process. After a final centrifugation step, a clean, moist paste was isolated and used in further processing steps. The paste was malleable, but will hold its own shape (does not easily settle or flow, highly viscous). Material was white in colour. 30 apples produced ˜150 g of moist paste (95% water). Chemicals at the concentrations used were considered GRAS (SDS, NaOH, HCL, H₂O₂, CaCl₂), H₂O). Final product was designed to contain zero, or only trace amounts of any of those chemicals due to extensive washing and neutralization steps. Although not as extensively analyzed as apple, Pear and Asparagus also responded in a similar fashion as decellularized AA in the above process. It is contemplated that many different plant types may be used.

A variety of different aerogel, foam, and hydrogel products, and precursors thereof, may be prepared according to methods as described herein.

By way of example, the resultant product obtained from mercerization of the decellularized plant or fungal tissue may be provided, the product comprising single structural cells, groups of structural cells, or both, as described herein, and the product may be provided as a paste or gel, or may be provided as a dry sticky powder (when lyophilized or otherwise dried without a carrier hydrogel). Such products may be generally stable, may be sterilized with EtOH, or it is contemplated that such products may in certain embodiments be sterilized by gamma sterilization. In certain embodiments, such products may be mixed with other liquids or gels. Generally, such products did not readily dissolve in aqueous or alcohol-based solutions.

Aerogel products, or aerogel precursors, may also be provided. By way of example, in certain embodiments, the paste, gel, dry sticky powder, or other such products as described in the paragraph immediately above may be mixed with one or more (optionally food grade) hydrogels such as, but not limited to, Gelatin, Agar, Pluronic Acid, Alginate, Pectin, Methylcellulose (MC) and/or Carboxymethylcellulose (CMC) hydrogels, providing an aerogel precursor. In certain embodiments, the aerogel precursor products may comprise about 10% to about 50% (such as about 10%, about 20%, or about 50%) (m/m) of the paste, gel, dry sticky powder, or other such products as described in the paragraph immediately above, but other concentrations are also contemplated as this is controllable over a full range. The aerogel precursor may then be placed into any suitable size of container or mold, which will dictate its final size and thickness. Aerogel precursor products may be frozen (typically at −20° C. overnight), and then lyophilized (typically for at least about 24 hours), resulting in a highly porous dry aerogel or foam product. Controlling freezing temperature (for example, −20° C., −80° C., −130° C.) and formulation % (m/m) may allow for control over porosity of the resultant aerogels and foams. Results indicate control over porosity may be achieved from a level equivalent to the original AA scaffold and down. The 10% (m/m) formulations were very low porosity but very fragile, and so may be reserved for applications where fragility is not a concern. The 50% formulation provided the best experience for the user for most applications. Freezing method (directional vs non-directional) may be used to provide control over microarchitecture geometry (aligned-porous vs homogenous-porous), as desired for the particular application or product. Initial studies indicate that that solvent (e.g. DMSO vs H₂O) may also allow for control over porosity and microarchitecture, for example. Such products may be sterilized with EtOH, and it is contemplated that gamma sterilization may also be possible.

Additional products are also contemplated, such as rehydrated aerogels or foams as described herein to which liquid, such as water or an aqueous solution (such as a cell buffer) or another liquid or solution (such as an alcohol) have been introduced. In studies described herein, rehydration of aerogels and foams resulted in stable hydrogels with microarchitectures intact. Alginate and Pectin-based aerogels and foams could be rehydrated in CaCl₂) in order to provide crosslinking. Rehydrated aerogels and foams were stable under shaking in aqueous and ethanol based solutions for hours/days. Pectin-based aerogels and foams were not stable in 0.9% saline and underwent rapid degradation, however these were stable in PBS, H₂O and EtOH. Such rehydrated aerogels and foams have also been cell culture validated with NIH3T3 and C2Cl2 cells for up to at least 2 weeks in ongoing studies. Indeed, results indicate that rehydrated aerogels and foams as described herein are expected to behave similarly to decellularized plant-derived scaffold biomaterials as described in WO2017/136950 with respect to cell culture. Alginate and Gelatin based rehydrated aerogels and foams were superior to Pectin based aerogels and foams (which break down over time) under the conditions tested. Alginate and Pectin based rehydrated aerogels and foams are expected to be well-suited for implantation in vivo (for bone tissue engineering applications, for example). Such products may be sterilized with EtOH (for example, by 60 min shaking in EtOH), and it is contemplated that gamma sterilization may also be possible.

Results described herein indicate that aerogels and foams, and rehydrated aerogels and foams, as described herein may allow for control of surface biochemistry, particularly in that aerogels and foams, and rehydrated aerogels and foams, may be formulated with defined biochemistries (gelatin, alginate, pectin, MC, CMC, etc. . . . ) as desired. Various “plant-based” hydrocarbon polymers primarily composed of sugar may be used as hydrogel or carrier. Results also indicate that control over mechanics of aerogels, foams, rehydrated aerogels, and rehydrated foams may also be achieved. Aerogels, foams, rehydrated aerogels, and rehydrated foams as described herein may have controllable mechanical properties that may vary as a function of formulation %. In general the mechanical properties have been observed to vary from about 1 to about 200 kPa under the conditions tested. Exact values may depend on the hydrogel type and dry vs wet format/state of the aerogel or foam. An observed rule of thumb is that rehydrated aerogels and foams were about 10× softer than their dry aerogel or foam equivalent. Control over porosity may also be achieved. Results indicate that porosity may be controlled by altering the formulation %, freezing temperature, freezing method, and/or solvent used. An observed rule of thumb was that under the conditions tested porosity was either equivalent to original AA scaffolds (following decellularization, before further processing by mercerization, etc. . . . ), or may be decreased from there (i.e. higher density, lower pore sizes). Results indicate that control over size may also be achieved. The freezing container/mold for the hydrogel mixture prior to lyophilization and crosslinking dictated the final size of the resulting aerogel or foam product. It is additionally contemplated that sterilization may also be readily achieved. It is contemplated that generally all product types may be EtOH sterilized, and it is contemplated that gamma sterilization may also be possible. Results further indicate that rehydrated aerogels and foams as described herein may be suitable for in vitro cell culture. Cell culture was successful on alginate, pectin and gelatin based rehydrated aerogels and foams. Agar and pluronic acid based rehydrated aerogels and foams do not appear to be compatible with cell culture under the conditions tested so far, however this may also be beneficial for applications were cell growth is not desired, for example. Alginate based rehydrated aerogels and foams were the best performing products so far for in vitro cell culture applications under the conditions tested.

### Example 2-Additional Aerogels and Foams Prepared from Decellularized Plant Tissues

In this example a library of different aerogels and foams was prepared and tested, using various hydrogels and single structural cells and/or groups of structural cells obtained from decellularized plant tissue by mercerization treatment.

### Materials and Methods

Stock solutions:
5% Alginate stock solution:
   10 g Sodium Alginate (Modernist Pantry; LOT #14933)
   200 ml Distilled water
A. Weigh out 10 g of Sodium Alginate.
B. Mix powder into 200 mL of distilled water.
C. Autoclave solution for ˜1 hour to sterilize.
D. Keep warm in a water bath (37-50° C.) prior to usage.
5% Pectin stock solution:
   10 g Low Methoxyl Pectin (Modernist Pantry; LOT #14896)
   200 ml Distilled water
A. Weigh out 10 g of Low Methoxyl Pectin.
B. Mix powder into 200 mL of distilled water.
C. Autoclave solution for ˜1 hour to sterilize.
D. Keep warm in a water bath (37-50° C.) prior to usage.

Final 5% alginate and pectin stock solutions are shown in FIG. 36.
5% Agar stock solution:
   10 g Super Agar (Modernist Pantry; LOT #13198)
   200 ml Distilled water
A. Weigh out 10 g of Low Methoxyl Pectin.
B. Mix powder into 200 mL of distilled water.
C. Autoclave solution for ˜1 hour to sterilize.
D. Keep warm in a water bath (37-50° C.) prior to usage.
40% Pluronic stock solution:
   40 g Pluronic F-127 (Sigma Aldrich; LOT #BCCC2327)
   100 ml Distilled water
   Ice bath (around 0° C.)
A. Weigh out 40 g of Pluronic F-127.
B. Add 100 mL of distilled water to a 300 mL beaker and place in an ice bath until the water temperature is between 4-10° C.
C. Remove the beaker from the ice bath and place on a stir plate with a stir bar; slowly pour in the 40 g of Pluronic while stirring.
D. Continue to stir until the Pluronic powder has completely mixed into solution (no large chunks visible); ensure that the solution remains at a cool temperature by placing the beaker back into the ice bath frequently.
E. Keep cool (˜4-10° C.) prior to usage.

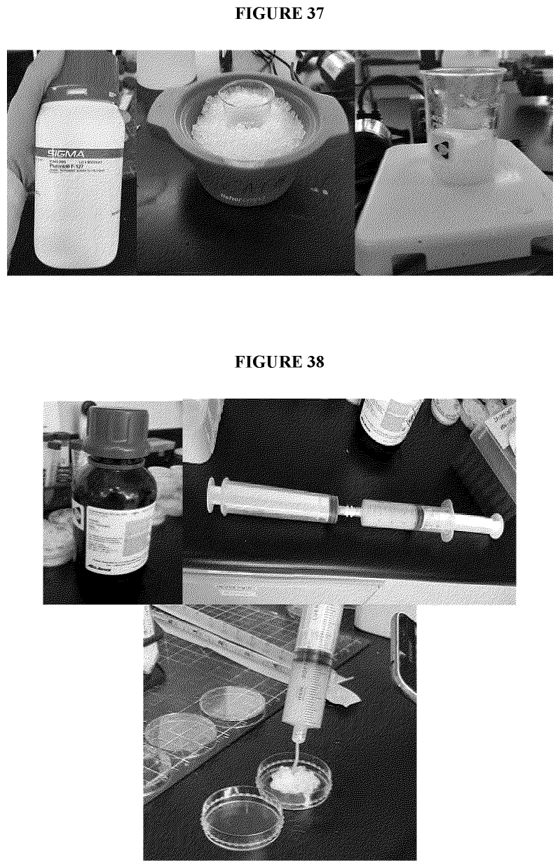

Pluronic stock solution preparation procedure is shown in FIG. 37.
Methylcellulose stock solution:
   Methylcellulose
   NaOH solution (Sodium hydroxide, extra pure, 50 wt % solution in water, Acros Organics, CAS 1310-73-2, LOT #A0408208)
   Glycine (Glycine, Fisher, BP381-1, CAS 56-40-6, LOT #121382)
A. Weigh 4 g of methylcellulose and mix it with 80 mL of 2M NaOH in a beaker.
B. Place the beaker containing the mixture in an ice-bath and stir for one hour.
C. Add 40 mL of 10% w/v glycine solution prepared in 2M NaOH to the sample.
D. Place the beaker again in an ice-bath and stir for one hour.
E. Collect the sample.
20% Gelatin stock solution:
   40 g Fish Gelatin Powder (250 Bloom; Modernist Pantry; LOT #14048)
   200 ml Distilled water
A. Weigh out 40 g of Low Methoxyl Pectin
B. Mix powder into 200 mL of distilled water
C. Autoclave solution for ˜1 hour to sterilize
D. Keep warm in water bath (37-50° C.) prior to usage
20% Pluronic stock solution:
   20 g Pluronic F-127 (Sigma Aldrich; LOT #BCCC2327)
   100 ml Distilled water
   Ice bath (around 0° C.)
A. Weigh out 20 g of Pluronic F-127.
B. Add 100 mL of distilled water to a 300 mL beaker and place in an ice bath until the water temperature is between 4-10° C.
C. Remove the beaker from the ice bath and place on a stir plate with a stir bar; slowly pour in the 20 g of Pluronic while stirring.
D. Continue to stir until the Pluronic powder has completely mixed into solution (no large chunks visible); ensure that the solution remains at a cool temperature by placing the beaker back into the ice bath frequently.
E. Keep cool (˜4-10° C.) prior to usage.

AA (apple) mercerization and neutralization:
NaOH solution (Sodium hydroxide, extra pure, 50 wt % solution in water, Acros Organics, CAS 1310-73-2, LOT #A0408208)
Hydrogen peroxide solution (Aqueous solution 30%, Fisher chemical, CAS 7722-84-1, LOT #197718)
Decellularized AA material
HCl solution for neutralization (Hydrochloric Acid solution 6N, Fisher scientific, CAS 7732-18-5, LOT #116660, Exp: 11/2013, dilution to 1N with dH₂O)
A. Remove excess water from decellularized AA samples by pressing the samples and weigh 100 grams of this material.
B. Prepare 500 mL of 1M NaOH and heat to 80° C. (300 RPM).
C. Add the AA samples to the NaOH solution and add 25 mL of H₂O₂ (maintain the solution to 80° C. throughout the mercerization).
D. After 20 minutes, add another 25 mL of H₂O₂.
E. After another 20 minutes, add another 25 mL of H₂O₂.
F. After 1 hour of mercerization, let the sample cool off. At this point, the AA samples are mercerized and the solution should be an off-white colour.
G. Neutralize (pH 7) the sample with a 1M HCl solution.
H. Centrifuge the solution at 5000 RPM for 15 minutes to pellet the mercerized AA.
I. Remove the supernatant. Verify that the pH is still at 7 by mixing the pelleted sample with dH2O and using the pH-meter. If the sample is not neutral, re-neutralize it and repeat step H.
J. Repeat steps H and I until the samples are neutralized. Keep the samples at 4° C. until further use for the aerogel preparation.

### Aerogel Preparation-Mixing, Freeze-Drying, Processing:

Alginate aerogels:
A. Weigh the appropriate mass of mercerized AA (according to Table 2 below) and mix in the appropriate volume of alginate stock solution and distilled water (final volume of 15 mL).
B. Place each AA sample solution (6 total: 3×1.5 g AA and 3×7.5 g AA) directly into a 60 mm culture dish.
C. Mix thoroughly with syringes connected with a luer lock connector to combine the AA with the gel.
D. Freeze the samples in a −20° C. freezer.
E. Mount the samples on the LabConco freeze-dryer for a 48 hours freeze-drying at 55 mBar and −47° C.
F. Remove the samples from the freeze-dryer after 48 hours.
G. With a 10 mm biopsy punch, cut samples from the freezed aerogels.
H. Place the punches in 0.1 M CaCl₂) for one hour for crosslinking.
I. Sterilize the samples 30 minutes in 70% ethanol.
J. Wash the aerogels 3 times with PBS, then place in DMEM media (10% FBS, 1% P/S). The aerogels that will be used for wet mechanical testing are placed in a 60 mm culture dish (5-6 samples) and the samples that will be used for cell seeding are placed in a 24-well plate (6 samples).
Pectin aerogels:
A. Weigh the appropriate mass of mercerized AA (according to Table 2 below) and mix in the appropriate volume of pectin stock solution and distilled water (final volume of 15 mL).
B. Place each AA sample solution (6 total: 3×1.5 g AA and 3×7.5 g AA) directly into a 60 mm culture dish.
C. Mix thoroughly with syringes connected with a luer lock connector to combine the AA with the gel.
D. Freeze the samples in a −20° C. freezer.
E. Mount the samples on the LabConco freeze-dryer for a 48 hours freeze-drying at 55 mBar and −47° C.
F. Remove the samples from the freeze-dryer after 48 hours.
G. With a 10 mm biopsy punch, cut samples from the freezed aerogels.
H. Place the punches in 0.1 M CaCl₂) for one hour for crosslinking.
I. Sterilize the samples 30 minutes in 70% ethanol.
J. Wash the aerogels 3 times with PBS, then place in DMEM media (10% FBS, 1% P/S). The aerogels that will be used for wet mechanical testing are placed in a 60 mm culture dish (5-6 samples) and the samples that will be used for cell seeding are placed in a 24-well plate (6 samples).
Agar aerogels:
A. Weigh the appropriate mass of mercerized AA (according to Table 2 below) and mix in the appropriate volume of agar stock solution and distilled water (final volume of 15 mL).
B. Place each AA sample solution (6 total: 3×1.5 g AA and 3×7.5 g AA) directly into a 60 mm culture dish.
C. Mix thoroughly with syringes connected with a luer lock connector to combine the AA with the gel.
D. Freeze the samples in a −20° C. freezer.
E. Mount the samples on the LabConco freeze-dryer for a 48 hours freeze-drying at 55 mBar and −47° C.
F. Remove the samples from the freeze-dryer after 48 hours.
G. With a 10 mm biopsy punch, cut samples from the freezed aerogels.
H. Place the punches in 0.1 M CaCl₂) for one hour for crosslinking.
I. Sterilize the samples 30 minutes in 70% ethanol.
J. Wash the aerogels 3 times with PBS, then place in DMEM media (10% FBS, 1% P/S). The aerogels that will be used for wet mechanical testing are placed in a 60 mm culture dish (5-6 samples) and the samples that will be used for cell seeding are placed in a 24-well plate (6 samples).
Pluronic aerogels:
A. Weigh 40 grams of Pluronic F-127 that will be used to achieve a final concentration of 40% (w/v) Pluronic solution.
B. Weigh the appropriate mass of mercerized AA (according to Table 2 below) and mix in the appropriate volume of distilled water.
C. Pour 100 mL of distilled water into a beaker and place in an ice bath until a temperature of 4-10° C. has been reached.
D. Once at the appropriate temperature, remove the beaker from the ice bath and place onto a stir plate; mix 40 g of Pluronic powder into the distilled water and stir well until there are no large chunks of Pluronic, ensuring that the Pluronic solution remains at a cool temperature.
E. Mix thoroughly with syringes connected with a luer lock connector to combine the AA with the gel.
F. After the solution has been thoroughly mixed, pour the required volume of 40% (w/v) Pluronic (as described in Table 2 below) into each of the 6 dishes.
G. Store all dishes into an incubator or oven for 1 hour at ˜37° C.
H. Finally, store all dishes in a −20° C. freezer to freeze for 1 hour, then mount on the freeze-dryer for lyophilization.
I. Note: final freeze-dried foams could not be sterilized due to disintegration in 70% EtOH, therefore were not used for cell culture.
Methylcellulose aerogels:
A. Weigh the appropriate mass of mercerized AA (according to Table 2 below) and mix in the appropriate volume of methylcellulose gel (final volume of approximately 15 mL).
B. Mix thoroughly with syringes connected with a luer lock connector to combine the AA with the gel.
C. Place each AA sample solution (6 total: 3×1.5 g AA and 3×7.5 g AA) directly into a 60 mm culture dish.
D. Let the sample incubate at room temperature for 24 hours.
E. Freeze the samples in a −20° C. freezer.
F. Mount the samples on the LabConco freeze-dryer for a 48 hours freeze-drying at 55 mBar and −47° C.
G. Remove the samples from the freeze-dryer after 48 hours.
H. With a 10 mm biopsy punch, cut samples from the freezed aerogels.
I. Note: final freeze-dried foams could not be sterilized due to disintegration in 70% EtOH, therefore were not used for cell culture.
Gelatin aerogels:
A. Weigh the appropriate mass of mercerized AA (according to Table 2 below) and mix in the corresponding volume of distilled water within a 15 mL Falcon tube.
B. Next, transfer the AA solution into a 50 mL plastic syringe and add the appropriate volume of 20% Gelatin stock solution (refer to Table 2) and ˜150 uL of Glutaraldehyde (GA) for crosslinking.
C. Attach a second, empty 50 ml syringe to the first one using a Luer Lock connector and transfer the total volume (˜15 mL) of AA solution back and forth (˜30 times) between the two syringes, mixing thoroughly.
D. Dispense the final AA solution (total volume of 15 mL) into a 60 mm culture dish and store in a 4° C. fridge for 24 hours.
E. Incubate with 10 mg/mL of sodium borohydride on ice for 1 h, then wash 3× with PBS.
F. Store in the −20° C. fridge for 4 hours.
G. Mount the samples on the LabConco freeze-dryer for 48 hours, freeze-drying at 55 mBar and −47° C.
H. Remove the samples from the freeze-dryer after 48 hours.
I. With a 10 mm biopsy punch, cut samples from the freezed aerogels.
J. Sterilize the samples 30 minutes in 70% ethanol.
K. Wash the aerogels 3 times with PBS, then place in DMEM media (10% FBS, 1% P/S). The aerogels that will be used for wet mechanical testing are placed in a 60 mm culture dish (5-6 samples) and the samples that will be used for cell seeding are placed in a 24-well plate (6 samples).

FIG. 38 shows preparation of gelatin-AA aerogel aerogels as described above.
Pluronic+Pectin aerogels:
   Making AA sample solution:
   A. Weigh out 7.5 g of mercerated AA; repeat until there are 3 total portions of 7.5 g AA.
   B. Prepare a stock solution of 5% Pectin (w/v) and a second 20% Pluronic stock solution.
   C. Add 4.5 mL of 5% Pectin to a syringe.
   D. Next, add 7.5 mL of 20% Pluronic stock solution, giving a total volume of 19.5 mL.
   E. Mix thoroughly with syringes connected with a luer lock connector to combine the AA with the gel.
   Making Pluronic-AA gels:
   G. Once all components have been mixed together in their respective culture dishes, store all dishes in an incubator or oven to sit at 37° C. for 1 hour.
   H. Remove dishes from the incubator and add 2 mL of 0.1M CaCl₂) to each dish for crosslinking; mix well and leave all dishes to gel at room temperature for 1 hour.
   I. Finally, store all dishes in a −80° C. freezer to thoroughly freeze for 2-3 days before mounting on the freeze-dryer to lyophilize.
   J. After lyophilization, remove foams from the freeze-dryer and cut out small foam aerogels with a 10 mm Biopsy Punch to use for dry mechanical testing.
   K. Note: final freeze-dried foams could not be sterilized due to disintegration in 70% EtOH, therefore were not used for cell culture.
Pluronic+Alginate aerogels:
Making AA sample solution:
A. Weigh out 7.5 g of mercerated AA; repeat until there are 3 total portions of 7.5 g AA.
B. Prepare a stock solution of 5% Alginate (w/v) and a second 20% Pluronic stock solution.
C. Place each portion of 7.5 g AA into a syringe.
D. Add 4.5 mL of 5% Alginate.
E. Next, add 7.5 mL of 20% Pluronic stock solution, giving a total volume of 19.5 mL.
F. Mix thoroughly with syringes connected with a luer lock connector to combine the AA with the gel.
Making Pluronic-AA gels:
G. Once all components have been mixed together in their respective culture dishes, store all dishes in an incubator or oven to sit at 37° C. for 1 hour.
H. Remove dishes from the incubator and add 2 mL of 0.1M CaCl₂) to each dish for crosslinking; mix well and leave all dishes to gel at room temperature for 1 hour.
I. Finally, store all dishes in a −80° C. freezer to thoroughly freeze for 2-3 days before mounting on the Labconco freeze-dryer. Leave the samples for 48 hours at 55 mBar and −47° C.
J. Remove the samples from the freeze-dryer after 48 hours.
K. With a 10 mm biopsy punch, cut samples from the freezed aerogels.
L. Place the punches in 0.1 M CaCl₂) for one hour for crosslinking.
M. Sterilize the samples 30 minutes in 70% ethanol.
N. Wash the aerogels 3 times with PBS, then place in DMEM media (10% FBS, 1% P/S). The aerogels that will be used for wet mechanical testing are placed in a 60 mm culture dish (5-6 samples) and the samples that will be used for cell seeding are placed in a 24-well plate (6 samples).

### Mixing

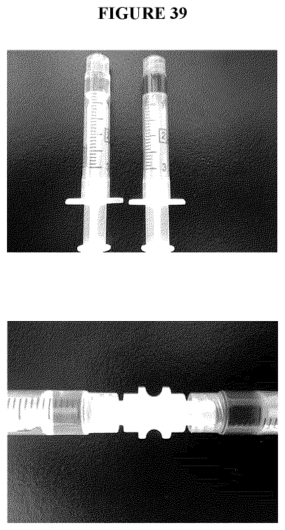

In order to make uniform mixtures of the mercerized material and the base hydrogel, the components were loaded into syringes connected by F/F luer lock connectors. The solutions were passed across the syringe 30×. A syringe-based mixing apparatus is shown in FIG. 39.

### Cell Culture

The sterilized and hydrated aerogels were placed in a 24-wells plate (1 sample per well) with 2 mL of DMEM. GFP 3T3 cells were cultured in 100 mm Petri dishes in DMEM media (10% FBS, 1% P/S) at 37° C., 5% CO2. The cells were washed with PBS and trypsinized with 0.25% trypsin. The cells were pelleted and resuspended in DMEM at a concentration of 2×10⁶ cells/mL. 25 μL of the cell resuspension were pipetted on each sterilized and hydrated aerogel, meaning each aerogel was seeded with 50,000 cells. After a 4 hour incubation at 37° C., 5% CO2, 2 mL of DMEM were added to each well containing a seeded aerogel and the plates were placed back in the incubator.

The aerogels were seeded again with GFP 3T3 cells using the same method described above after 7 days of incubation. After a total of two weeks since the first seeding (there has been two seedings—on day 1 and day 7), the cells were fixed on the aerogels. The samples were washed twice with 1 mL of PBS. The cells were incubated 10 minutes in 3.5% paraformaldehyde for fixation. The samples were again washed twice with 1 mL of PBS and stained with 0.1% Congo Red for 10 minutes. Finally, the samples were washed with PBS and stored in 2 mL of PBS at 4° C.

### Mechanical Testing

10 mm biopsy punches of all the different aerogels formulations have been mechanically tested (dry samples) with CellScale's Univert instrument. Additionally, the formulations that were seeded with GFP 3T3 cells were also mechanically tested wet with the same settings/compression cycle.

The samples were compressed at 90% of their height (1 repetition) during 20 seconds (stretch duration). 5 or 6 samples were mechanically tested per formulation.

### Results:

### AA Mercerization

About 30-40 g of mercerized AA material are obtained from the maceration of 100 g of wet decellularized AA.

Using the hydrogen peroxide during the mercerization rather than separating the mercerization and discolouring steps saved time (the same amount of mercerized AA material was obtained in about 4× less time). When combining the two steps at the same time, it takes about one hour to obtain the off-white mercerized material. FIGS. 1 and 2 described in Example 1 above show AA mercerization and discoloring, as well as starting material and resultant product from mercerization.

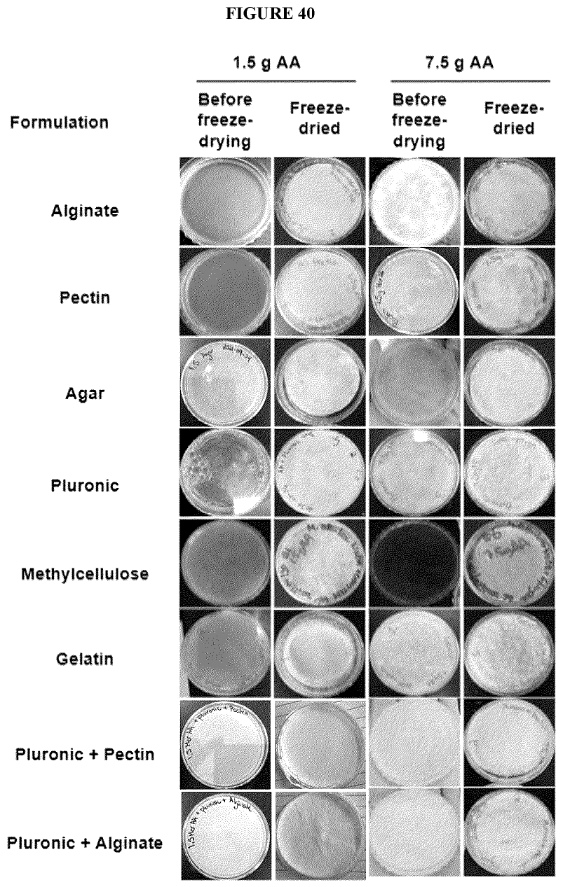

Table 2 shows various aerogel formulations that were prepared for the library in this Example. FIG. 40 depicts a representation of the different aerogel formulations that were prepared as part of the library produced in this example. aerogels are shown before and after freeze-drying of the samples. Table 3 shows a summary of cell culture results for the aerogels that were tested in this example.

TABLE 2
Various Aerogel Formulations Prepared for the Library

| | | | Volume of | | | |
|---|---|---|---|---|---|---|
| Mass of | | Stock gel | stock gel | Volume of | Final | |
| mercerized | Type | conc. | solution | dH₂O | volume | |
| AA (g) | of gel | (% m/v)) | (mL) | (mL) | (mL) | Crosslinker |
| 1.5 | Alginate | 5 | 3 | 10.5 | 15 | 0.1M CaCl₂ |
| 7.5 | Alginate | 5 | 3 | 4.5 | 15 | 0.1M CaCl₂ |
| 1.5 | Pectin | 5 | 3 | 10.5 | 15 | 0.1M CaCl₂ |
| 7.5 | Pectin | 5 | 3 | 4.5 | 15 | 0.1M CaCl₂ |
| 1.5 | Agar | 5 | 3 | 10.5 | 15 | thermos |
| 7.5 | Agar | 5 | 3 | 4.5 | 15 | thermos |
| 1.5 | Pluronic | 40 | 7.5 | 6 | 15 | thermos |
| 7.5 | Pluronic | 40 | 7.5 | 0 | 15 | thermos |
| 1.5 | Methyl | 3.33 | ~13.5 | 0 | 15 | glycine |
| | cellulose | | | | | |
| 7.5 | Methyl | 3.33 | ~7.5 | 0 | 15 | glycine |
| | cellulose | | | | | |
| 1.5 | Gelatin | 20 | 3.75 | 9.6 | 15 | GA |
| 7.5 | Gelatin | 20 | 3.75 | 3.6 | 15 | GA |
| 1.5 | Pluronic + | 40 (Pluronic), | 7.5 (Pluronic), | 3 | 15 | 0.1M CaCl₂ |
| | Pectin | 5 (Pectin) | 3 (Pectin) | | | |
| 1.5 | Pluronic + | 40 (Pluronic), | 7.5 (Pluronic), | 3 | 15 | 0.1M CaCl₂ |
| | Alginate | 5 (Alginate) | 3 (Alginate) | | | |
| 7.5 | Pluronic + | 20 (Pluronic), | 7.5 (Pluronic), | 0 | 19.5 | 0.1M CaCl₂ |
| | Pectin | 5 (Pectin) | 4.5 (Pectin) | | | |
| 7.5 | Pluronic + | 20 (Pluronic), | 7.5 (Pluronic), | 0 | 19.5 | 0.1M CaCl₂ |
| | Alginate | 5 (Alginate) | 4.5 (Alginate) | | | |

TABLE 3
Summary of Cell Culture Results

| Mass of mercerized AA (g) | Type of gel | Cell Growth (Y, S, N, N/A) |
|---|---|---|
| 1.5* | Alginate | Y |
| 7.5** | Alginate | Y |
| 1.5*** | Pectin | Y |
| 7.5** | Pectin | Y |
| 1.5* | Agar | S or N |
| 7.5**** | Agar | S or N |
| 1.5 | Pluronic | N/A |
| 7.5 | Pluronic | N/A |
| 1.5 | Methyl cellulose | N/A |
| 7.5 | Methyl cellulose | N/A |
| 1.5 | Gelatin | Y |
| 7.5 | Gelatin | Y |
| 7.5 | Pluronic + Pectin | N/A |
| 7.5 | Pluronic + Alginate | S or N |

*5 pucks were imaged
**6 pucks were imaged
***1 puck was imaged
****3 pucks were imaged

The agar and pectin (1.5) aerogels were very fragile once hydrated and they crumbled into smaller pieces.

Some formulations were not well suited for cell growth as they were either too fragile; they crumbled once hydrated; or they disintegrated during the sterilization step. This was the case for the pluronic, methylcellulose, and pluronic+pectin aerogels. Accordingly, these staples were not seeded with GFP 3T3 cells.

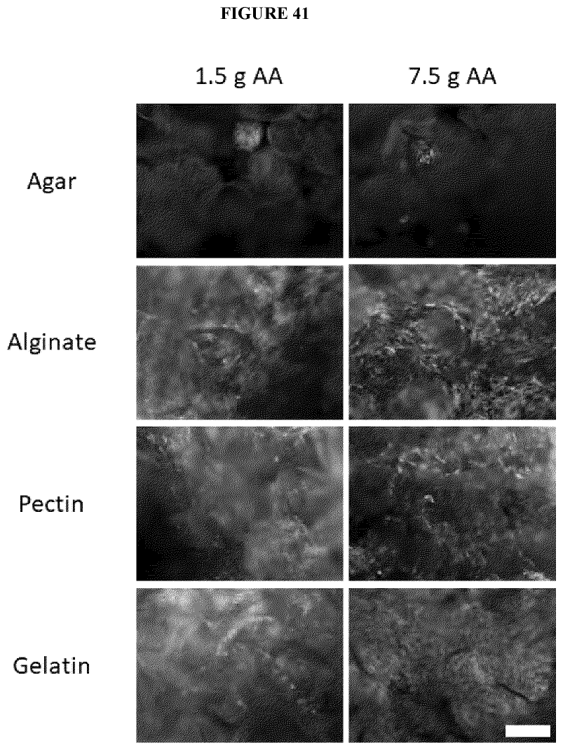

FIG. 41 shows results in which GFP 3T3 cells (green) were seeded onto certain aerogel aerogels (as shown) stained with Congo Red (red). Agar, alginate, pectin, and gelatin hydrogels were used in combination with 1.5 g of decellularized, mercerized apple (10%) or 7.5 g of decellularized, mercerized apple (50%) (Scale-200 μm). Images were acquired on the BX53 upright microscope at 10× with the GFP filter for the cells and the TXRED filter for the scaffold.

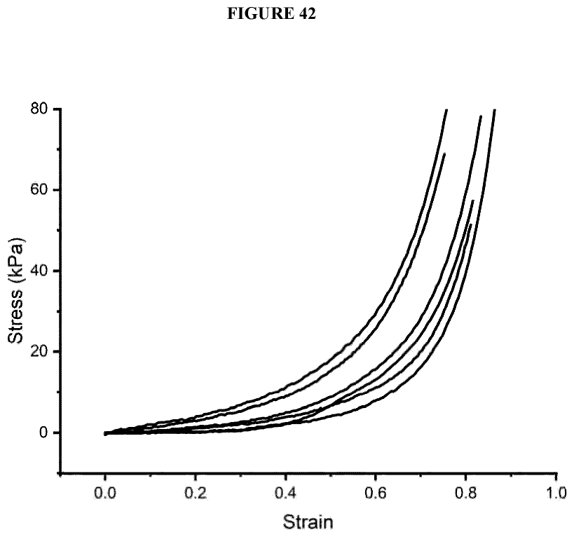

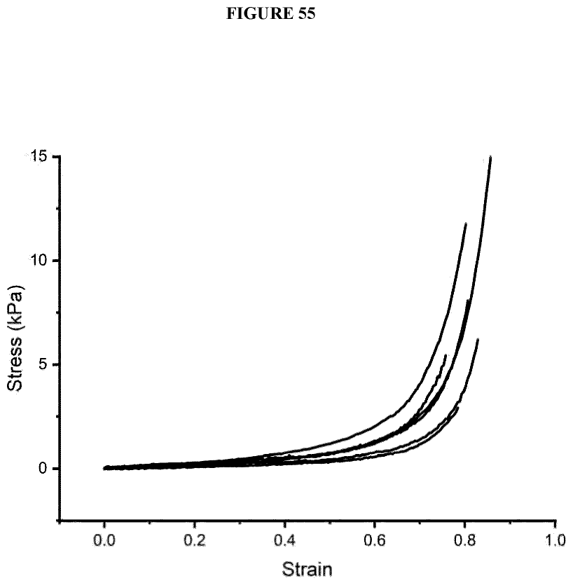

Results for mechanical testing of dry aerogel samples are shown in FIGS. 42-54, and results for mechanical testing of hydrated aerogel samples are show in FIGS. 55-64, as follows:

FIG. 42 shows stress-strain curves for the dry agar based gels with 1.5 g of mercerized AA;

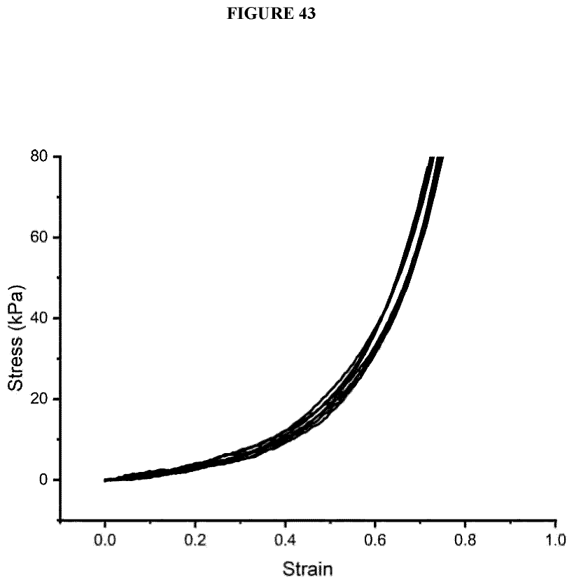

FIG. 43 shows stress-strain curves for the dry agar based gels with 7.5 g of mercerized AA;

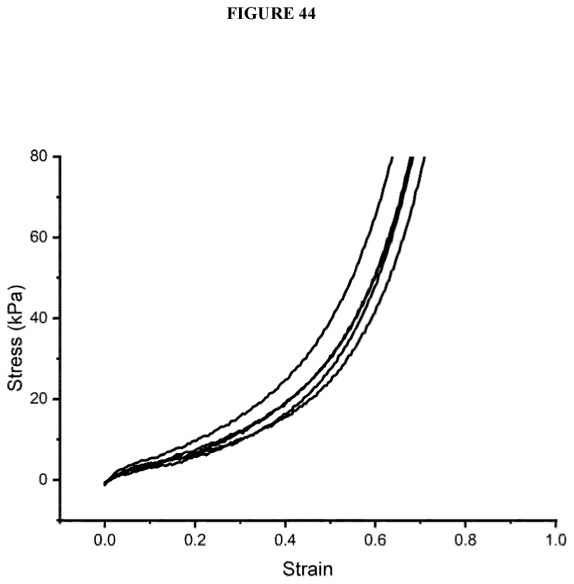

FIG. 44 shows stress-strain curves for the dry alginate based gels with 1.5 g of mercerized AA;

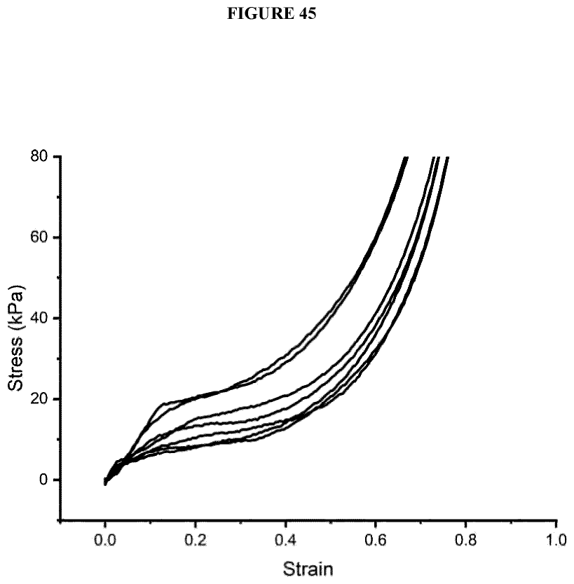

FIG. 45 shows stress-strain curves for the dry alginate based gels with 7.5 g of mercerized AA;

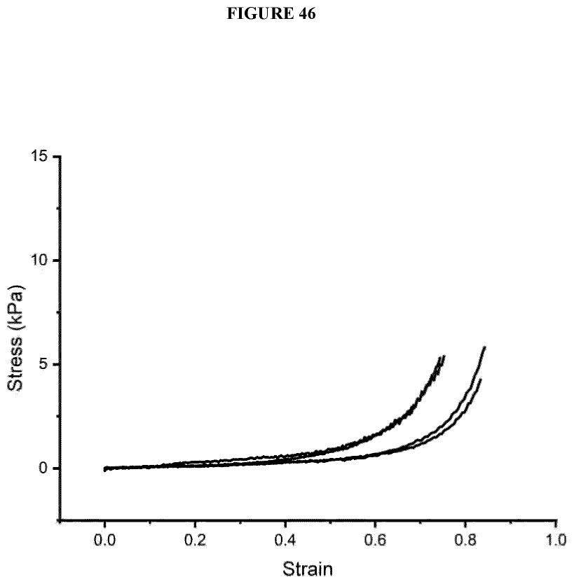

FIG. 46 shows stress-strain curves for the dry pectin based gels with 1.5 g of mercerized AA;

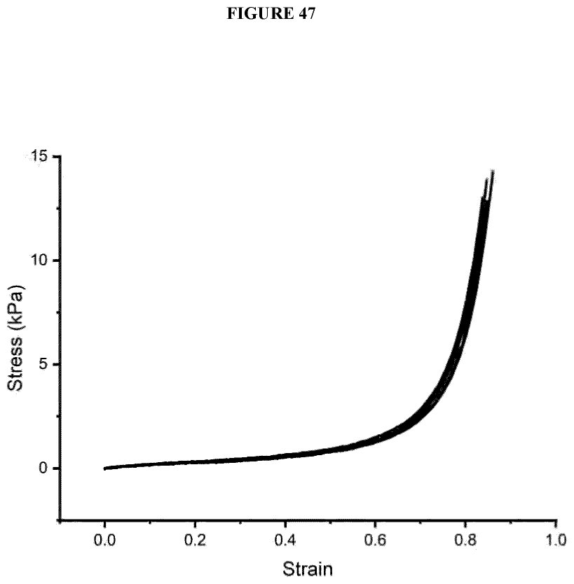

FIG. 47 shows stress-strain curves for the dry pectin based gels with 7.5 g of mercerized AA;

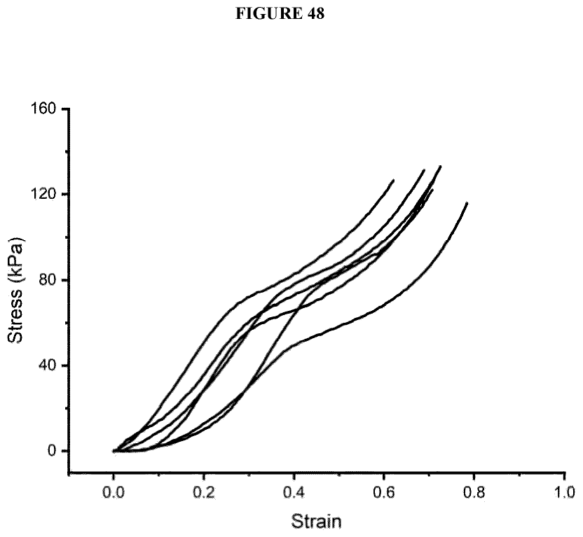

FIG. 48 shows stress-strain curves for the dry gelatin based gels with 1.5 g of mercerized AA;

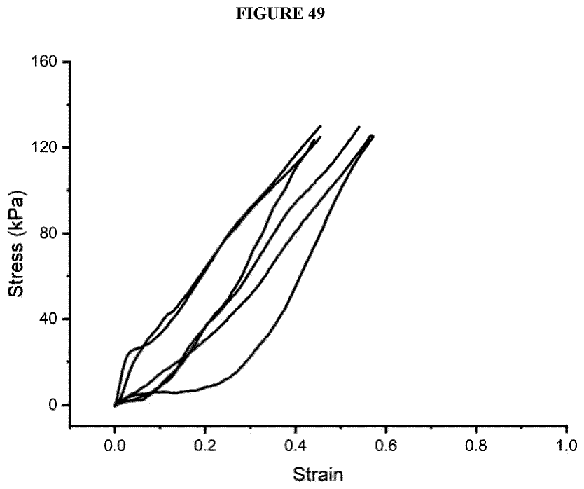

FIG. 49 shows stress-strain curves for the dry gelatin based gels with 7.5 g of mercerized AA;

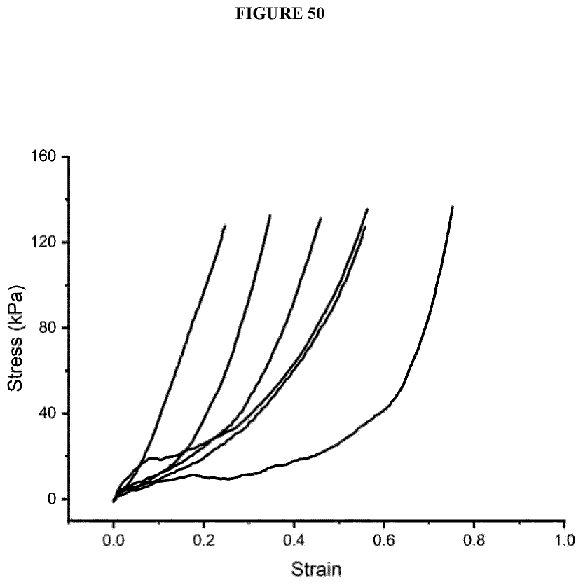

FIG. 50 shows stress-strain curves for the dry methylcellulose based gels with 1.5 g of mercerized AA;

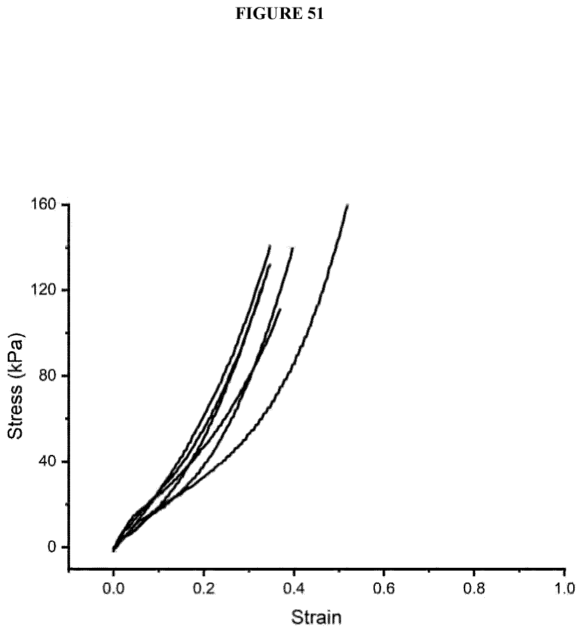

FIG. 51 shows stress-strain curves for the dry methylcellulose based gels with 7.5 g of mercerized AA;

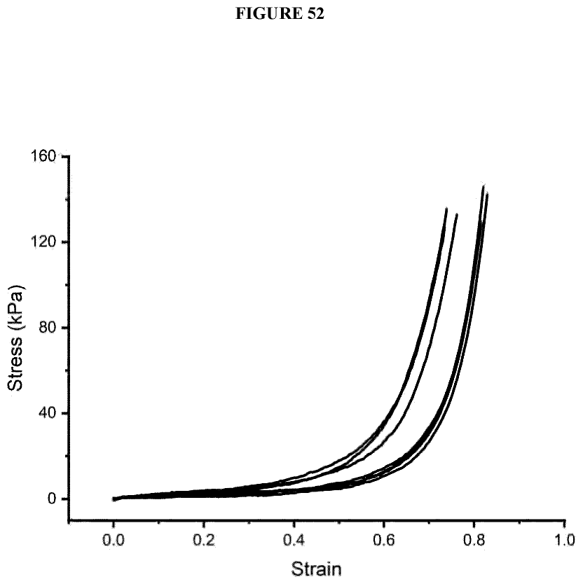

FIG. 52 shows stress-strain curves for the dry pluronic based gels with 1.5 g of mercerized AA;

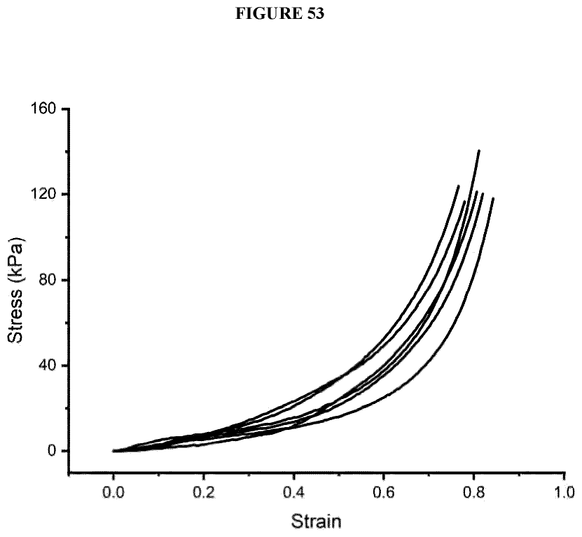

FIG. 53 shows stress-strain curves for the dry pluronic and alginate based gels with 7.5 g of mercerized AA;

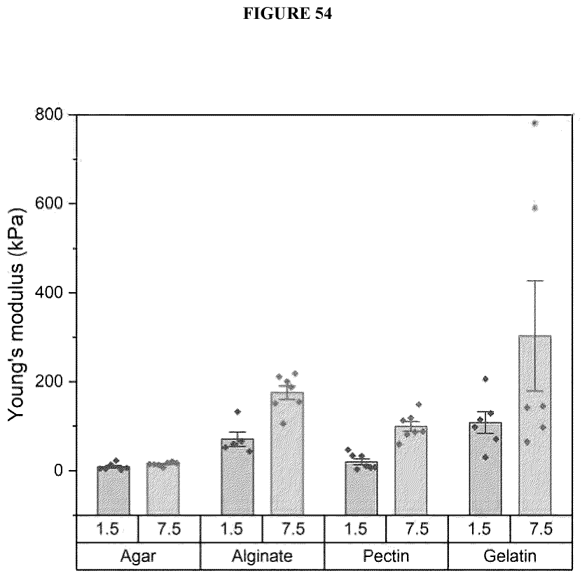

FIG. 54 shows Young's moduli for the dry samples that have a hydrate counterpart. The volumes of the mercerized AA are indicated by 1.5 and 7.5; both are in grams and correspond to a 10% and 50% solution respectively. The base hydrogels of 1% agar, alginate and pectin were used. Gelatin was a 5% final solution;

FIG. 55 shows stress-strain curves for the hydrated agar based gels with 1.5 g of mercerized AA;

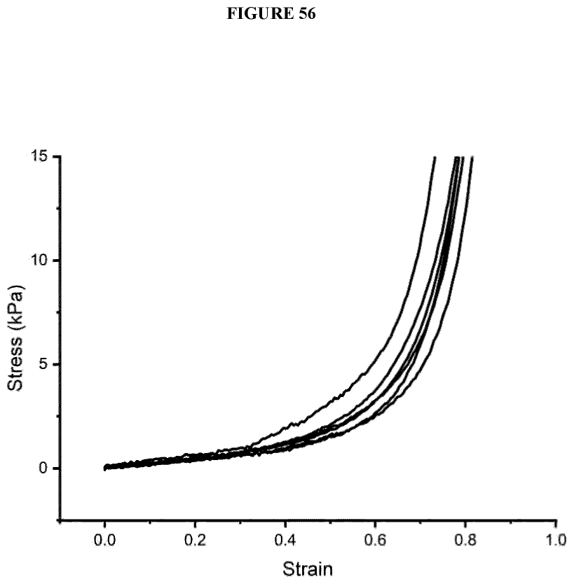

FIG. 56 shows stress-strain curves for the hydrated agar based gels with 7.5 g of mercerized AA;

FIG. 57 shows stress-strain curves for the hydrated alginate based gels with 1.5 g of mercerized AA;

FIG. 58 shows stress-strain curves for the hydrated alginate based gels with 7.5 g of mercerized AA;

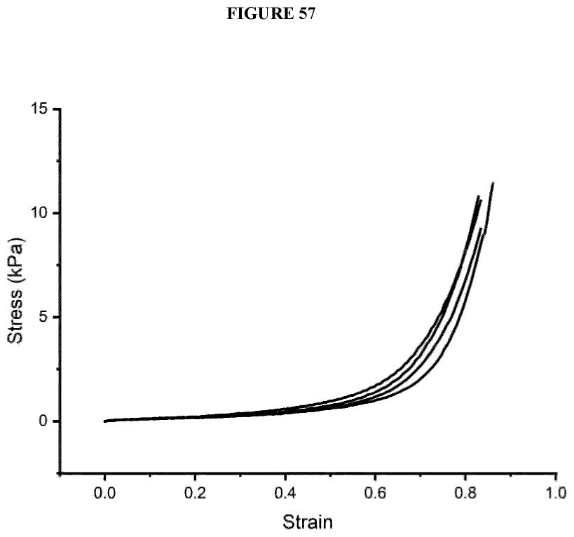

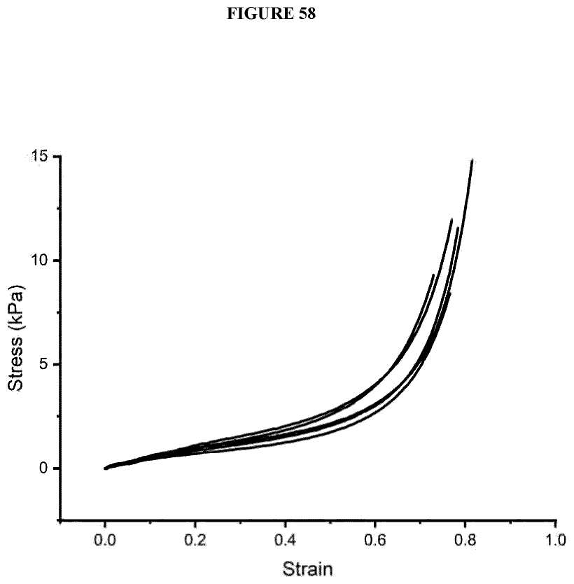

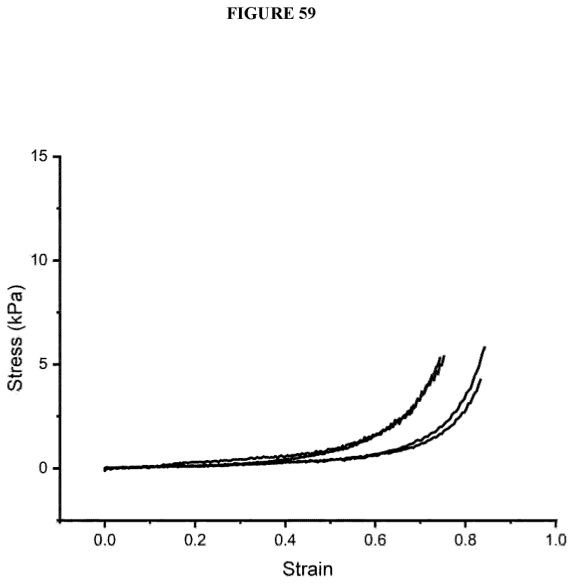

FIG. 59 shows stress-strain curves for the hydrated pectin based gels with 1.5 g of mercerized AA;

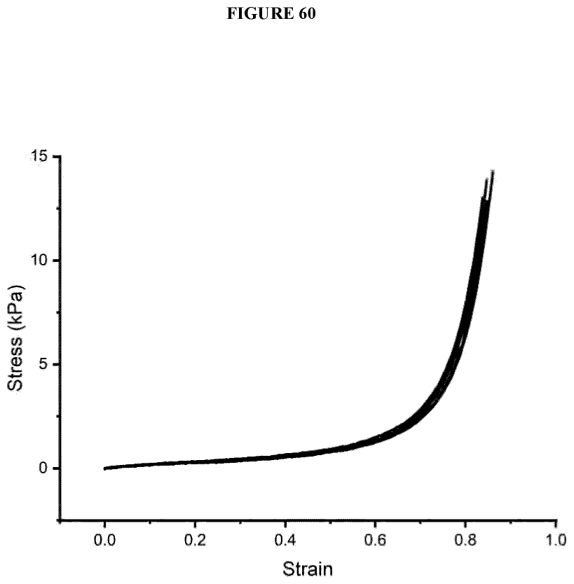

FIG. 60 shows stress-strain curves for the hydrated pectin based gels with 7.5 g of mercerized AA;

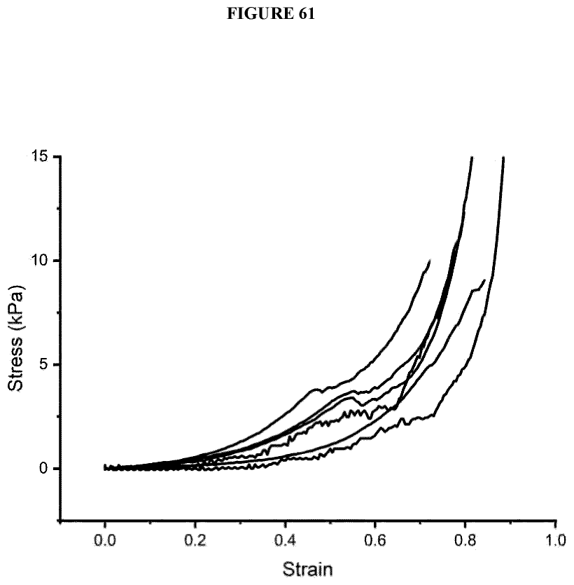

FIG. 61 shows stress-strain curves for the hydrated gelatin based gels with 1.5 g of mercerized AA;

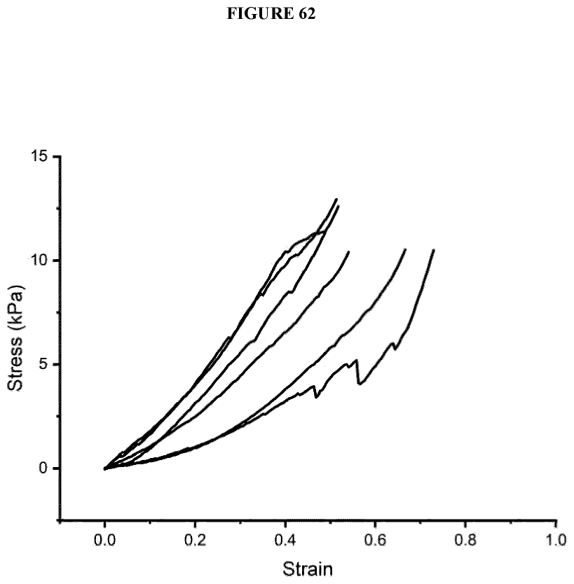

FIG. 62 shows stress-strain curves for the hydrated gelatin based gels with 7.5 g of mercerized AA;

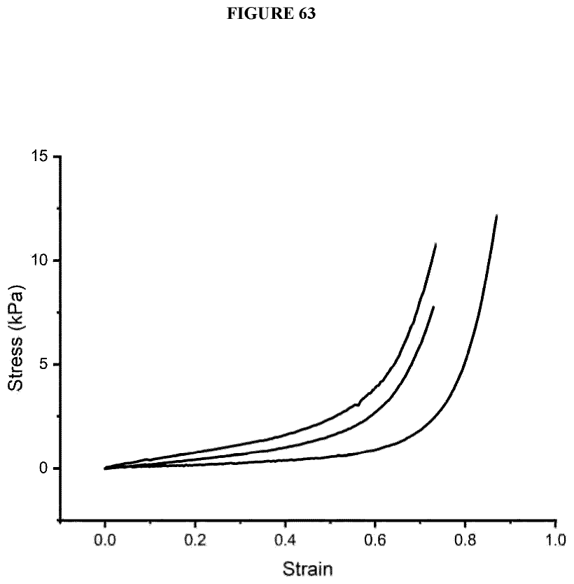

FIG. 63 shows stress-strain curves for the hydrated pluronic and alginate based gels with 7.5 g of mercerized AA; and

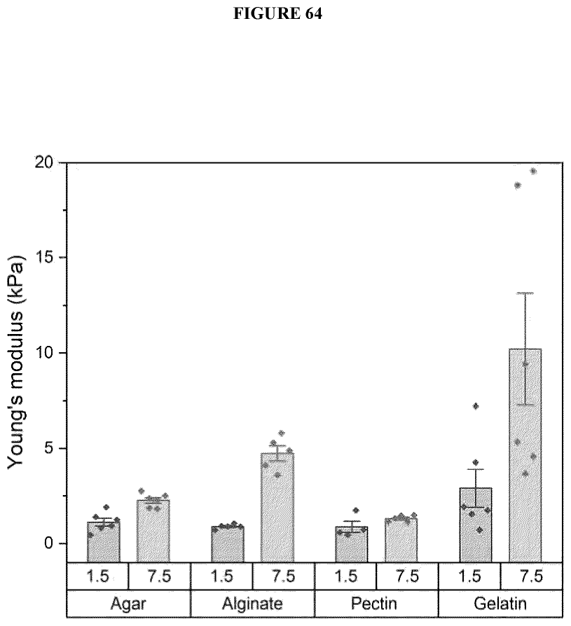

FIG. 64 shows Young's moduli for the hydrated samples. The volumes of the mercerized AA are indicated by 1.5 and 7.5; both are in grams and correspond to a 10% and 50% solution respectively. The base hydrogels of 1% agar, alginate and pectin were used. Gelatin was a 5% final solution.

Results in FIGS. 42-64 show that mechanical properties of the material may be controlled. These results also show bi-modal mechanical properties, in which the stiffness increases between a lower value at small strain and a higher value at high strain (i.e. the mechanical properties change during compression). Ability to have different regimes is of interest. The linear elastic regime is of interest; however, the mechanics of the different plastic regimes and the failure points may be of greater interest for certain applications, such as for food applications and tailored mouth feel, for example.

### Swelling

To determine the significance of swelling on aerogel size, the approximate diameter of aerogels used for dry and wet mechanical testing were measured. The effects of liquid retention were observed after foam aerogels were stored in DMEM in an incubator for several days. Diameter data was not obtained for all aerogel formulations due to some aerogel types disintegrating during sterilization (i.e., methylcellulose and Pluronic). Dry and wet diameter measurements were obtained for agar, alginate, and pectin AA formulation aerogel. From these measurements, the approximate area, height and volume of AA aerogels was determined and shown below in Tables 4, 5, and 6.

For statistical analysis, t-tests were performed between each aerogel type's dry and wet measurements. With the exception of alginate and gelatin aerogels (both 1.5 g and 7.5 g conditions), all aerogel area measurements decreased in size once wet, likely due to the samples somewhat breaking down and losing some stability in liquid. An analysis of variance (ANOVA) was also performed to determine the significance of interactions between gel type and AA concentration (i.e., 1.5 g AA and 7.5 g AA). Overall, there was no significant difference observed between AA concentrations used for the aerogels. When comparing gel type, however, there was a significant difference.

For aerogel height, the alginate and gelatin formulations were the only AA aerogel types to demonstrate significant increase due to aerogel swelling after submersion in DMEM. An increase in volume was similarly observed with the alginate and gelatin aerogels as well after hydration; the remaining aerogel formulations all demonstrated a significant decrease in aerogel volume once wet, likely due to some aerogel degradation. Furthermore, an ANOVA for the height revealed a significant difference between the different concentrations, indicating that the freeze-drying process or the variability in the filling method may influence aerogel height in some way. Moreover the gel type and interaction effects were also significant at the 0.05 level. These findings can be observed below in Tables 4, 5, and 6.

TABLE 4
Approximate area measurements of several AA aerogel types used for
dry and wet mechanical testing. T-test results are demonstrated
below as p-values, where the threshold for significance is <0.05

| | | Mean | Standard | Standard |
|---|---|---|---|---|
| | | Area | Deviation | Error of |
| | | (mm) | (SD) | the Mean (SEM) |
| Agar 1.5 g AA | Dry | 124.86 | 25.820 | 10.541 |
| | Hydrated | 46.652 | 20.074 | 8.1950 |
| | p Value | 0.00020157 | | |
| Agar 7.5 g AA | Dry | 113.01 | 10.520 | 3.9760 |
| | Hydrated | 28.298 | 9.6680 | 3.9470 |
| | p Value | 0.000000011400 | | |
| Alginate 1.5 g AA | Dry | 82.112 | 6.8154 | 3.0480 |
| | Hydrated | 88.278 | 19.505 | 8.7227 |
| | p Value | 0.53437 | | |
| Alginate 7.5 g AA | Dry | 75.720 | 5.7165 | 2.1606 |
| | Hydrated | 92.010 | 21.048 | 9.4128 |
| | p Value | 0.16004 | | |
| Pectin 1.5 g AA | Dry | 81.164 | 11.919 | 4.5050 |
| | Hydrated | 37.136 | 5.7964 | 2.8982 |
| | p Value | 0.000018642 | | |
| Pectin 7.5 g AA | Dry | 80.688 | 2.4705 | 0.93377 |
| | Hydrated | 74.508 | 4.2650 | 1.9073 |
| | p Value | 0.0274 | | |
| Gelatin 1.5 g AA | Dry | 79.180 | 4.1013 | 1.6743 |
| | Hydrated | 93.866 | 11.102 | 4.5322 |
| | p Value | 0.0213 | | |
| Gelatin 7.5 g AA | Dry | 78.983 | 1.6822 | 0.68675 |
| | Hydrated | 88.096 | 8.2013 | 3.3482 |
| | p Value | 0.0411 | | |

TABLE 5
Approximate height measurements of several AA aerogel types used
for dry and wet mechanical testing. T-test results are demonstrated
below as p-values, where the threshold for significance is <0.05

| | | Mean | Standard | Standard |
|---|---|---|---|---|
| | | Height | Deviation | Error of |
| | | (mm) | (SD) | the Mean (SEM) |
| Agar 1.5 g AA | Dry | 3.418 | 0.8267 | 0.3375 |
| | Hydrated | 3.002 | 0.8155 | 0.3329 |
| | p Value | 0.4001 | | |
| Agar 7.5 g AA | Dry | 4.817 | 0.5564 | 0.2103 |
| | Hydrated | 3.825 | 0.4229 | 0.1726 |
| | p Value | 0.003907 | | |
| Alginate 1.5 g AA | Dry | 2.851 | 0.1786 | 0.07986 |
| | Hydrated | 4.262 | 0.6963 | 0.3114 |
| | p Value | 0.008919 | | |
| Alginate 7.5 g AA | Dry | 6.264 | 0.5252 | 0.1985 |
| | Hydrated | 5.949 | 0.8409 | 0.3760 |
| | p Value | 0.4867 | | |
| Pectin 1.5 g AA | Dry | 5.437 | 0.3733 | 0.1411 |
| | Hydrated | 2.630 | 0.6745 | 0.3372 |
| | p Value | 0.001429 | | |
| Pectin 7.5 g AA | Dry | 5.985 | 0.3315 | 0.1253 |
| | Hydrated | 5.511 | 0.7656 | 0.3424 |
| | p Value | 0.2489 | | |
| Gelatin 1.5 g AA | Dry | 5.007 | 0.9632 | 0.3932 |
| | Hydrated | 5.792 | 0.6963 | 0.2843 |
| | p Value | 0.1397 | | |
| Gelatin 7.5 g AA | Dry | 6.919 | 1.213 | 0.4950 |
| | Hydrated | 7.178 | 1.369 | 0.5587 |
| | p Value | 0.7356 | | |

TABLE 6
Approximate volume measurements of several AA aerogel types used
for dry and wet mechanical testing. T-test results are demonstrated
below as p-values, where the threshold for significance is <0.05

| | | Mean | Standard | Standard |
|---|---|---|---|---|
| | | Volume | Deviation | Error of |
| | | (mm) | (SD) | the Mean (SEM) |
| Agar 1.5 g AA | Dry | 436.39 | 185.71 | 75.815 |
| | Hydrated | 152.50 | 109.45 | 44.683 |
| | p Value | 0.011932 | | |
| Agar 7.5 g AA | Dry | 543.78 | 72.229 | 27.300 |
| | Hydrated | 109.46 | 44.646 | 18.227 |
| | p Value | 0.00000010185 | | |
| Alginate 1.5 g AA | Dry | 233.21 | 10.464 | 4.6795 |
| | Hydrated | 374.07 | 98.443 | 44.025 |
| | p Value | 0.032454 | | |
| Alginate 7.5 g AA | Dry | 473.56 | 46.413 | 17.543 |
| | Hydrated | 539.91 | 101.83 | 45.538 |
| | p Value | 0.22994 | | |
| Pectin 1.5 g AA | Dry | 441.79 | 76.359 | 28.861 |
| | Hydrated | 98.346 | 32.278 | 16.139 |
| | p Value | 0.0000035528 | | |
| Pectin 7.5 g AA | Dry | 483.42 | 38.270 | 14.465 |
| | Hydrated | 410.40 | 59.001 | 26.386 |
| | p Value | 0.048984 | | |
| Gelatin 1.5 g AA | Dry | 397.22 | 85.648 | 34.966 |
| | Hydrated | 540.40 | 64.122 | 26.178 |
| | p Value | 0.0091976 | | |
| Gelatin 7.5 g AA | Dry | 546.50 | 97.021 | 39.609 |
| | Hydrated | 626.74 | 103.98 | 42.450 |
| | p Value | 0.19719 | | |

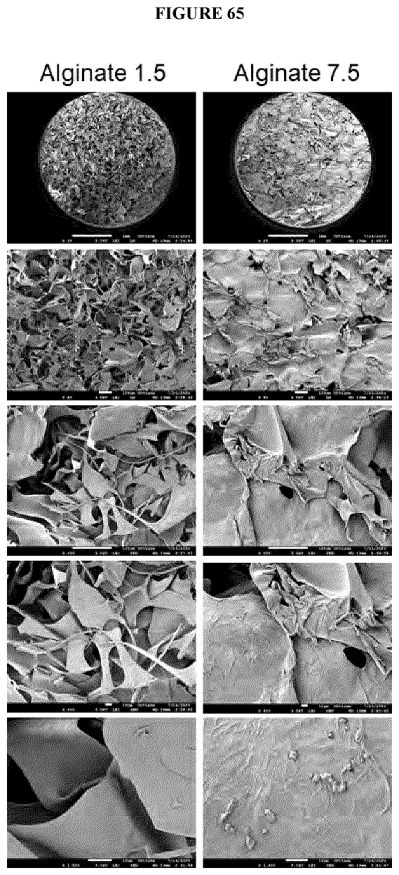

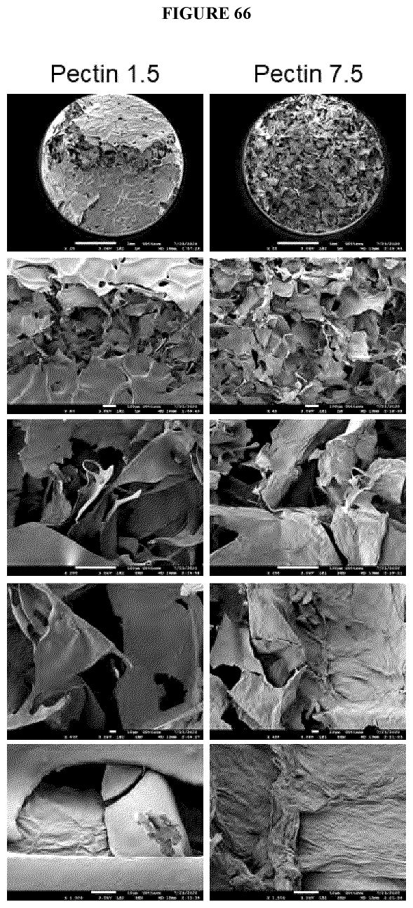

FIG. 65 shows SEM of alginate based aerogels with 1.5 g (10%) and 7.5 g (50%) of decellularized and mercerated AA. FIG. 66 shows SEM of pectin based aerogels with 1.5 g (10%) and 7.5 g (50%) of decellularized and mercerated AA.

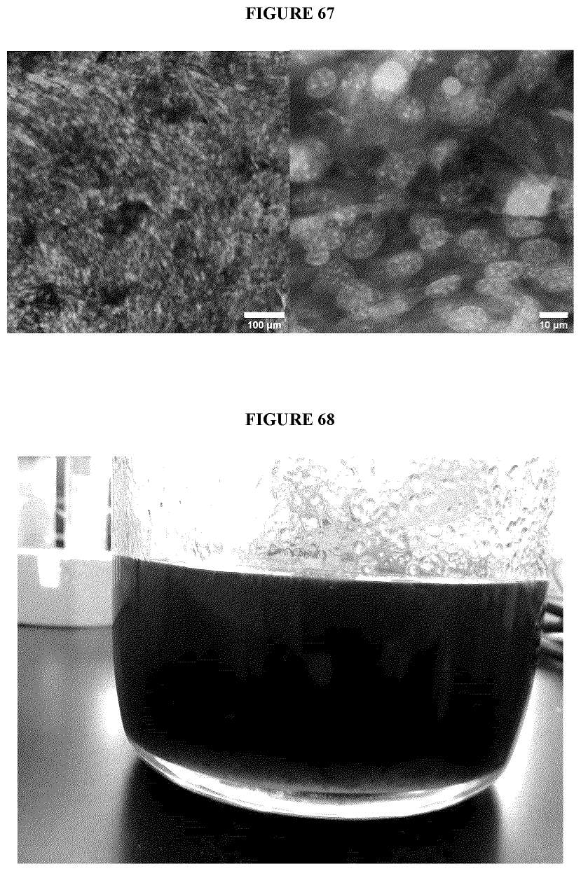

The alginate aerogel with 7.5 g of AA was imaged with confocal microscopy. FIG. 67 shows maximum intensity z-projections of confocal images of alginate foams with 7.5 g of mercerized AA (50%). The red is the scaffold stained with Congo Red. The green is the GFP of the stably transfected 3T3 cells, and blue is the nucleus of the GFP 3T3 cells.

This example provides data indicating that an array of different formulations for aerogels and foams with different properties may be prepared. The front-runners for aerogels and foams for a wide variety of applications are the 50% decellularized and mercerated AA in alginate and gelatin gels, followed by the pectin gels. Moreover, most of these gels were manually mixed by hand stirring, with the exception of the gelatin gels. The gelatin samples were more thoroughly mixed with a luer lock connection system with two syringes. It is contemplated that applying this technique to additional formulations would result in less sample variation and a more uniform gel.

### Example 3-Dissolved Cellulose Hydrogels Prepared from Decellularized Plant Tissue and/or Other Cellulose Sources

This example describes a number of approaches for creating dissolved cellulose-based hydrogels from decellularized plant tissues and other synthetic cellulose sources. In the examples below, a goal was to combine mercerized plant cellulose materials such as the structural cells as described above with newly developed cellulose-based hydrogels to create composite aerogels, foams, and other scaffolds. This may be desirable in a number of different applications, as the resulting aerogels (e.g. both the structural cells and the carrier/hydrogel) will be entirely produced from decellularized plant tissues.

### Cellulose Dissolution and Regeneration:

This example describes the dissolution of cellulose from decellularized apple scaffolds using dimethylacetamide and lithium chloride, and its regeneration by solvent exchange using 95% ethanol.

### Materials and Methods

### Solvent Preparation:

Dry dimethylacetamide (DMAc) at 115° C. for 15 min.
Dry LiCl at 180° C. for 48 h and then maintain at 80° C.

### Solvent Exchange:

Obtain 50 g of decellularized apple.
Transfer to 50 mL Falcon tubes with acetone.
Put in an ultrasonic bath for 15 min.
Remove acetone
Repeat 3×
Remove acetone and add DMAc
Put in an ultrasonic bath for 15 min.
Centrifuge to exchange acetone and DMAc
Remove acetone
Repeat 3×

### Licl Addition:

Ratio: 6 g of LiCl for every 50 mL of DMAc
Transfer scaffolds to dried DMAc in a Duran bottle with a magnetic stir bar.
Maintain at 100° C. for 1 h, and then reduce to 50° C. for 72 h with continuous stirring.

### Regeneration of Cellulose:

Exchange with 95% ethanol
The dissolution solution was centrifuged to remove undissolved material. The dissolved cellulose was then poured into a 60 mm Petri dish to cover the bottom surface. 95% ethanol was then poured on top and a thin wafer began to form.
Small wrinkles and perturbations could be seen in the film.
The film could be pushed with a spatula and bunched into a gel clump.
If left undisturbed a flat disk forms that can be manipulated.
The material was soft but gel-like.
Alternatively, the dissolved cellulose was put into a falcon tube with a hole cut in the lid. A dialysis membrane was placed on the tube opening and then the cut lid was secured on top. The tube was inverted in 95% ethanol to allow for solvent exchange.

### Results

Contemplated mechanism for dissolution of cellulose in DMAc/LiCl as proposed by McCormic et al. (a) and Morgenstern et al. (b) is as follows:

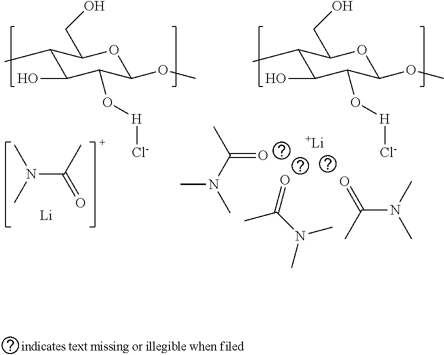

Possible reaction scheme for cellulose dissolution with DMAc and LiCl may also proceed as follows (showing interaction among Li+ cation, Cl-anion, and DMAc when cellulose dissolves into DMAc/LiCl system):

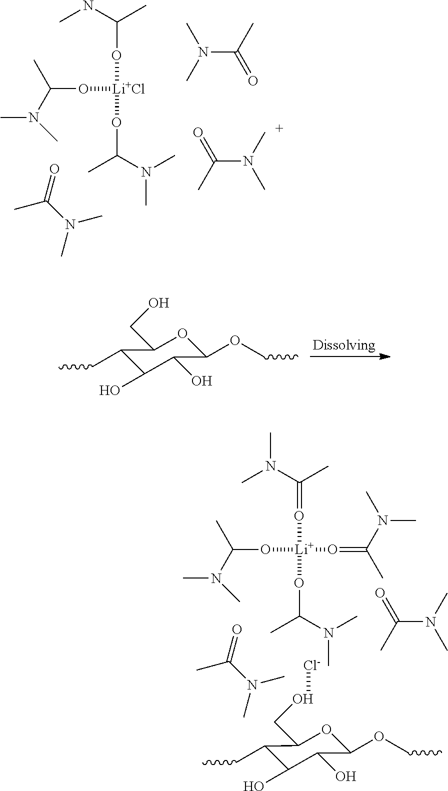

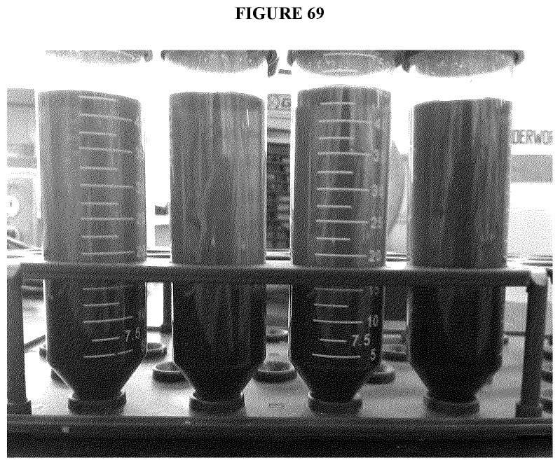

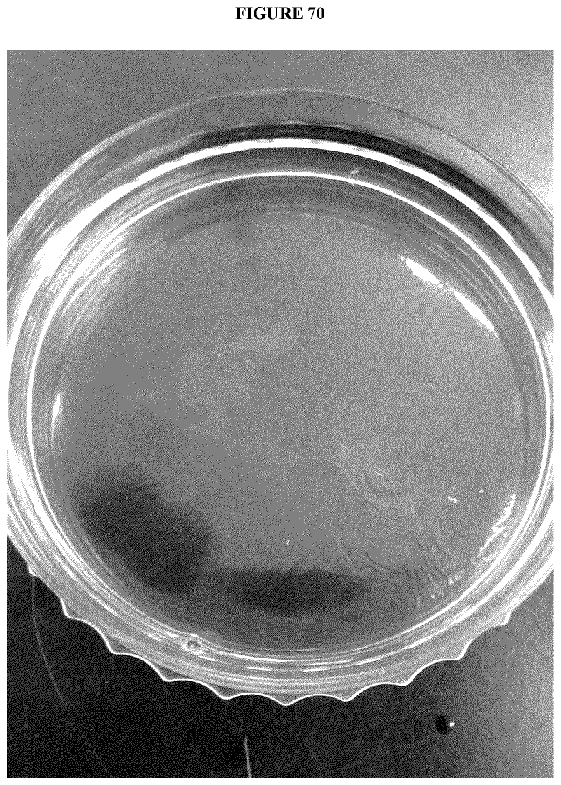

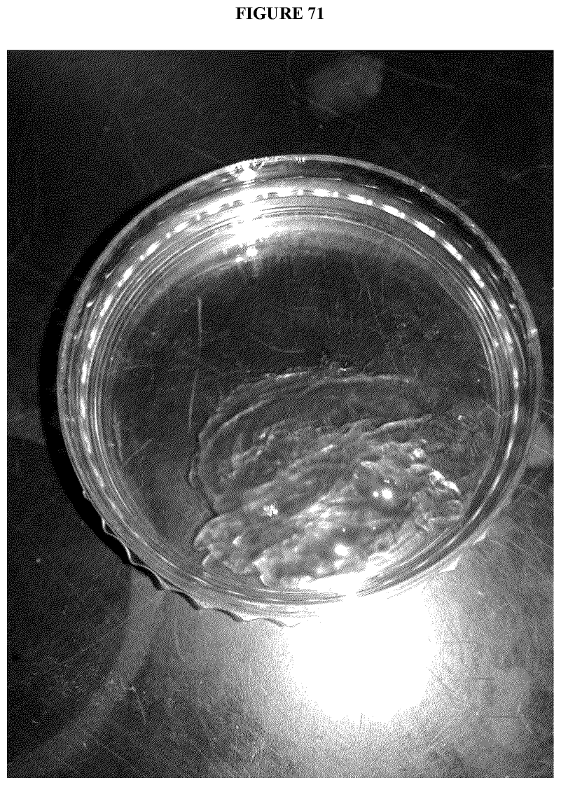

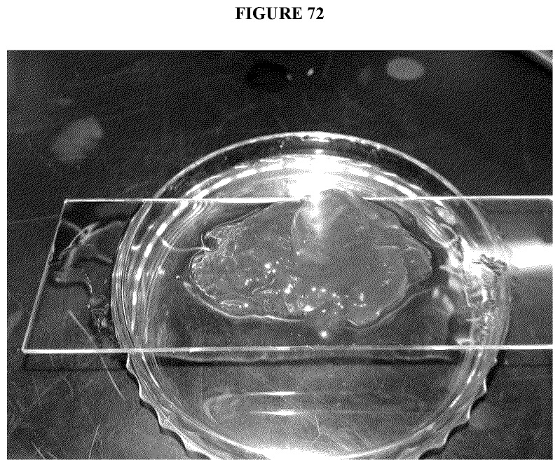

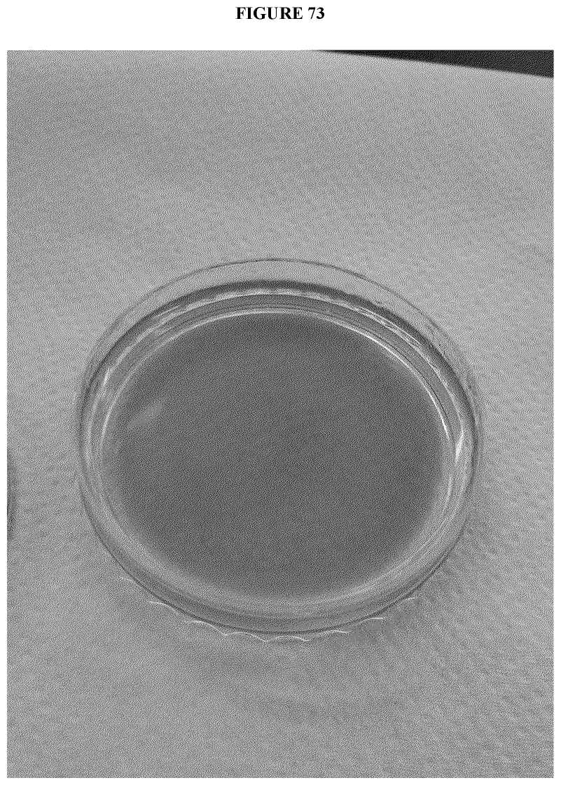

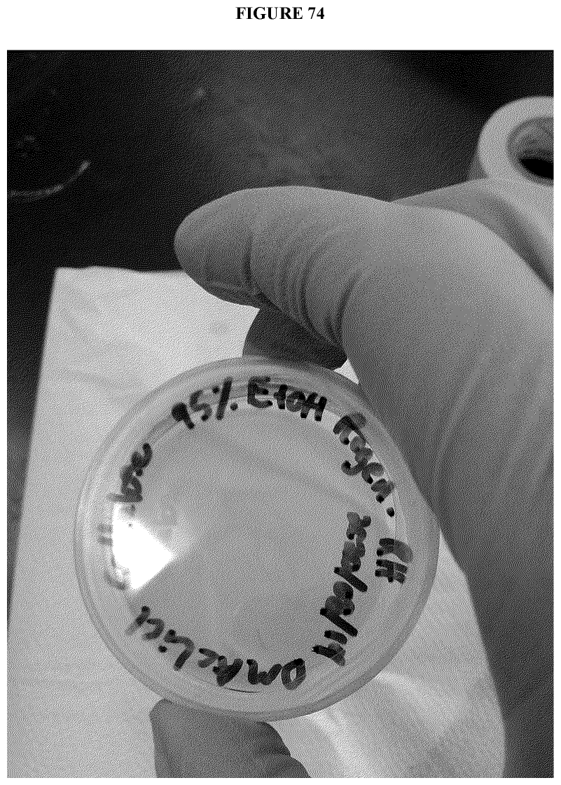

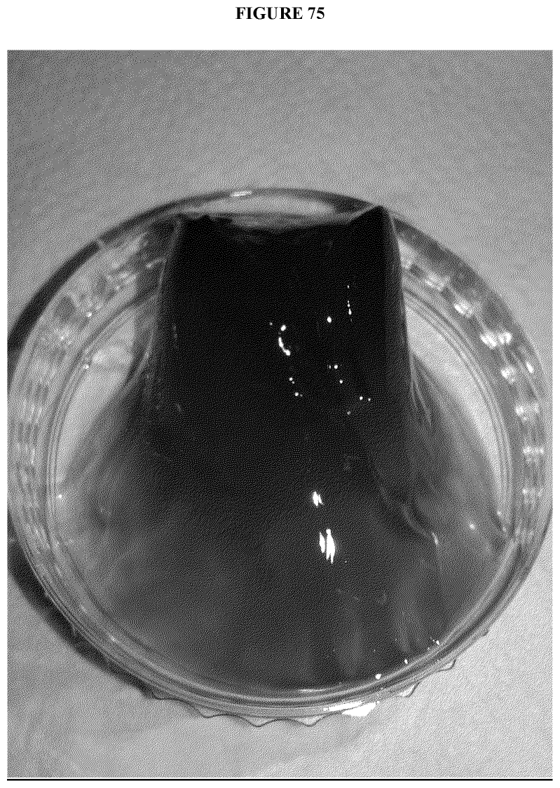

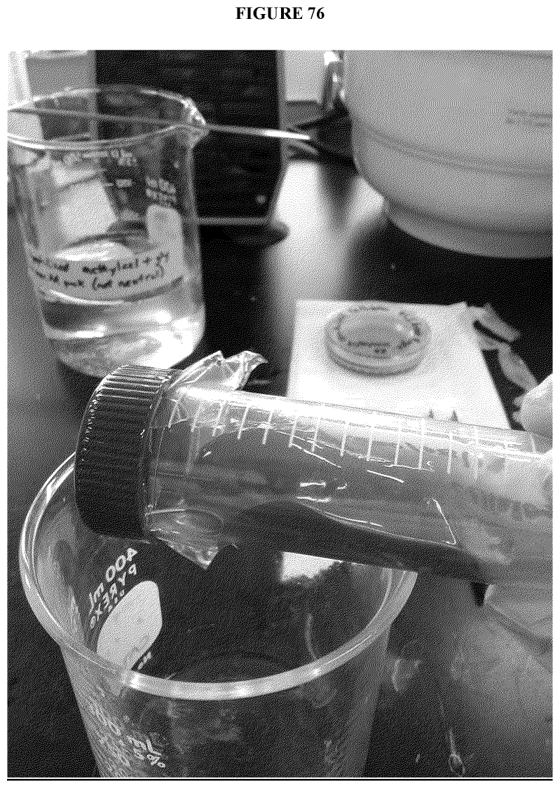

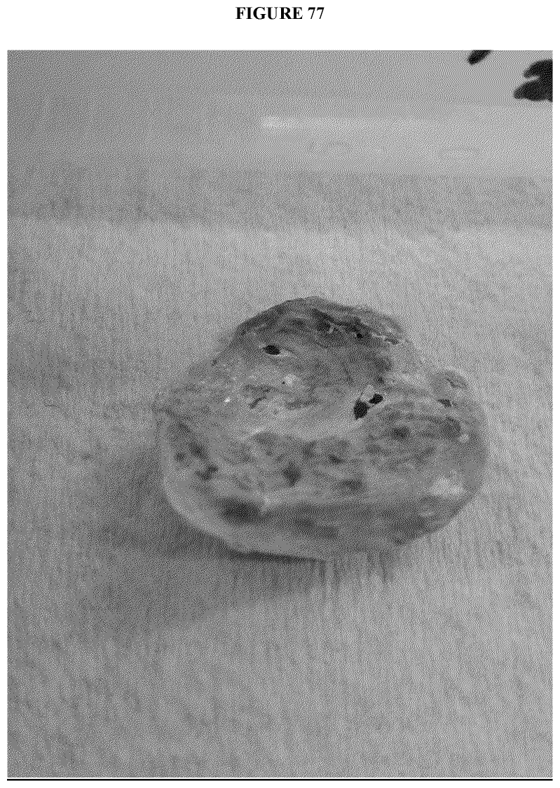

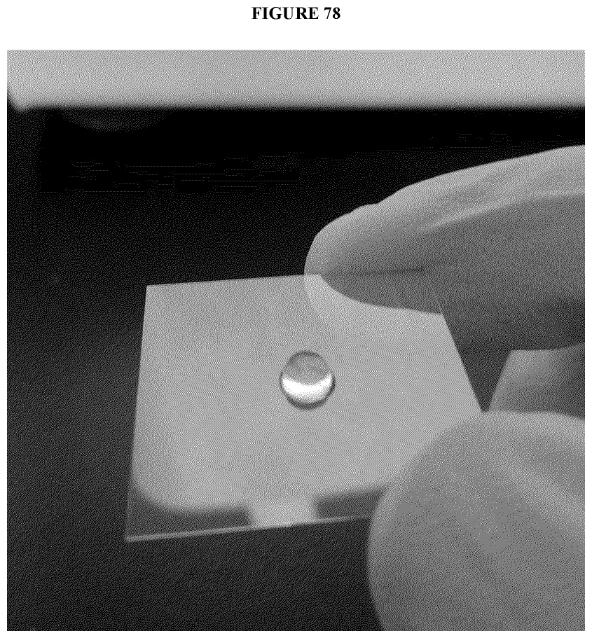

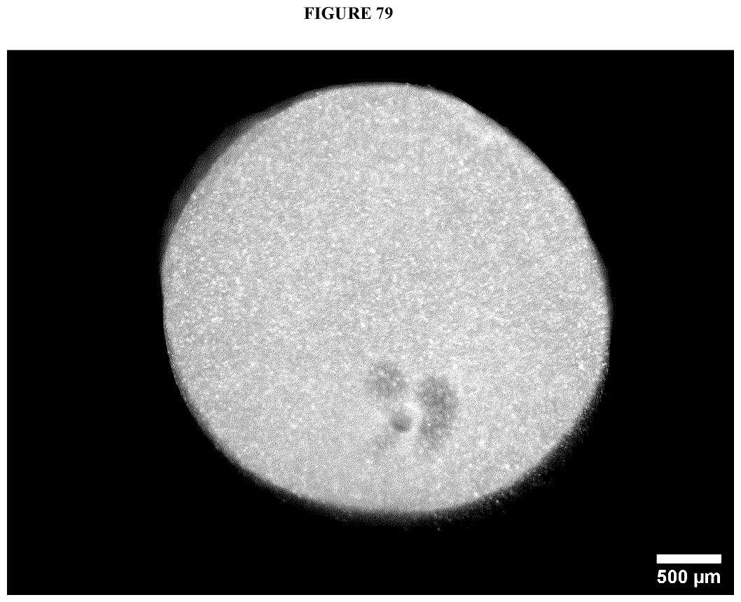

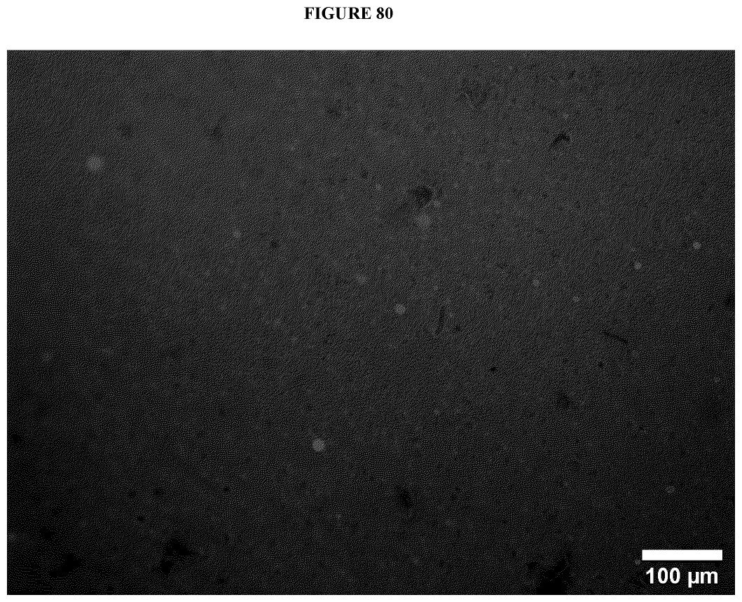

FIG. 68 shows dissolution solution of DMAc and LiCl with decellularized apple after the 72 h reaction. FIG. 69 shows dissolution solution of DMAc and LiCl with decellularized apple after centrifugation to remove undissolved material. FIG. 70 shows cellulose film regeneration. Dissolved cellulose was poured into a 60 mm Petri dish to cover the bottom surface. 95% ethanol was poured on top of the dissolution solution to promote solution exchange and regenerate the cellulose. Wrinkles are observed as the film forms. FIG. 71 shows that within 5 minutes of the ethanol addition, the film could be pushed and bundled with a spatula. FIG. 72 shows regenerated cellulose gel that was collected. FIG. 73 shows regenerated cellulose film, when left undisturbed. FIG. 74 shows regenerated cellulose file, titled to show the wafer slide in the petri dish. FIG. 75 shows regenerated cellulose with a density gradient. Solvent exchange was accomplished with a dialysis membrane. The regeneration occurred in a 50 mL falcon tube. The cylindrical end was in contact with the membrane and had the greatest amount of solution exchange. It was stiffer and held its shape compared to the less stiff and less dense tail region. FIG. 76 shows regenerated cellulose film set-up with the dialysis membrane secured by the lid with a hole cut out of the centre. FIG. 77 shows a lyophilized section of the dense region from FIG. 76. The lyophilization led to scaffold collapse. FIG. 78 shows a regenerated cellulose 5 mm punch from a regenerated film bleached with H₂O₂ (30%). The materials were light brown before treatment, and after treatment with peroxide they were clear. In fact, they were difficult to see because of their clarity. FIG. 79 shows a regenerated cellulose 5 mm punch from a regenerated film bleached with H₂O₂ (30%) imaged with dark-field imaging. FIG. 80 shows a regenerated cellulose 5 mm punch from a regenerated film bleached with H₂O₂ (30%) stained with Congo Red to visualize the micro-structure. The surface was very flat with small pores. This is a fluorescence image with TRITC.

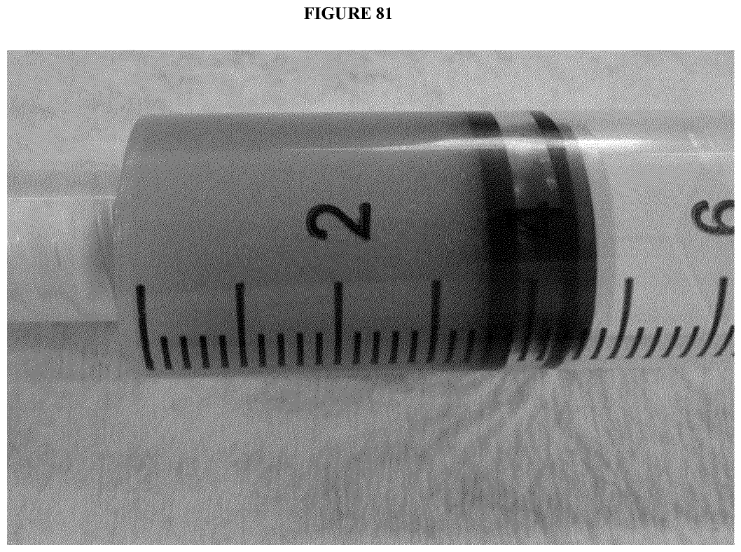

Mercerized material (i.e. single structural cells, groups of structural cells, or both, obtained from mercerization treatment of a decellularized apple tissue as described hereinabove) was mixed with the regenerated cellulose obtained as described above in this example. In an attempt to avoid premature regeneration, mercerized material (1 g) was mixed with acetone and sonicated for 5 minutes. The material was then centrifuged at 5000 rpm for 7 min. The mercerized cellulose (water exchanged for acetone) was mixed with the DMAc LiCl dissolved cellulose mixture (5 mL). The combination was mixed in a dual syringe, luer lock connector set-up. FIG. 81 shows DMAc LiCl dissolved cellulose mixed with mercerated AA (the colour comes from the DMAc LiCl dissolved cellulose solution; the mercerized material was white).

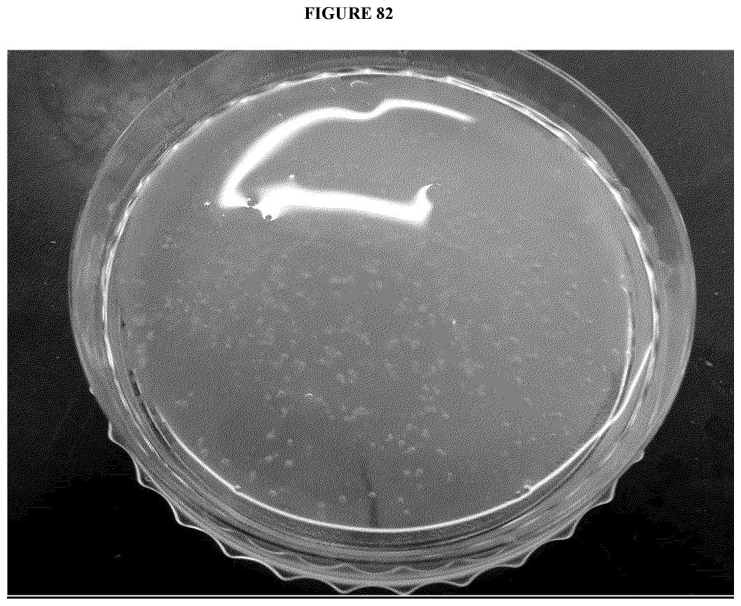

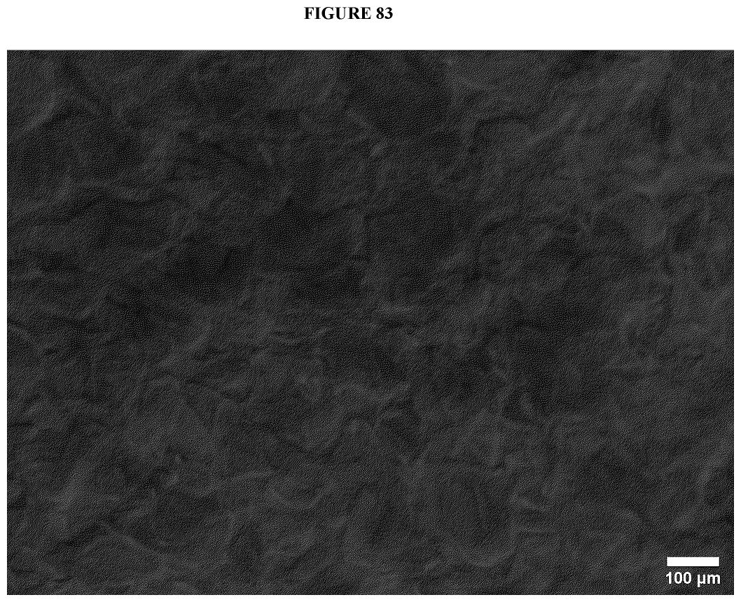

FIG. 82 shows dissolved cellulose with mercerized AA mixed into it. The membrane was regenerated by coating with a layer of 95% ethanol overnight. A composite film is obtained. FIG. 83 shows a fluorescence microscopy image of the regenerated cellulose with the mercerized material mixed into it. The apple structural cells from the mercerized material can be seen tightly packed together. This topography is distinct from the smooth material obtained from pure regenerated cellulose.

Lyophilized Composite Aerogels Produced from Mixtures of Regenerated Cellulose and Mercerized Materials:

After producing mixtures of regenerated cellulose and mercerized materials, the hydrogels were frozen and lyophilized (as described above) to produce aerogels/foams which could be left dry or rehydrated. Several formulations were prepared as follows:

| | Dissolved | Mercerized | | |
|---|---|---|---|---|
| | Cellulose | AA | 5% | 5% |
| Sample | (% v/v) | (% v/v) | Alginate | Agarose |
| P1 | 100 | 0 | 0 | 0 |
| P2 | 80 | 20 | 0 | 0 |
| P3 | 50 | 50 | 0 | 0 |
| P4 | 0 | 100 | 0 | 0 |
| P5 | 50 | 0 | 50 | |
| P6 | 50 | 0 | 0 | 50 |
| P7 | 20 | 80 | 0 | 0 |

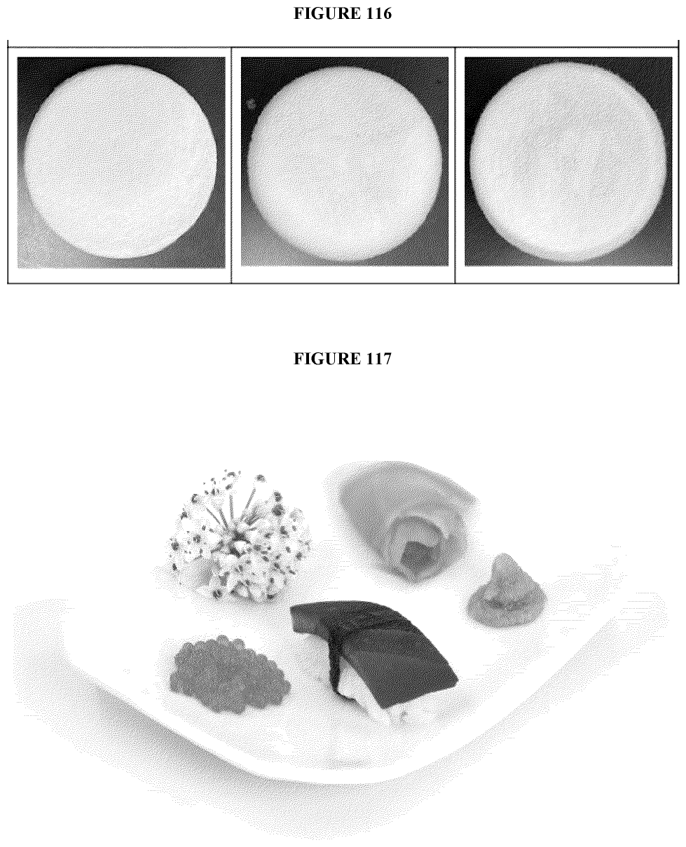

FIG. 115 shows lyophilized aerogels produced with the formulations listed above (samples P1, P2, P3, P4, P5, P6), about 1 cm in diameter. FIG. 116 shows larger scale lyophilized (3 cm diameter) aerogels produced with the formulations listed above; P2 (Left), P7 (Middle), P3 (Right) images.

Methylcellulose-based Gels were also prepared. In this next example, an all-cellulose material was prepared using methylcellulose and mercerized material (i.e. single structural cells, groups of structural cells, or both, obtained from mercerization treatment of a decellularized apple tissue as described hereinabove).

### Preparation of the Mercerized Decellularized Material

Decellularized apples were mercerized in 1 M NaOH for 1 h. In order to lighten the colour, hydrogen peroxide (stock 30%) was added to the mixture (5% v/v of the 30% stock) during the 1 h mercerization process. The solution was then neutralized with HCl and centrifuged to collect the material. To ensure the pH remained stable, the pellet was resuspended in dH2O, and the solution was neutralized again. This process was repeated until the pH remained between 6.8 and 7.2 for subsequent cycles.

### Gel Formulation

The gelation process involved dissolving the methylcellulose in 10 mL of 2 M NaOH for 1 h with stirring on ice. A glycine solution was also prepared by dissolving glycine in 2 M NaOH. After 1 h, 5 mL of the glycine solution was added, and the mixture stirred on ice for an additional hour. The mercerized apple was introduced at one of two different stages. One method of introduction involved mixing in the mercerized apple with the viscous solution after the second hour of glycine treatment. This particular mixing method involved using syringes connected with an F/F luer lock system. For the higher methylcellulose concentration (1 g), the mixing with syringes was exceedingly difficult. As a result, a second preparation method was developed. In this approach, the mercerated apple was added directly to the 2 M NaOH with the methylcellulose at the start of the reaction. Mixing was accomplished with magnetic stirring. See Table 7 for the formulations tested. It was observed that when the glycine was added, the viscosity of the mixture increased; however, it appeared that this was largely due to physical crosslinking induced by temperature increases. The gels were left at room temperature overnight to crosslink.

TABLE 7
Gel Formulations using Methylcellulose
and Mercerized Apple Structural Cells

| | Glycine | Mercerized | Mercerized |
|---|---|---|---|
| | stock % | AA | AA |
| Methycellulose (g) | (w/v) | (g) | addition |
| 0.5 | 20 | 1.5 | after glycine |
| 0.5 | 30 | 1.5 | after glycine |
| 1 | 20 | 1.5 | after glycine |
| 1 | 30 | 1.5 | after glycine |
| 1 | 30 | 1 | before glycine |
| 1 | 0 | 1 | with methylcellulose |

### Results:

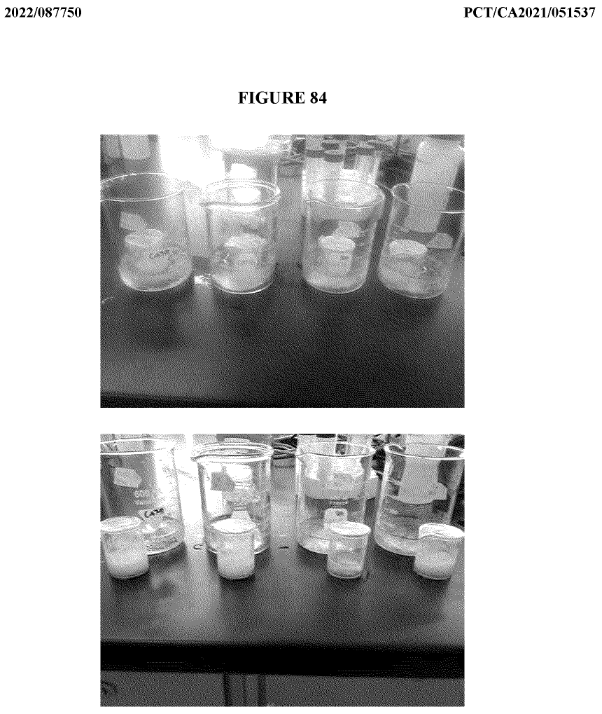

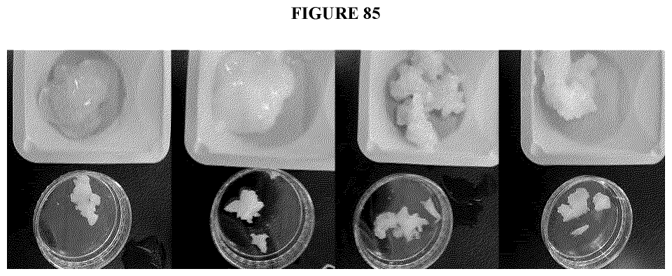

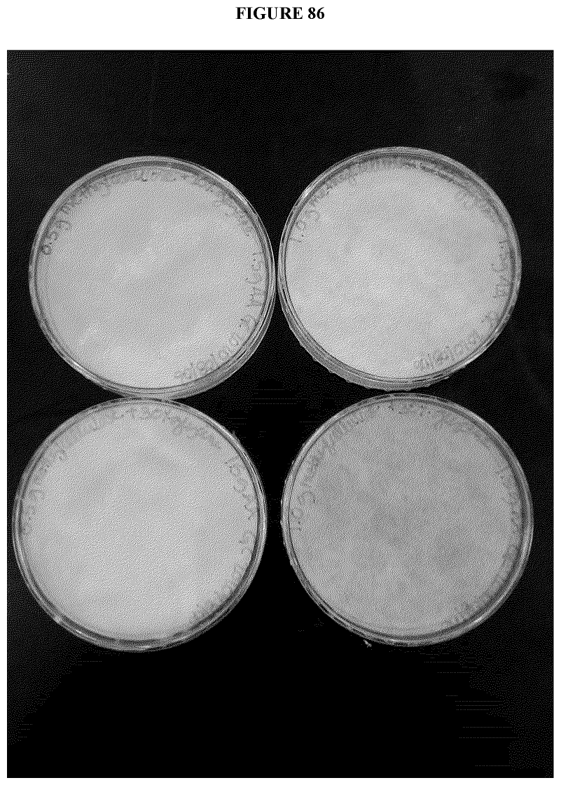

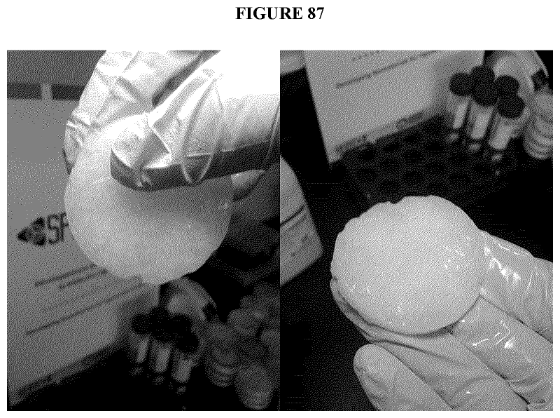

FIG. 84 shows the reaction arrangement. The reaction was carried out in small beakers with a magnetic stir bar. These beakers were covered with parafilm and put in a larger beaker which contained an ice bath. FIG. 85 shows methylcellulose and mercerized AA. The methylcellulose mixed with glycine (upper in the weigh boats) and the mercerized AA (lower in the Petri dishes). The 1 g of methylcellulose was more viscous (right two images) compared to the 0.5 g (left two images). FIG. 86 shows methylcellulose gels with mercerized AA (apple) and glycine (AA introduced after glycine addition) after incubation at room temperature overnight to crosslink. The gels could be removed from the Petri dishes and maintain their shape. The 1 g methylcellulose gels were more stiff. FIG. 87 shows methylcellulose and mercerized AA gel. 1 g of methylcellulose, 1 g of AA mixed in 10 mL of 2 M NaOH for 1 h mixed by magnetic stirring in an ice bath, then 5 mL of 30% glycine in 2 M NaOH was added for an additional hour of stirring on ice. Crosslinking at room temperature overnight in a 60 mm Petri dish. The gels can be handled and maintain their shape. FIG. 88 shows the same gel from FIG. 87 cut with a scalpel blade into two halves. One was kept, and the other was used to test the neutralization. The neutralization was 5% acetic acid for 1 h followed by 10 water washed. It was also tested whether after doing this there would be a slow release of NaOH which would result in the pH increasing. This did occur. As a result, the half-aerogel was washed 70 times and was also neutralized with 30% acetic acid.

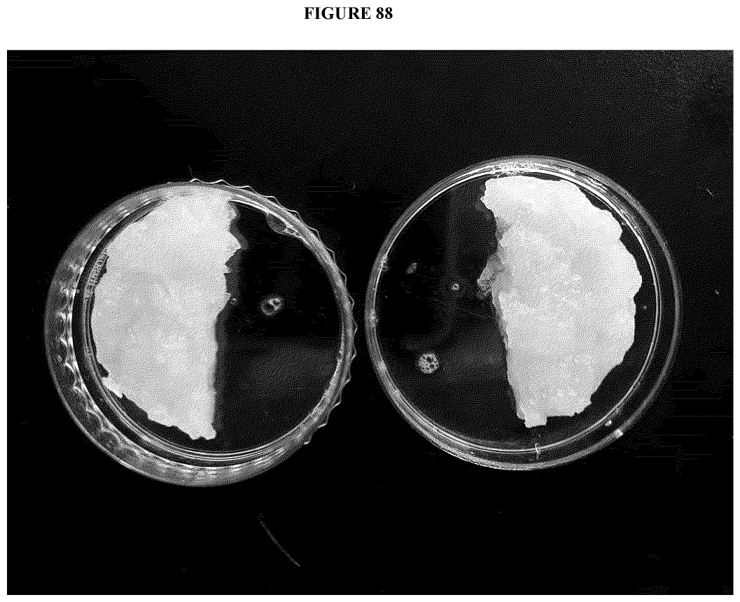

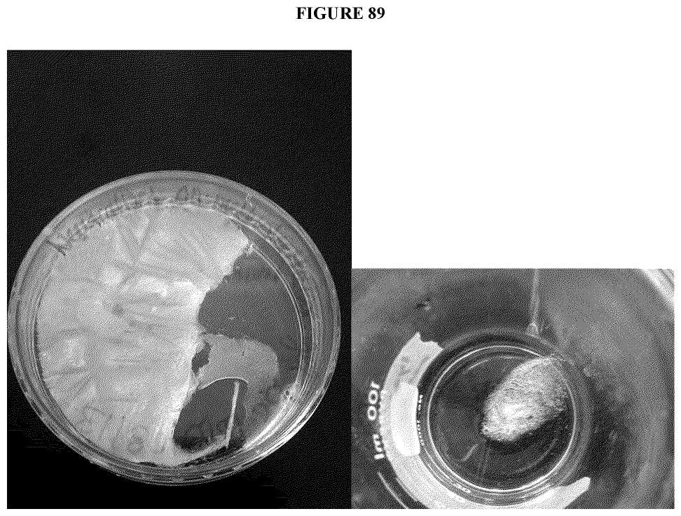

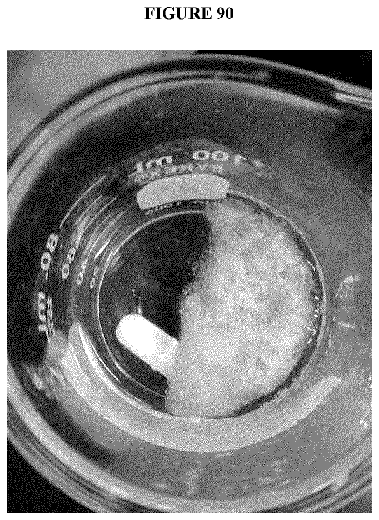

FIG. 89 shows the excessively washed “half-aerogel” from FIG. 88 was frozen at −20° C. and then lyophilized at −46° C. and 0.050 mbar (upper). The dried material appears fragile, but was actually fairly stiff to the touch. Directional freezing was also observed. A section was then torn off and immersed in dH2O (lower image). It remained intact and had a soft, sticky texture. FIG. 90 shows the second half of the aerogel cut from FIG. 88 was neutralized. The neutralization was performed with 30% acetic acid right away. This had a similar, but opposite consequence: the pH would drift to acidic values and the slow release of the acetic acid made the pH drift to lower values over time. This was corrected with a slow titration with 1 M NaOH. Nevertheless this indicates an optimal neutralization step somewhere between 5% and 30% acetic acid will likely be a faster, more efficient approach. The neutral sample was kept for future dye testing.

It was found that neutralization for aerogels this size was a challenge as there was a slow release of NaOH or the neutralizing solution. The 5% acetic acid was insufficient to fully neutralize the NaOH (as shown above). This was surprising. Comparatively, the addition of 30% acetic acid had the opposite effect: the acid used for neutralization slowly released from the aerogel and required additional base treatment. It was found that 12-15% acetic acid was an appropriate concentration for neutralization without making the pH swing too far to one extreme.

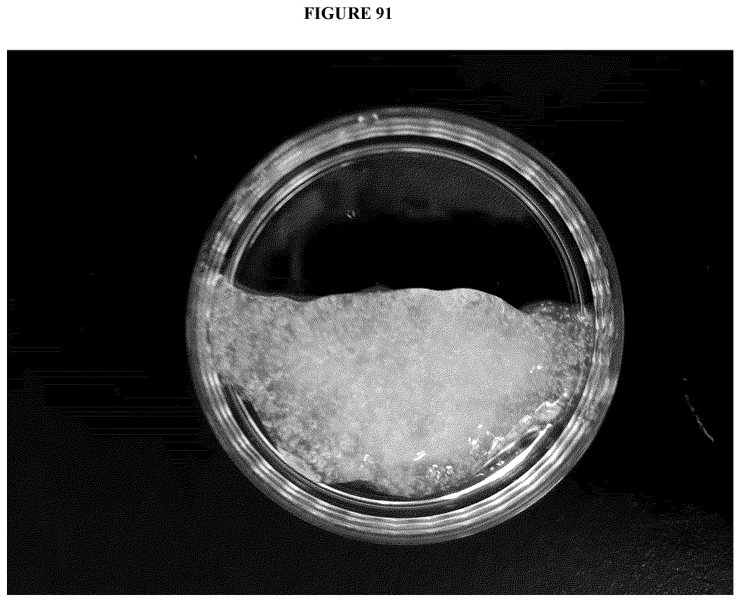

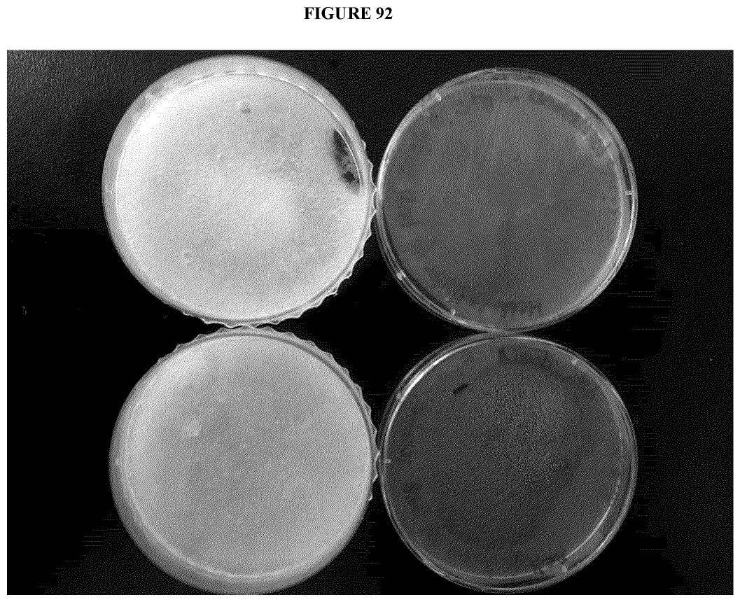

FIG. 91 shows methyl cellulose with mercerized AA (1:1) half-aerogel neutralized with 15% acetic acid. It was also found that the methyl cellulose gels (with and without the AA) swelled greatly. This can occur while freezing and freeze drying as well. FIG. 92 shows Methyl cellulose with mercerized AA (1:1) half-aerogel neutralized with 15% acetic acid. The aerogels shown in FIG. 92 were neutralized as half-aerogels (FIG. 91). During the freezing, they expanded to fill the 60 mm petri dish. Once freeze-dried, they produce a white foam that is easily handled and relatively stiff. Once hydrated, they expand and if they keep expanding, they turn into a loose material with a sticky consistency.

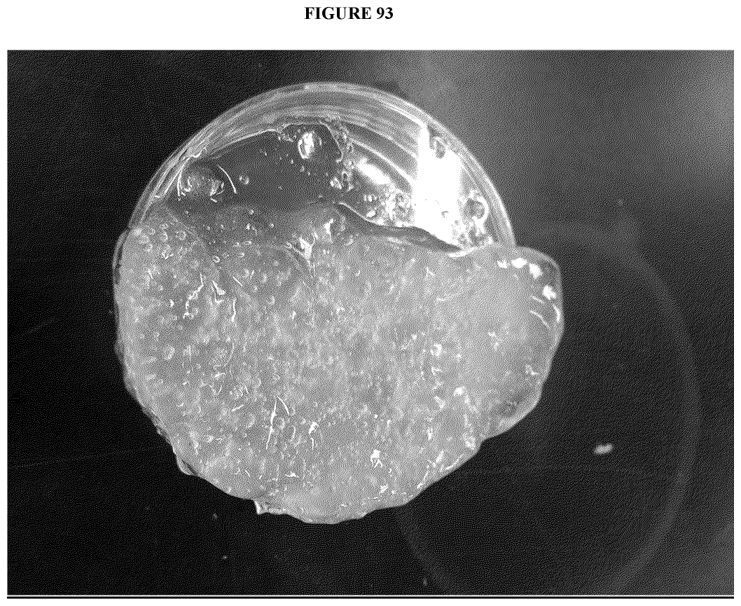

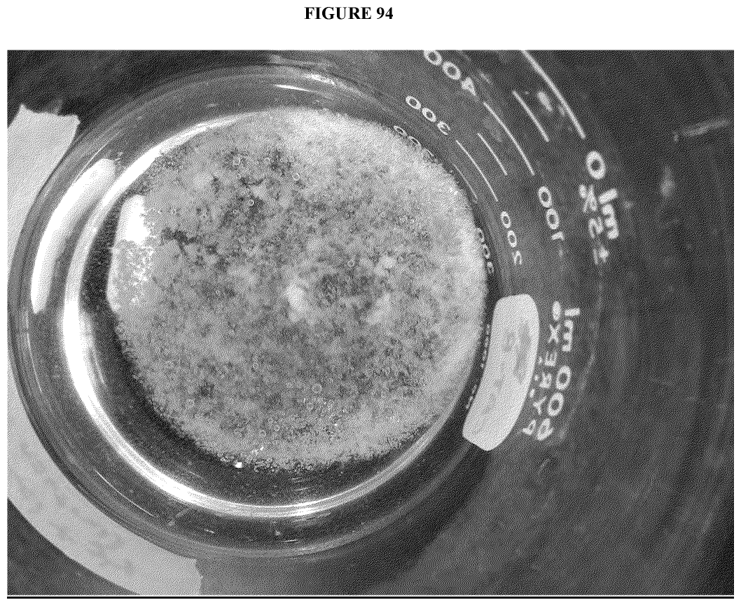

FIG. 93 shows Methyl cellulose with mercerized AA (1:1) expansion. The half-aerogel was placed on it's original 60 mm dish for comparison. FIG. 94 shows Methyl cellulose with mercerized AA (1:1) continued expansion into a loose material.

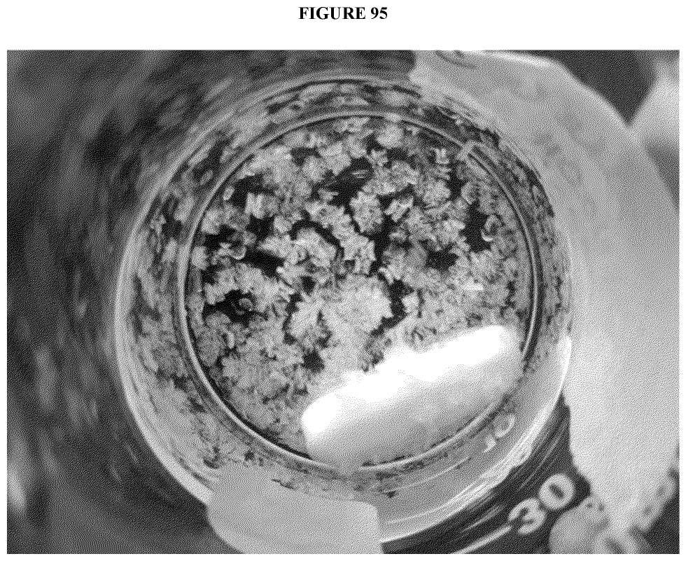

In an attempt to control the swelling, it was sought to increase the concentration of glycine. It was found that the 30% glycine solution was approaching the saturation point. At 40%, heat was required to dissolve the glycine. As the gel is thermoresponsive, the glycine needed to be cooled prior to addition to the methyl cellulose and mercerized AA mixture; however, upon cooling the solution, the glycine crashed out of the solution. FIG. 95 shows crystallization of glycine at reduced temperatures (˜4° C.) from a 40% solution. The drastic effect of temperature on the methyl cellulose as well as the increased hydrophobicity as compared to the microcrystalline cellulose, led to testing the role of the glycine. Glycine can crosslink microcrystalline cellulose; however, it was tested whether we could obtain a similar gel simply with temperature effects. This was achieved.

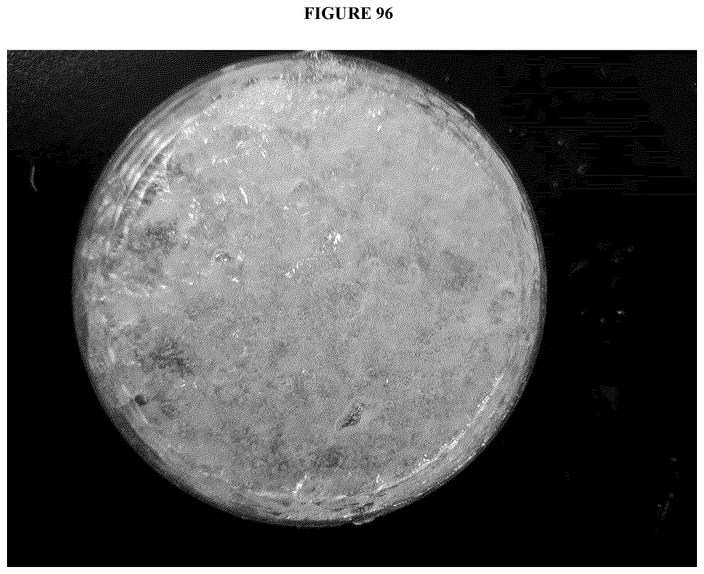

FIG. 96 shows carboxymethyl cellulose gel in the absence of glycine gives a similar physically crosslinked material.

### Example 4-Use of Aerogels and Foams for Bone Tissue Engineering

This example describes use of aerogels and foams as described herein, such as those prepared in Examples 1 and 2, for bone tissue engineering.

### SUMMARY

This Example describes standard operating procedures for implantation and resection of decellularized biomaterials into trephinated calvarial defects. The study was conducted to evaluate the potential of aerogels and foams as described herein for bone regeneration applications, in a rat critical-size, bilateral defect model. The biomaterials (alginate and pectin based aerogels) were implanted in rats for periods of 4 and 8 weeks. 5 mm bilateral, circular defects were created in the rat calvarium. Once the bone defects were excised, the aerogel (alginate or pectin aerogel formulations, Table 2 provides formulations for the 5% alginate aerogel and the 5% pectin aerogel used in the bone tissue engineering example) biomaterials (5 mm diameter by 1 mm thickness) were placed within the defect. Overlying skin was sutured, and the rats were left to recover for a period of 4 to 8 weeks. Specimens were collected at each time points and computational tomography (CT scan), implant dislocation mechanical testing, and histology were performed.

### Key Chemicals and Solutions

1. Mercerized apple paste made from decellularized McIntosh apples, neutralized
2. 5% Alginate
3. 5% Pectin
4. Calcium Chloride

Table 2 provides formulations for the 5% alginate aerogel and the 5% pectin aerogel used in this bone tissue engineering example.

### Procedure-Implantation

1. The rat is prepared for anesthesia and isoflurane is administered until unconsciousness is observed
2. The rat is then transferred to the preparation area, saline administered via syringe and tear gel applied over the eyes to reduce corneal dryness.
3. The top of the head is shaved from the bridge of the snout between the eyes to the caudal end of the skull, then the fur vacuumed off.
4. The rat is then transferred to the surgical area and secured to the stereotactic equipment.
5. The skin is washed with water and sterilized with chlorhexidine.
6. The biomaterials are photographed in sterile saline next to a ruler.
7. A trephine is secured to the drill and placed next to the surgical area.
8. Once the researcher dons a sterile gown and gloves, an incision is made with a scalpel down the periosteum over the scalp from the nasal bone to just caudal to the middle sagittal crest.
9. Using 5.5 mm alm retractors the skin is exposed to the underlying bone.
10. The periosteum is divided down the sagittal midline and dissected.
11. The bone is cleaned with a sterile cotton swab,
12. The left parietal bone is scored with a 5 mm trephine under constant irrigation of sterile normal saline, under 1500 rpm
13. Moving circumferentially around the defect margin with the elevator blade, the defect is completed by applying gentle pressure
14. The blade of the elevator is used to slide under to remove the bone
15. The right bone is similarly removed
16. A non-sterile researcher brings the biomaterials to the surgical area and places them where instructed
17. Carefully, each biomaterial is placed in the defect of each parietal bone.
18. The biomaterials are photographed next to a ruler.
19. The alm retractors are removed and the incision is closed using interrupted sutures.
20 Bupivacaine is applied to the sutures and the rat is transferred to the recovery station.

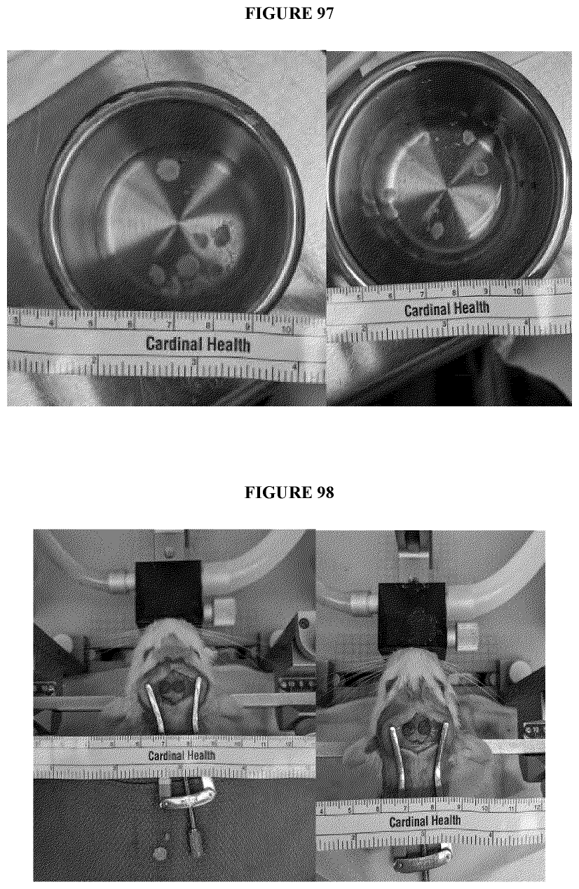

FIG. 97 shows alginate (left) and pectin (right) aerogel scaffolds prior to implantation into trephinated defects. FIG. 98 shows alginate (left) and pectin (right) aerogel biomaterials implanted in the trephinated defects of the parietal bone.

### Resection Procedure:

After the rats were allowed to recover for the desired amount of time (e.g. 8 weeks), specimens were collected and scanned with computational tomography (CT). Histology was then also performed.
1. The animal is transferred to the CO2 euthanasia box and the correct flow rate is set
2. After at least 5 minutes, and the rat has been determined to stop breathing for at least one minute, it is removed from the box.
3. Vital signs are examined and a thoracotomy followed by exsanguination is performed
4. Turning the rat on its stomach, the skin above the cranium is lifted and cut off with scissors to expose the implants
5. Using a scalpel, the muscles on either side of the cranium are cut away
6. Then, using a drillbit, the front of the calvarium is severed from the rest of the skull
7. The calvarium is then lifted using tweezers, cutting away tissue from underneath.
8. Once removed, a small notch is made on the bottom left of the calvarium to indicate directionality of the sample, and a picture taken of the implants within the trephinated area
9. The calvarium is then placed into a tube with formalin solution for 72 hours, followed by 70% ethanol, then stored at 4 C.
10. Once in ethanol, the samples were delivered for CT scanning. Each sample was rotated 180° and imaged every 0.7°

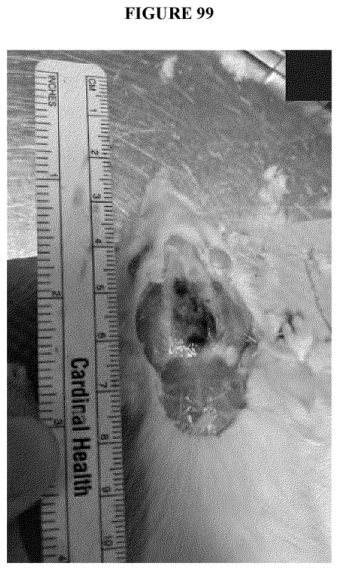

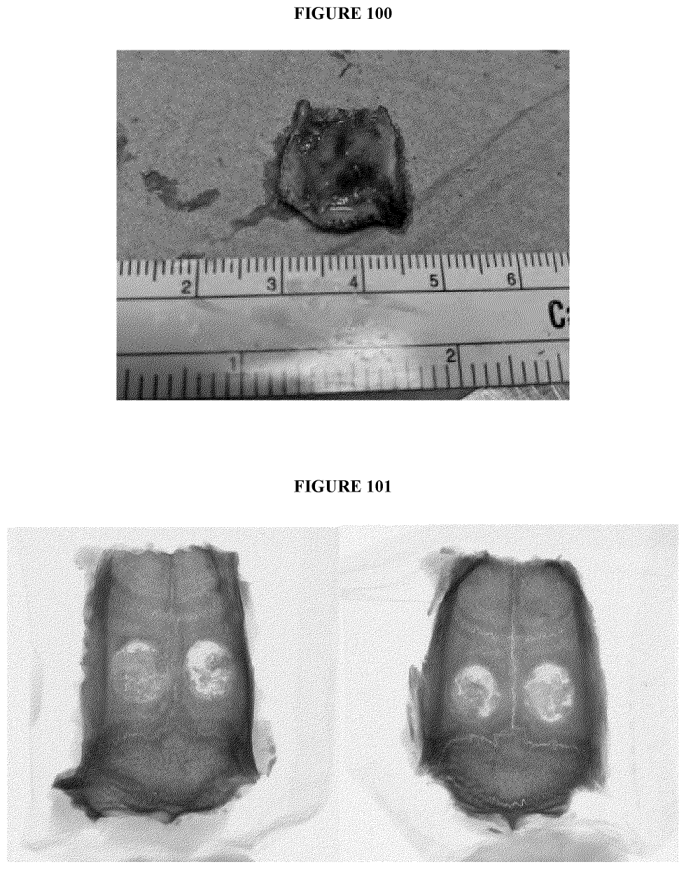

FIG. 99 shows alginate aerogel implants in the rat calvarium prior to resection. FIG. 100 shows resected rat calvarium. FIG. 101 shows rat calvariums with trephinated defects resected after 8 weeks and scanned with Computational Tomography (CT). Alginate biomaterials (left) and Pectin biomaterials (right). The results reveal the aerogel biomaterials support cellular infiltration and regeneration in vivo.

### Example 5-Peroxide Ratios for Mercerization

This example describes studies of peroxide ratios for mercerization processes as described herein, such as those used for preparing the structural cells of the aerogels and foams as described herein, such as those in Examples 1 and 2.

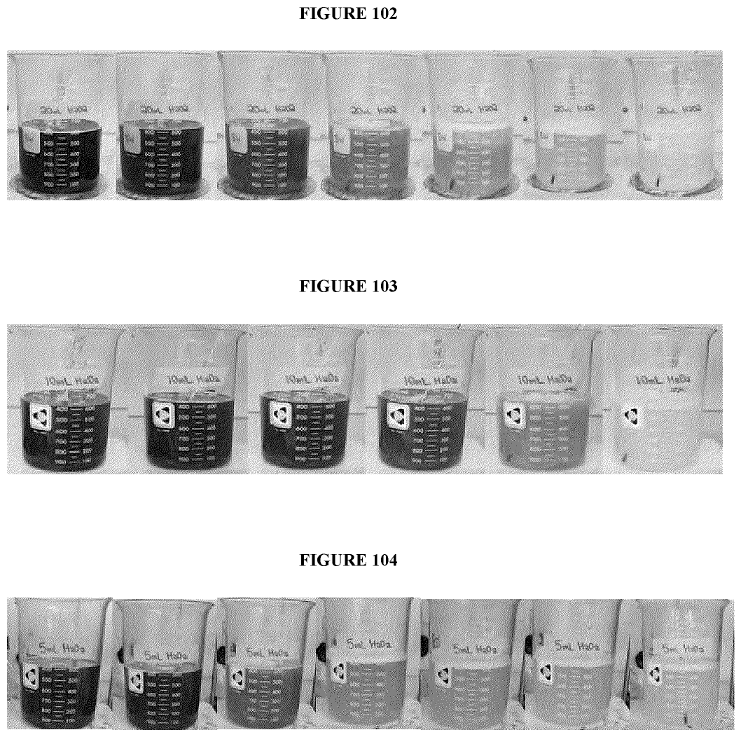

In order to test different ratios of peroxide, a constant ratio of decellularized apples to NaOH was used. This was 100 g of decellularized apple for 500 mL of 1 M NaOH. The amount of 30% stock hydrogen peroxide added to this constant ratio was 20 mL, 10 mL, and 5 mL. These amounts correspond to a final concentration of 1.15%, 0.58%, and 0.30% respectively (in regards to the concentration in the 1 M NaOH). Clearly, as shown in FIGS. 102-105, the volume of the apples modifies these concentrations slightly when they are added to the solutions.

FIG. 102 shows bleaching during mercerization with 20 mL of hydrogen peroxide over the course of 1 h. FIG. 103 shows bleaching during mercerization with 10 mL of hydrogen peroxide over the course of 1 h. FIG. 104 shows bleaching during mercerization with 5 mL of hydrogen peroxide over the course of 1 h.

It should be noted that the peroxide treatment may be done after the mercerization as well; however, it is observed that the high temperature and basic conditions of the mercerization speeds up the lightening process.

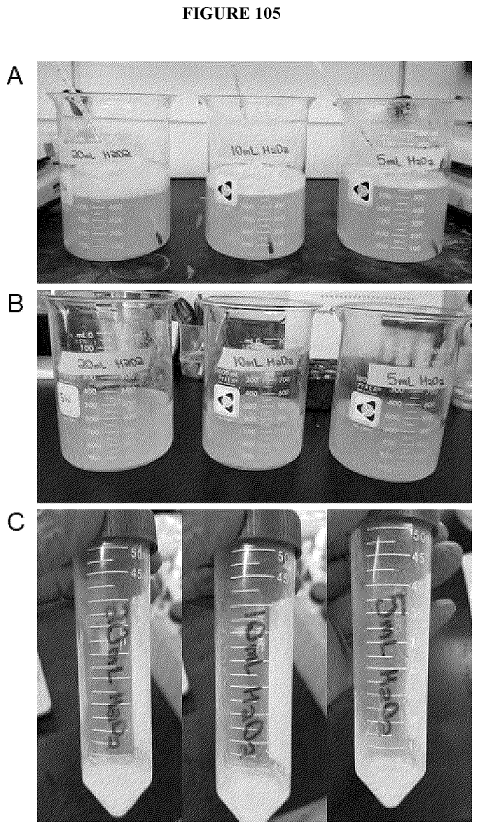

FIG. 105 shows that (A) after the 1 h mercerization with different amounts of peroxide, the colour is slightly more clear for the higher peroxide concentrations; (B) after neutralization, the slight colour variations disappear and all three have a clear/off-white colour; and (C) the final concentrated product was comparable for the three hydrogen peroxide ratios.

Results indicate that different peroxide ratios may be used to achieve a similar final product. Reducing the concentration of peroxide from 1.15% to 0.3% did not affect the bleaching of the final product after neutralization.

### Example 6-Mass Proportions of Structural Cells in Aerogels and Foams

This example describes studies of mass proportions of structural cells of the aerogels and foams as described herein, such as those in Examples 1 and 2. As described herein, in certain embodiments the aerogel or foam may comprise about 10-50% m/m (or more) single structural cells, groups of structural cells, or both, when the aerogel or foam is in hydrated form.

As will be understood, the mass of the aerogels and foams when dry will be significantly different from the mass of the rehydrated (or wet) aerogels and foams. In some experimentally measured examples, the dry mass of prepared aerogels was about 5.18% of the wet mass of the same aerogels.

As will further be understood, the structural cells of the aerogels and foams as described herein may have a wide range of sizes, and may be substantially uniform of may be a mixture of different sizes. In certain embodiments, the structural cells may have a size ranging from about 20 μm to about 1000 μm. If desired, it is contemplated that larger particles may be obtained by reducing the mercerization time or changing the source material with a different size range of plant cells, for example. Typically, for apple decellularized tissue, the structural cells provide a mean particle size of ˜200 μm, however it will be understood that other sizes are also contemplated.

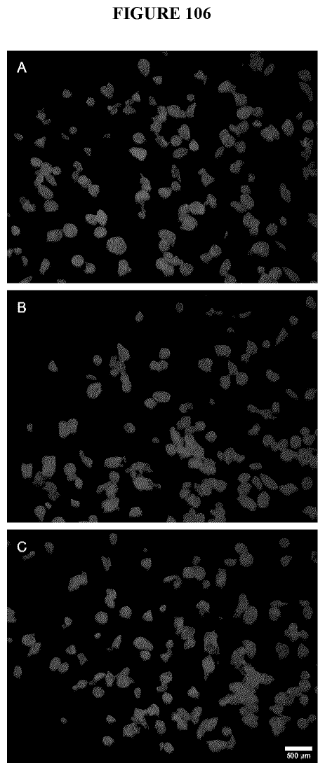

FIG. 106 shows fluorescent microscopy images of the three different AA:NaOH ratio conditions (i.e. mercerization conditions). (A)—1:5, (B)—1:2, and (C)—1:1. Images were captured with the Olympus SZX16 microscope at 2.5× magnification using the BV filter and Congo red stain.

### Particle Size Analysis:

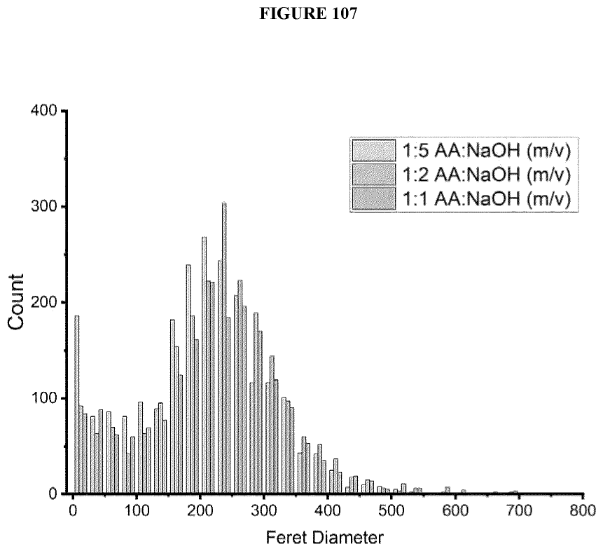

The images were thresholded and segmented using Fiji ImageJ. A watershed was applied to the image after it was converted to binary. The Feret diameter was calculated with the Analyze Particles plugin. The histograms of the particle sizes were very similar for each case (FIG. 107). FIG. 107 shows a histogram of the particle size distributions from the mercerization of decellularized AA in different ratios with 1 M NaOH.

To further investigate the particle sizes, an ANOVA was performed with a Tukey post-hoc analysis at a significance level of 0.05. The data is summarized in Table 8 and Table 9.

TABLE 8
Descriptive Statistics of Particle Size Distributions

| | | | Standard | SE of |
|---|---|---|---|---|
| Ratio (AA:NaOH) | N | Mean | Deviation | Mean |
| 1:5 | 2237 | 199.81148 | 106.08425 | 2.24294 |
| 1:2 | 2158 | 228.0239 | 106.3168 | 2.28863 |
| 1:1 | 1878 | 219.72194 | 109.23839 | 2.52074 |

TABLE 9
ANOVA of the Particle Size Distributions

| Ratios | MeanDiff | SEM | q Value | Prob | Alpha | Sig | LCL | UCL |
|---|---|---|---|---|---|---|---|---|
| 1:2 and 1:5 | 28.21242 | 3.23208 | 12.34451 | 3.33E−16 | 0.05 | 1 | 20.63741 | 35.78744 |
| 1:1 and 1:5 | 19.91046 | 3.35247 | 8.39907 | 8.60E−09 | 0.05 | 1 | 12.05327 | 27.76764 |
| 1:1 and 1:2 | −8.30197 | 3.38036 | 3.47323 | 0.03739 | 0.05 | 1 | −16.2245 | −0.37942 |

These results also show that different ratios of decellularized apples to NaOH can lead to similar end products. This flexibility in the production process allows for practical adjustments during scale-up, for example.

### Example 7-Protocol for Mechanical Testing of Aerogels and Foams

This example describes a protocol used for mechanical testing of aerogels and foams as described herein, such as those in Examples 1 and 2. In certain embodiments, the aerogels or foams as described herein may have a bulk modulus within a range of about 0.1 to about 500 kPa, such as about 1 to about 200 kPa, as determined by the protocol described in this example.

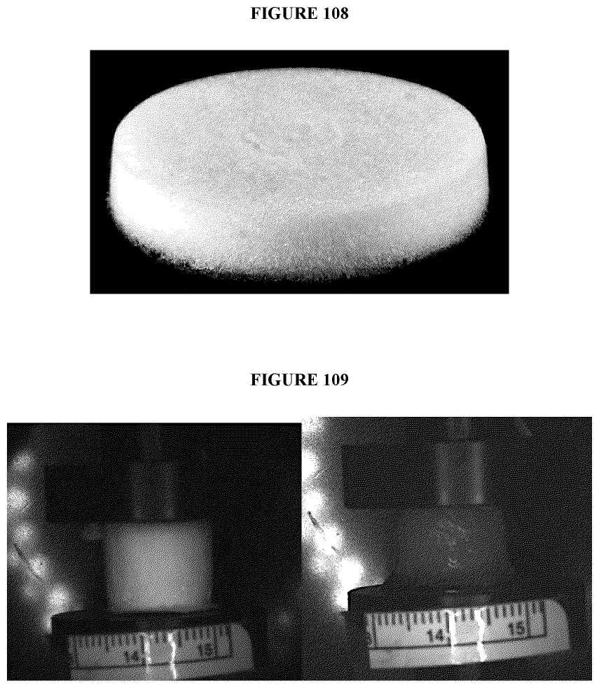

Sample preparation
1. A sample is prepared from an aerogel foam such as that shown in FIG. 108.
2. A biopsy punch of a desired size is used to cut out discs of the sample (e.g. 10 mm biopsy punch)
3. The sample is either kept dry (FIG. 109—left) or crosslinked with Calcium Chloride (FIG. 109—right)
4. The samples are then loaded onto the mechanical tester and compressed to a desired size or % of the sample size.

### Mechanical Testing Procedure

1. The sample for mechanical testing is prepared and all dimensions measured
2. The sample is mounted on the platons
3. The actuators are moved to the specified size, directly above the sample prior to making contact
4. The load cell is zeroed at the start of every test to ensure that the force measurements are accurate
5. The samples are placed as flat as possible to make full contact with the platons
6. The test is initiated and completes after the required number of compressions. Depending on the mechanical properties of interest the sample can be compressed from 5-100% of its initial thickness at a rate that is user specified (typically <0.1 mm/sec to >10 mm/sec). During compression, force data is measured with a load cell with an appropriate maximum (typically between 0.1-100N).
7. Once finished, the sample is discarded
8. The data is collected and the bulk modulus assessed in the initial elastic region of the curve.

### Bulk Modulus Extraction

For calculation of the bulk modulus, the raw data is provided as a force-extension curve which is converted into a stress-strain plot based on the measured dimensions of the sample. The linear portion of the compression curve is assessed and the slope determined in kPa.

### Example 8-Hydrogel Crosslinking in Aerogels and Foams

This example describes approaches for cross-linking of hydrogels in aerogels and foams as described herein, such as those in Examples 1 and 2 and elsewhere herein. In certain embodiments, at least some cellulose and/or cellulose derivative(s) of the aerogel or foam may be cross-linked by:
physical cross-linking (e.g. using glycine); or
chemical cross-linking (e.g. using citric acid in the presence of heat); or
wherein at least some cellulose and/or cellulose derivative(s) of the aerogel or foam is functionalized with a linker (e.g. succinic acid) to which one or more functional moieties are optionally attached (e.g. amine-containing groups, wherein cross-linking may further optionally be achieved with one or more protein cross-linkers such as formalin, formaldehyde, glutaraldehyde, and/or transglutaminase);
or any combinations thereof.

The cellulose-based materials may be cross-linked in a variety of ways. Broadly, crosslinking may be by physical cross-linking or chemical cross-linking, or both.

### Physical Cross-Linking:

An example of physical cross-linking is the use of glycine, which may, by way of illustrative example, be implemented as follows:

### Gel Formulation

The gelation process may involve dissolving methylcellulose in 10 mL of 2 M NaOH for 1 h with stirring on ice. A glycine solution was also prepared by dissolving glycine in 2 M NaOH. After 1 h, 5 mL of the glycine solution was added, and the mixture stirred on ice for an additional hour. The mercerized apple structural cells were introduced at one of two different stages. One method of introduction involved mixing in the mercerized apple with the viscous solution after the second hour of glycine treatment. This particular mixing method involved using syringes connected with an F/F luer lock system. It should be noted that for the higher methylcellulose concentration (1 g), the mixing with syringes was exceedingly difficult. As a result, a second preparation method was developed. In this approach, the mercerated apple was added directly to the 2 M NaOH with the methylcellulose at the start of the reaction. Mixing was accomplished with magnetic stirring. See Table 7 for the formulations tested. It was observed that when the glycine was added, the viscosity of the mixture increased. The gels were left at room temperature overnight to crosslink.

Such physical cross-linking with glycine has already been described in detail in Example 3 above, with reference to Table 7 and FIGS. 84-90.

### Chemical Cross-Linking

An example of chemical cross-linking is the use of citric acid and heat wherein the carboxylic acid groups may react with carboxymethylcellulose to form a chemically cross-linked matrix, which may, by way of illustrative example, be implemented as follows:

Preparation of carboxymethyl cellulose gels crosslinked with citric acid in combination with mercerized apple material (e.g. structural cells):

### CMC Preparation

2% CMC solution in water (w/w)
20% Citric acid (on polymer weight)
For combinations with the mercerized material an equal mass to the CMC was added
Stir at room temp until clear
Heat at 80° C. for 5, 8, and 16 h

### Results

After 5 h, a small gel began to form; however, the majority of it was still liquid.
After 8 h, a moderate amount of gel had formed. The CMC control was clear, whereas the CMC with mercerized material was translucent with a white tint. These gels had the consistency of low concentration collagen gels commonly used in cell culture experiments.

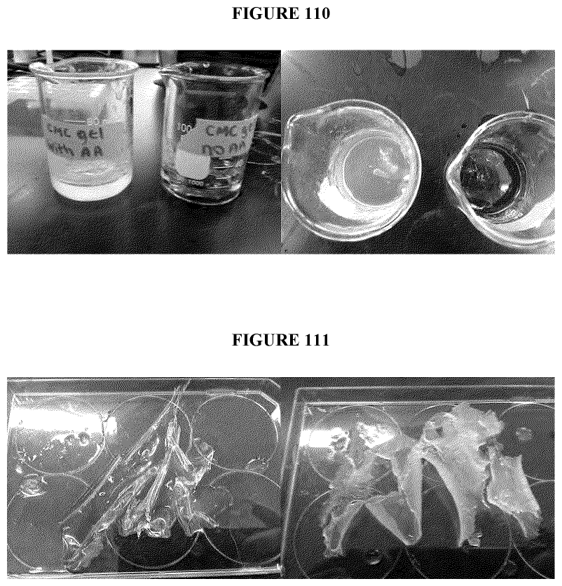

FIG. 110 shows results in which CMC cross-linked with citric acid is depicted. The CMC control was a clear gel, whereas the CMC with mercerized material (structural cells) was a translucent white gel.

After 16 h, a hard “glass or epoxy” like material was obtained. This was rehydrated and formed membrane materials.

FIG. 111 shows results for CMC crosslinked with citric acid membranes. The CMC control (left) was a clear membrane, whereas the CMC with mercerized material (structural cells) was a translucent white membrane that was more stiff—it had the texture of shrimp shells.

Expanding on chemical cross-linking, it is also contemplated herein that the cellulose structure may be functionalized with linker molecules that may be then used to add specific moieties to the cellulose chain for cross-linking purposes (among other purposes). For example, it is contemplated that succinic acid may be used to add a carboxylic acid group to the cellulose structure. The succinylated material may be used to add amine groups. It is contemplated that the amine groups may then be crosslinked with available protein cross-linkers such as formalin, formaldehyde, glutaraldehyde, and/or transglutaminase.

### Illustrative Example of a Succinylation Protocol:

a) Solvent and sample preparation
Solvent
   Dimethylacetamide (DMA) was dried in the fume hood to remove water
   Temperature: 115° C.
   Time: 45 minutes
LiCl
   LiCl was kept in the oven all times to prevent hydration.
   Temperature: 210° C.
Samples
   Decellularized apple-solvent exchange
   Step 1. The apple pieces were kept in ethanol or acetone for 20 minutes in an ultrasound bath. Three cycles of solvent exchange with ethanol were performed.
   Step 2: The pieces of apple were immersed in DMA for 20 minutes in an ultrasound bath. Three cycles of solvent exchange with DMA were performed.

### b) Succinylation of Cellulose Using DMA and LiCl

Scaffold: Mass of apple cellulose (after) solvent change=360 mg
Chemicals and reagents:
   DMA=30 mL
   LiCl=271 mg
   AS (succinic anhydride)=3.1 g
Conditions
   Temperature: 80° C.
   Duration: 6 h (under rotational agitation)

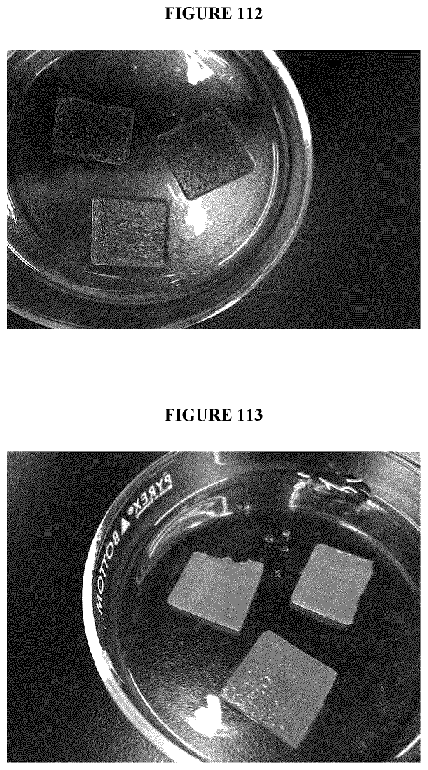

After solvent preparation (DMA and LiCl) the cellulose pieces are immersed in DMA. The LiCl is then added. The mixture was stirred for 30 minutes. After 30 minutes, the succinic anhydride (AS) was added. The mixture is placed in the oven at 80° C. for 6 h. After this process, the solvent is removed, and the cellulose is washed intensely with water until the scaffold is clean and free of visible residue. The images in FIG. 112 show the cellulose immediately after the reaction and after excessive washing.

### c) Results

Cellulose after reaction is complete is shown in FIG. 112. Cellulose after intensely washing with water is completed is shown in FIG. 113.

### Ftir Analysis:

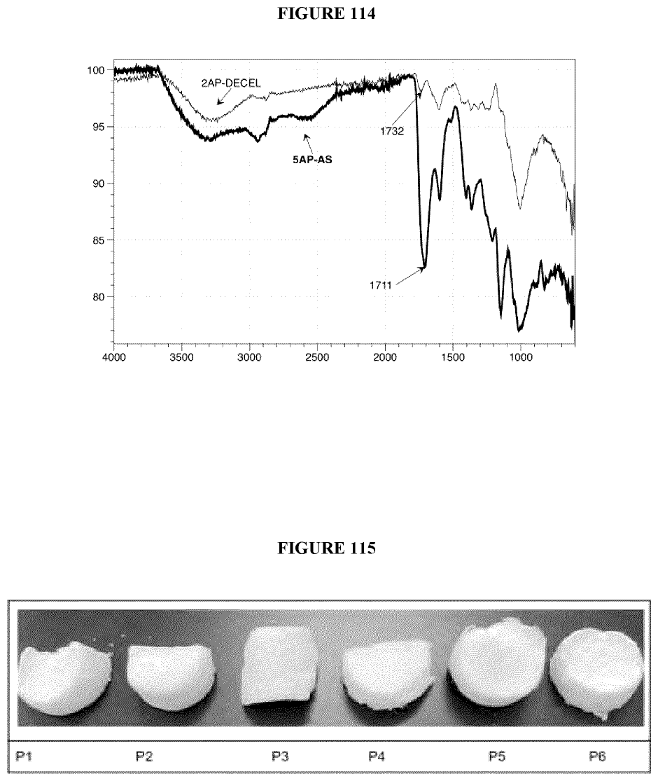

In order to confirm the chemical crosslinking was accomplished, Fourier Transform Infrared Spectroscopy was used. The spectral shifts reveal that the succinic anhydride was successfully chemically bonded to the scaffold. This linker molecule may be used to attach other molecules such as collagen. FTIR spectra is shown in FIG. 114, showing FTIR spectra of decellularized scaffolds (2AP-DECEL) and the chemically bonded composite of succinylated plant-derived cellulose (5AP-AS).

### Chemical Modification and Functionalization Process Example

### First Step-Acylation of Cellulose

Methodology: Through homogenous succinylation using succinic anhydride

### Method 1:

Cellulose suspended in DMAc/LiCl allowed to stir in the presence of CO2 (2-5 bar) to obtain a clear solution. Afterwards, succinic anhydride is added to the clear solution of cellulose and stirred at room temperature. The resulting reaction mixture is poured to vigorously stirring water (200 mL). Then, water was slightly acidified with a diluted (0.05 M) hydrochloric acid solution to obtain white precipitates. The crude product is collected and dissolved in DMAc/LiCl and reprecipitated in water (200 mL). The white product is further washed with water (200×3 mL) to remove DMAc/LiCl. The pure white product is dried under vacuum at 70° C. for further esterification process.

### Method 2:

Cellulose succinylation using succinic anhydride, pyridine in dichloromethane in a static system. The reaction is performed under reflux. After the period of reaction, the reaction is quenched by adding of MeOH. Then, the material is washed several times to remove pyridine.

### Second Step-Esterification Process

Chemical modifications of cellulose, such as esterification, etherification and grafting (from or onto) are among the most common techniques. Such derivatizations may be achieved via heterogenous or homogenous approaches. Heterogenous modifications on the surface of cellulose fibres have been more common due to the solubility challenge of cellulose. Homogenous modifications of cellulose, on the other hand, are desirable as the latter may enable to control the DS by adjusting the reaction conditions. Table 10 shows examples of esterification processes.

TABLE 10
Examples of Esterification Processes

| Reaction types | Esterifying agents | Catalysts |
|---|---|---|
| Inorganic | Sulfuric acid, phosphoric acid | N/A |
| esterification | | |
| Fischer | Acetic acid, butyric acid, citric | Hydrochloric |
| esterification | acid, malic acid, malonic acid | acid |
| Mechanochemical | Succinic anhydride, ndodecyl | N/A |
| esterification | succinic anhydride, hexanoyl | Pyridine |
| | chloride | |
| | Pentafluorobenzoyl chloride | |
| Transesterification | Vinyl acetate, vinyl cinnamate, | Potassium |
| | canola oil fatty acid methyl | carbonate |
| Solvent-free | Palmitoyl chloride | N/A |
| esterification | Iso-octadecenyl succnic | N/A |
| | anhydride, ntetradecenyl | |
| | succinic anhydride | |
| | Acetic anhydrid | Citric acid |
| | Aromatic carboxylic acids | Sulfuric acid |

### Methodology

Esterification may involve high temperature and a crosslinker agent. 1-Ethyl-3-(3-dimethylaminopropyl)carbodiimide (EDC), citric acid, and fumaric acid are commonly used in an esterification reaction to form a hydrogel.

EDC promotes crosslinking between carboxyl groups and hydroxyl or amine groups with the formation of nontoxic, water-soluble urea derivative. EDC is preferable for crosslinking reaction because of high conversion efficiency, mild reaction conditions, nontoxicity, and easily separable byproducts and compatibility with materials. Also, during the esterification of cellulose and citric acid, anhydride intermediate is formed.

So, before transglutaminase application, the cellulose may be submitted to chemical modification using succinylation (acylation) followed by preparation of activated NHS-esters and their reaction with nucleophilic amino acid residues present in proteins.

### Transglutaminase Protocol:

The following procedure outlines illustrative examples of general usage for MooGloo (i.e., the transglutaminase enzyme) in food applications, such as for the binding and gluing of AA aerogels and other biomaterials.

### Key Chemicals and Solutions

1. MooGloo RM Transglutaminase Formula (Modernist Pantry)
2. MooGloo TI Transglutaminase Formula (Modernist Pantry)
3. MooGloo GS Transglutaminase Formula (Modernist Pantry)
4. Distilled water

### Procedure-RM Formula

1. Open the vacuum-sealed packaging containing the transglutaminase powder.
2. Make a 1:4 ratio (weight/volume, w/v) solution of the transglutaminase powder with distilled water (i.e., 5 g of dry powder mixed with 20 mL of distilled water); mix thoroughly.
3. Pour the slurry onto the designated areas of the biomaterial to be glued and spread evenly.
   a. If the biomaterial is a gel/liquid solution, the transglutaminase powder may also be mixed directly into the solution at a 0.5-1.0% w/v concentration to thicken or bind the biomaterial into a uniform shape.
4. After mixing or glueing the biomaterial, wrap tightly with parafilm or store in an airtight container.
5. Transfer the material to the fridge and store at ˜4° C. for 6-24 hours to bind.
6. Store the remaining transglutaminase powder in the freezer for up to 6 months.

### Procedure-TI Formula

1. Open the vacuum-sealed packaging containing the transglutaminase powder.
2. Make a 1:4 ratio (weight/volume, w/v) solution of the transglutaminase powder with distilled water (i.e., 5 g of dry powder mixed with 20 mL of distilled water); mix thoroughly.
3. Pour the slurry onto the designated areas of the biomaterial to be glued and spread evenly.
   a. If the biomaterial is a gel/liquid solution, the transglutaminase powder may also be mixed directly into the solution at a 0.5-1.0% w/v concentration to thicken or bind the biomaterial into a uniform shape.
4. After mixing or gluing the biomaterial, wrap tightly with parafilm or store in an airtight container.
5. Transfer the material to the fridge and store at ˜4° C. for 6-24 hours to bind.
6. Store the remaining transglutaminase powder in the freezer for up to 6 months.

### Procedure-GS Formula

1. Open the vacuum-sealed packaging containing the transglutaminase powder.
2. Make a 1:4 ratio (weight/volume, w/v) solution of the transglutaminase powder with distilled water (i.e., 5 g of dry powder mixed with 20 mL of distilled water); mix thoroughly.
3. Pour the slurry onto the designated areas of the biomaterial to be glued and spread evenly.
   a. Note: the GS formulation cannot be used as a dry powder; if the biomaterial is a gel/liquid solution, mix the slurry directly into the solution at a 0.75-1% v/v concentration.
4. After mixing or gluing the biomaterial, wrap tightly with parafilm or store in an airtight container.
5. Transfer the material to the fridge and store at ˜4° C. for 6-24 hours to bind.
6. Store the remaining transglutaminase powder in the freezer for up to 6 months.

### Example 9-Cell-Based and Plant-Based Meat Products and Meat Mimic Food Products

In certain embodiments, aerogel and/or foam materials as described herein may be used for preparing cell-based and/or plant-based meat products, meat mimics, cell cultured meat, cell-based meat, other food applications, cellular agriculture applications, etc. Mercerized materials (such as structural cells) as described herein may be combined with a number of different hydrogels (as described above), for example, alginate, pectin, gelatin, methylcellulose, carboxymethylcellulose, dissolved and regenerated cellulose, etc. The mixtures may then be placed into a container of generally any suitable size, frozen and then lyophilized to remove all water. Once completely dehydrated, the material may be crosslinked with (for example) calcium chloride or another crosslinking agent. This step may be performed either before or after lyophilization, and may result in a desirable format of the material. This example shows use of the aerogel for the production of a plant-based meat product.

In certain embodiments, the aerogel may be used to produce a plant-based meat product by customizing its method of preparation, such as the vessel in which it is prepared. By way of example, to produce a piece of tuna sashimi, the mercerized material was molded into a 100 mm dish, then frozen for 24 hours at −20° C. The frozen material was then lyophilized for 48 hours, and then crosslinked with calcium chloride. In an embodiment, once crosslinked, the material may then be stained, dyed or cut to any desired shape such as for a piece of sashimi. The material was stained with food coloring by adding a few drops (2-6 drops, or desired amount for desired colour) to a container with water such that the water was covering the material entirely (e.g. a material used to form a 100 mm dish-10-20 mL of coloured water or food dye). Once stained, the material was cut into a rectangular shape with a scalpel, knife or another sharp blade. Vertical slices may be cut across the slice to mimic the white lines found in tuna or salmon. A food-grade gluing agent such as agar, agar-agar, gelatin or similar agents may be used to fill in the lines cut by the blade. In one embodiment, 1-5% agar may be used to glue pieces of material together, or fill in lines made by cutting the material, approximately half to ¾ of the way through. The agar may also be combined with food grade titanium dioxide (Pan Tai, PTR-630) (0.1-1 g per 100 mL of agar) to color the lines white.

FIG. 117 shows a “Tuna” sushi mimic (red coloured) which was created by layering and gluing pieces of aerogel (cross-linked 50% Alginate) scaffolds with more alginate. The construct was then cut into a 3×2 cm piece (approx) and coloured with red food dye to mimic real tuna. Small diagonal slices were cut along its length to mimic the interface between muscle layers.

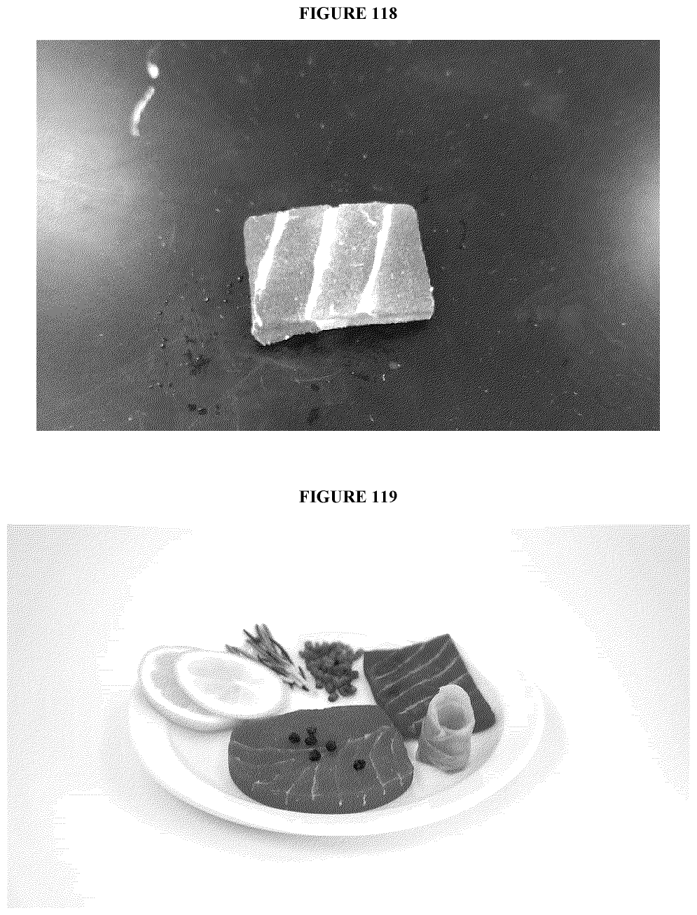

FIG. 118 shows a “Tuna” sushi mimic (red coloured) which was created by layering and gluing pieces of previously dyed aerogel (cross-linked 50% Alginate) with an agar “glue” that had been coloured with titanium dioxide (TiO₂), a common white food colorant. This construct allowed to more convincingly mimic the fascia that exists between distinct layers of muscle tissue in real tuna.

FIG. 119 shows a “Tuna” (red coloured) which was created by layering and gluing pieces of previously dyed aerogel (cross-linked 50% Alginate) with an agar “glue” that had been coloured with titanium dioxide (TiO₂), a common white food colorant. The agar glue may be placed between layers, or into thin grooves cut along the surface of the aerogel to produce the linear pattern of fascia which exists between muscle layers.

### Methods for Cooking:

The resulting biomaterial may be cooked once cut, stained and prepared to a desired appearance, and texture. The material may be pan-fried, baked, or prepared in another method (e.g. under vacuum, sous vide), or left uncooked. In one embodiment, the material may be pan-fried for about 1-10 minutes on each side with a small amount (¼ tsp) of butter on a cast-iron skillet heated to 200° C. (see FIGS. 9-12). The material was fried until golden (approximately 3 minutes for a 10 mm-100 mm diameter material), then removed from the pan onto a dish.

### Mechanical Testing Post Cook:

Once cooked, the material may be mechanically tested to determine its bulk compression modulus. In one embodiment, the material was cut to a 1 cm×1 cm size piece. It was placed atop a platen on a mechanical tester (such as a Univert by CellScale) and compressed. The rate (compression force or size per second), maximum load (depending on the load cell), displacement of compression (from 5% to 95% of its size), among other features may be customized. In one embodiment, a round sample of approximately 10 mm diameter and 5 mm thickness was mechanically tested by compression in both axial and radial directions. The biomaterial was cut to the specified size, and placed on the mechanical tester. Size measurements were recorded then the material was compressed three times to 50% of its size.

Measured bulk modulus from 10 cm cooked and uncooked biomaterials were as follows:

| | Dry (kPa) | Wet (kPa) | Cooked (kPa) |
|---|---|---|---|
| Axial | 168.12 ± 39.34 | 4.10 ± 2.06 | 5.61 ± 3.06 |
| Radial | n/a | 9.58 ± 4.41 | 9.58 ± 4.96 |

The present data shows that the mercerized aerogel may be used to produce a plant-based scaffold for the development of a meat-free alternative for a food product. The resulting material may be customized wherein its size, shape, appearance, texture and/or mechanical properties may all be tuned for the production of the desired material, such as a piece of fish (e.g. salmon or tuna) or a steak or piece of chicken, for example. Additionally, the data supports that cooking may result in an increase in the bulk modulus or a stiffening of the material. This may be a desirable property, as cell-based meat may also stiffen from cooking. These results are promising for the development of a variety of plant-based food products. Finally, as the aerogel may be compatible for cell culture (see FIG. 41), it may easily be employed as a scaffold for cell-based meat, cell cultured meat, cultivated meat in cellular agriculture applications, for example.

### Example 10-Dermal Filler Products

In another embodiment, mercerized materials, structural cells, aerogels, foams, dissolved celluloses, and/or other such materials as described herein may be used for dermal filler applications. By way of example, the mercerized material may be used on its own as a dermal filler hydrogel, or it may be combined with other carrier gels and/or fluids such as saline, collagen, hyaluronic acid, methylcellulose, and/or dissolved plant-derived decellularized cellulose, for example. Such formulations may be used in combination with lidocaine, for example, or another such agent relevant for dermal filler applications. This Example provides data for the use of the mercerized decellularized apple structural cells as particles for dermal fillers.

Needle Occlusion Test for merAA (Mercerized Apple):

The size analysis of the mercerized material revealed that the process (as already described in detail hereinabove) may separate the scaffolds into single structural cells. These apple cell walls have sizes that are comparable to HA particle sizes used in dermal fillers. Therefore, the mercerized material was selected as a candidate for dermal filler applications. This may be used as a material on its own, or in combination with other gels/formulations as discussed below. The first test was an occlusion test through a 27 G needle. It was found that the needle did not occlude, aside from large chunks that were filtered out. This is a significant result, as HA fillers typically use 27-30 G needles, whereas BellaFill uses 26 G needles.

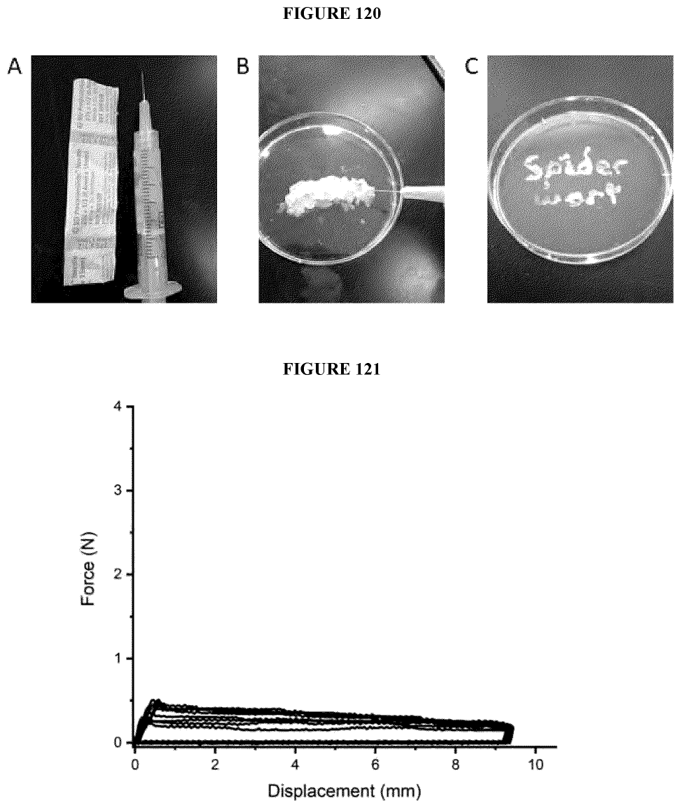

FIG. 120 shows the needle occlusion test with mercerized AA. In (A), a 27 G needle and syringe is shown. (B) shows extrusion of mercerized AA. (C) shows an example for 3D printing or controlled injection/extrusion, for example.

The mixtures were made in 5 ml syringes and transferred to 1 cc syringes for a volume of 0.3 ml. With the use of interlock connectors on the 5 ml syringes, we could mix the mercerized AA with the saline solution thoroughly by mixing 30×.

Formulations were as Follows:

| Ratio ID | Components |
|---|---|
| Water | Water |
| Mer20Sal80 | 20% (v/v) mercerized, decellularized |
| | AA diluted in 0.9% saline |
| Mer100 | Undiluted mercerized, decellularized AA |

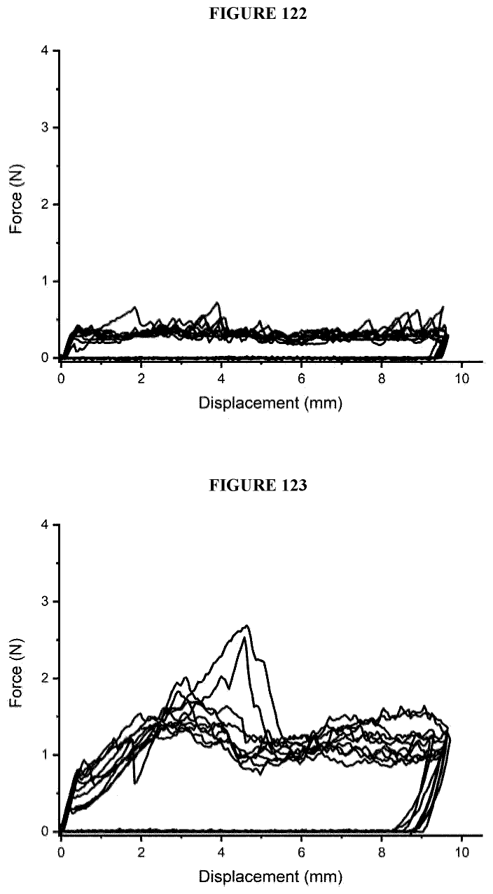

FIGS. 121, 122, and 123 show force extension curves. FIG. 121 shows force-displacement curves for N=10 extrusions of water from a 1 cc syringe. FIG. 122 shows force-displacement curves for N=10 extrusions of a 20% mercerized AA in saline mixture from a 1 cc syringe. FIG. 123 shows force-displacement curves for N=10 extrusions of undiluted mercerized AA from a 1 cc syringe.

### Maximum Force:

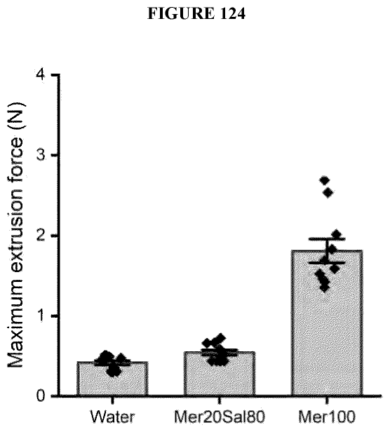

The maximum extrusion force was used to compare the three formulations. The descriptive statistics are displayed below, and visually compared in FIG. 124 which shows maximum extrusion force for water only, a 20% mercerized AA solution diluted in 0.9% saline, and undiluted mercerized material.

Descriptive Statistics of the Maximum Extrusion Force from a 1 cc Syringe:

| | | | Standard | Standard |
|---|---|---|---|---|
| Solution | N | Mean | deviation | error |
| Water | 10 | 0.41 | 0.09 | 0.03 |
| 100% Mer AA | 10 | 1.81 | 0.47 | 0.15 |
| 20% Mer AA + | 10 | 0.54 | 0.11 | 0.04 |
| 80% Saline | | | | |

This data shows that the undiluted mercerized material had a significantly greater extrusion force than the diluted mercerized material and the water control. Moreover, the results reveal that there was no significant difference between the diluted mercerized material and the water control. This provides insight into the sensitivity of the measurement. The viscosity of the diluted material is different from distilled water; however, this slight difference was not discernable with the current method.

### Dermal Filler Applications-Rat Model Injections and Size Measurements:

As the occlusion test was successful, the material was implicated as a dermal filler in a test rat study. The injection volumes were 600 μL, and 4 injections per animal were performed. All the formulations contained 0.3% lidocaine. Lidocaine was used in this case but a number of other anesthetics are also possible. Typical anesthetics may include, for example, 2% lidocaine gel and a triple anesthetic gel composed of 20% benzocaine, 6% lidocaine, and 4% tetracaine, (BLTgel). Dental blocks with 3% Polocaine are given with a 30 G needle to anesthetize the upper and lower lips and perioral region prior to injection. Also mixes of 2% lidocaine with epinephrine may be used.

The first formulation was mercerized AA alone. The second filler was mercerized material diluted with 0.9% saline to give a final concentration of 20% volume of the mercerized material. Similarly the third formulation comprised a 20% mercerized material and 3.5% collagen mixture. Lastly, the fourth filler was a 20% mercerized material and regenerated cellulose mixture. The regenerated cellulose was derived from decellularized apple tissue and was dissolved according to the methods previously described above with DMAc and LiCl. The formulations were coded as MER, MER20SAL80, MER20COL80, and MER20REG80 respectively.

Dermal Filler Formulation Components were as Follows:

| | Mercerized | 0.9% | | Regenerated | |
|---|---|---|---|---|---|
| | material | Saline | Collagen | cellulose | Lidocaine |
| Code | (v/v %) | (% v/v) | (% m/v) | (% v/v) | (m/v %) |
| MER | 85 | 0.0 | 0.0 | 0.0 | 0.3 |
| MER20SAL80 | 20 | 65 | 0.0 | 0.0 | 0.3 |
| MER20COL80 | 20 | 11 | 3.5 | 0.0 | 0.3 |
| MER20REG80 | 20 | 11 | 0.0 | 54 | 0.3 |

* Note the volume of the lidocaine was 15% of the formulation. A 2% liquid lidocaine stock was used.

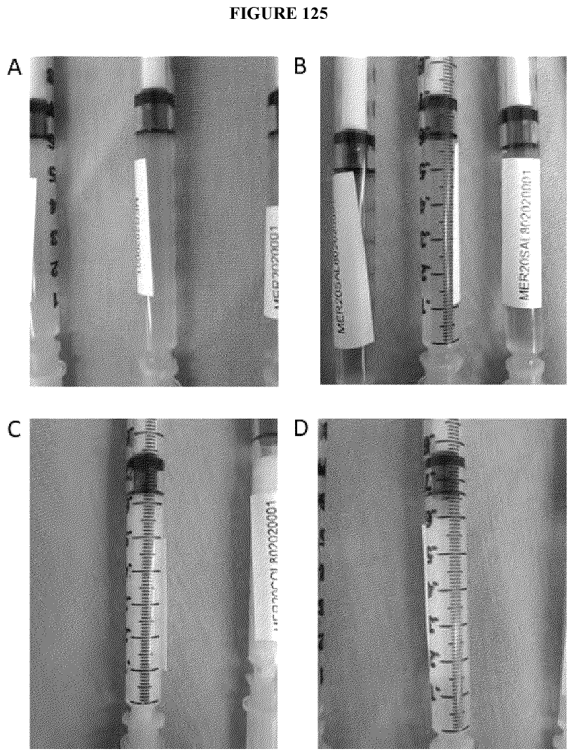

FIG. 125 shows generation II dermal fillers. (A) shows MER, (B) shows MER20SAL80, (C) shows MER20COL80, and (D) shows MER20REG80. The injections contained 0.3% lidocaine and were prepared as 600 μL injections in 1 cc syringes.

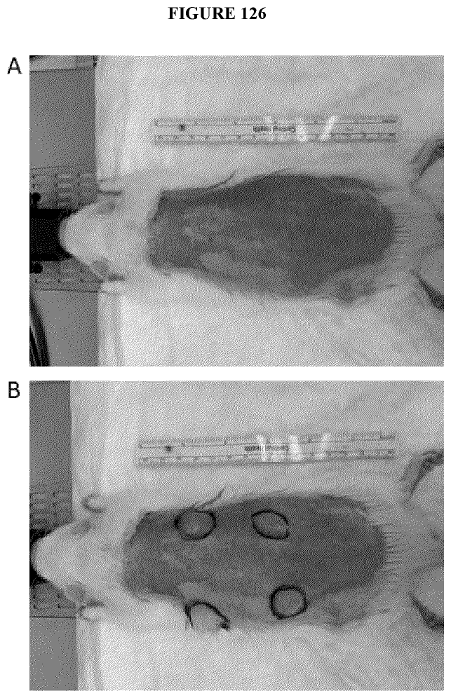

FIG. 126 shows results for generation II dermal fillers used as dermal filler in a rat model. (A) shows Pre-injection, and (B) shows Post-injection. The black outline was used to track the implant sites from week to week. The bumps under the skin were measured. The bump sizes were measured using Vernier calipers. The ellipsoid estimate was used to estimate the area and volume of the injections.

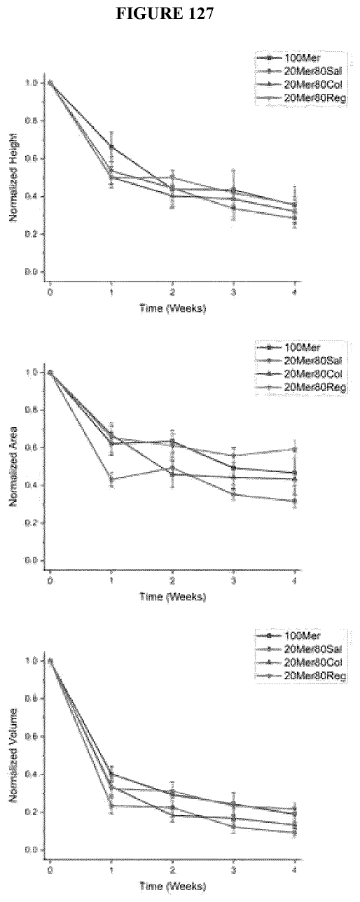

FIG. 127 shows dermal filler size measurements for the rat model injections. (A) shows the normalized height, (B) shows the normalized ellipse area, and (C) shows the normalized ellipsoid volume.

### Example 11-Crosslinking with Citric Acid

Cellulose is a polymer that presents abundant hydroxyl groups and can be used to prepare hydrogels with fascinating structures and properties. On the basis of the cross-linking method, the hydrogels can be divided into chemical gels and physical gels. Physical gels are formed by molecular self-assembly through ionic or hydrogen bonds, while chemical gels are formed by covalent bonds.

Cellulose and cellulose derivatives define their extensive usage in different applications, and cellulose esters and cellulose ethers are two main groups of cellulose derivatives with different physicochemical and mechanical properties. Cellulose esters are water-insoluble polymers with good film-forming characteristics which find a variety of applications.

The attachment of the polyfunctional carboxylic acids via esterification with a cellulosic hydroxyl group and its esterification with another cellulosic hydroxyl group produce crosslinked cellulose chains. Attachment of the carboxylic acid moiety to cellulose's hydroxyl group via esterification reaction of the first cyclic anhydride would expose a new carboxylic acid unit in carboxylic acid, which has the proper chemical connectivity to form a new intramolecular anhydride moiety with the adjacent carboxylic acid unit.

Citric acid (CA) is regarded as a non-toxic and relatively inexpensive crosslinking agent that has been used to modify polysaccharides such as cellulose.

The suggested mechanism for the cross-linking process of cellulose with citric acid with regard to the conventional cross-linking of cellulose using citric acid in the presence of acid catalysts is shown below:

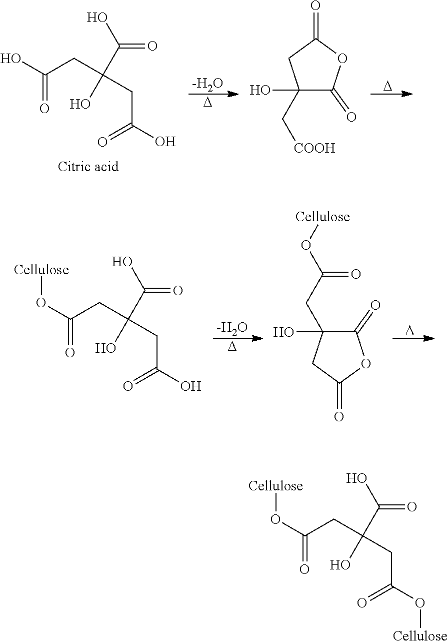

Aerogels Produced from Cellulose Crosslinked with Citric Acid

In this example, various characteristics of aerogels produced from cellulose crosslinked with citric acid were evaluated such as the pore size and alignment along with the stability of aerogels. The aerogels were prepared from regenerated cellulose or from mercerized cellulose crosslinked with citric acid to compare the effect of the two forms of cellulose in aerogel preparation for downstream applications. The goal was to produce an aerogel which can be used as a scaffolds for bone repair and/or spinal cord regeneration. The first aerogel was produced from a mixture of 1) regenerated cellulose (ADICLS) crosslinked with citric acid and 2) succinylated cellulose generated by unidirectional freezing. The second aerogel was produced from a mixture of 1) mercerized cellulose (Merc. AA) crosslinked with citric acid (S4) and 2) succinylated cellulose generated by unidirectional freezing. The stability of the two aerogels was then compared.

### Methodology

Crosslinking with Citric Acid

Four different samples were prepared by mixing mercerized cellulose (Merc. AA) (0.1 g/mL) and succinylated cellulose (0.1 g/mL) in different proportions according to the table below, and mixed with 2% citric acid to evaluate if 2% citric acid was enough to produce a stable aerogel in PBS. Briefly, the polymers were suspended in 50 mL of water to form a gel before citric acid was added and kept under vigorous stirring with a glass rod for 15 minutes. The reaction was performed at 100° C. for 20 minutes on a hot plate shaker and all surface moisture was removed. The material was removed from the oven and kept at room temperature overnight. The next day, the material was incubated in the oven for 2 h at 105° C. The reaction products were then slurried in water (60 ml) for 30 min, adjusted to pH 7, and washed three times by centrifuging at 4500 rpm for 10 minutes to remove the unreacted components.

Name of crosslinked samples, celluloses
and citric acid concentration used.

| | Citric acid | Name of |
|---|---|---|
| Polymer (%) | concentration | sample |
| Mercerized AA - 50 | 2% | S1 |
| Succinylated mercerized - 50 | | |
| Succinylated mercerized - 75 | 2% | S2 |
| Mercerized AA - 25 | | |
| Succinylated mercerized - 25 | 2% | S3 |
| Mercerized AA - 75 | | |
| Mercerized AA - 100 | 2% | S4 |

### Production of Aerogels

Agarose (1%) was liquefied in hot water (70° C.) and mixed with 40% hydroxyapatite (HP). Crosslinked Mer. AA (20 g) and succinylated cellulose were loaded in a syringe together with 12 mL of the liquefied agarose and HP. The polymers were mixed using two 60 mL syringes with a Luer Lock connector. The amount of succinylated cellulose was adjusted according to the proportion described in Table 1.

The resulting material was placed in a 60 mm TC dish and incubated at −20° C. for at least 2 hours to completely freeze the material which was then lyophilized for 24 hours.

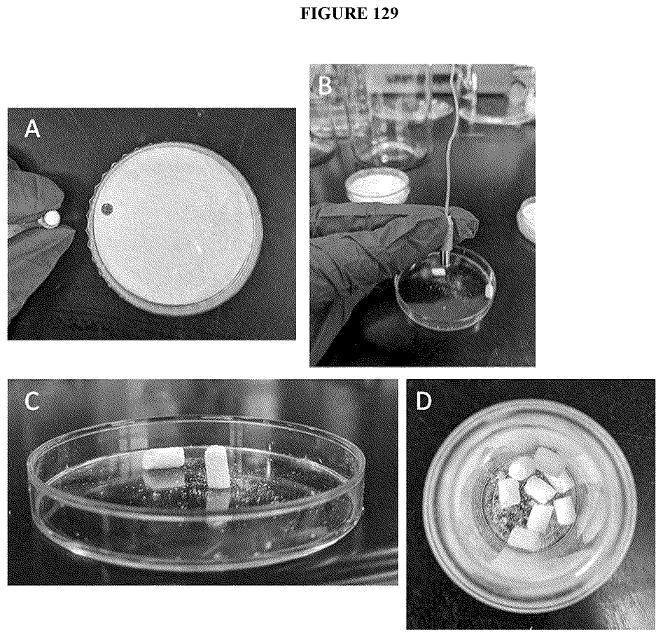

As shown in FIG. 129, the freeze-dried aerogel was then punched out using a 5 mm biopsy punch (A), then removed using a thin wire (B), and the short-term stability of the aerogel in PBS was evaluated.

Aerogels were prepared using regenerated cellulose to compare the porosity with the other aerogel prepared from Mer. AA CL.

The particle size of the aerogels was assessed by comparing aerogels produced from crosslinked regenerated cellulose (D1CL) and crosslinked mercerized cellulose (S4), both mixed with succinylated mercerized cellulose. These materials were evaluated with the goal to obtain more homogeneous suspension and further aligned porous formation under directional freezing. The samples were prepared as described in Table 2 below.

Formulation and name of aerogel samples.

| | | | Name of |
|---|---|---|---|
| Polymer 1 | Polymer 2 | H₂O | Aerogel |
| Succinylated | Mercerized cellulose | 4 mL | SS4 |
| mercerized | crosslinked with citric acid | | |
| cellulose - 5 g | (2%) (S4) - 5 g (Mercerized | | |
| | cellulose - 0.1 g/mL) | | |
| Succinylated | D1(D1CL) - 5 g | 4 mL | AD1CLS |
| mercerized | Crosslinked (citric acid (2%)) | | |
| cellulose - 5 g | regenerated cellulose | | |

The aerogels were prepared as previously described above. Briefly, the polymers were mixed in the amount indicated in the table above using two 50 mL syringes connected with an f/f Luer Lock. The previously prepared sample (S4-crosslinked mercerized cellulose) was mixed with water (4 mL) and inserted into the syringes together with succinylated cellulose and the polymers were mixed at least 30×. The same procedure was realized for the crosslinked regenerated cellulose (D1). The samples were placed into steel tubes onto the directional freezer for 2 hrs.

After directional freezing the material was transferred to the freezer −20° C. and kept for 24 h, followed by lyophilization for at least 24 h.

### Results

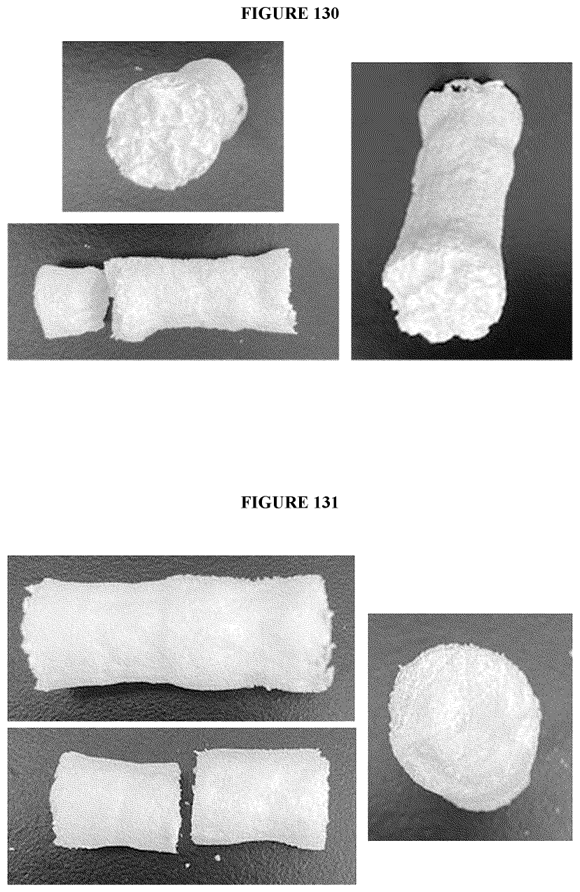

FIG. 130 shows an aerogel produced with crosslinked regenerated cellulose (D1) and succinylated cellulose.

FIG. 131 shows an aerogel produced with crosslinked mercerized cellulose (AS4) and succinylated cellulose.

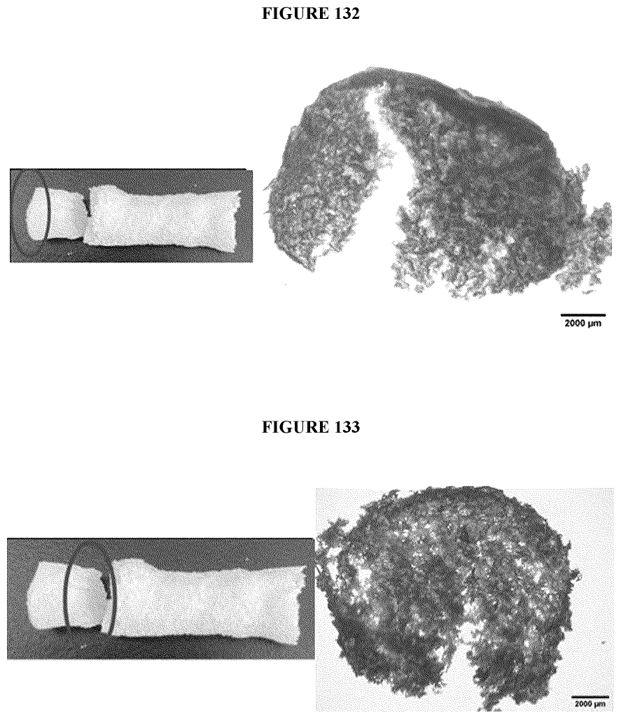

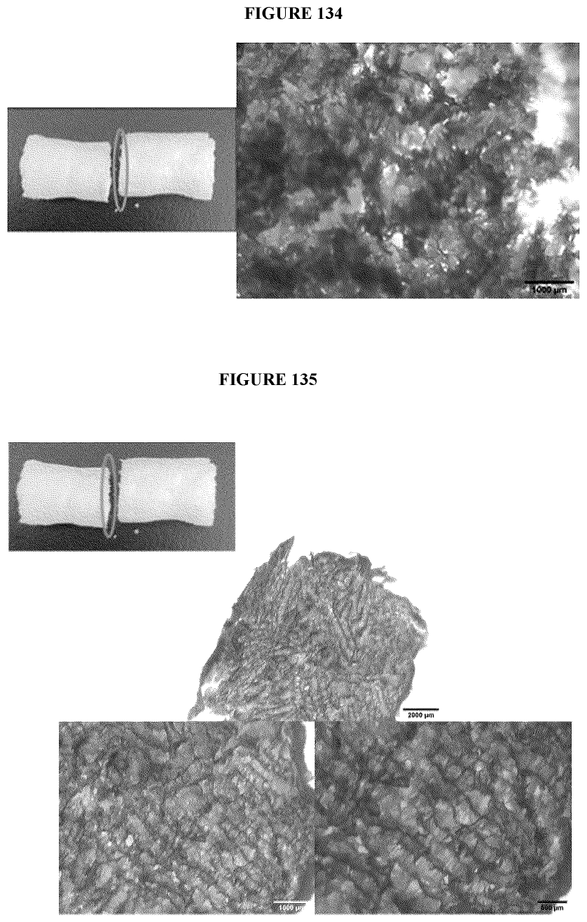

The aerogel prepared from crosslinked regenerated cellulose (D1) was named “ADICLS” and presented two layers. Previous results have shown that aligned pores are usually only formed in the bottom portion, so the microscopy imaging was performed in the bottom portion (portion directly in contact with the copper plate used for directional freezing) and in the top surface of the bottom layer for analysis as indicated by the circled region in FIGS. 132 to 133 for the ADS1 aerogel. Other regions were examined for the aerogel prepared from crosslinked mercerized cellulose (AS4) as depicted in FIGS. 134-135 because they broke after submitted to directional freezing and lyophilised.

FIG. 132 shows a brightfield microscopic image of the circled bottom surface of the bottom layer of an aerogel prepared from crosslinked regenerated cellulose (AD1CLS).

FIG. 133 shows a brightfield microscopic image of the circled top surface of the bottom layer of the aerogel of FIG. 132.

FIG. 134 shows a brightfield microscopic image of the circled bottom surface of the top layer of an aerogel prepared from crosslinked mercerized cellulose (AS4).

FIG. 135 shows a brightfield microscopic image of the circled top surface of the bottom layer of the aerogel of FIG. 134.

The results demonstrate that sample S4 produced a more porous and organized structure with a region showing aligned porousness. However, the samples were highly unstable in water when the stability was assessed. Further optimisation in the crosslinking process was therefore performed.

### Example 12-Optimization of Citric Acid Concentration for Crosslinking

The density (or the porosity) of the final material depends on the initial mass of mercerized cellulose. As such, the cellulose mass was increased and the effect of citric acid concentration on the aerogel during the crosslinking process was evaluated. Mercerized cellulose was selected to prepare these samples.

### Methodology

Mercerized cellulose (Merc. AA) was prepared in water as described above. Citric acid with different concentrations were dissolved in a minimal amount of water (3 mL) in a beaker. The citric acid was then added in the Merc. AA gel and mixed vigorously. The crosslinking process was performed as described above. The different concentrations of citric acid used to prepare samples S6-S10 is shown the table below.

Names of Crosslinked Samples According
to Citric acid Concentration

| | Name of cellulose cross | |
|---|---|---|
| Citric acid % | linked samples | Composition |
| 2 | S6 | merAA + 2% citric acid |
| 4 | S7 | merAA + 4% citric acid |
| 5 | S8 | merAA + 5% citric acid |
| 10 | S9 | merAA + 10% citric acid |
| 20 | S10 | merAA + 20% citric acid |

Reaction products were slurried in water and the pH was adjusted to pH 7. The cellulose citrate product was air dried.

Hydrogels were then produced by mixing S6, S9 and S10 with succinylated mercerized cellulose. Since gels produced using S7 and S8 were relatively unstable, these samples were discarded and not used in subsequent experiments. Succinylated mercerized cellulose was selected because it has more hydrophilic characteristics which allows production of more homogeneous and uniform hydrogels which can improve alignment of pores under directional freezing.

The polymers were prepared as described above. Briefly, the two polymers (crosslinked Merc. AA+succinylated mercerized cellulose) were mixed using two 50 mL syringes connected with an f/f luer lock connector. The crosslinked mercerized cellulose (S6, S9 or S10) were mixed with water and the succinylated cellulose added thereafter and inserted into the syringes and the polymers were mixed. The samples were placed into the directional freezer. The aerogels were prepared and named according to the table below.

Polymer Concentration in Each Aerogel

| Polymer | Polymer | Name of Aerogel |
|---|---|---|
| Succinylated mercerized | Mercerized cellulose citric | AS6 |
| cellulose | acid (2%) = S6 | |
| Succinylated mercerized | Mercerized cellulose citric | AS9 |
| cellulose | acid (10%) = S9 | |
| Succinylated mercerized | Mercerized cellulose citric | AS10 |
| cellulose | acid (20%) = S10 | |

### Results

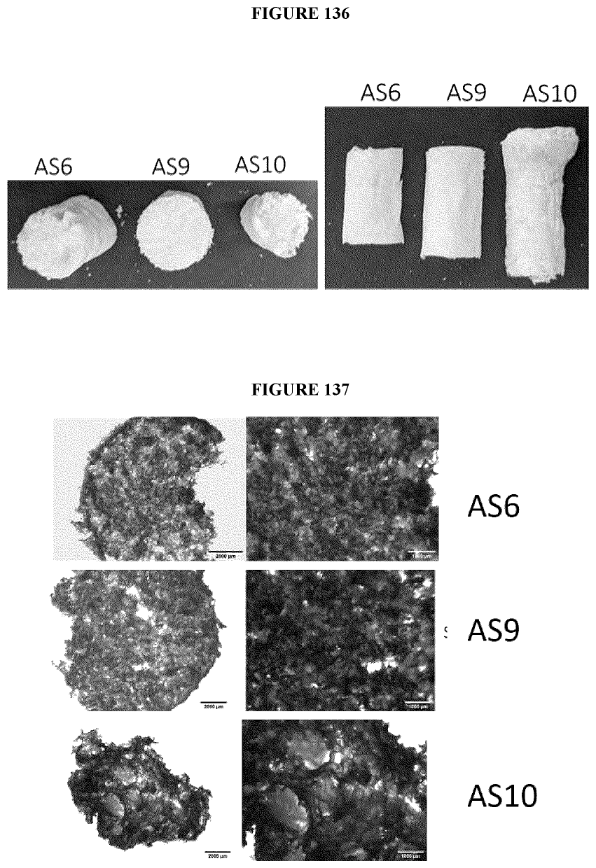

FIG. 136 shows aerogels AS6, AS9 and AS10 prepared from crosslinked mercerized cellulose (samples S6, S9 and S10) mixed with succinylated mercerized cellulose.

FIG. 137 shows microscope images of the bottom surface of the bottom layer of each aerogel AS6, AS9 and AS10.

As shown in FIG. 136, these formulations resulted in aerogel hard and brittle characteristics with all samples presenting two layers (bottom and top) after directional freezing and lyophilization.

FIG. 137 shows microscope images of the bottom surface of the bottom layer of each aerogel AS6, AS9 and AS10. The bottom surface of the bottom layer is the layer directly in touch with the directional freezing plate. The images show pores of different sizes but that are not aligned.

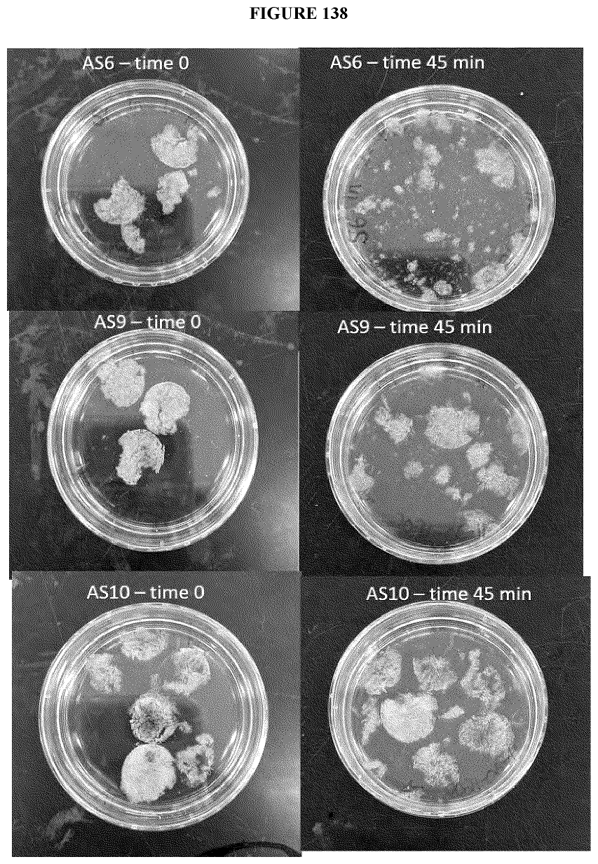

The stability of each aerogel in PBS was then evaluated. FIG. 138 shows stability of each aerogel AS6, AS9 and AS10 after 45 minutes in PBS compared to the aerogel at t=0. The images show that increasing the citric acid concentration increased the stability of the aerogels. Indeed, FIG. 138 show that after 45 minutes the sample AS6 (citric acid 2%) was completely fragmented into small pieces whereas the sample AS9 (citric acid 10%) was more stable although it began to dissolved. In contrast, the sample AS10 (citric acid 20%) was stable throughout the 45 minute, demonstrating a higher crosslinking efficacy when using higher citric acid concentration.

### Example 13-Crosslinking after Directional Freezing (Lyophilisation)

All previous samples were prepared by crosslinking the polymers with citric acid before aerogel production. The following aerogels were prepared by crosslinking the aerogel to assess the effect of crosslinking after the aerogels are formed. The crosslinking reaction was thus performed as the last step, after lyophilization.

### Methodology

Certain parameters were adjusted according to the findings described above. Briefly, the concentration of cellulose was raised, the citric acid concentration was adjusted to 10% according to the results described above. In addition, the viscous suspensions were dispersed using FisherBrand 850 Homogenizer in an attempt to improve homogeneity.

The hydrogels were prepared from either mercerized cellulose, regenerated cellulose, succinylated mercerized cellulose or combinations thereof according to the table below.

Polymer combinations used for aerogel preparation.

| Cellulose | H₂O (mL) | Name of Aerogel |
|---|---|---|
| Mercerized cellulose | 8 mL | Merc.AA |
| Regenerated cellulose | 8 mL | D1A |
| Mercerized cellulose | 8 mL | Merc.AA + D1A |
| Regenerated cellulose | | |
| Mercerized cellulose | 8 mL | Merc.AA + Succinylated |
| Succinylated mercerized cellulose | | cellulose |

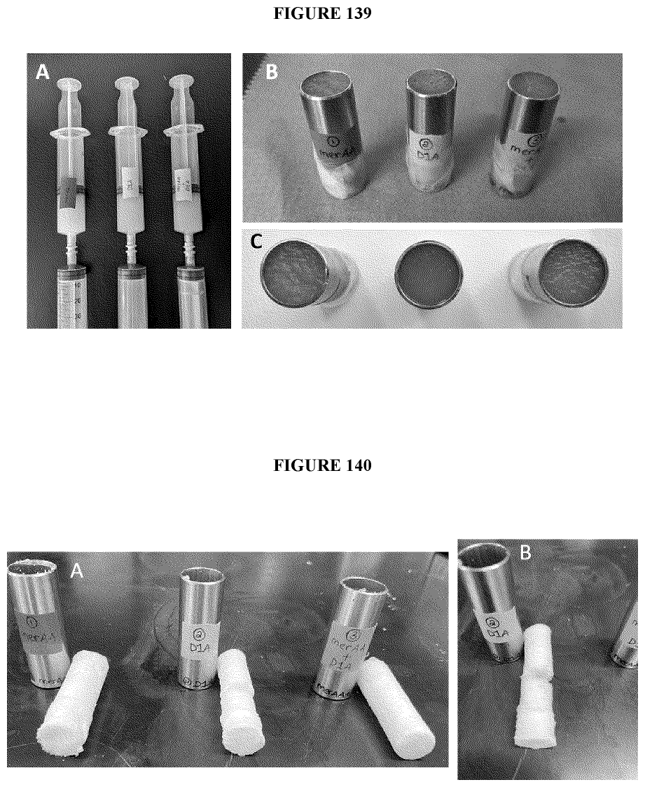

The hydrogels described in the table above were prepared as previously described but without crosslinking. FIG. 139 shows the hydrogels mixed in two 50 mL syringes connected with an f/f luer lock connector (A) and inserted into steel tubes before directional freezing (B and C).

### Results

FIG. 140 shows the aerogels Merc.AA, D1A and Merc.AA+D1A after directional freezing, before crosslinking.

In contrast to the previous aerogels in which the polymers were crosslinked before preparation of the aerogels, FIG. 140 shows that the aerogels produced without crosslinking remained intact after directional freezing except for the aerogel produced with regenerated cellulose (lower size particle) which was broken (B).

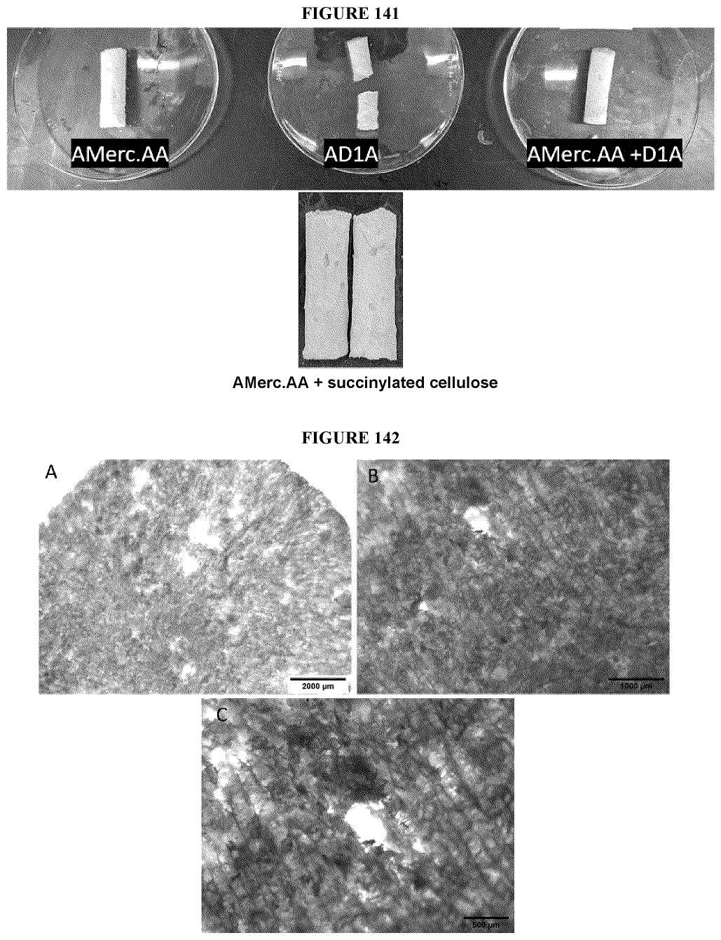

The crosslinking process was then performed as described above using a 10% citric acid solution with the resulting aerogels shown in FIG. 141.

FIG. 141 shows the aerogels Merc.AA, D1A and Merc.AA+D1A after crosslinking.

FIG. 142 shows microscope images of the Merc.AA aerogel of FIG. 141.

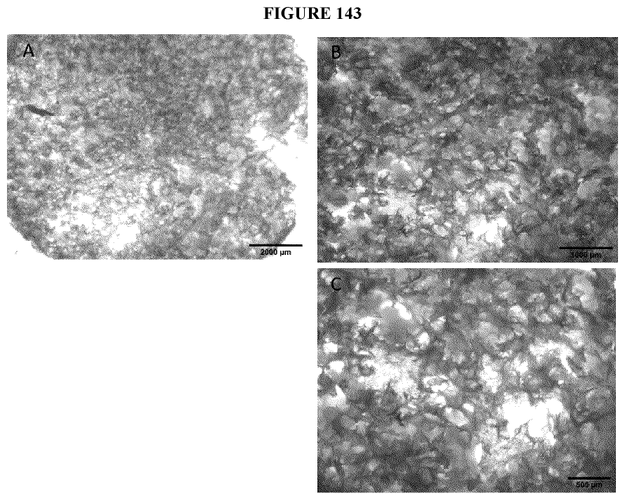

FIG. 143 shows microscope images of the D1A aerogel of FIG. 141.

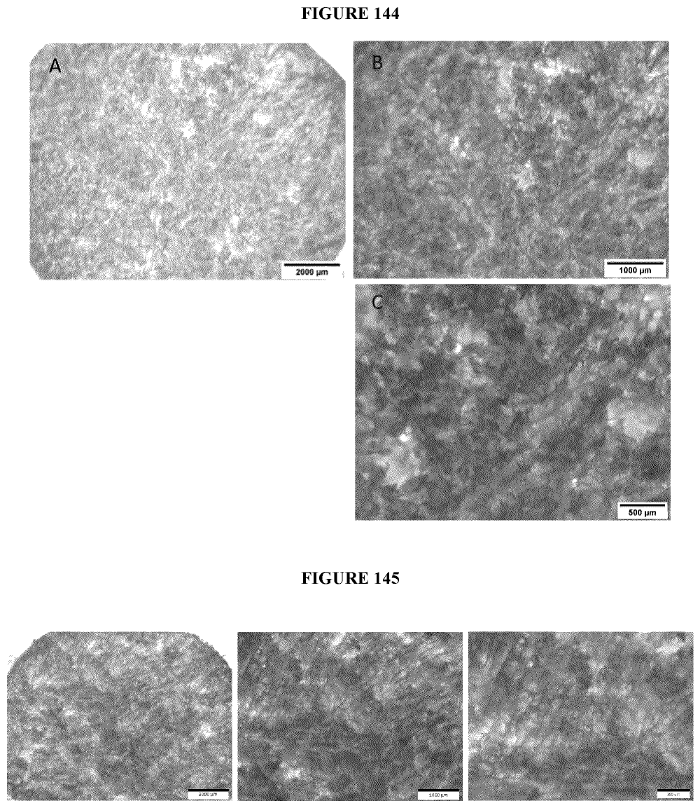

FIG. 144 shows microscope images of the Merc.AA+D1A aerogel of FIG. 141.

The microscopy images highlights the differences in morphology between each aerogel. The aerogel produced with Merc. AA presented some aligned structure which could not be observed in the other two aerogels (D1A and Merc. AA+D1A). The morphology of the aerogel produced with only regenerated cellulose showed larger pores and the material was less compacted compared to the aerogel prepared with Merc. AA+D1A, which showed a more compact and uniform structure.

The aerogel prepared with Merc.AA+succinylated cellulose was then examined.

FIG. 145 shows microscope images of the Merc.AA+succinylated cellulose aerogel of FIG. 141. This particular aerogel shows the presence of aligned pores at the edge of samples which was absent in the other aerogels.

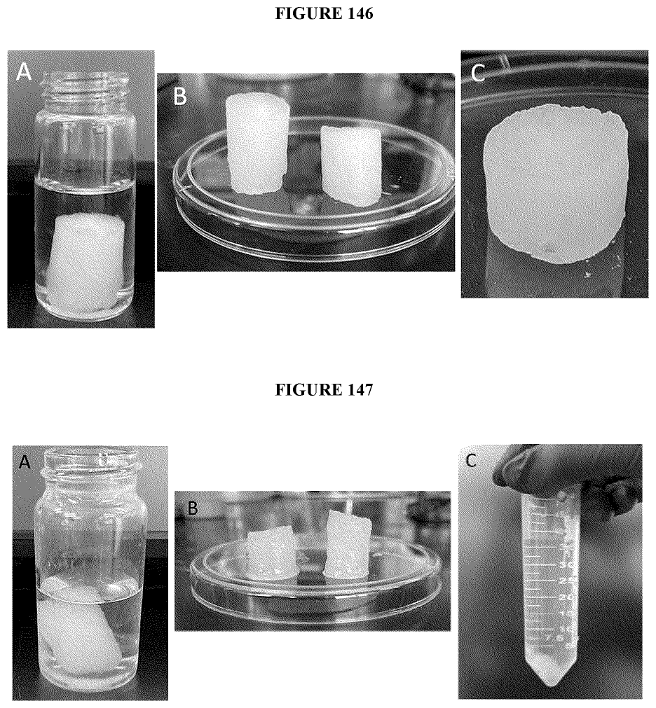

The stability of each aerogel described in the table above was analyzed and images are shown in FIGS. 146-149. The stability of these samples was quite different from the aerogels prepared with crosslinked cellulose, when the crosslinking was performed before lyophilization. The stability of the aerogels was evaluated after incubation in phosphate-buffered saline (PBS), pH 5.5 for 5 minutes, 6 hours or 24 hours.

FIG. 146 shows aerogels prepared with Merc.AA crosslinked after lyophilisation, after 5 minutes (A and B) and 6 h (C) incubation in PBS.

FIG. 147 shows aerogels prepared with D1A crosslinked after lyophilisation, after 5 minutes (A and B) and 6 h (C) incubation in PBS.

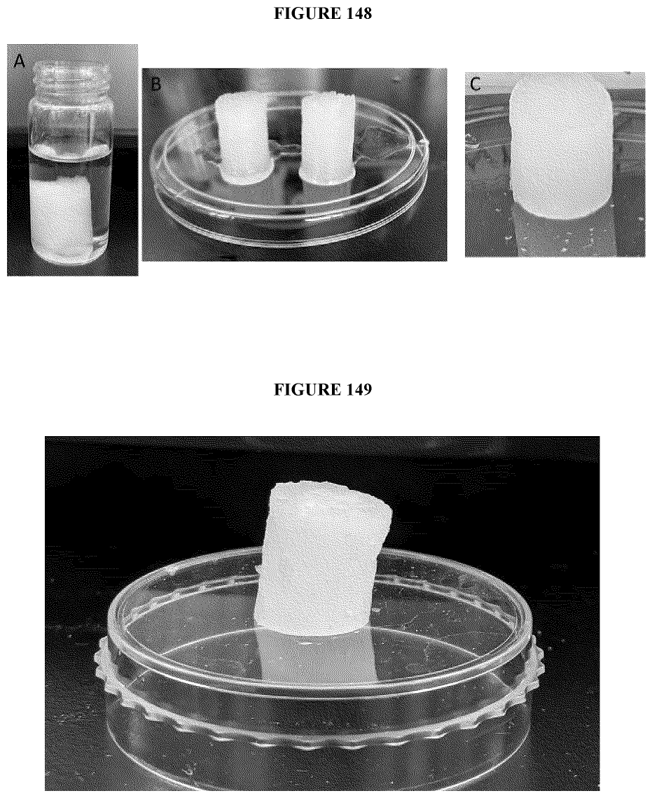

FIG. 148 shows aerogels prepared with Merc.AA+D1A crosslinked after lyophilisation, after 5 minutes (A and B) and 6 h (C) incubation in PBS.

FIG. 149 shows aerogels prepared with Merc.AA+succinylated cellulose crosslinked after lyophilisation, after 24 h incubation in PBS.

Since succinylated material is more hydrophilic, a longer incubation time of 24 hours in PBS was performed to confirm the aerogel stability.

The images of the aerogels shows that all aerogels were stable in saline solution, PBS and water using the reticulation in the final step of the process.

### Example 14-Use of Needles after Aerogel Production to Obtain a Porous Structure

In this example, needles were used to generate porous structures in the aerogels.

### Methodology

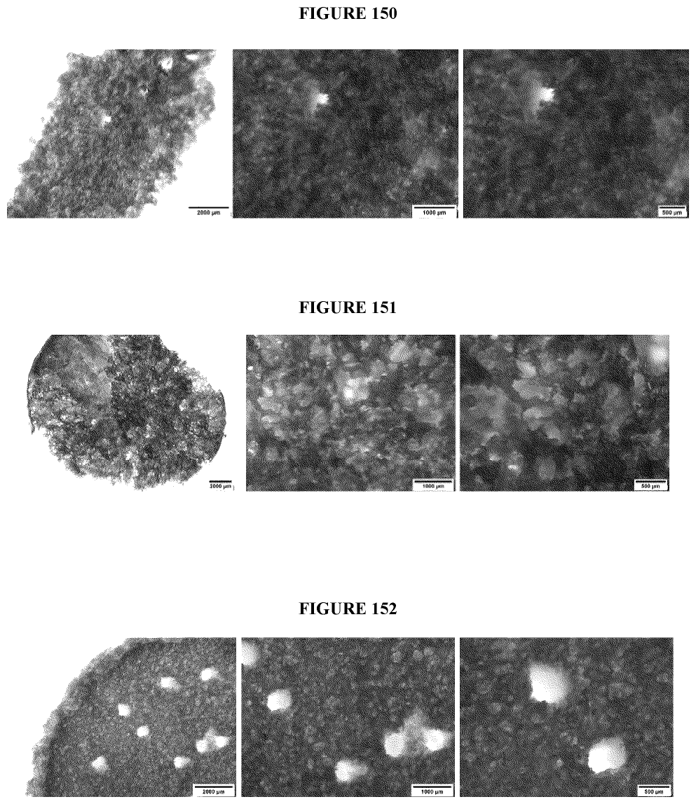

Four hydrogels were prepared from 1) Merc.AA, 2) D1A, 3) Merc.AA+D1A, and 4) Merc.AA+succinylated cellulose and pore size was assessed in FIGS. 150-152. The hydrogels were prepared according to the formulation presented in the table below and dispersed using a FisherBrand 850 Homogenizer set to 10.000 rpm for 10 minutes using a tip attachment 7×110 mm plastic probe. The polymers were then inserted in two 50 mL syringes connected with an f/f luer lock connector and mixed at least 30×.

The samples were then placed into steel tubes and onto the directional freezer for 2 hrs.

Crosslinking with citric acid was then performed by incubating the samples in the oven at 110° C. for 2 h. The crosslinking was also performed for 1.5 hours and the stability of the resulting was examined. Since no significant differences could be observed in terms of morphology and stability of the aerogels PBS, the duration of the crosslinking reaction was standardized at 110° C. in the oven for 1.5 h. The needles were then used to obtain a porous structure. A circular 4 cm silicone mold was inverted and the 30 G needle tips were carefully inserted (perpendicularly) into its base; the needles were arranged into 3 groups of 4 to ensure that each aerogel would contain 4 ‘pores’. To create a base, the bottom of each HDMC mold/metal tube was wrapped with two layers of parafilm, then placed directly over each group of needles. The size of each needle is indicated in the table below.

Name and Formulation of the Aerogels

| | Citric | H₂O | Name of | Needle |
|---|---|---|---|---|
| Cellulose (2.5 g/mL) | acid (%) | (mL) | Aerogel | Size |
| Mercerized cellulose - 20 g | 10 | 8 | Merc.AA | 25G |
| | | | | 0.5 mm |
| Regenerated cellulose - | 10 | 8 | D1A | 30G |
| 20 g | | | | 0.5 mm |
| Mercerized cellulose - 10 g | 10 | 8 | Merc.AA + | 30G |
| Regenerated cellulose | | | D1A | 0.3 mm |
| (D1A) - 10 g | | | | |
| Mercerized cellulose - 10 g | 10 | 8 | Merc.AA + | 30G |
| Succinylated mercerized | | | Succinylated | 0.3 m |
| cellulose - 10 g | | | cellulose | |

### Results

The aerogels were examined by microscopy to evaluate the porous structures.

FIG. 150 shows microscopy images of aerogel prepared with Merc. AA crosslinked with citric acid for 2 h.

FIG. 151 shows microscopy images of aerogel prepared with regenerated cellulose (D1A) crosslinked with citric acid for 2 h.

FIG. 152 shows microscopy images of aerogel prepared with Merc. AA+regenerated cellulose (D1A) crosslinked with citric acid for 2 h.

The microscopy images shows differences in the morphology of each aerogel. The aerogel produced with Merc. AA shows well defined pores produced by the needles which are readily observed in FIG. 150. The pores in the aerogel produced from a mixture of Merc. AA+regenerated cellulose are also well defined.

### Example 14-Use of Needles Before Aerogel Production to Obtain a Porous Structure

In this example, needles (30 G/0.3 mm) were inserted in a silicone mold and the hydrogel was added in the mold. The pores within the hydrogels were then examined.

### Methodology

Four hydrogels were prepared from the same formulation as in example 13 and described in the table above. The four aerogels are: 1) Merc.AA, 2) D1A, 3) Merc.AA+D1A, and 4) Merc.AA+succinylated cellulose and are shown in FIGS. 154-157. Crosslinking with citric acid was performed for 1.5 hours at 110° C. Pore size was assessed using brightfield microscopy and scanning electron microscopy (SEM) in FIGS. 158-163.

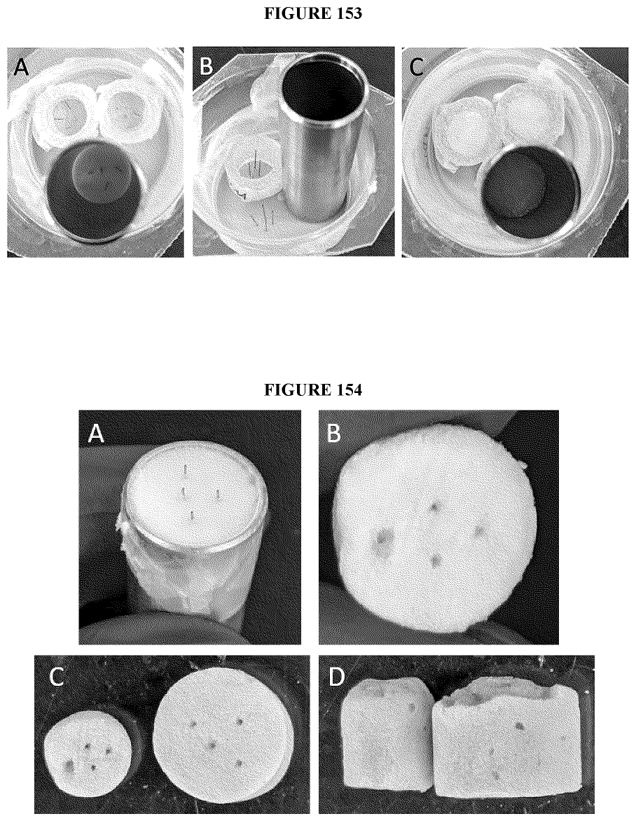

FIG. 153 shows the silicone molds and needles (30 G) used to optimize pore formation in the aerogels, which were prepared as described above.

### Results

The aerogels prepared using the silicone molds and needles (30 G) are shown in FIGS. 154-157.

FIG. 154 shows an aerogel prepared from Merc. AA using silicone mold needles before crosslinking (A, B) and after crosslinking with citric acid (C, D).

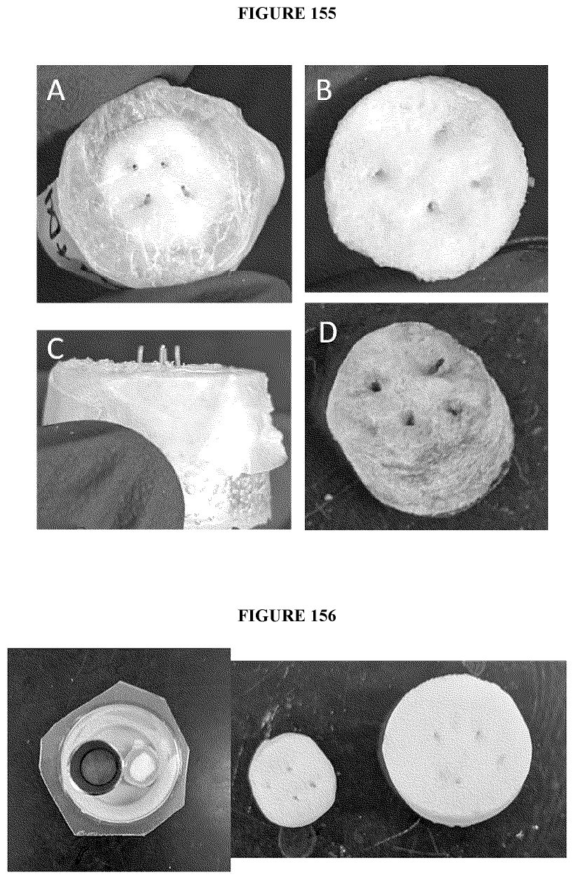

FIG. 155 shows an aerogel prepared from Merc. AA+regenerated cellulose using silicone mold needles before crosslinking (A, B, C) and after crosslinking with citric acid (D).

FIG. 156 shows an aerogel prepared from Merc.AA+succinylated cellulose using silicone mold needles after lyophilization (left) and after removal from the needle mold (right).

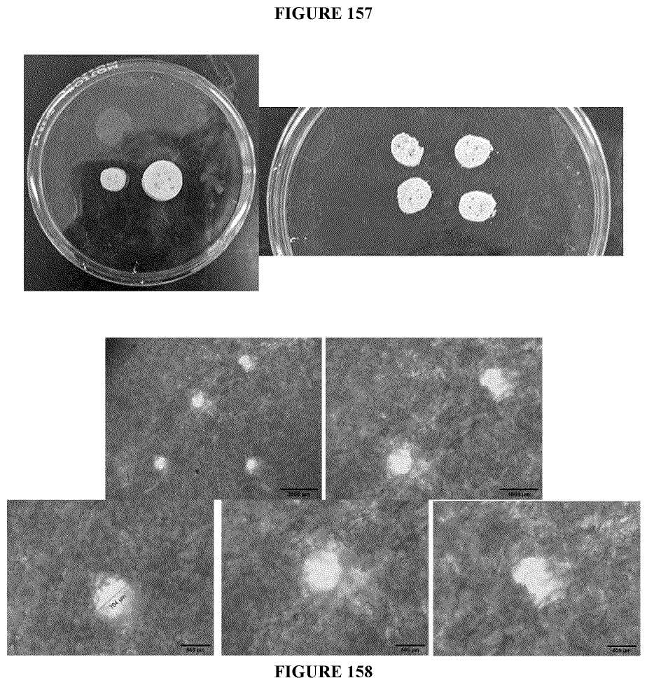

FIG. 157 shows the crosslinked aerogel of FIG. 156 (left) cut into thin slices (right) for subsequent imaging.

The aerogels were then examined by microscopy to evaluate the porous structures.

FIG. 158 shows microscopy images of the aerogel prepared from Merc. AA crosslinked with citric acid with scale bar=2000 μm (A), 1000 μm (B) and 500 μm (C, D, E).

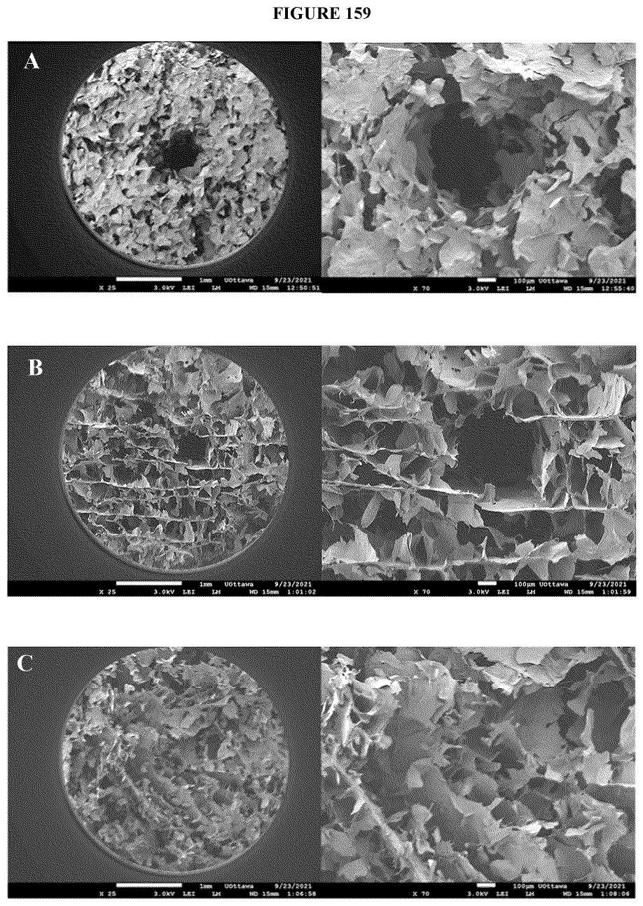

FIG. 159 shows scanning electron microscopy (SEM) images of a top view (A) of the aerogel of FIG. 158 showing a cross-section perpendicular (B) and parallel (C) to the axis of the aerogel.

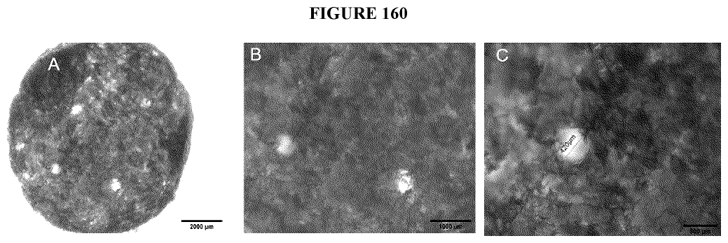

FIG. 160 shows microscopy images of the aerogel prepared from Merc. AA+regenerated cellulose (D1A) crosslinked with citric acid with the scale bar=2000 μm (A), 1000 μm (B) and 500 μm (C).

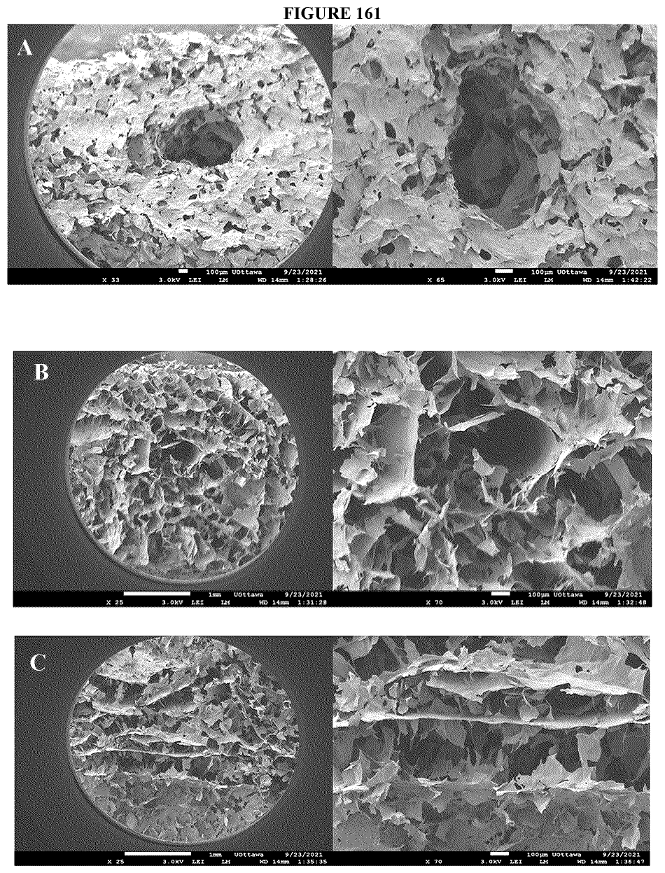

FIG. 161 shows scanning electron microscopy (SEM) images of a top view (A) of the aerogel of FIG. 160 showing a cross-section perpendicular (B) and parallel (C) to the axis of the aerogel.

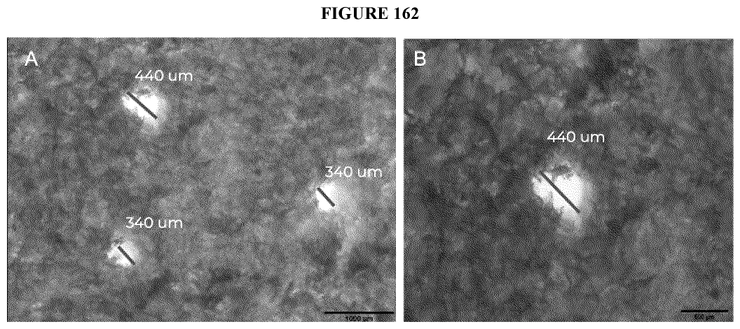

FIG. 162 shows microscopy images of the aerogel prepared from Merc. AA+succinylated cellulose crosslinked with citric acid with the scale bar=1000 μm (A) and 500 μm (B).

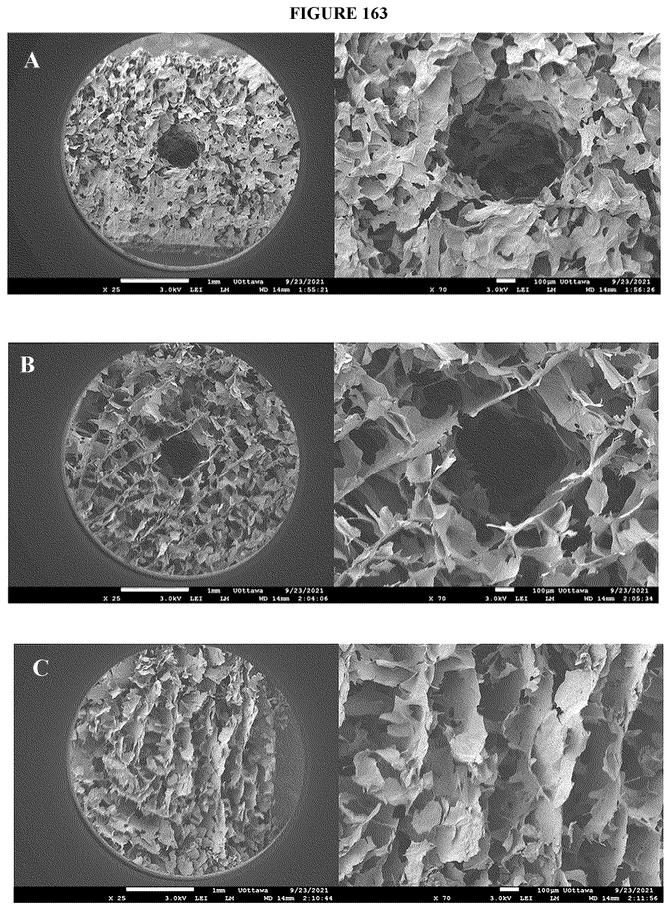

FIG. 163 shows scanning electron microscopy (SEM) images of a top view (A) of the aerogel of FIG. 162 showing a cross-section perpendicular (B) and parallel (C) to the axis of the aerogel.

The microscopy images shows that the pores obtained in the aerogels prepared from Merc. AA and Merc. AA+regenerated cellulose (D1A) are well defined, demonstrating that the needles are stably positioned and yields improve pore formation. More specifically, the morphology of the aerogel produced from Merc. AA presented larger pores having a size of about 700 μm whereas the aerogel produced from AMerc. AA+regenerated cellulose displayed a different structure with smaller pore size of about 420 μm. These results highlight the effect of regenerated cellulose on the morphological organization of the aerogels.

Moreover, the images shows that larger pores are formed when using needles in the preparation of the aerogels.

### Example 15-Aerogel Structural Evaluation-FTIR Analysis

In this example, various samples were examined by Fourier-transform infrared spectroscopy (FTIR) to evaluate the effect of citric acid concentration and if the conditions used were effective in getting cellulose which is covalently crosslinked by ester linkage.

### Methodology

The valent vibrations of CH and OH groups of organic acids are detected between 2800 cm-1 and 3500 cm-1. The complex absorption band in the region of 3500-3200 cm-1 originates from the valent vibrations of OH groups. The valent vibrations of the citric acid free OH group yields a band with a maximum at 3495 cm-1, while valent vibrations of the OH groups involved in intramolecular and intermolecular hydrogen bonds are observed at 3448 cm-1 and 3293 cm-1, respectively (2).

Valent vibrations of C═O group from acid group yield a band at about 1760 cm-1, which in the case of citric acid appears at 1753 cm-1 (accordance with the reference data (2,3)) and if C═O groups are involved in the formation of hydrogen bonds or molecules are dimerized, vibrations occur at lower frequencies, a bond at 1714 cm-1 (4).

There are mainly three sites of hydroxyls on a glucose ring of cellulose, i.e., O(2)-H(2)(the 2-hydroxyl group), O(3)-H(3) (the 3-hydroxyl group), and O(6)-H(6) (the 6-hydroxyl group). The region between 3700-3000 cm-1 is the most interesting and clearly visible changes related to development of intra- and intermolecular hydrogen bonds.

### Results

The samples S6-S10 analyzed are as previously described in example 12 and according to the table below.

Mercerized cellulose and corresponding citric acid concentration

| | Name of cellulose cross |
|---|---|
| Citric acid % | linked samples |
| 2 | S6 |
| 4 | S7 |
| 5 | S8 |
| 10 | S9 |
| 20 | S10 |

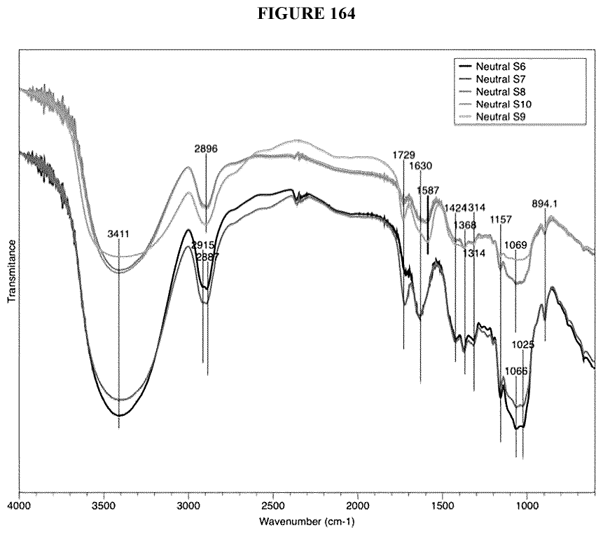

FIG. 164 shows Fourier-transformed infrared spectra (FTIR) of Mercerized cellulose crosslinked with different concentrations of citric acid.

The FTIR profile of all samples are similar but clearly can be observed that samples with low concentration of citric acid (2 and 4%) are similar and others with 5, 10 and 20% of citric acid originate very similar FTIR mainly S8 and S10. All samples show the band at 1730 cm-1 (ester carbonyl) that confirms the ester linkage. Additionally the band at 1587 cm-1 in samples (S8, S9 and S10) show the presence of carboxylate ion. In samples S6 and S7 the symmetric oscillations of O═C—OH groups are observed at the wave numbers 1630 cm-1 and neither asymmetric oscillations of O═C—OH groups at 1392 cm-1, nor valent vibrations of citric acid C—OH group at 1082 cm-1 are present.

Additional samples according to the formulations described in the table below were analyzed by FTIR. According to previous results described above, 10% citric acid was selected as the concentration for crosslinking, which provided the best relation of morphology/stability in PBS.

Name and formulations of the aerogels analyzed by FTIR

| | Citric | H₂O | Name of |
|---|---|---|---|
| Cellulose | acid (%) | (mL) | Aerogel |
| Mercerized cellulose - 20 g | 10 | 8 mL | Merc.AA |
| Mercerized cellulose - 10 g | 10 | 8 mL | Merc.AA + |
| Regenerated cellulose (D1A) - 10 g | | | Regenerated |
| Mercerized cellulose - 10 g | 10 | 8 mL | Merc.AA + |
| Succinylated mercerized cellulose - 10 g | | | Succinylated |

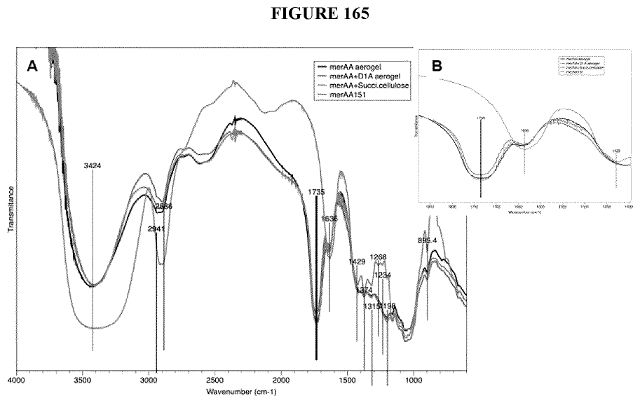

FIG. 165 shows Fourier-transformed infrared spectra (FTIR) of aerogels prepared from Merc.AA, Merc.AA+regenerated cellulose, and Merc.AA+succinylated cellulose crosslinked with 10% citric acid and compared to mercerized cellulose (Merc.AA 151).

Mercerized cellulose (Merc.AA 151) presents a broad band which is reduced after crosslinking with citric acid at 110° C. As mentioned above, the valent vibrations of the citric acid free OH group yield a band with a maximum at 3495 cm-1, while valent vibrations of the OH groups involved in intramolecular are observed at lower wavenumber.

A narrower shift to 3411 cm-1 (FIG. 164) can be observed in all samples, demonstrating the release of free hydroxyls and OH groups involved in intramolecular bonds after reaction.

Referring to FIG. 165, a narrower peak can be seen ranging from 3000 to 3500 cm-1 with a maximum at about 3424 cm-1 which comes from the valent vibrations of OH groups which did not participate in the process of esterification.

Referring to FIG. 164, a band appears at 1729 cm-1 originating from the ester carbonyl group formed in the esterification reaction of cellulose and citric acid. Also, there are bands ranging from 1000-1260 cm-1, which are the result of C—O valence vibrations.

A change in the position of the absorption band of C—O group from citric acid (1753 cm-1) into the band with a maximum at 1729 cm-1 (Commercial sources-Literature data) and 1735 cm-1 (FIG. 165) (ester C═O group) clearly indicates the reaction of the carboxyl groups of citric acid with the hydroxyl groups of cellulose and the formation of an ester bond in all samples prepared.

The synthesis reaction of aerogels from cellulose and citric acid is based on the esterification reaction of carboxyl groups of citric acid with hydroxyl groups of cellulose a shown below.

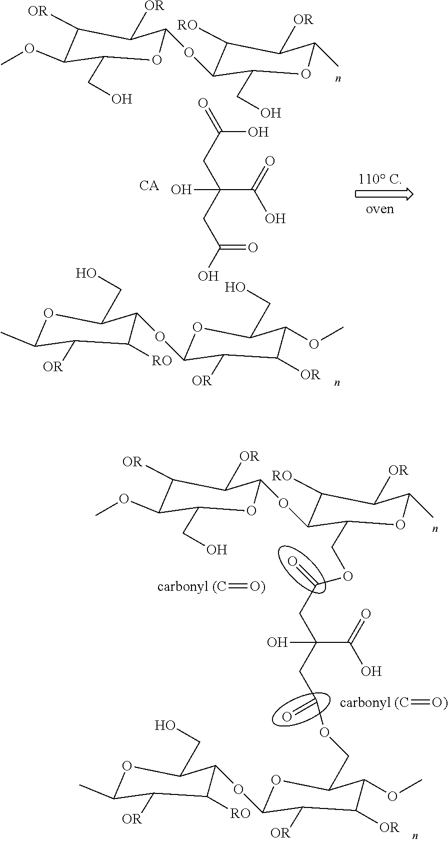

Yang & Wang, 1996 (5) showed that citric acid (CA) first loses one molar of water to form aconitic acid (AA) isomers, which will then release a second molar of water to form AA anhydrides for crosslinking reactions under an elevated temperature. The band at 1630 cm-1, referring to C═C, also appears. This band was clearly observed in samples S6 and S7 (FIG. 165).

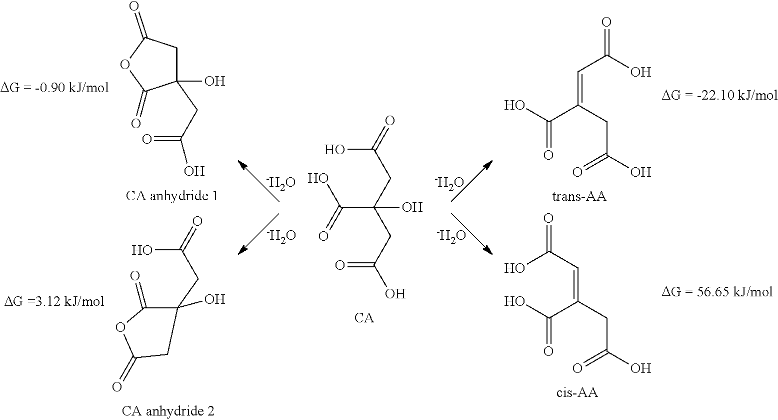

Yang et al. (1996) analysed that pure CA does not form C═C bond and anhydride structure under a temperature at 150° C. The samples were heated from room temperature to 160° C. and measured by FT-IR again. New peaks at 1858 cm-1 and 1793 cm-1, representing the formation of anhydride groups can appear in the CA sample (5). These peaks are not present in any samples as only a band at 1630 cm-1 is observed, referring to C═C, appears (FIG. 164).

Lu & Yang, (1999) (6) have proven that the yellowing problem on citric acid (CA)-treated cotton fabrics was caused by the formation of unsaturated acids, cis- or trans-aconitic acid (AA), under high temperature. In the analyzed samples, a yellowing was observed after the crosslinking and neutralization process. The materials were all yellow to some degree, but there was a transition from a less intense, pale, translucent yellow to a darker more vibrant yellow such that (S6=S9=S10) <AMerc.AA<AMerc.AA+succinylated <AMerc.AA+regenerated). This observation needed to be confirmed by preparing more samples.

### Key Takeaways

The microscopy images showed that there are differences in the morphology obtained for each aerogel and the use of needles was better when the hydrogel was lyophilized with the needles in the mold.
The aerogel produced with Merc. AA (Merc. AA) and succinylated mercerized cellulose presented some aligned structure in the edge of material and this aerogel is stable in saline solution,
The pore size prepared with needles was different after lyophilization in function of type of cellulose. Samples with regenerated cellulose presented smaller pores than aerogel produced only with mercerized cellulose.
Both methodology used for crosslinking with citric acid (after and before lyophilization) was effective to produce ester linkage bonds.
More experiments will be realized to evaluate the degree of substitution, the presence of unsaturated acids that can lead the formation of chromophores that are formed later upon/yellowing aging.

### Example 16-Mechanical Testing of Aerogel Scaffolds

In this example, mechanical testing was conducted using dry and wet samples from each aerogel formulation (Merc.AA, Merc.AA+Succinylated cellulose and Merc.AA+regenerated cellulose).

### Methodology

### Conditions:

Compression 90%
Dry aerogel 10N load cell
Wet aerogel 1N load cell
2.5 strain %/s

Name and Formulations of samples analysed.

| | | Anhydrous | |
|---|---|---|---|
| Sample | Cellulose component(s) | citric acid | ddH₂O |
| Merc.AA | 10 g merAA151 | 1 g | 4 mL |
| Merc.AA + | 5 g merAA151 | 1 g | 4 mL |
| Succinylated | 5 g succinylated cellulose | | |
| Merc.AA + | 5 g merAA151 | 1 g | 4 mL |
| Regenerated | 5 g regenerated cellulose | | |

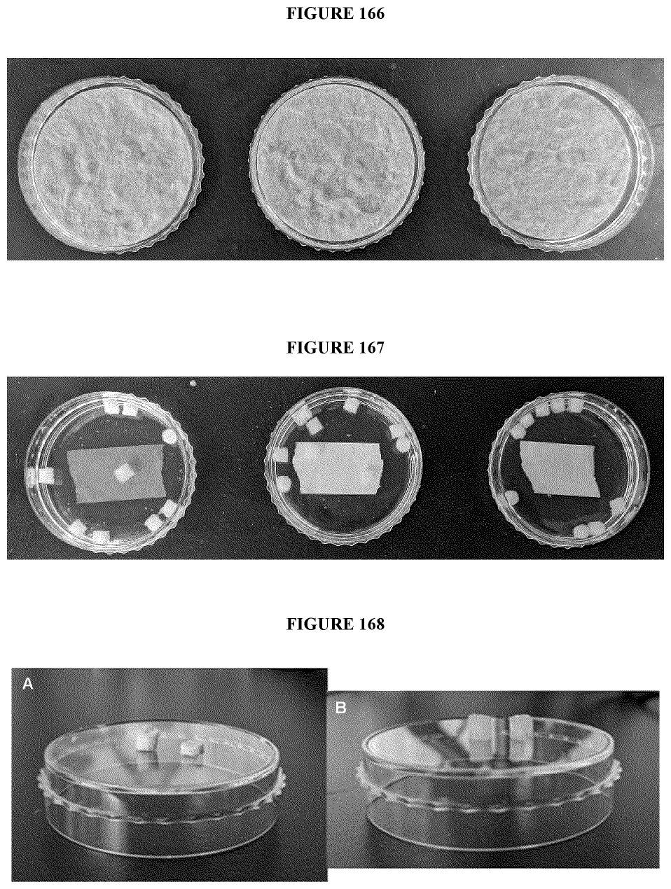

FIG. 166 shows the aerogels prepared from Merc.AA, Merc.AA+Succinylated cellulose and Merc.AA+regenerated cellulose in a 60 mm TC dish, then crosslinked for 1.5 hrs at 110° C.

All samples were cutted using a 5 mm biopsy punch (n=16 per formulation) and the 5 mm wet samples were soaked in saline for 30 min prior to mechanical testing.

FIG. 167 shows the 5 mm wet aerogel samples of FIG. 166 soaked in saline for 30 min prior to mechanical testing.

FIG. 168 shows the dry Merc.AA+regenerated cellulose (A) and wet Merc.AA+regenerated cellulose (B) scaffolds before (left) and after (right) compression testing.

### Results

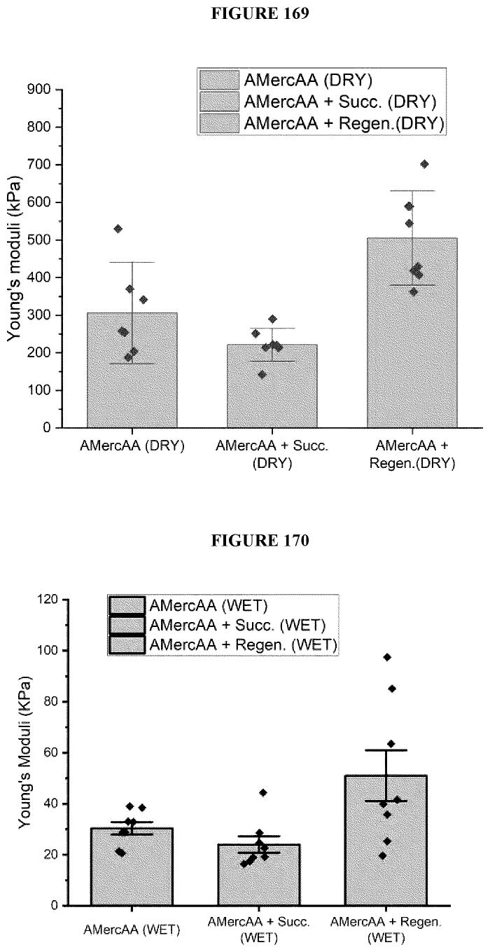

FIG. 169 shows the mechanical properties of dried aerogels which were calculated using the slope of the linear portion of the strain-stress curves obtained with uniaxial compression tests.

FIG. 170 shows the mechanical properties of wet aerogels which were calculated using the slope of the linear portion of the strain-stress curves obtained with uniaxial compression tests.

The mechanical analysis showed high Young′ moduli of all dried samples. The aerogel prepared with mercerized cellulose and regenerated cellulose (AMerAA+Regen.) presented the higher Young' modulus (504 kPa), followed by an aerogel prepared with only mercerized cellulose (AMerc.AA-306 kPa) and the aerogel with succinylated cellulose (AMerc. AA+succ.) showed Young's modulus 220 kPa. A possible explanation for higher Young's modulus with regenerated cellulose can be attributed to change of particle size of cellulose and also the modifications of crystalline conformation (that will be evaluated by NMR) could contribute to easier cross link reaction with citric acid and the production of more resistant compression structure.

After immersion for 30 minutes in saline solution the wet aerogels showed a significant decrease in Young's moduli (FIG. 3). One interesting observation was that for all three samples the decrease of Young's moduli was 10 kPa for each sample. The stress-strain data revealed the elastic modulus for wet samples MercAA+Regen. 50 KPa, MercAA 30 kPa and MercAA+Succ. 30 KPa.

Summary of Young's modulus obtained for each sample

| Samples | Young's moduli (DRY) | Young's moduli (WET) |
|---|---|---|
| Merc.AA | 306 kPa | 30 kPa |
| Merc.AA + Succ. | 220 kPa | 21 kPa |
| Merc.AA + Regen. | 504 kPa | 50 kPa |

The three-dimensional hydrogel networks formed by covalently linking cellulose chains using citric acid as a crosslinker can be an alternative to biomaterial prepared as preformed scaffolds for traditional surgical implantation. The biocompatibility was further evaluated in in the examples below which tested different post-treatment processes to remove any potential toxicity of the crosslinking agent.

### Example 17-Biocompatibility Testing of Aerogel Scaffolds

In this example, the pH was examined to evaluate the biocompatibility of the samples for downstream uses. The samples examined are described in the table below.

### Methodology

| | | Anhydrous | |
|---|---|---|---|
| Sample | Cellulose component(s) | citric acid | ddH₂O |
| Merc.AA | 4 g merAA151 | 0.4 g | 1.6 mL |
| Merc.AA + | 2 g merAA151 | 0.4 g | 1.6 mL |
| Succinylated | 2 g succinylated cellulose | | |
| Merc.AA + | 2 g merAA151 | 0.4 g | 1.6 mL |
| Regenerated | 2 g regenerated cellulose | | |

The aerogels were prepared in a 24-well TC dish, then sterilized and seeded with MC3T3 cells. The samples were grown in culture for 4 weeks and stained for fluorescent microscopy imaging in order to measure cell adhesion and proliferation as described in example 18.

The aerogels were prepared and placed in 24-well plates, lyophilized and carefully removed from the plates for the crosslinking reaction with citric acid.

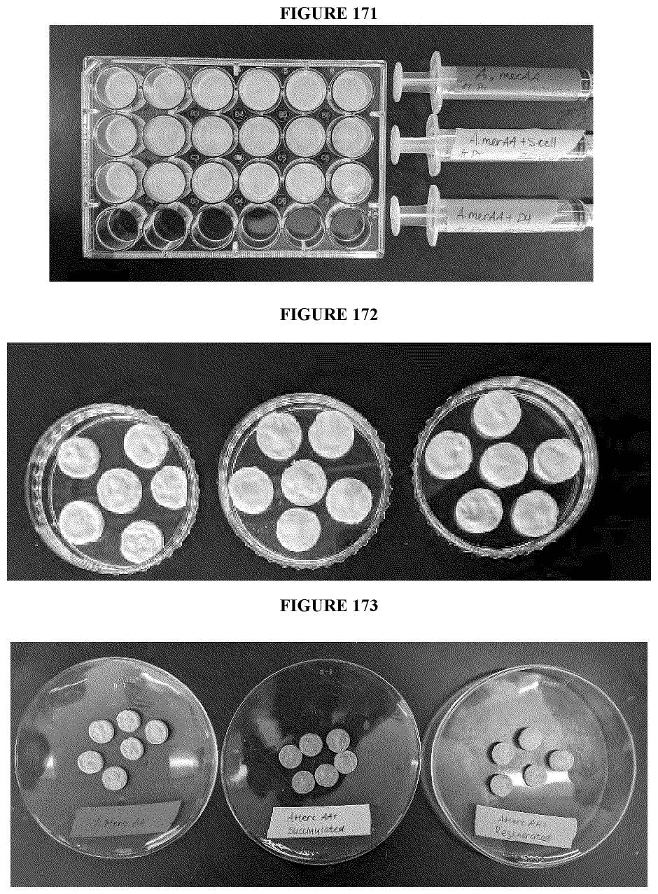

FIG. 171 shows each aerogel formulation plated along one row (n=6) of a 24-well TC dish.

FIG. 172 shows the lyophilized aerogel before crosslinking.

FIG. 173 shows the lyophilized aerogel after crosslinking.

### Results

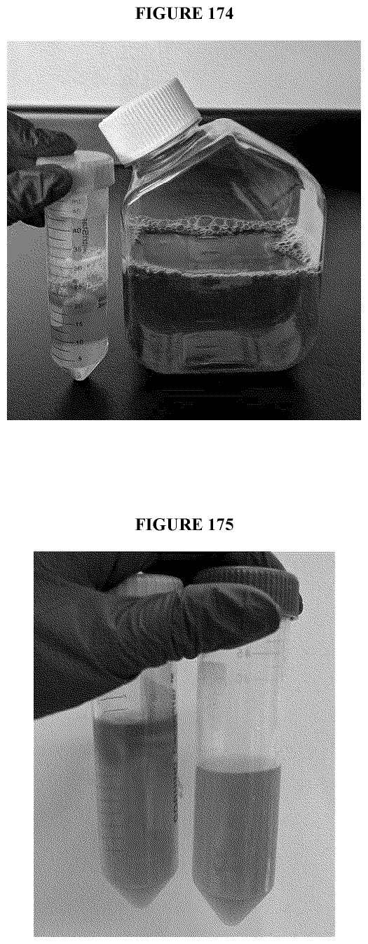

The aerogels were first sterilized and placed into growth media (GM) to assess the acidity before neutralization. As shown in FIG. 174, the GM changed from a bright red to a bright yellow colour after about 10 minutes of incubation.

FIG. 174 shows the change in the colour of the growth media from red to yellow within 10 min of incubation with the aerogels.

### Neutralization of Residual Citric Acid

The lyophilized aerogels were incubated in a sodium bicarbonate solution (28.8 g/L) overnight at 4° C. in order to neutralize any residual citric acid.

Since the cell culture media contains both sodium bicarbonate (2.2 g/L) and phenol red, a pH indicator dye, is believed that the sodium bicarbonate reacted with residual citric acid and, thus, released it into solution. As a result, the overall pH of the media dropped below normal levels and this triggered a rapid change in colour.

The scaffolds were then incubated in DMEM overnight, at 4° C., and the pH of the solutions were measured the following morning. The pH of all 3 solutions were approximately 6.52.

The sodium bicarbonate solution was then removed and the tubes were filled with 30 mL of water. The samples were then placed onto a platform rocker and the water was changed every 5 min, until a total of 5 water washes were completed.

FIG. 175 shows the absence of colour change when the aerogels were incubated in MEM alpha (left) for 24 hrs after neutralization and subsequent water washes. No colour change was observed relative to the tube of stock media (right).

To sterilize the aerogels, each set of samples (n=5 per aerogel formulation) were incubated in 20 mL of 70% EtOH for 30 min.

The GFP-NIH3T3 cells were grown for 1 week prior to staining and imaging. The aerogels were seeded two additional times (on Days 4 and 6) after the initial seeding. Hoechst dye was used to stain the cell nuclei.

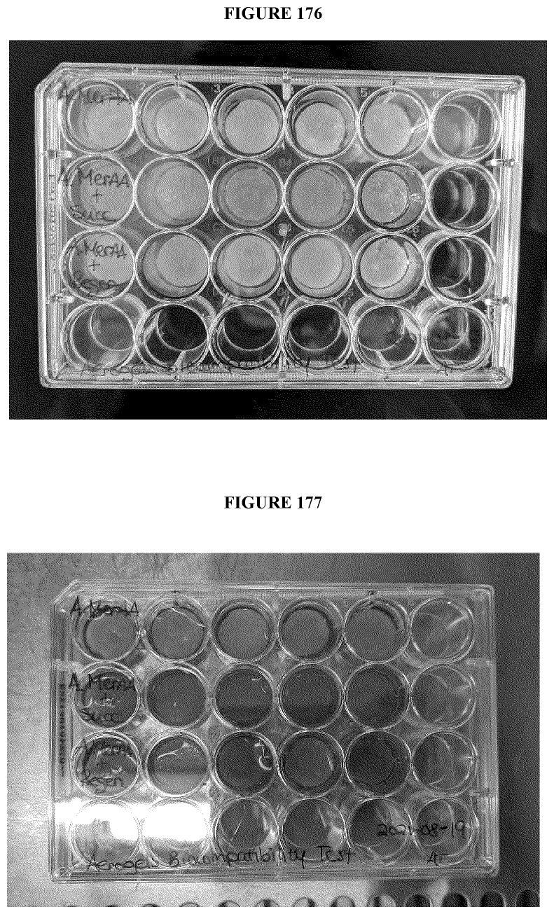

FIG. 176 shows the resulting aerogels prepared from Merc.AA, Merc.AA+Succinylated cellulose and Merc.AA+regenerated cellulose.

FIG. 177 shows the aerogels of FIG. 176 on which 100 μL of the final cell suspension was plated and incubated for 2.5 hrs, then topped up with 1.5 mL of growth media per well.

The aerogels with GFP-NIH3T3 cells were then stained with Hoechst and subjected to microscopy imaging.

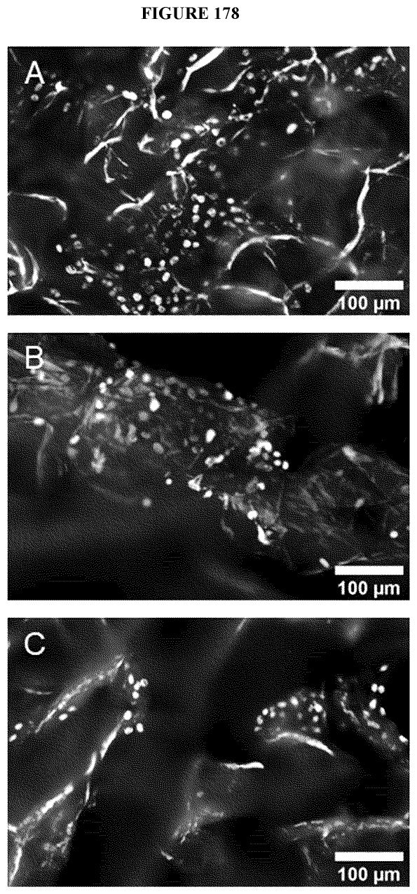

FIG. 178 shows GFP-NIH3T3 cells stained with Hoechst on (A) MercAA aerogel, (B) MercAA+Succinylated cellulose aerogel, and (C) MercAA+regenerated cellulose aerogel with scale bar=100 μm. Purple=scaffold, Yellow Dots=cell nuclei.

For all samples, individual cells as well as cell clusters were observed indicating the aerogels support cell growth and are thus biocompatible in vitro.

### Development and Characterization of a Biomaterial Considered Safe for Food Consumption

### Food-Grade Biomaterial Fabrication

Establishing a completely food-safe process was undertaken by performing the entire fabrication in a shared kitchen (apple processing, decellularization, mercerization, scaffold fabrication and cooking) utilizing all chemicals in the food-grade category to create biomaterials compatible with food industry. Mercerization is a key step in the process of creating the biomaterial. Currently, this step uses high temperatures along with concentrated acids and bases, which require a fume hood, with the utilization of different types of personal protective equipment (PPEs), and cannot be safely performed in the kitchen. In order to solve this hurdle, food-safe chemical alternatives like sodium bicarbonate (NaHCO₃), commonly referred to as baking soda, acetic acid (CH₃COOH), commonly referred to as vinegar (when diluted), and citric acid (C₆H₈O₇) widely utilized on food product formulation, will be used to replace NaOH and HCl respectively. Mukhtar et al. (2018) demonstrated that the sodium bicarbonate has significant effect on the physicochemical properties of sugar palm fiber, thus this chemical could be an alternative in comparison with established alkaline chemicals for treating cellulose fibers, representing a cost-effective and environmentally friendly option for the food-safe biomaterial version.

Once crystalline cellulose chains are highly ordered and tightly packed in a microfibril these microfibrils must be disrupted or swollen to make cellulose chains more accessible to interact with dyes, flavours, proteins, fibers, oils, fatty acids, etc. Sodium bicarbonate (NaHCO₃) is used in the pretreatment process of cellulose and showed to be effective not only to disintegrate the cellulose structure but also to facilitate the amorphization of the crystalline cellulose as well as the extended removal of integrated lignin (Morehead, 1950). The new method with sodium bicarbonate used to prepare our “base material” is a process applied to remove lignin and to disintegrate microfibrils with less degradation/loss of cellulose. Other research already showed that the pretreatment by sodium bicarbonate was effective to swell the fiber structure (Kahar et al., 2013) and the swelling of macro- and microfibrils is useful to make cellulose chains more accessible.

Also, the substitution of medical grade chemicals represents a key step in the development of a food-safe version of the biomaterial. Moreover, the utilization of ingredients or additives considered generally recognized as safe (GRAS), was necessary to produce a material considered safe as a food alternative. According to the FDA, “GRAS” is considered any substance intentionally added to food (food additive), that is subject to premarket review and approval by FDA, unless the substance is generally recognized, among qualified experts, as having been adequately shown to be safe under the conditions of its intended use, or unless the use of the substance is otherwise excepted from the definition of a food additive. In addition, different trials were performed to efficiently substitute the equipment utilized in the laboratory with equipment from the kitchen to create a process for the food safe biomaterial fabrication, feasible to replicate in a conventional kitchen (FDA, 2019).

### Example 18-Use of Sodium Bicarbonate to Mercerize Decellularized AA at 80° C.

In this example, sodium bicarbonate was used to mercerize decellularized AA as described in Mukhtar et al., 2018 with modifications as described below in order to develop a kitchen-safe mercerization process. The mercerization step was adapted to kitchen-safe alternative by using 10% sodium bicarbonate (NaHCO₃) at 80° C. instead of 1 M NaOH for the alkaline treatment. The goal was to create a mercerization step which would be completely food grade.

### Methodology

10% Sodium bicarbonate (2.5 L) was added to a labelled 4 L beaker with an initial pH of 7.84 and was heated until reaching 80° C.
After reaching 80° C., 250 g of decellularized AA was added and mixed to the 10% sodium bicarbonate solution.
For the bleaching process, 25 mL of 30% hydrogen peroxide (H₂O₂) stock solution was added every 15 min until reaching a total of 125 mL while being stirred for 1 hour.
The heat was turned off and the pH was 8.65.
For the neutralization reaction, glacial acetic acid (CH₃COOH) was added to the solution until reaching pH 7.10.
Due to the high degree of carbonation from the sodium bicarbonate and acetic acid reaction, the solution was agitated by shaking and pouring between beakers to minimize the chance for pressure buildup during handling and centrifugation. 25 mL aliquots in 50 mL falcon tubes were centrifuged at 5000 rpm for 5 min to see if any residual pressure buildup was present in the tubes after centrifugation. No bubbling or gas release occurred.
Samples were then spun at 8000 rpm for 15 minutes in 1 L tubes. A checking step for any pressure buildup after 5 minutes was added.
The supernatant was discarded, distilled water was added, the pH was checked with a calibrated pHmeter, and the solution was neutralized again.
The centrifugation process was repeated 4 times. The pH values were:
   7.10 after the first neutralization, before centrifugation
   8.76 measured first thing next day morning, after the first centrifugation (neutralized to 6.82)
   7.70 after the second centrifugation (neutralized to 6.91)
   6.88 after the third centrifugation
The material was spun down once more after this and stored in a 50 ml falcon tube.
The final weight for a 250 g decellularized apple was 55.30 g of mercerized apple (mer AA).

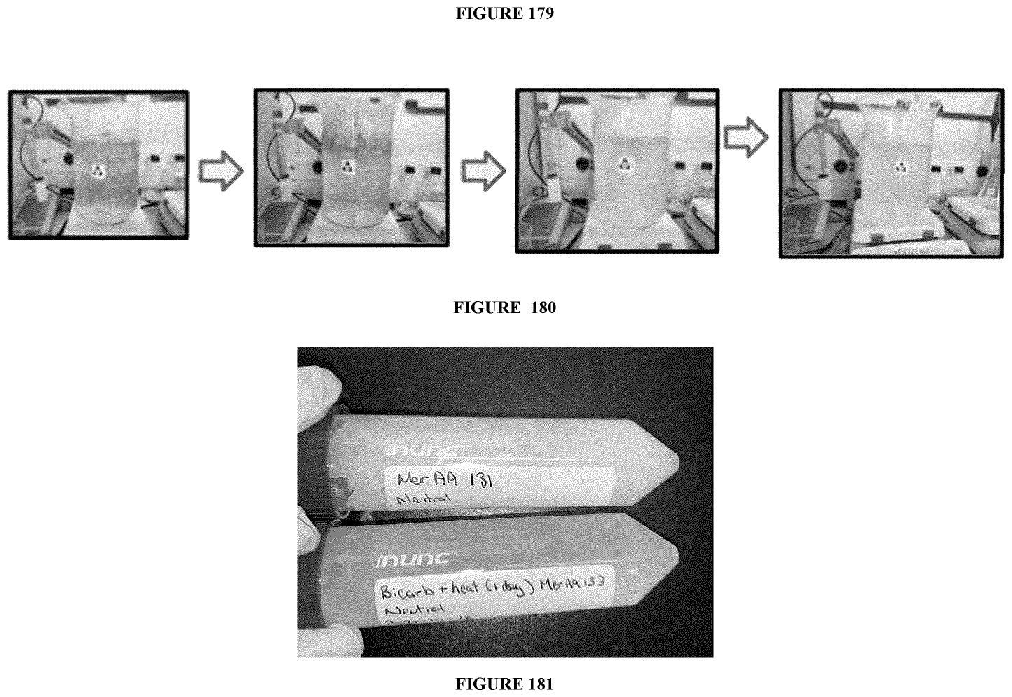

FIG. 179 shows the one hour mercerization using 10% bicarbonate solution at 80° C.

FIG. 180 shows bicarbonate mercerized apple (bottom) compared to NaOH mercerized apple (top).

### Example 19-Use of Sodium Bicarbonate to Mercerize Decellularized AA at Room Temperature

In this example, sodium bicarbonate was used to mercerize decellularized AA as described in Mukhtar et al., 2018 with modifications as described below in order to develop a kitchen-safe mercerization process. The mercerization step was adapted to kitchen-safe alternative by using 10% sodium bicarbonate (NaHCO₃) at room temperature for five days instead of 1M NaOH for the alkaline treatment. The goal was to create a mercerization step which would be completely food grade.

### Methodology

10% Sodium bicarbonate (2 L) was added to a labelled 4 L beaker containing decellularized apples (277.40 g). and mixed at room temperature for 5 days.
pH measurements were taken on days 0, 1, 2, and 5 and were as follows:
   day 0: 7.94
   day 1: 8.25
   day 2: 8.54
   day 5: 8.98
After 5 days, 25 ml of 30% hydrogen peroxide (H₂O₂) stock solution was added every 15 minutes for a total of 125 ml.
After the addition of 125 mL no bleaching was noticed, so the temperature was ramped up to 80° C. in 1 h (slow increase of the temperature).
For the neutralization reaction, glacial acetic acid (CH₃COOH) was added to the solution until reaching pH 7.10.
Due to the high degree of bubbles from the sodium bicarbonate and acetic acid reaction, the solution was agitated by shaking and pouring between beakers to minimize the chance for pressure buildup during handling and centrifugation.
Samples were then spun at 8000 rpm for 15 minutes in 1 L tubes. The samples were spun as a total of 8 times to reach the target pH, potentially due to the sodium bicarbonate buffer capacity.
The supernatant was discarded, distilled water was added, the pH was checked with a calibrated pHmeter, and the solution was neutralized again.
The centrifugation process was repeated 8 times. The pH values were:
   7.10 after the first neutralization, before centrifugation
   8.62 measured first thing next day morning, after the first centrifugation (neutralized to 7.10)
   7.63 after the second centrifugation (neutralized to 7.17)
   7.85 after the third centrifugation (neutralized to 7.12)
   7.69 after the fourth centrifugation (neutralized to 6.87)
   7.29 after the fifth centrifugation (neutralized to 6.87)
   7.53 after the sixth centrifugation (neutralized to 6.77)
   7.10 after the seventh centrifugation
The material was spun down once more after this and stored in a 50 ml falcon tube.
The final weight for a 277.40 g decellularized apple was 63.90 g of mercerized apple (mer AA).

FIG. 181 shows the five days mercerization reaction using 10% bicarbonate solution at room temperature.

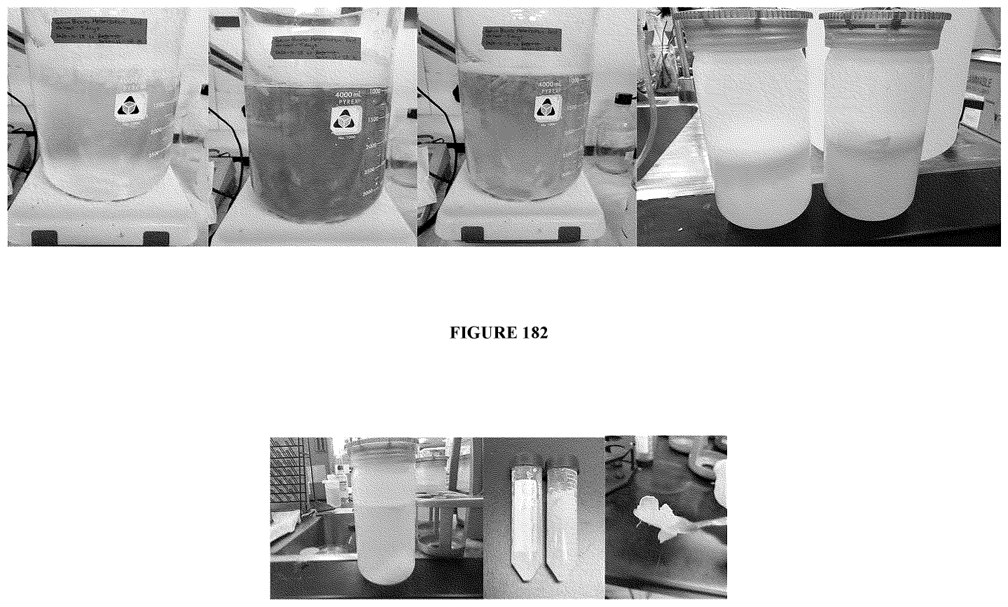

FIG. 182 shows the bicarbonate mercerized apple mercerized apple (mer AA) product. The tubes contain the centrifuged Mercerized produced utilizing the sodium bicarbonate.

### Example 20-Comparison of the Mercerization Procedures

In this example, three mercerization procedures were compared: 1) 1 h at 80° C. using sodium bicarbonate, 2) 5 days at room temperature using sodium bicarbonate, and 3) 1 h at 80° C. using NaOH. The objective was to develop a kitchen-compatible process of mercerization that achieves a similar product as the NaOH mercerized counterpart. Sodium bicarbonate, commonly known as baking soda, is a potential alternative to 1M NaOH that cannot be used in a kitchen. Acetic acid, also known for many applications in food, was used as the acid to neutralize the mercerized product.

The goal was to evaluate if 10% sodium bicarbonate is a viable alternative to 1M NaOH. Microscopy images of the three samples were compared to determine particle size. FTIR was also performed on the three samples.

### Methodology

### Treatments

Treatment A: 5 day mercerization at room temperature in 10% bicarbonate, heated to 80° C., 25 mL 30% H₂O₂ stock solution added every 15 min (125 mL total)
Treatment B: 1 hour heating at 80° C. in 10% bicarbonate, 25 mL 30% H₂O₂ stock solution added every 15 min (125 mL total)
Treatment C (Control): 1 hour heating at 80° C. in 1M NaOH, 25 mL 30% H₂O₂ stock solution added every 15 min (125 mL total)

### Procedure

The 3 treatments (Mer AA from 5 days RT Bicarb, 1 h 80° C. Bicarb, and 1 h 80° C. NaOH) were mixed with 1% alginate solution and Mercerized apple.
A mixture of 1% sodium alginate solution (3 mL), distilled water (4.5 mL), and Mercerized apple (7.5 g) was manufactured.
The mixture from each treatment was frozen.
The frozen samples were freeze-dried for 48 h.
The freeze-dried samples were analyzed in the microscopy, and through FTIR.
Microscopy:
   The samples were visualized in the dark field using two different magnifications: 1× and 6.3×.
   After microscopy the image scale was added utilizing the Image J program.
Single mercerized cellulose particle imaging (Feret size):
   A mix of 0.5 mL Congo Red (0.2%) with 0.5 g MerAA (tube 1) was prepared.
   The mixture was diluted using: 1 mL of tube 1 in 7 mL dH₂O (tube 2).
   The mixture on tube 2 was diluted using: 1 mL of tube 2 in 7 mL dH₂O (tube 3).
   Few drops of tube 3 were added to a glass slide and covered with a cover slip.
   The appropriate fluorescent filter was utilized to image (TXRED).
   The Image was treated and the red was added in ImageJ.
   The ferret diameter was obtained using Image J.
Fourier-transform infrared spectroscopy (FTIR):
   First a potassium bromide (KBr) sample was prepared and left in the oven for at least 24 h and then formed into a tablet.
   The KBr was analyzed and utilized to eliminate the background.
   The samples were then prepared and analyzed utilizing the following setting: Range-start 4000.0 and End: 400.0; scan: 32; Resolution:2.

### Results

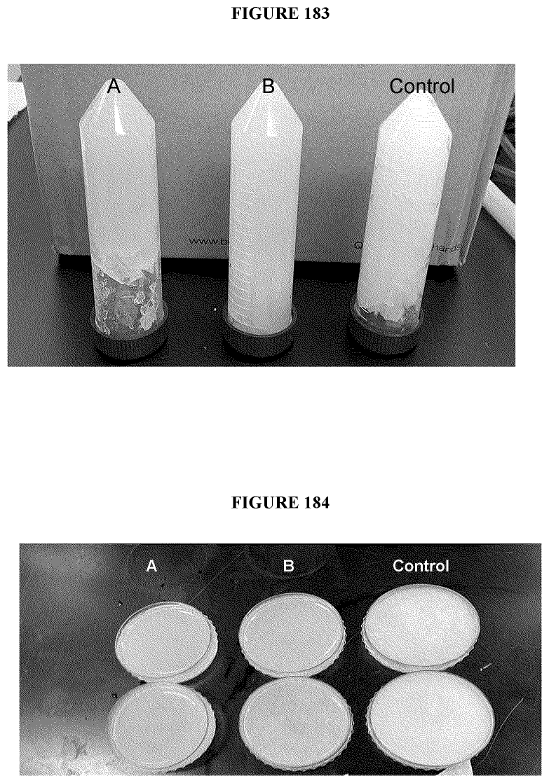

FIG. 183 shows mercerized AA for 5 days at room temperature using bicarbonate (A), for 1 h at 80° C. using bicarbonate (B) and 1 h at 80° C. using NaOH (control).

FIG. 184 shows 1% alginate pucks of mercerized AA for 5 days at room temperature using bicarbonate (A), for 1 h at 80° C. using bicarbonate (B) and 1 h at 80° C. using NaOH (control).

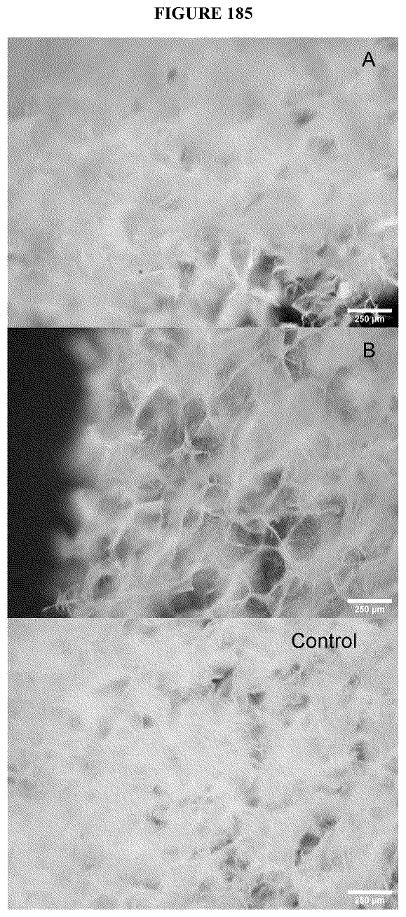

FIG. 185 shows dark field microscopy images of mercerized AA for 5 days at room temperature using bicarbonate (A), for 1 h at 80° C. using bicarbonate (B) and 1 h at 80° C. using NaOH (control) after lyophilization (6.3×).

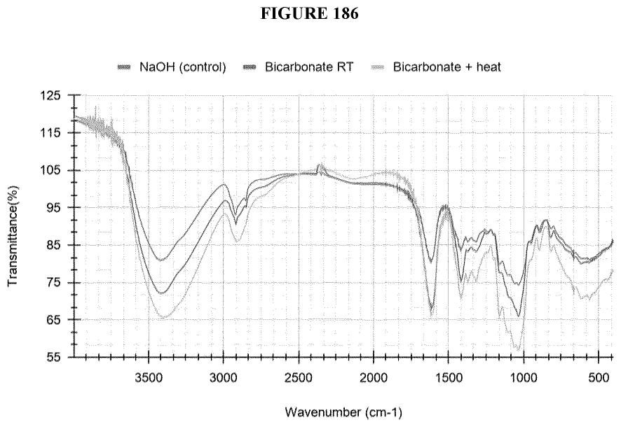

FIG. 186 shows FTIR of mercerized AA for 5 days at room temperature using bicarbonate (red), for 1 h at 80° C. using bicarbonate (yellow) and 1 h at 80° C. using NaOH (blue).

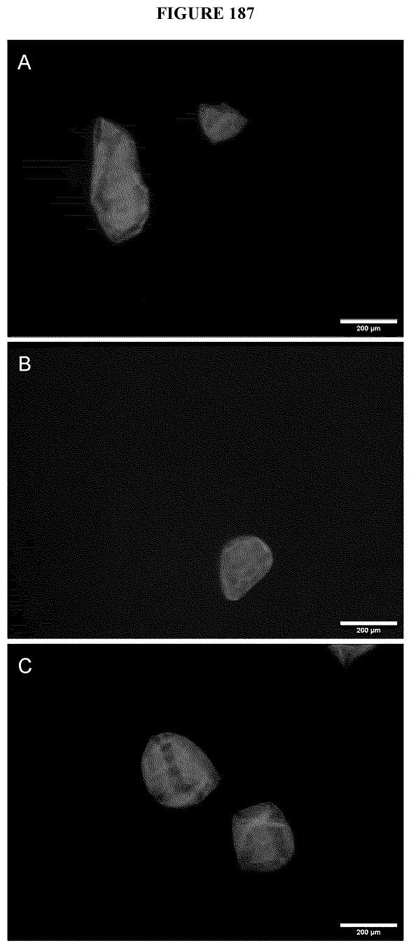

FIG. 187(197) shows fluorescent microscopy images of single particles of mercerized AA for 5 days at room temperature using bicarbonate (A), for 1 h at 80° C. using bicarbonate (B), and 1 h at 80° C. using NaOH (C).

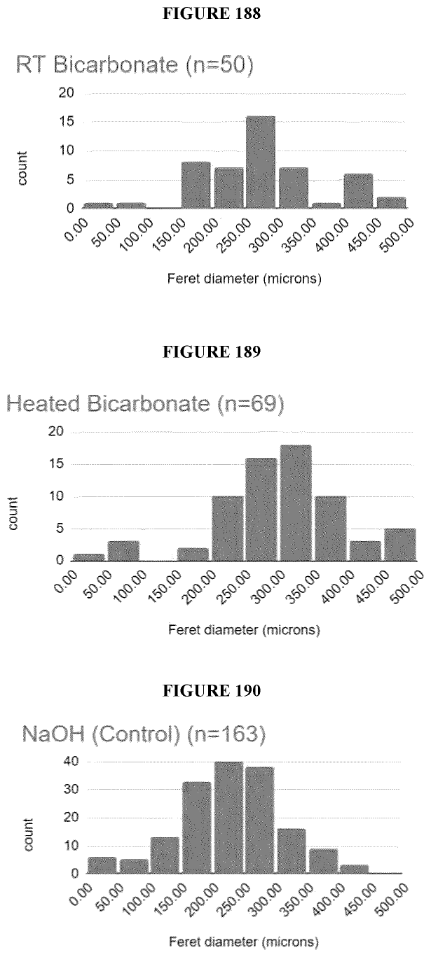

FIG. 188(198) shows an histogram of the particle size distribution of mercerized AA for 5 days at room temperature using bicarbonate.

FIG. 189(199) shows an histogram of the particle size distribution of mercerized AA for 1 h at 80° C. using bicarbonate.

FIG. 190 shows an histogram of the particle size distribution of mercerized AA for 1 h at 80° C. using NaOH.

The characterization of the products shown in FIGS. 183-190 used different technical analysis, including microscopy, yield, FTIR, and cellulose particle Feret diameter.

The treatment utilizing 10% Sodium bicarbonate for Mercerization during 1 h at 80° C. created a lot of carbonation after neutralization with acid, needing a decarbonization step before the centrifugation. The physical appearance was similar to previous NaOH mercerized AA samples with minor differences. Sodium bicarbonate mercerized AA was slightly more liquid, had a minor green/yellow colour, and was less opaque than NaOH mercerized AA.

The treatment utilizing 10% Sodium bicarbonate for Mercerization during 5 days at room temperature also demonstrated the need for pressure release before the centrifugation. On the fifth day, the mixture of decell apple and 10% sodium bicarbonate exhibited a more pronounced colour (red/brown) compared to the 10% Sodium bicarbonate for Mercerization during 1 h at 80° C. treatment. Also larger apple chunks were observed, and more precipitation than treatment B.

Taken together, the mercerization step utilizing 10% Sodium Bicarbonate resulted in a Mer AA having a similar structure to the NaOH treatment according to microscopy images and chemical structure analysis. In addition, no difference (P>0.05) could be observed between the two sodium bicarbonate treatments.

### FTIR

FT-IR analysis showed similar trends in all 3 samples, indicating that there are similarities in the functional groups present, meaning bicarbonate mercerization could be used as a viable alternative to NaOH mercerization in the kitchen. FTIR analysis presents the peaks in the range 3600 to 2925-cm-1 which are associated with free O—H stretching vibration of the OH groups in the cellulose molecules and hydrogen bonded OH stretching vibration. Peaks between 2925 to 2880 cm-1 correspond to aliphatic saturated C—H stretching associated with methylene groups in cellulose. Lignin can also be assigned a broad region including an interval 3300-3600 cm-1 (intramolecular hydrogen bond in phenolic groups, OH stretching of alcohols, phenols, acids and weakly bounded absorbed water). In addition, lignin is composed of three basic units, namely p-hydroxyphenyl (H), guaiacyl (G) and syringyl (S) [78]. Guaiacyl (G) and syringyl (S) are the main units of lignin, but the ratio of S/G varies from one to another plant.

The bands at the 1241 cm-1 and 1317 cm-1 can be assigned as G-ring stretching and S ring stretching respectively. The presence of the band at 1241 cm-1 (C—O stretching vibration in xyloglucan) only in raw and decellularized cellulose shows the process realized with bicarbonate was effective to remove lignin.

The absorption in FTIR spectra including an interval between 1750-1700 cm-1 (C—O stretching in unconjugated groups) reflects changes in various functional groups in lignin and hemicelluloses (carbonyls, ester groups, ketones, aldehydes, carboxylic acids). The absence of the band at 1740 cm-1 in mercerized and bicarbonate material also confirms that our process was effective to remove lignin and hemicellulose from the raw and decellularized material.

The band at 1628 cm-1 can be assigned to the absorbed water and the band at 897 cm-1 which is specific to glucose ring stretching vibration decreased slightly in samples obtained from both processes. It may be due to thermal degradation of β-(1,4) glycosidic bonds. Decreasing the absorption at this band indicates a decrease of the amorphous form of cellulose.

### Ferret Diameter

Single particle Feret diameter comparison
among three different Mer AA methods

| Mer AA method | Average Feret diameter (μm) | SD Feret diameter(μm) |
|---|---|---|
| NaOH/80° C. | 227.0^{B} | 82.8 |
| Bicarb/80° C. | 300.6^{A} | 89.7 |
| Bicarb/RT | 278.2^{A} | 94.8 |

SD—standard deviation; Xlstat 2014

The Mer AA single cells Feret diameter was smaller (P<0.05) in NaOH-Control when compared to both bicarbonate mercerized samples. In addition, the bicarbonate treatments did not demonstrate a difference in feret diameter with each other (P>0.05). The NaOH control demonstrated a normal distribution, whereas a slight left skew was observed for the RT and heated bicarbonate. For all three mercerizations, every particle was under 500 μm.

### Example 21-Comparison of 15% H₂O₂ with 30% H₂O₂ for Bleaching Mercerized Apples

In this example, the sample preparation was adapted to the kitchen (Corer and food processor) and the resulting Mer AA was characterized. The bleaching step was also adapted by comparing 15% H₂O₂ stock solution to 30% H₂O₂ stock solution.

### Methodology

9 McIntosh apples (955 g) were inspected (AA 136), washed, peeled, and the core was taken out using a corer.
The apples were cut in quarters and ground using a food processor.
The ground apple (700 g) was added to a 4 L beaker.
The decellularized step was performed using the shaker (130 rpm).
The Mercerization step was performed with 10% Sodium bicarbonate and 15% hydrogen peroxide stock solution with 1 h heating. The Glacial acetic acid was utilized for the acidification step.
Instead of the centrifugation step, the 25 μm sieve was utilized. The material was passed through the sieve until stabilizing/neutralizing the pH in the range between 6.8-7.2.
Single mercerized cellulose particle imaging (Maximum Feret Diameter):
   A mix of 0.5 mL Congo Red (0.2%) with 0.5 g MerAA (tube 1) was prepared.
   The mixture was diluted using: 1 mL of tube 1 in 7 mL dH₂O (tube 2).
   The mixture on tube 2 was diluted using: 1 mL of tube 2 in 7 mL dH₂O (tube 3).
   Few drops of tube 3 were added to a glass slide and covered with a cover slip.
   The appropriate fluorescent filter was utilized to image (TXRED).
   The Image was treated and the red was added in ImageJ.
   The ferret diameter was obtained using Image J.
Fourier-transform infrared spectroscopy (FTIR):
   First a potassium bromide (KBr) sample was prepared and left in the oven for at least 24 h and then formed into a tablet.
   The KBr was analyzed and utilized to eliminate the background.

The samples were then prepared and analyzed utilizing the following setting: Range-start 4000.0 and End: 400.0; scan: 32; Resolution:2.

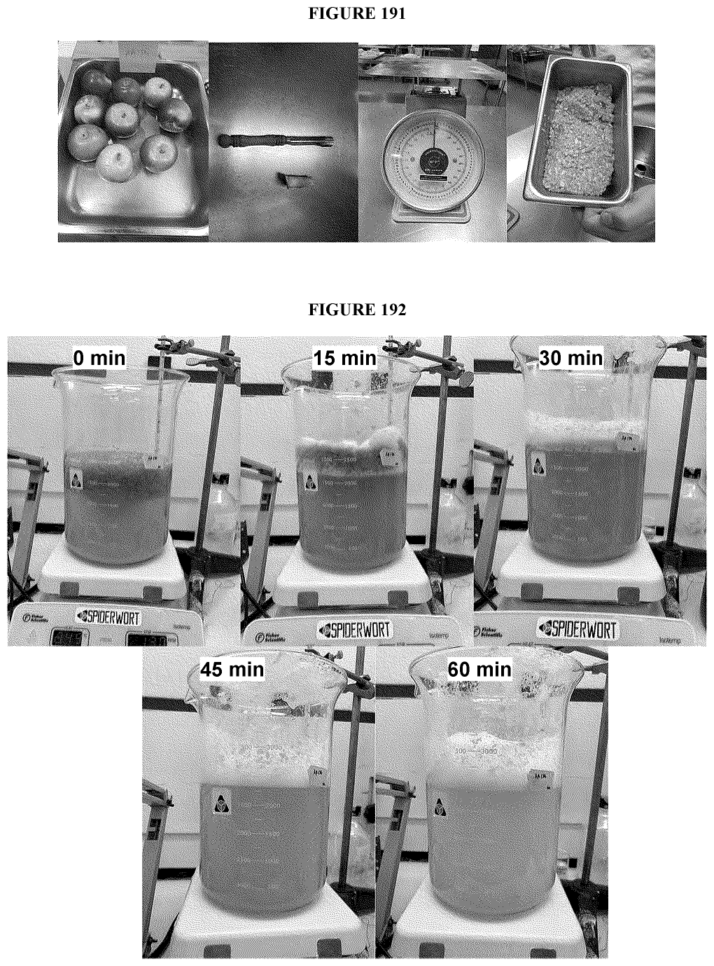

FIG. 191 shows MacIntosh apples processed using a food processor in the kitchen prior to the decellularization.

FIG. 192 shows the mercerization of AA 136 at 15 minutes interval for 60 minutes using 10% bicarbonate at 80° C. and 15% H₂O₂ stock solution.

### Results

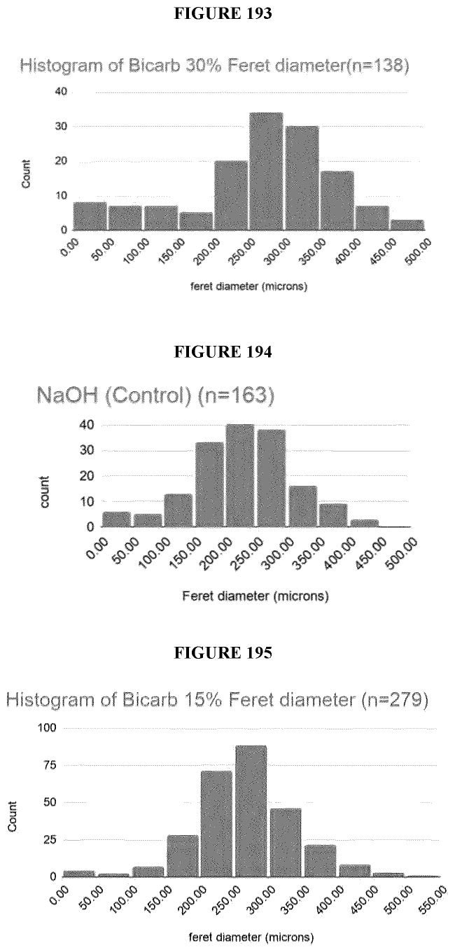

FIG. 193(201) shows an histogram of the particle size distribution of mercerized AA using bicarbonate and bleached with 30% H₂O₂ stock solution.

FIG. 194 shows an histogram of the particle size distribution of mercerized AA using NaOH and bleached with 30% H₂O₂ stock solution.

FIG. 195 shows an histogram of the particle size distribution of mercerized AA using bicarbonate and bleached with 15% H₂O₂ stock solution.

Single particle Feret diameter of three different Mer AA methods

| | Average Feret | SD Feret |
|---|---|---|
| Mercerization/Bleaching Method | Diameter (μm) | Diameter(μm) |
| NaOH, 30% H₂O₂ | 227.0^{B} | 82.8 |
| Sodium Bicarbonate, 30% H₂O₂ | 267.9^{A} | 105.6 |
| Sodium Bicarbonate, 15% H₂O₂ | 266.9^{A} | 76.3 |

SD—standard deviation; Xlstat 2014

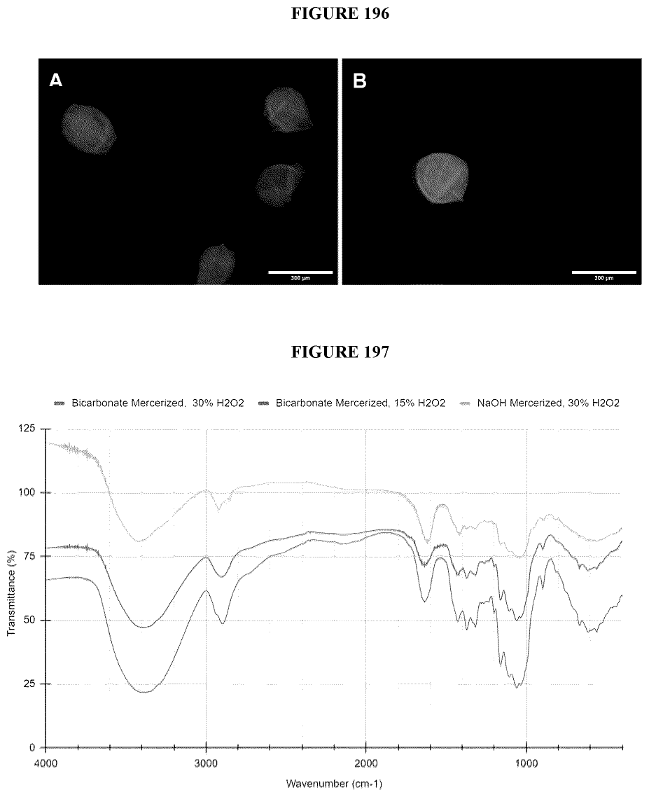

FIG. 196 shows fluorescent microscopy images of single cells of mercerized AA with bicarbonate bleached with 30% H₂O₂ (A) and 15% H₂O₂ (B) stock solutions stained with Congo red under 10× magnification.

FIG. 197 shows FTIR of mercerized AA using bicarbonate bleached with either 15% H₂O₂ or 30% H₂O₂ compared to mercerized AA using NaOH and bleached with 30% H₂O₂.

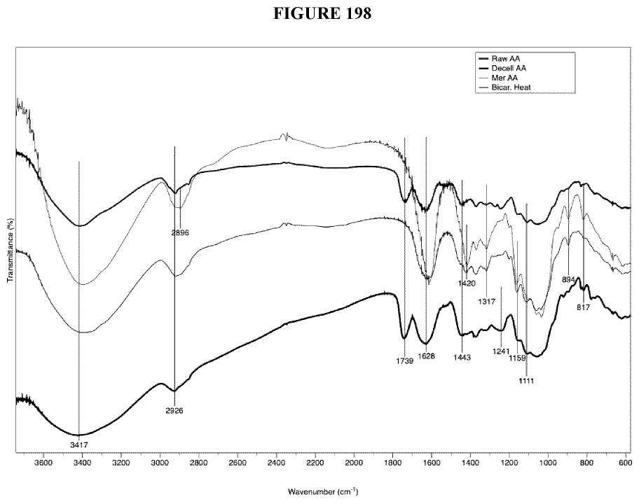

FIG. 198 shows FTIR of mercerized AA using bicarbonate bleached with either 15% H₂O₂ or mercerized AA using NaOH using decellularized or raw apples.

The Reduction of H₂O₂ stock solution concentration in the bleaching step of the apple mercerization was attempted and the characterization of the final material was performed. Based on the results, it is possible to infer that the yield of the Mer AA aerogel prepared with 15% H₂O₂ stock solution was 25%. Also comparing 10% Bicarb with 15% H₂O₂ stock solution, 10% Bicarb with 30% H₂O₂ stock solution, and 1M NaOH with 30% H₂O₂ stock solution, the FTIR analysis demonstrated that the chemical structure of the Bicarb Mer AA treated with different stock solutions concentration of H₂O₂ was similar. Moreover, the particle cells analysis demonstrated that the Mer AA single particles Feret diameter was smaller (P<0.05) in NaOH-Control when compared to Bicarb 30% and Bicarb 15% hydrogen peroxide stock solutions. Also, the two bicarbonate treatments did not differ between each other with regards to feret diameter (P>0.05). Thus, the reduction of H₂O₂ final concentration for the bleaching step did not demonstrate any major differences compared to the control.

### Example 22-Entire Fabrication of a Food Grade Biomaterial in the Kitchen

In this example, entire fabrication of a food grade biomaterial was performed in the kitchen with kitchen equipment and food grade chemicals. The steps for the edible biomaterial included:
(A) Decellularization
(B) Mercerization
(C) Biomaterial fabrication

### Methodology

A-Decellularization-Performed in a 5-day process

### Day 1

The McIntosh apples were purchased from Fresh Start Foods Supplier.
Raw apples were inspected, washed with tap H₂O (macintosh apple), sanitized with 0.1 ml/L of chlorine solution (Stevanato et al., 2020) and a batch code was provided.
All the kitchen instruments were thoroughly washed.
The apples were peeled with the peeler, the core was taken out, the apples were cut into quarters and chopped using a Hobart buffalo chopper.
8 L of 0.1% SDS (FCC) was prepared in a large Hobart stand mixer bowl and mixed on speed 1 using the dough hook.
The apples were transferred to a 10 L mixer bowl, containing 8 L of FCC 0.1% sodium dodecyl sulfate solution (SDS).
The container was labelled and identified with both the material and solution's Spiderwort lot number and expiry date, along with the researcher's initials, and the date.

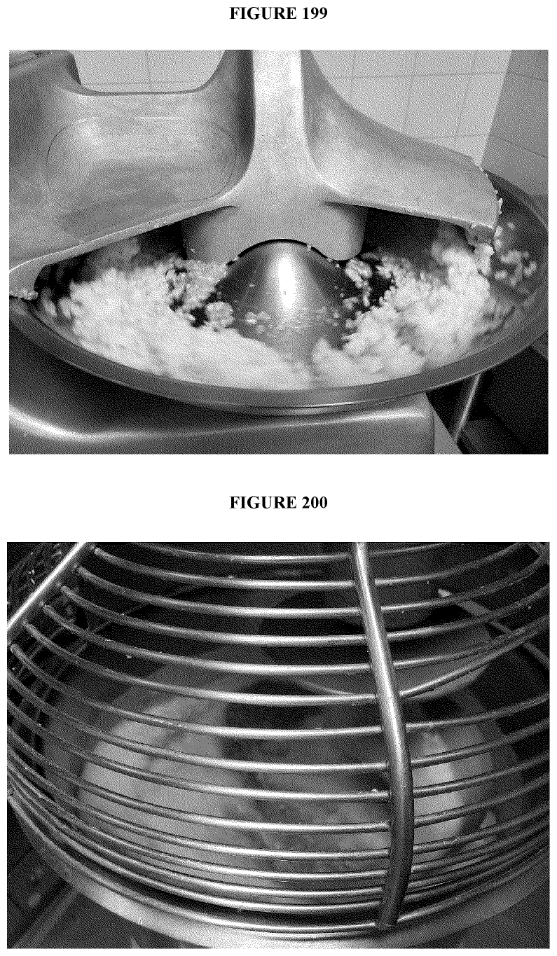

FIG. 199 shows raw apple processing in a large Hobart stand mixer bowl.

### Day 2

After 24 h, the mixer was stopped, and the SDS solution was poured onto the sieve which was set on top of a waste container.
The container was filled with 8 L of freshly made food grade 0.1% SDS solution.

The container is labelled and identified with both the material and solution's Spiderwort lot number and expiry date, along with the researcher's initials, and the date.

FIG. 200 shows processed apple in 0.1% SDS during the decellularization process.

### Day 3

After 24 h, the mixer was stopped, and the SDS solution was poured onto the sieve which was set on top of a waste container.
The container was filled with 8 L of freshly made food grade 0.1% SDS solution.
The container is labelled and identified with both the material and solution's Spiderwort lot number and expiry date, along with the researcher's initials, and the date.

### Day 4

After 24 h, the mixer was stopped, and the SDS solution was poured onto the sieve which was set on top of a waste container.
The mixer bowl was filled with 8 L of water and poured onto the sieve which was set on top of the waste container. This step was repeated 7 times until no soapy residue remained.
The chopped raw apple was added to a freshly prepared 8 L solution of 0.1M CaCl₂) (FCC) in the mixer bowl, and mixed with the dough hook attachment at speed 1.
The container is labelled and identified with both the material and solution's Spiderwort lot number and expiry date, along with the researcher's initials, and the date.

### Day 5

After 24 h, the mixer was stopped, and the CaCl₂) solution was poured onto the sieve which was set on top of the waste container.
The mixer bowl was filled with 8 L of water and poured onto the sieve which was set on top of the waste container. This step is repeated 7 times, drained of excess water utilizing a skimmer, and weighed for mercerization.

### B-Mercerization of Decellularized Apple

The decellularized apple was mercerized in a large pot on a gas stove with a temperature probe to ensure minimal variance in mercerization temperature. The solution was mixed manually.
The extractor fan was turned on and PPEs were utilized before starting the mercerization process.
Using a sieve set atop a waste beaker, the water from the decellularized apple was manually pressed out. For every 500 g decellularized material, 2.5 L of mercerization solution was utilized.
The decellularized material was placed in a clean large pot.

A solution of a 10% Sodium bicarbonate was freshly manufactured and added to a clean pot. The temperature was raised to 80° C., followed by the addition of the decellularized apple.
Usually, the addition of the decellularized material onto the solution for mercerization decreases the temperature.
The temperature was raised to 80° C.
A 25 mL of 15% hydrogen peroxide stock solution was added five times, totaling 125 mL solution.
The solution was manually stirred in the pot for 1 h at 80° C.
The reaction should proceed until the colour disappears. The target colour was clear or off-white.
The heat was turned off, and the solution was placed in the fridge to cool down.
Using a pH meter, the solution was neutralized with acetic acid (30%) until the pH is 6.8-7.2
The pH value was recorded.
The solution was passed through a 25 μl stainless steel sieve, the supernatant was discarded by pouring the liquid into a clean waste container.
The pellet in the sieve was resuspended in water, and neutralized again.
A repeated neutralization and sieving cycle steps were performed until the pH stabilization within 6.8-7.2 for consecutives measurements after sieving and resuspension.
The final pH and the number of sieving cycles were recorded.
The mercerized apple is sieved one last time for 1 h, and passed through a cheesecloth to concentrate the material.
The material was centrifuged at 8000 rpm for 15 minutes, the supernatant was discarded, the pellet was transferred to a clean vacuum bag, and vacuum sealed.
The vacuum bad containing the mercerized material was properly labelled and identified with the Spiderwort lot number and expiry date.
The material was stored in the fridge at 4° C.

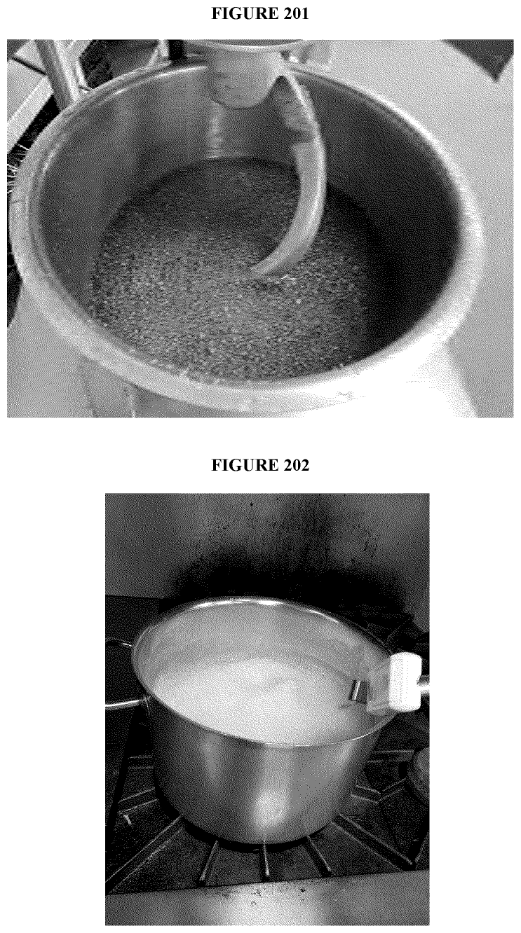

FIG. 201 shows processed apple in 0.1M CaCl₂) solution.

FIG. 202 mercerization of the decellularized apple on stovetop.

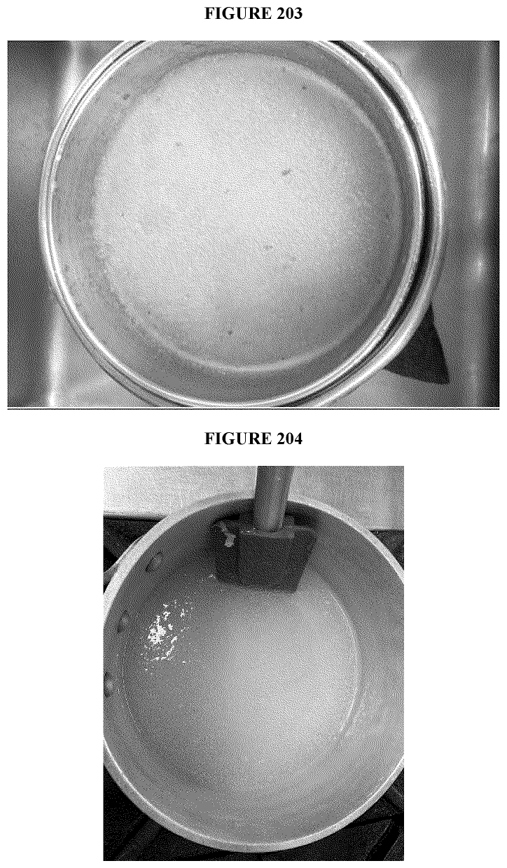

FIG. 203 shows sieving of decellularized apple, using a 25 μl stainless steel sieve.

### C-Scaffold Fabrication

On this step, the mercerized apple was mixed with a texturizing agent, followed by a cross-linking.
The mercerized apple was mixed with 2% sodium alginate solution (texturizing agent) in a 1:1 proportion.
The biomaterial was placed in silicone molds, the molds were wrapped using plastic wrap, and placed in the kitchen freezer overnight.
Frozen samples were then lyophilized in the Buchi L-200 lyophilizer at −55° C., 0.100 mbar for 48 hours.
The biomaterial was then crosslinked in a 1% (w/v) CaCl₂) dihydrate (FCC) bath overnight in the refrigerator.

FIG. 204 shows 2% alginate solution being prepared on the stovetop.

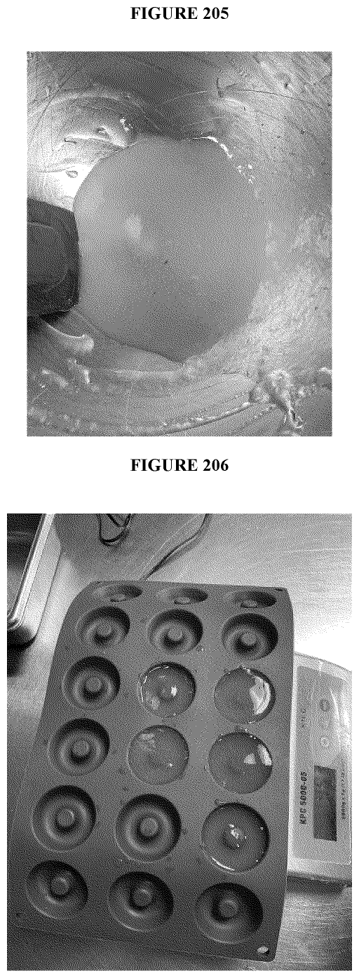

FIG. 205 shows mixture of mercerized apple and 2% alginate via standmixer.

FIG. 206 shows depositing of biomaterial into silicone molds.

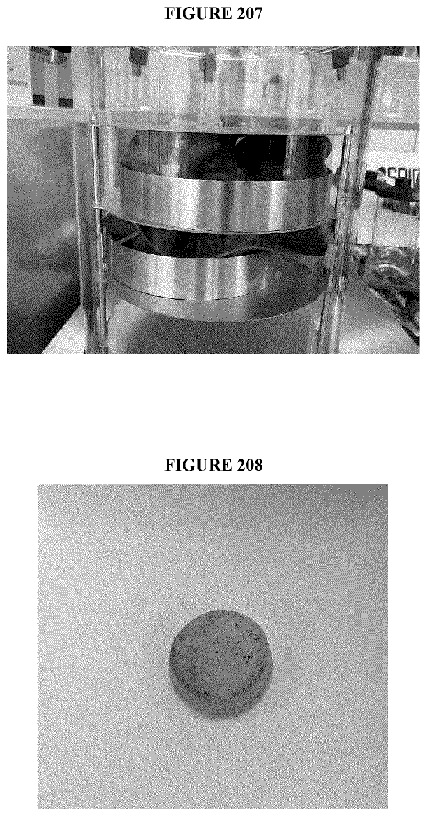

FIG. 207 shows silicone molds with frozen biomaterial in lyophilizer.

FIG. 208 shows cooked biomaterial.

### Example 23-Cooking Methods of the Biomaterial

In this example, different cooking methods on the biomaterial were tested to investigate the effect of physical and organoleptic properties including mass yield, visual appearance, and microscopy.

### Methodology

### Treatments (in Duplicate):

Pan fry-medium heat
Baking—350° F./40 min
Sous Vide—46° C./30 min

### Procedure

A 5% Sodium alginate solution stock was manufactured.
A mixture of 5% sodium alginate solution (3 mL), distilled water (4.5 mL), and mercerized apple (7.5 g)
was manufactured.
The mixture was cross-linked with 0.1M CaCl₂) for 30 min.
The scaffolds were brought to the kitchen, weighed, and 3 different cooking methods were applied.
For the pan fry method, the scaffolds were fried with vegetable oil using medium heat for 15 min.
For the sous vide, the settings were the same utilized for fish—46° C./30 min, and then the samples were
seared until reaching a browning in the surface.
For the baking method, the scaffolds were baked at 350° C. for 40 min. In this method, the samples were
checked every 15 min until they were considered fully cooked.
After each cooking method, the samples were weighed.
The yield was calculated.
The microscopy was performed using the dark field.

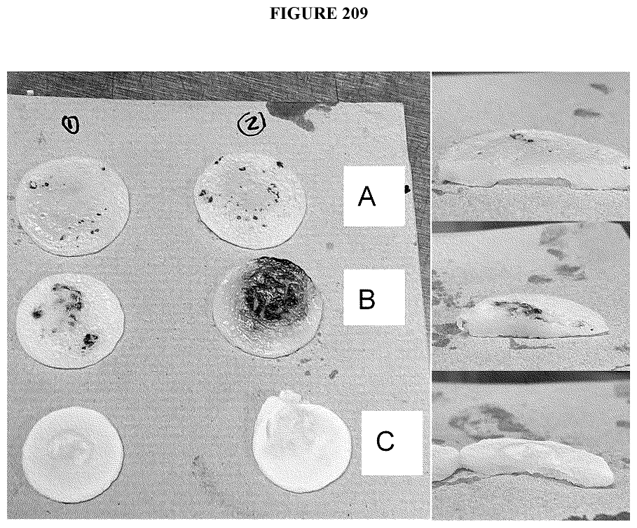

FIG. 209 shows cooked 60 mm alginate/merAA pucks via sous vide (A), pan frying (b), and baking (C).

### Results

Different cooking methods on the scaffold were performed to investigate the effect on physical and organoleptic properties including mass yield, visual appearance, and microscopy. Three different cooking methods were tested (Sous vide, pan-fry, and baking) and the yield, and microscopy were analyzed. The mass yield had a large variation between methods with the following numbers: sous vide-74.29%, pan-cooking-59.94%, and baking-32.87%, with the Sous Vide demonstrating the greater yield (p<0.05). The results are summarized in the table below. Moreover, a browning was observed when fried with oil. Specifically, the highlights for each treatment considering the yield, microscopy and visual characteristics were for baking: samples began shrunk in size, no browning occurred, had the lowest average mass yield of the three after cooking was 32.87%, samples were opaque white, cooked puck was dry on the outside while the interior was still gel-like. Pan-cooking: non-uniform shape of pucks lead to uneven browning, average mass yield of 59.94%. Sous vide: little change in appearance after vacuum sealing and cooking, puck was translucent white in colour, browning occurred during searing, greatest average mass yield of 74.29%. All samples were gel-like internally after cooking.

Yields comparison of 3 different cooking methods

| Cooking method | Average Yield % | SD % |
|---|---|---|
| Sous vide | 74.29^{A} | 4.93 |
| Pan-cooking | 59.94^{B} | 4.45 |
| Baking | 32.87^{B} | 8.90 |

SD—standard deviation; Xlstat 2014

### Example 22-Sensory Characterization of the Food-Grade Biomaterial

In this example, the apple processing was performed in the kitchen and the decellularization, and mercerization in the lab. The goal was to evaluate the colour, odour, and tactile characteristics (texture) of the biomaterial produced with 10% sodium bicarbonate and 15% hydrogen peroxide stock solution.

### Methodology

43 McIntosh apples were washed, peeled, and the core was taken out.
The apples (3.5 Kg) were cut in quarters and ground using a commercial food chopper—Hobart (3.4 kg) for 20 seconds
The ground apple (3.4 Kg) was divided evenly and added to four different 4 L beakers (850 g for each beaker).
The decellularized step was performed using the shaker (130 rpm).
For the Mercerization step, a total of 1,860 g of decell apple was divided into four different beakers: 2 beakers containing 500 g decell apple each, and another 2 containing 430 g decell apple each. All four beakers were treated with 10% Sodium bicarbonate, and 15% hydrogen peroxide stock solution, and were heated for 1 h. The Glacial acetic acid was utilized for the acidification step.
Instead of the centrifugation step the 25 μm sieve was utilized. The material was passed through the sieve until stabilizing/neutralizing the pH between 6.8-7.2.
At the end of the mercerization process a total of 854.5 g of Mer AA 138 was fabricated.

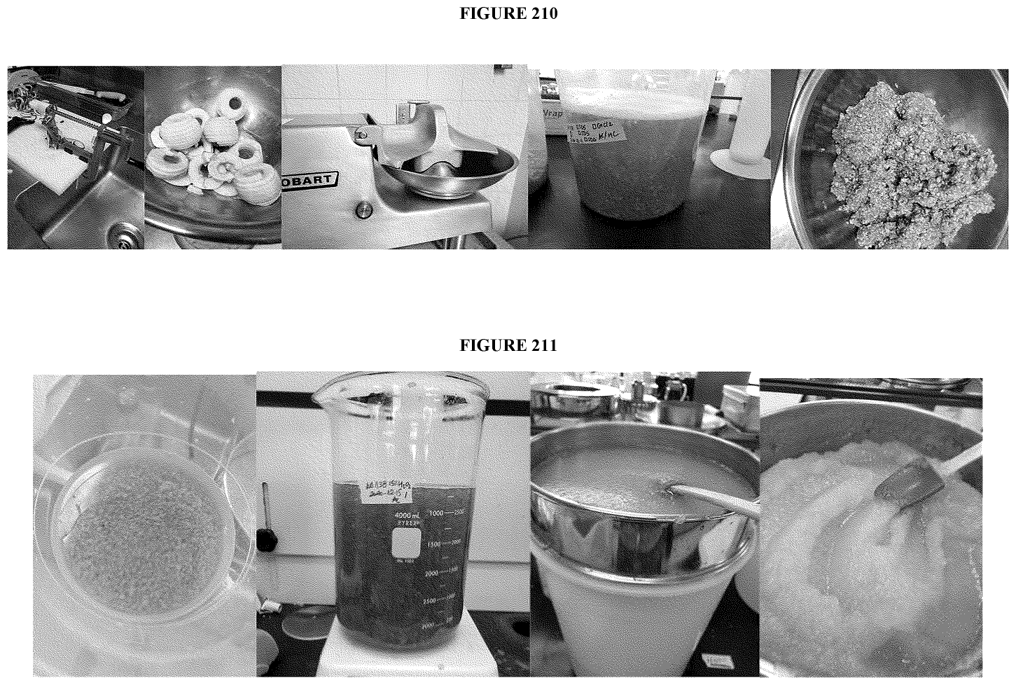

FIG. 210 shows apple (AA138) processing.

FIG. 211 shows decellularization and mercerization of the processed apples (Mer 138).

### Scaffold Fabrication

2% of sodium alginate (1 L) was manufactured in the Kitchen.
854.5 g of Mer AA 138 was homogenized with 854.5 g of 2% sodium alginate solution using the mixer (KitchenAid Custom Stand Mixer-4.5 Qt) for 5 min (200 mL) at speed 6.
For this trial, round, oval, and “squid” mold shapes were utilized.
The molds were frozen (24 h), and then freeze-dried (48 h).
The biomaterial samples were cross-linked for 1 h.

FIG. 212 shows scaffold fabrication.

### Sensory Testing

For the sensory test were utilized: 18 “squid” molds; 22 round molds, and 8 oval molds.
The references utilized for the test were: cod (1 Kg) and squid (0.7 Kg).
A circular metal mold was utilized to cut the references in the same size of the biomaterial.
The sensory test was composed of two different parts: First one called “trick station” where the panellists did not know which one of two samples displayed was the biomaterial.
An adapted paired comparison test was performed to analyze the ability of the panellists to guess correctly the biomaterial identity in the two different cooking methods.
The two cooking methods utilized were: deep fry and Sous vide with subsequent searing with butter.
For the deep fry method the squid was utilized as the pair for the biomaterial. The two different treatments were marinated in fish broth with the objective to add flavor to the biomaterial, deep in the batter (wheat flour, rice flour, sodium bicarbonate, salt, pepper, and perrier water), and deep fry for 2 min. Then the two treatments were displayed in the plates with codes, so the panellists did not know the identification for each one. For this analysis, the biomaterial was shaped in the “squid” mold.
For the Sous vide treatment the pair utilized was the cod which was cut in round shape to look similar to the biomaterial. Both treatments were placed into vacuum bags, fish broth was added to add flavor to the biomaterial, the bags were vacuum sealed, and placed into the Sous Vide machine at 46° C. for 30 min. After 30 min, the treatments were seared with butter for 2 min, and placed into plates to be analyzed. For this analysis, the biomaterial was shaped in the round mold.
On the second part of the sensory analysis test, the panellists had the identification of the biomaterial, references (cod and squid), and the objective was to use the references to help in the sensory analysis and description.
The colour, odour, and tactiles characteristics were analyzed on the second sensory test.
For each sensory parameter, both references were utilized.
The samples were analyzed first raw, and after cooked. Always from the left to the right in the following order: biomaterial, cod, and squid.
For the cooked samples the cooked method utilized was the Sous vide using the setting for fish (46° C./30 min).
For the colour parameter, one raw sample and other cooked were utilized for the Biomaterial and references. For this parameter, the biomaterial was shaped in a round mold.
For the odour parameter the samples (biomaterial, cod, and squid) were cut in small pieces and placed into cups with lids. Ten cups were utilized for each treatment, totaling 30 cups. Between samples the panellists smelled a coffee powder to clean the palate. The samples were analyzed first raw and after cooked, and always using the following order: biomaterial, cod, and squid. For this parameter, the biomaterial was shaped in the oval mold. For odour, the oval shape biomaterials were cut into strips before the cross-link.
For the tactile parameters, both references were cut in round shape and all 3 treatments were placed in plates to be analyzed. A knife and a fork were also placed with the samples to help the panellists to better characterize the samples.
All panellists utilized the form https://docs.google.com/document/d/1a22Kk6yrkmycCyK1 g-hxJsUW2B46a0n_-8Hu7B5wJxI/edit?usp=sharing to input the adapted paired comparison guess, sensory parameters descriptions, and comments.

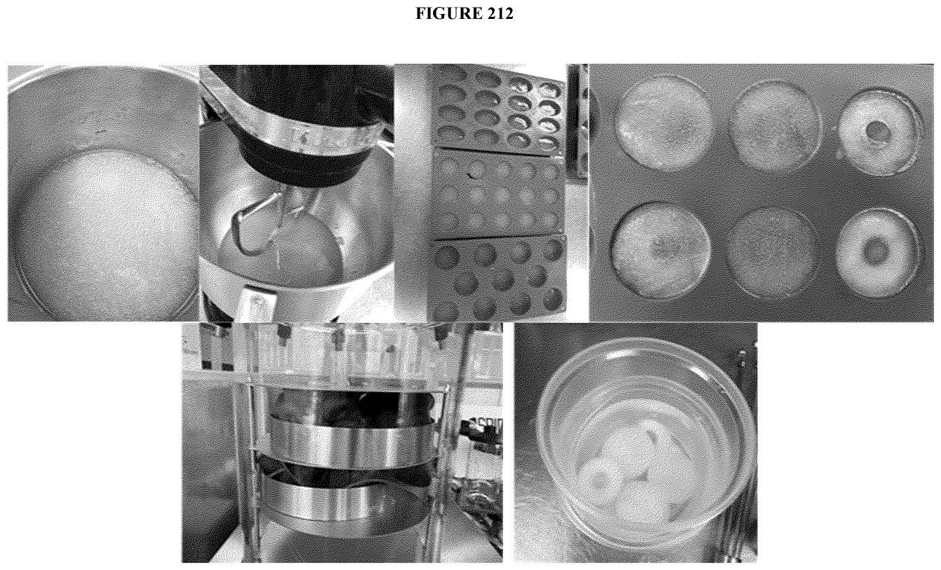

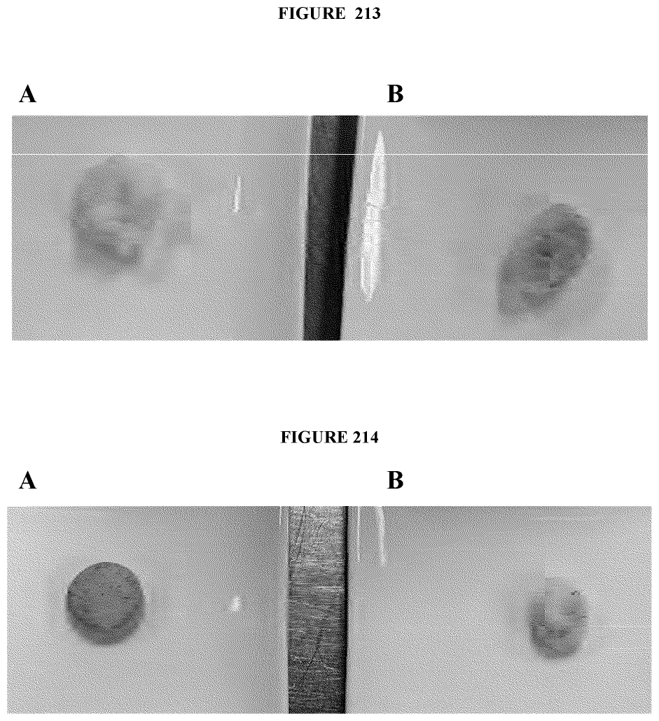

FIG. 213 shows deep fried biomaterial (A) and calamari (B)

FIG. 214 shows sous vide, seared biomaterial (A) and cod (B).

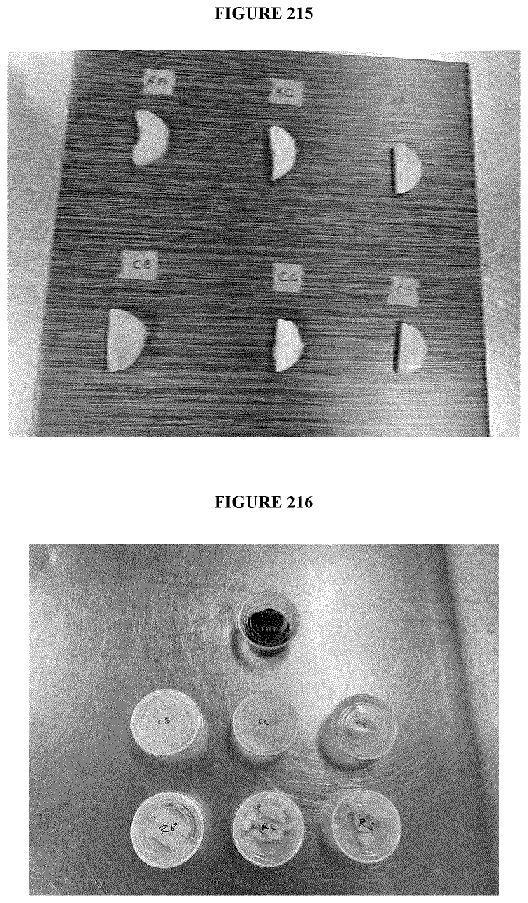

FIG. 215 shows colour test of raw biomaterial (RB), cooked biomaterial (CB), raw cod (RC), cooked cod (CC), raw calamari squid (RS) cooked calamari squid (CS).

FIG. 216 shows odour station of 6 samples and ground coffee.

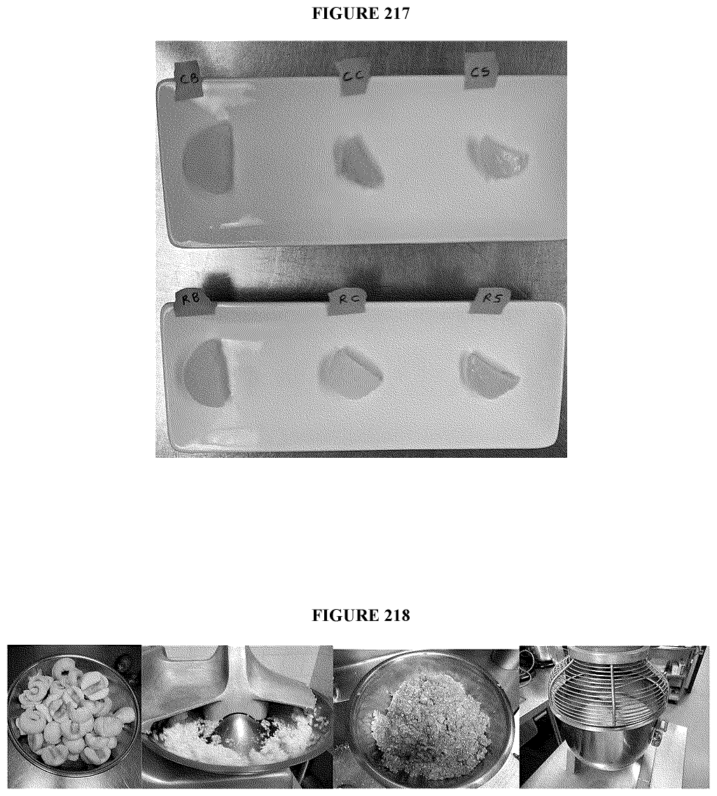

FIG. 217 shows texture comparison station of raw and cooked biomaterial compared to cod and squid.

### Results

A sensory analysis panel was conducted to gain insight into the sensory characteristics of the Spiderwort Inc. biomaterial to determine the characteristics of colour, odour, tactile parameters, flavour and texture. Also, analyze the potential presence of any residual flavours from processing such as SDS, CaCl₂), sodium bicarbonate, and any acidic neutralizing agent (acetic acid, citric acid), as well as to understand how various cooking methods affect the flavour and mouthfeel of the product. For this objective two different sensory analyses were conducted. For the second analysis, the entire process for the biomaterial fabrication was performed in the kitchen.

The first sensory test (the biomaterial was fabricated utilizing the Mer AA 138) was composed of two different parts: First one called “trick station” where the panellists did not know which one of two samples displayed was the biomaterial and an adapted paired comparison test was performed. On the second part of the sensory analysis test, the panellists had the identification of the biomaterial, references (cod and squid), and the colour, odour, and tactiles characteristics were analyzed. The Mer AA 138 utilized for the first sensory analysis scaffold fabrication presented a yield of 25.13%, compared to raw apple and 45.94%, compared to decell apple. On the first test, 9 panelists participated (4 Female and 5 Male, ranging from <20 to 50 years old). From the panelists that participated on the adapted paired comparison test, over 50% of the total panelists were not able to get the perfect scoring combination for both cooking methods, with 28.54% of wrong answers in each cooking treatment. Also, it is possible to infer that the cooking treatment was not an effect (Deep fry and Sous Vide). In addition, for the characterization of colour, odour, and tactile parameters (raw and cooked), words were generated and the most frequent were selected to describe the parameter as follows:

Based on the results, the baseline formulation had a good starting point. Over 50% of the total panelists were not able to get the perfect scoring combination for both cooking methods, demonstrating the potential of the material to mimic the meat matrix. Also, analyzing the most common comments it is possible to infer that the addition of vegetable protein would be beneficial to the formulation, giving the material more elasticity, more natural shape, and a less translucent colour.

The results are summarized in the tables below.

Words with higher prevalence in the sensory parameters description
c

| Raw colour | cooked colour | raw odour | cooked odour | raw tactile | cooked tactile |
|---|---|---|---|---|---|
| transparent/ | uniform colour | metallic/ | metallic/ | wet/moist (6) | skin layer (3) |
| translucent (8) | (6) | astringent (4) | astringent (5) | juicy/water release (3) | wet/moist (3) |
| porous/ | transparent/ | no smell (4) | no smell (4) | squishy/spongy (4) | juicy/water release (3) |
| bubbles (5) | translucent (5) | sweet (2) | weaker than | hard to cut/pierce (4) | squishy/spongy (4) |
| | | | uncooked (3) | easy to cut/pierce (2) | easy to cut/pierce (5) |

Words with higher prevalence in the cooking method description
Cooking methods comments

| Deep Fried | Sous vide |
|---|---|
| similar smell\(2) | fries/potato wedges (4) |
| Less rubbery/easier cutting, tearing (4) | soft/mushy interior (8) |
| similar appearance (4) | not flaky (5) |
| perfect shape (2) | similar smell (3) |
| little/hard to see biomaterial (3) | |

### Example 23-Taste Characterization of the Food-Grade Biomaterial

In this example, gustatory sense was used to evaluate the taste, and texture characteristics of the biomaterial produced with 10% sodium bicarbonate and 15% H₂O₂. All steps of decellularization and mercerization were adapted to the kitchen. The goal was to evaluate not only the presence of any residual taste of sodium bicarbonate, H₂O₂, and citric acid, with the goal of developing new formulations.

### Methodology

### Decellularization

For batch AA 139, the entire process including decellularization, mercerization, scaffold fabrication, cross-link, cooking, and tasting analysis was performed in the kitchen, using kitchen equipment, and FCC chemicals.
For Batch 139, 1.1 kg of decell material was not bleached due to a lack of information regarding the H₂O₂ residue. Only 123 g was bleached to test the H₂O₂ from the new supplier and test this step in the kitchen.
The solutions (0.1% SDS and 0.1 M CaCl₂)) were prepared using a dough hook in a large Hobart stand mixer bowl and mixed on speed 1.
42 McIntosh apples were washed, peeled, and the core was taken out.
The apples (3.6 Kg) were cut into quarters and ground using a commercial food chopper—Hobart (3.4 kg) for 20 seconds.
The chopped apple (3.6 Kg) was mixed with 8 L of 0.1% SDS in a large Hobart stand mixer bowl and mixed on speed 1 using the dough hook.
For 3 consecutive days, a new SDS solution was prepared and substituted in the mix with the chopped apple.
The apple was washed seven times until no soapy residue was observed.
After the washing step, 8 L of 0.1M CaCl₂) was added to the Hobart stand mixer bowl and mixed with the apple on speed 1 using the dough hook for 1 day.
After the CaCl₂) step, the apple was washed seven times and the Decell apple was ready for the mercerization process.

FIG. 218 shows apple chopping and decellularization of AA 139.

### Mercerization

For the Mercerization step, a total of 1,223 g of decell apple was divided into two different pots and performed on top of a stove: 1 containing 1.1 kg decell apple which did not receive the bleaching treatment, and other containing 123 g decell apple which was submitted to the bleaching treatment with 15% H₂O₂ stock solution.
Both samples were treated with 10% Sodium bicarbonate, the temperature was controlled using a thermometer appropriate for food, and were heated for 1 h. The citric acid (50%) was utilized for the neutralization step.
After 1 h heating, the pots were cooled off in the fridge for 15 min.
Instead of the centrifugation step, the 25 μm sieve was utilized. The material was passed through the sieve until stabilizing/neutralizing the pH between 6.8-7.2.
The final pH of the bleached treatment before neutralization was 9.1 and the final pH of the non-bleached one was 8.8.
In the final sieving step after pH neutralization, a cheesecloth was utilized applying moderate pressure to release the excess of water.
At the end of the mercerization process, a total of 767.5 g of Mer AA 139 was fabricated.

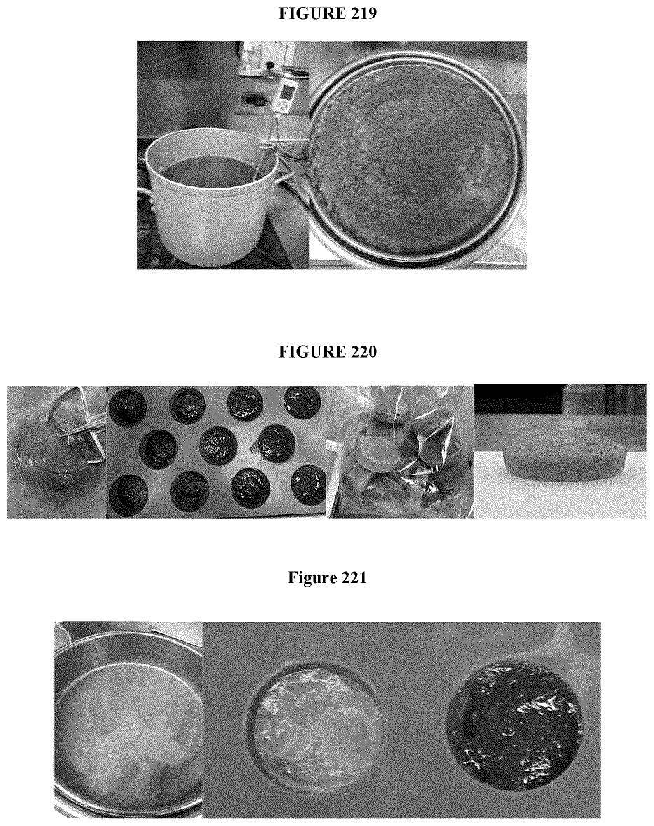

FIG. 219 shows mercerization of decell AA 139.

### Scaffold Fabrication

2% of sodium alginate (1 L) was manufactured in the Kitchen.
For the unbleached treatment, 750 g of Mer AA 139 was homogenized with 750 g of 2% sodium alginate solution using the mixer with the paddle attachment (KitchenAid Custom Stand Mixer-4.5 Qt) for 5 min at speed 6.
For this trial, just round and dome shapes (silicone molds) were utilized.
The molds were frozen (24 h), and then lyophilized in the Buchi L-200 at −55° C., 0.100 mbar (48 h).
The biomaterial samples were crosslinked for 1 h in 1% (w/v) CaCl₂) bath overnight in the refrigerator.

FIG. 220 shows scaffold fabrication.

### Bleaching AA139

For reference, a portion of AA139 was separately bleached during mercerization using 15% H₂O₂ stock solution as a comparison to the unbleached biomaterial. No other changes to the protocol were made.

FIG. 221 shows bleached MerAA139 (left) and unbleached (right) 1% Alginate/AA139 biomaterial before freezing.

### Cooking Methods Utilized for the Tasting Test

Sous vide: 50° C./30 min
Oven: 400° F./25 min
Deep-fry: 370° F./2 min

### Sensory Test

For the sensory test were utilized: 21 round molds, and 3 dome molds.
The taste references utilized for the test were:
   W—tap water
   DAS—diluted apple saucepan
   1SB—1% sodium bicarbonate
   0.5SB—0.5% sodium bicarbonate
   1CA—1% citric acid
The texture references utilized for the test were:
   SB—sous vide biomaterial (50° C., 30 min)
   SS—sous vide scallop (50° C., 30 min)
   BB—baked biomaterial (400° F., 25 min)
   BS—baked scallop (400° F., 25 min)
   DFB—deep fried biomaterial
   DFS—deep fried scallop
   SC—sponge cake
   J—jelly (14 g/L)
   M—merengue
A circular metal mold was utilized to cut the references in the same size as the biomaterial.
On the tasting analysis, the panellists had the identification of the biomaterial, and references. The objective was to use the references to help in the sensory analysis and description.
The taste and texture parameters were analyzed and the panellists utilized the form https://docs.google.com/document/d/1wTtGqmYHxZxh387O2WxTo_s_qLCh4SbcWnyhU2TkA d4/edit?usp=sharing to input the taste and texture parameters descriptions, and comments.

### Results

On the second sensory test (the biomaterial was fabricated utilizing the Mer AA 139), the panellists had the identification of the biomaterial, and references (different references for flavour and texture). The objective was to use the references to help in the sensory analysis and characterization of taste and texture parameters. The Mer AA 139 utilized for the second sensory analysis scaffold fabrication presented a yield of 21.30%, compared to raw apple and 62.71%, compared to decell apple. On the second test, 7 panellists participated in the sensory analysis test (4 Females and 3 Males, ranging from <20 to 50 years old). The beef flavour was the most frequently flavour observed in the scaffold submitted to the Sous vide cooking method, potentially due to an association with the Maillard reaction which can occur after the searing using butter (reaction between the carbonyl group on a sugar and amino group) (Boekel et al., 2006). Also, a fish/scallop flavour was noticed in the Sous Vide and deep fry treatments. The oil/butter was the most noticeable in the deep fry treatment, while a residual sodium bicarbonate taste was noticed in the oven treatment potentially due to the concentration of the bicarbonate, demonstrating the necessity for more washing steps to avoid this taste. Based on the results, regarding flavour, the scaffold demonstrated the ability to efficiently absorb flavours.

Regarding texture, the texture characteristic most noticed in the Sous Vide and Deep fry treatment was the succulence whereas, in the oven method was the dryness. In addition, a texture parameter detected in all three treatments was the cohesiveness. The deep fried scallops and biomaterial were the most similar ones. Analyzing the most common comments it is possible to infer that the addition of vegetable protein would be beneficial to the formulation, giving the material more elasticity, more natural shape, and a less translucent colour. Also a deeper investigation regarding the “beef” flavour should be valuable for product development.

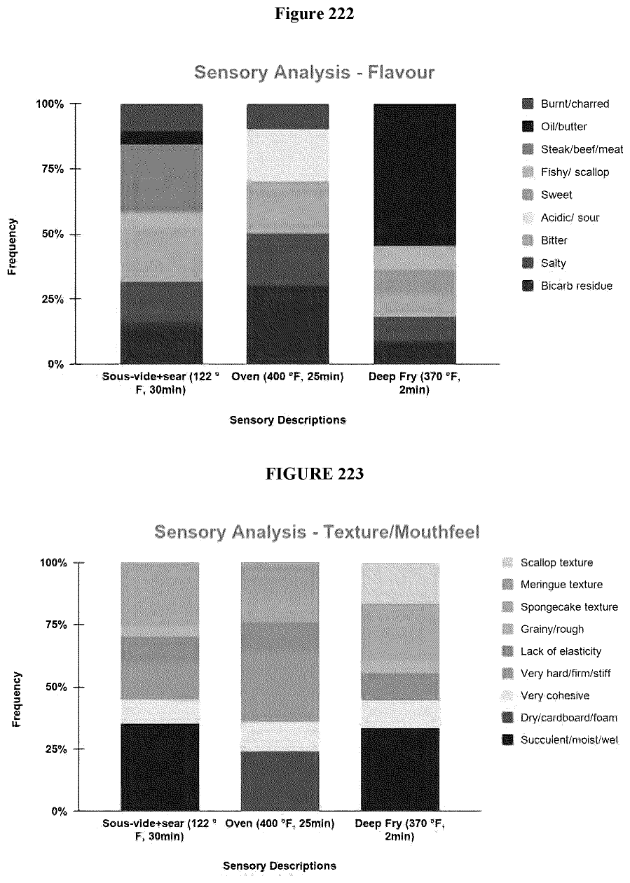

FIG. 222 shows sensory results for flavour-frequency of words.

FIG. 223 shows sensory results for texture/mouthfeel-frequency of words.

### Example 24-Development of Fibres in the Scaffold to Mimic Meat Fibres

In this example, different techniques to create fibres in the scaffold resembling the fibres found in meat were used to develop a product with a unique texture.

### Methodology

### Strategy:

### A-Unidirectional Freezing (UF)

The objective was to use the unidirectional freezing technique to create aligned porous resembling meat fibres.

### Treatments:

1% Sodium alginate+Mer AA (Trial 1)
UF-Mer AA:2% Sodium Alginate (1:1)—petri dish (Trial 2)
Treatment A: UF-Mer AA:2% Sodium Alginate (1:1)—inox cylinder mold (Trial 3)
Treatment B: UF-Mer AA:2% Sodium Alginate (coloured with red beet in a acidic pH) (1:1)—inox cylinder mold (Trial 3)

### Procedure:

### Trial 1

An entire canned palm heart was decellularized in a 5-day process.
The Sodium alginate treatment: 2% sodium alginate (7.5 g) and Mer AA (7.5 g) was fabricated.
A styrofoam support was created to receive the sample.
The treatment was placed in the unidirectional freezer and kept inside for 3 h.
After the unidirectional freezing, the treatment was transferred to a conventional freezer and kept inside for 48 h.
The treatment was Lyophilized (0.100 mbar at −55° C.) for 48 h and visualized in the microscope.

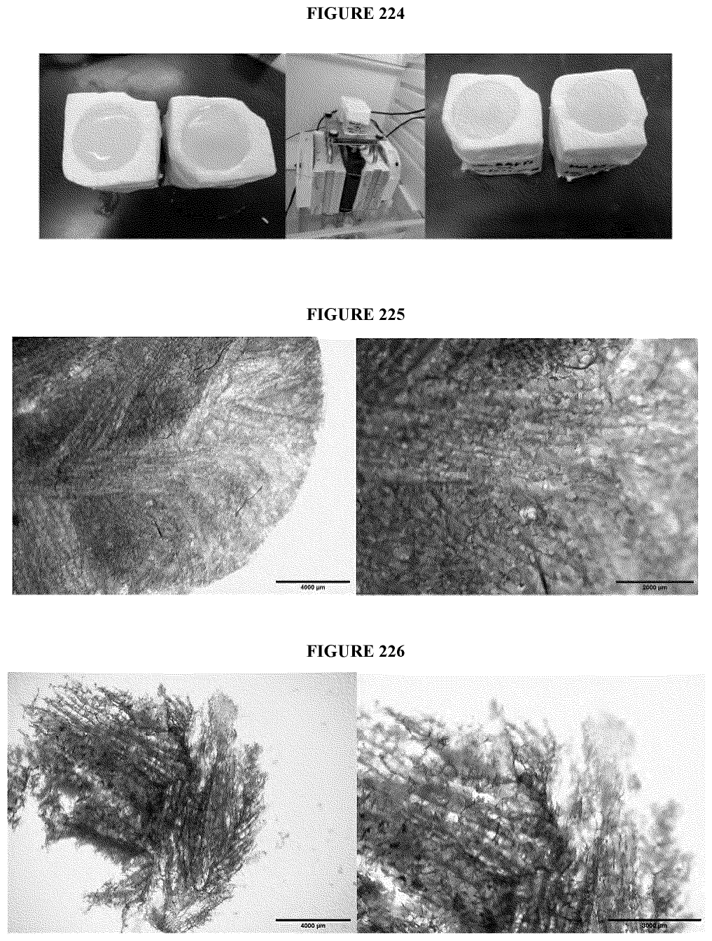

FIG. 224 shows unidirectional freezing of 1% Alginate treatment.

FIG. 225 shows microscopy images of the top side of the 1% Alginate biomaterial after unidirectional freezing in 0.7× (left), and 1.6× (right) magnifications

FIG. 226 shows microscopy images of the bottom side of the 1% Alginate biomaterial after unidirectional freezing in 0.7× (left), and 1.25× (right) magnifications.

### Procedure:

### Trial 2

The treatment was prepared using a 1:1 ratio of 2% sodium alginate and Mer AA and was poured into a petri dish.
For the treatment was utilized 20 mL of material with 1 mm height.
The treatment was placed in the unidirectional freezer and kept inside for 3 h.
After the unidirectional freezing, the treatment was transferred to a conventional freezer and kept inside for 48 h.
The treatment was Lyophilized (0.100 mbar at −55° C.) for 48 h and visualized in the microscope.

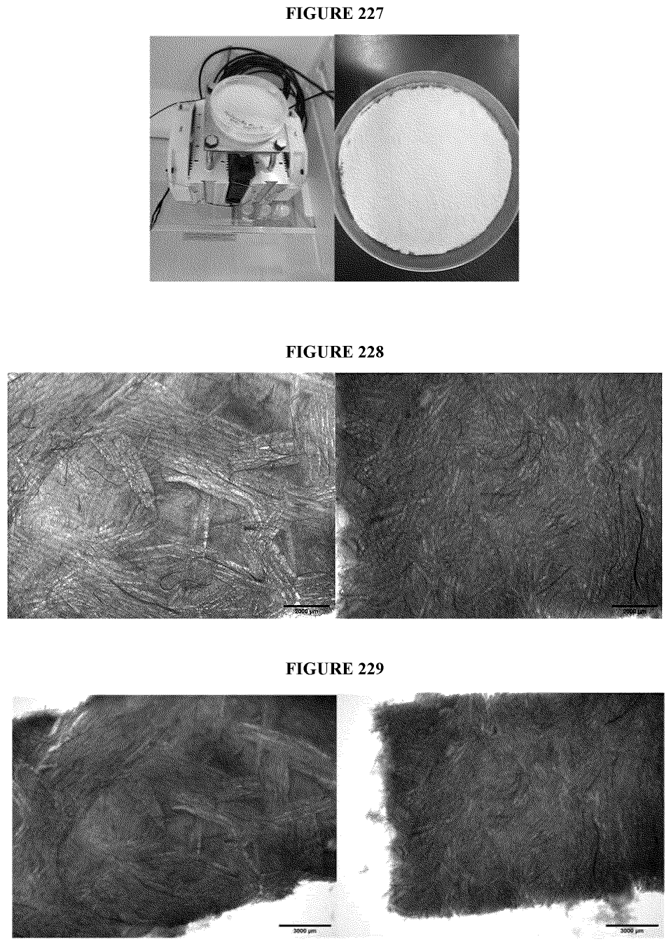

FIG. 227 shows unidirectional freezing of Mer AA:2% Sodium Alginate (1:1) in a petri dish.

FIG. 228 shows microscopy images of the edge (left) and center (right) of the “Mer AA:2% Sodium Alginate (1:1) in a petri dish” biomaterial after unidirectional freezing.

FIG. 229 shows microscopy images of the of the edge (left) and center (right) of the “Mer AA:2% Sodium Alginate (1:1)—petri dish” biomaterial after unidirectional freezing in 0.7× magnification.

### Trial 3

### Procedure:

The 2 different treatments were prepared using 1:1 ratio of 2% sodium alginate and Mer AA each, and were poured into an inox cylinder mold.
On treatment B, 2 g beet root powder was added directly to 20 mL of a 2% (w/v) alginate solution at a pH between 5-5.3. After the mixture with Mer PH was fabricated.
For the inox mold container, 30 mL of mixture was utilized.
The treatments were placed in the unidirectional freezer and kept inside for 4 h.
After the unidirectional freezing, the treatments were transferred to a conventional freezer and kept inside for 48 h.
The treatments were Lyophilized (0.100 mbar at −55° C.) for 48 h and visualized in the microscope.

### Results

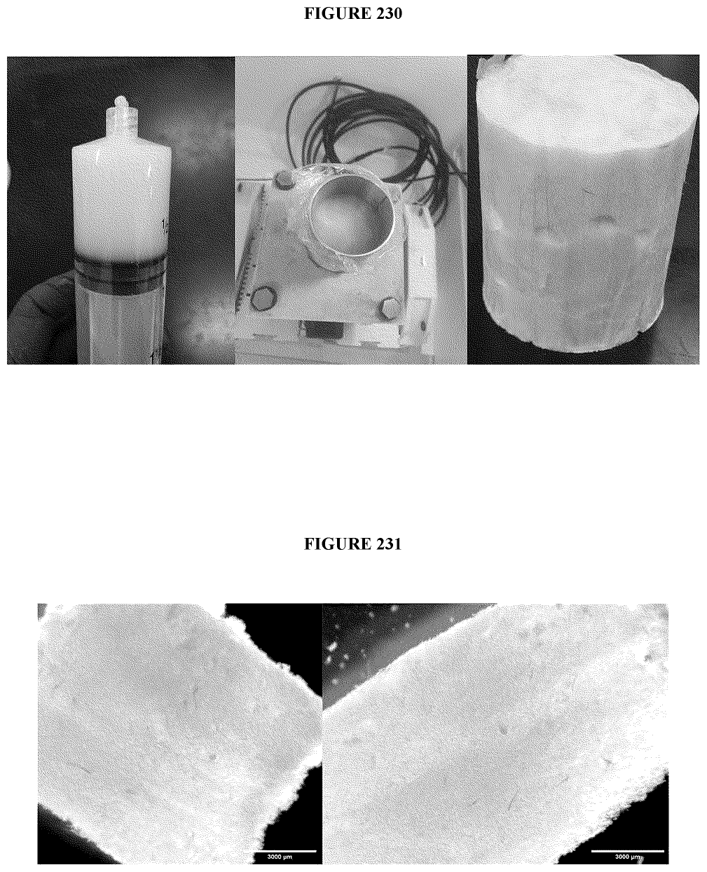

FIG. 230 shows biomaterial preparation of Treatment A (left), UF treatment (middle), and Lyophilized biomaterial (right).

FIG. 231 shows microscopy images of a longitudinal cut from Treatment A using the 1× magnification.

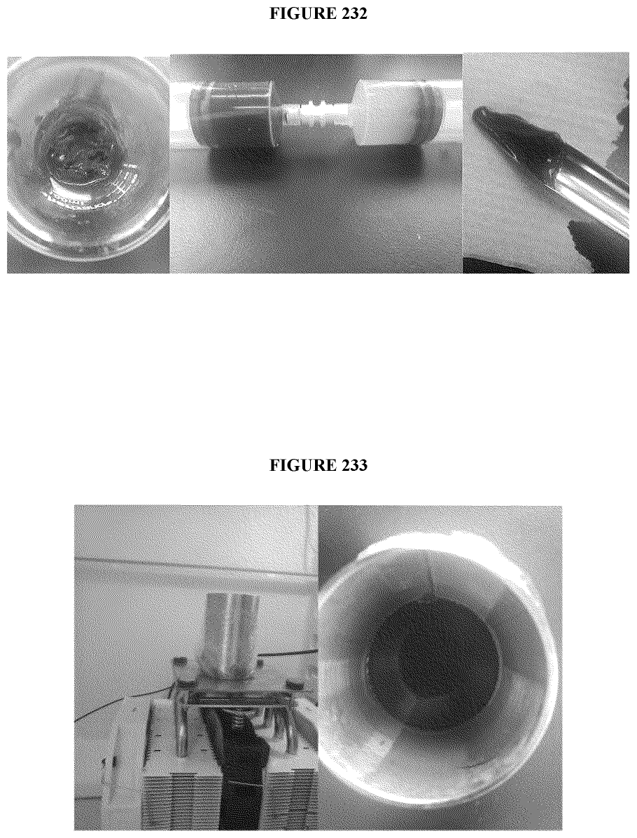

FIG. 232 shows biomaterial preparation of Treatment B.

FIG. 233 shows unidirectional freezing of Treatment B.

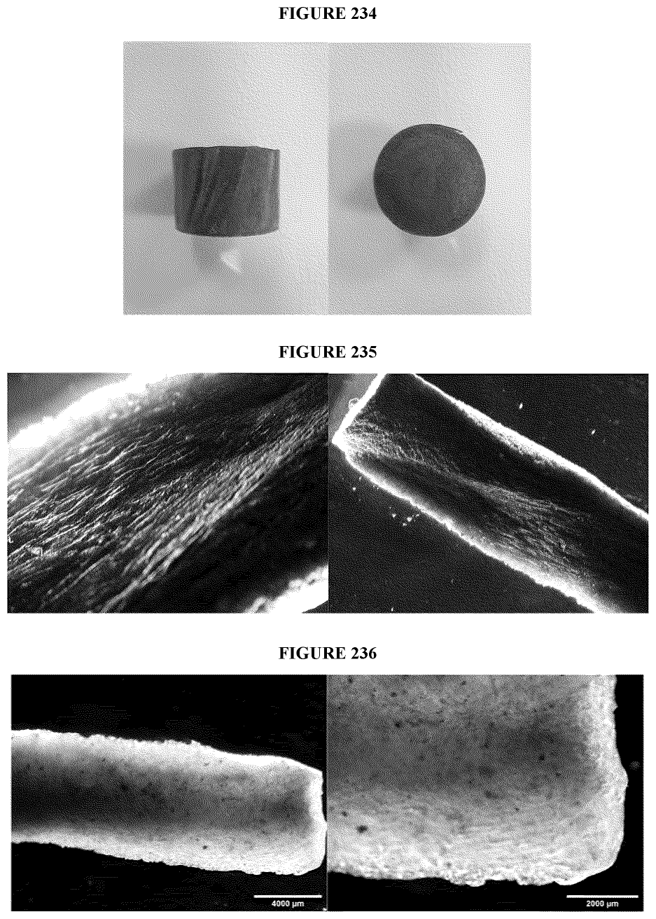

FIG. 234 shows lyophilized biomaterial of Treatment B.

FIG. 235 shows microscopy images of Lyophilized Treatment B in 1.6× (left) and 0.7× (right) magnifications.

FIG. 236 shows microscopy images of cross-linked Treatment B in 0.7× (left) and 1.6× (right) magnifications.

The unidirectional freezing represents a technique to create aligned porous resembling meat fibres. The freeze alignment is a technique utilized to produce porous materials with orientation structure in an aqueous solution or slurry of proteins. The dispersion of inorganic particles or polymer in water allied to the growth speed control and ice crystals orientation creates unidirectional porous scaffolds after ice crystals sublimation, leaving pores (Zhang et al., 2005). The concentrations of the polymer, as well as the concentration of the cross-linker, are factors that can affect the alignment and the size of the porous (Wu et al., 2010). The time, pH, and mold apparatus were the factors tested to establish the best condition to acquire the aligned porous looking fibers in the Spiderwort Inc. formulation. All the conditions tested resulted in a sort of aligned porous. However, the best formulation up to the present moment was the mixture of Red beet (10%) in 2% Sodium alginate with Mer palm heart with acidic pH and 4 h in the Unidirectional freezing and placed in a inox cylindrical mold. The inox mold (mold material) clearly affected positively the creation of horizontal aligned porous. Both formulations placed in the inox mold during the UF, demonstrated horizontal alignment porous through all the biomaterial. In the coloured material the horizontal aligned porous like-fibres were more evident and were noticed on the surface and on the center of the biomaterial.

### Example 24-Natural Fibres (Palm Hearts) to Mimic Meat Fibres

In this example, palm hearts were used to mimic meat.

### Methodology

The canned palm heart was cut in the longitudinal direction.
The palm heart was decellularized in a 5-day process.
Part of the decellularized palm heart fibres was kept to be utilized as the whole fibre in the scaffold and the other part was Mercerized using 10% Sodium bicarbonate and bleached using 15% H₂O₂ stock solution.
The decellularized longitudinal fibres (50 g) were combined with the Mer PH (70 g) and 2% Sodium Alginate (70 g).
The mixture was mixed using a whisk, and placed in two different mold shapes (circle and rectangle).
The biomaterial was frozen for 48 h with posterior lyophilization for 48 h (0.100 mbar at −55° C.).
The frozen biomaterial was cross-linked using 1% CaCl₂) in fish broth for 30 min.
The biomaterial was pan-fried for 2 min each side using butter.

### Results

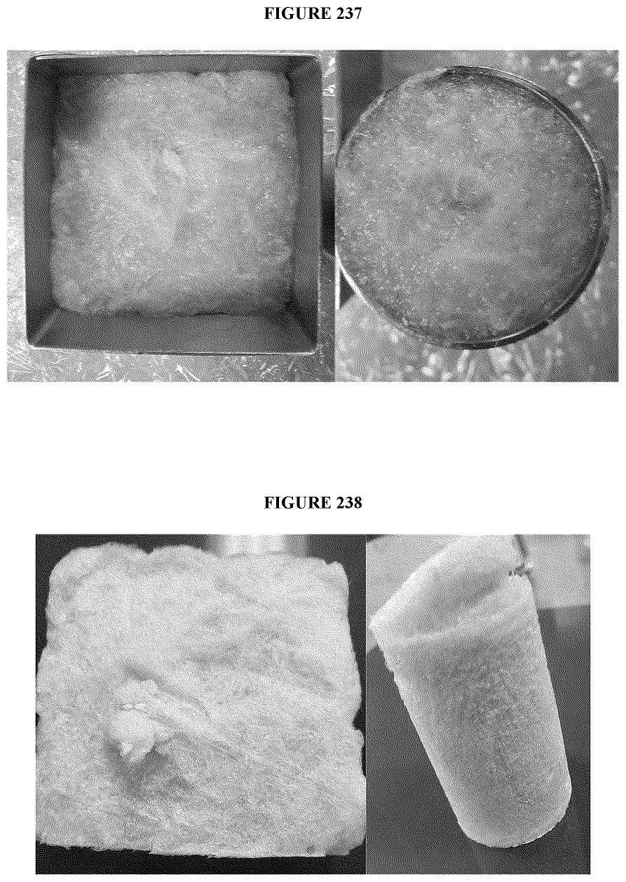

FIG. 237 shows Mercerized/decellularized palm heart blend in metal moulds.

FIG. 238 shows Lyophilized biomaterial of decellularized and mercerized palm heart before crosslinking.

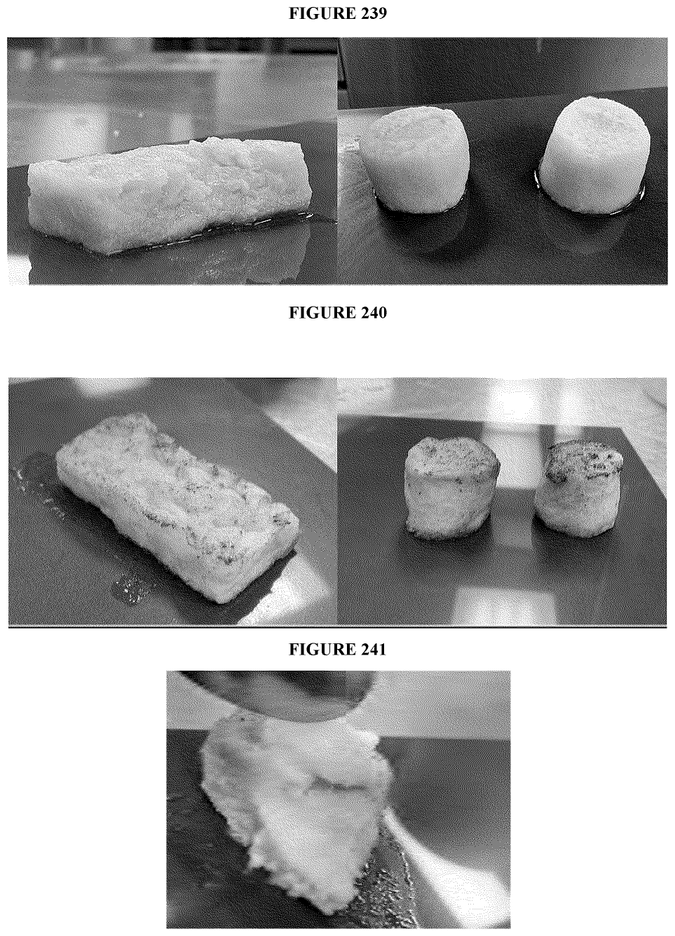

FIG. 239 shows raw, crosslinked biomaterial “fishstick” (left) and “scallop” (right) of decellularized and mercerized palm heart.

FIG. 240 shows cooked, crosslinked biomaterial “fishstick” (left) and “scallop” (right) of decellularized and mercerized palm heart.

FIG. 241 shows peeled back layer of cooked palm heart biomaterial.

Heart of palm is a vegetable harvested from the inner core and growing bud of certain palm trees and presents a natural fibrous appearance being broadly used as a vegan seafood option. The peach palm (Bactris gasipaes Kunth) is a tropical palm, source of fruit and heart of palm, the last one consisting of the edible inner core of the palm stem with the following characteristics: cylindrical, soft, tender, and slightly sweet. The peach palm heart is the central part of the palm heart, divided into three parts (basal, central, and apical), which differ hardness. The central portion, considered of higher quality and the most common sold in the market in canned versions, is rich in fibers. The total dietary fiber content of the central part is 45.62 (g 100 g-1), whereas cellulose, hemicellulose, and lignin are 37.76, 5.38, and 0.44 (g 100 g-1), respectively. In addition, a hardness of 2.21(N) and an elasticity of 8.47 (mm) are observed in the central part of the palm heart (Stevanato et al., 2020). Thus, canned decellularized fibres from palm heart (palm heart fibres) were utilized in a tentative way to mimic the fibres observed in the meat. Moreover, the mercerization of the palm heart (longitudinal) was also performed to observe if this process could fabricate a fibrous material which could be utilized in the future. The mercerized palm heart (PH) demonstrated a fibrous appearance with a milk colour, similar to the raw scallop. The fibers noticed in the Mer PH were not apparent in the scaffold.

Vegan products (fish fillet and scallop) were elaborated utilizing the mixture of decellularized PH fibres, Mercerized PH, and 2% Sodium alginate. The “fish fillet” and “scallop” made using the aforementioned demonstrated the appearance of biomaterial similar to the target conventional products and developed a flaky structure resembling fish meat. On the other hand, the biomaterial still presented a spongy texture in the cut potentially due to the concentration of alginate utilized. Alginate (Alg) is a linear copolymer of (1-+4)-linked ß-D mannuronic acid (M) and α-L-guluronic acid (G) residues in varying sequences, considered the main structural component in marine brown algae (Phaeophyceae). The alginate combined with a specific concentration of Ca2+ can create a cohesive or firm gel (Yang et al., 2020).

### Example 25-Use of Different Types of Glues to Mimic a Whole Muscle

In this example, sodium alginate was used to glue different pieces and/or layers of scaffold to create a “whole muscle” and test the efficacy of the glue in different types of cooking.

### Treatments

Treatment B: UF-Mer AA:2% Alginate-red beet (1:1): pan-cooked, and boiled
Treatment C: UF-Mer AA:2% Alginate-(1:1)-5 different replicates: pan-cooked.

### Procedure

The lyophilized treatment B was cut in four different parts and glued together using a thin layer of 2% Sodium alginate as glue in two different pieces of biomaterial simulating two “pieces of meat”.
The treatment C was fabricated using the formulation: Mer AA+2% SA-1:1 and divided in five different replicates to create five different layers. All replicates were placed in 60 mm petri dishes and frozen for 48 h.
After the freezing step, the replicates were Lyophilized and also glued together using a thin layer of 2% Sodium alginate.
Both treatments were cross-linked using 1% CaCl₂) for 1 h at room temperature and one of the coloured pieces of biomaterial continued the cross-link overnight (24 h) in the fridge.
Both treatments were pan-cooked using butter for 1 min each side.
The coloured piece cross-linked overnight in the fridge was boiled 100° C. for 8 min.

### Results

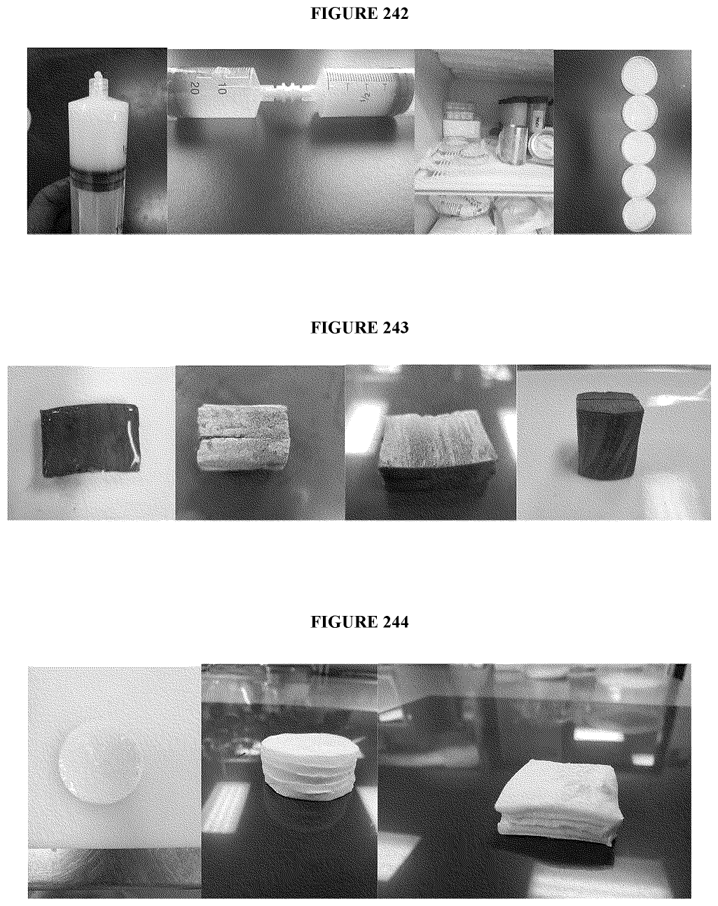

FIG. 242 shows preparation of the biomaterial and layers of Treatment C.

FIG. 243 shows gluing process and two different pieces fabrication from the treatment B.

FIG. 244 shows gluing process and two different pieces fabrication from the treatment C.

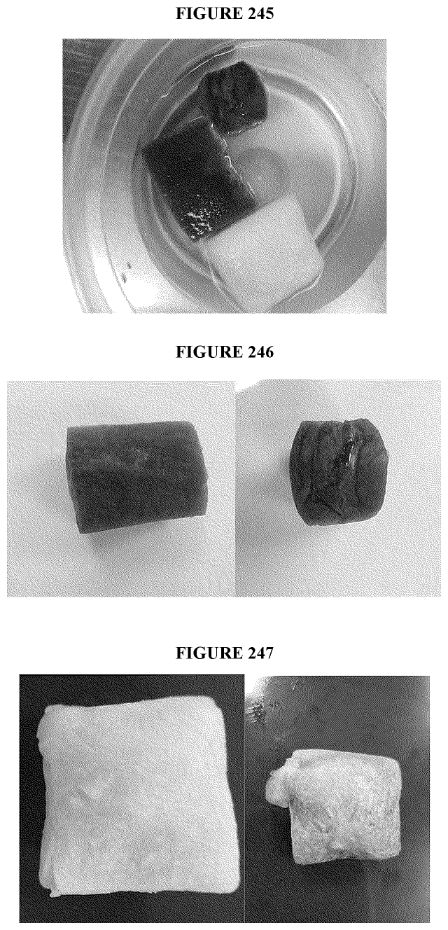

FIG. 245 shows cross-link step with 1% CaCl₂) for 1 h at room temperature or in the fridge for 24 h.

FIG. 246 shows Treatment B cross-linked for 1 h at room temperature.

FIG. 247 shows cross-linked (left) and pan-cooked treatment C.

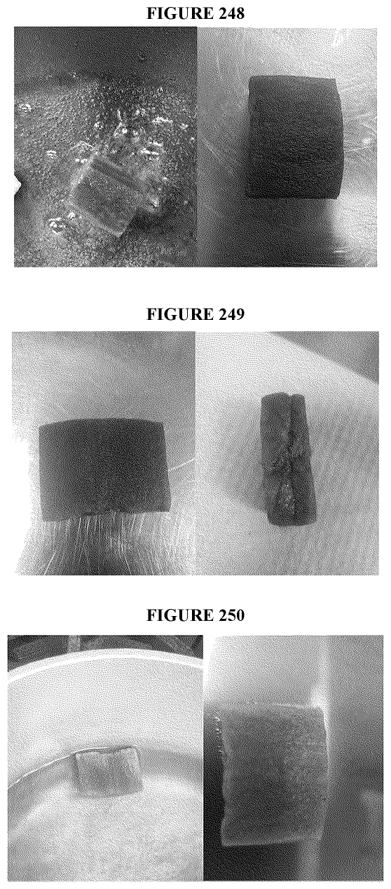

FIG. 248 shows pan-cooking process and pan-cooked treatment B.

FIG. 249 shows Treatment B cross-linked in the fridge for 24 h.

FIG. 250 shows boiling process and boiled Treatment B.

The utilization of different types of GRAS “glue” represents a key step for different plant-based formulations to develop a complex structure mimicking the meat one. The alginates are widely utilized in different food products, due to their unique properties as food additives. Recently, a study at Colorado State University, utilized the alginate to “glue” beef pieces together and demonstrated the capacity of this texturizing to hold the pieces together at ordinary temperatures allowing irregularly shaped pieces of meat to be restructured into a whole muscle, resulting in a more profitable product (Yimin et al., 2018). For that, the 2% sodium alginate was utilized to glue different pieces and/or layers of scaffold to create a “whole muscle”. Also, the efficacy of the “glue” was tested in different types of cooking. The 2% Sodium alginate solution demonstrated efficacy to glue together pieces and/or layers of lyophilized biomaterial. In addition, the sodium alginate as a glue was able to maintain the scaffold glued during pan-cooking and boiling. No colour denaturation was observed on treatment B in the pan-cooking whereas, a heat denaturation was observed during the boiling. The excess of colour was lost during the cross-link on treatment B, but the scaffold did not lose the colour even during the boiling process, confirming previous trials.

### Example 26-Vegan Formulations

In this example, a plant-based version of a fish fillet was developed using Mer AA.

### Methodology

### Treatments:

Formulation Fish A
Formulation Fish B

### Procedure:

The canned palm heart was cut in the longitudinal direction.
The palm heart was decellularized in a 5-day process.
One formulation was fabricated and subdivided in two different treatments:

### Fish A

Decellularized Palm heart
78 g Mer AA
15 g Pea protein
5 mL Sunflower oil
9 mL Sodium alginate
1 g NaCl
1 g Transglutaminase
0.1 g Tumeric
Sous Vide: 50° C./3 h

### Fish B

Decellularized Palm heart
78 g Mer AA
15 g Pea protein
5 mL Sunflower oil
9 mL Sodium alginate
1 g NaCl
1 g Transglutaminase
0.1 g Turmeric
0.1 g Turmeric
Freeze for 24 h
Lyophilize for 48 h
Cross-linked with 1% CaCl₂
Sous Vide 50° C./3 h
The ingredients were homogenized using a kitchen mixer, and the mixtures were placed into the Sous Vide.
The Fish B dough was placed in an inox mold inside a vacuum bag and was vacuum sealed before the frozen step.

After the Sous Vide, both treatments were pan-fried for 1 min each side.

### Results

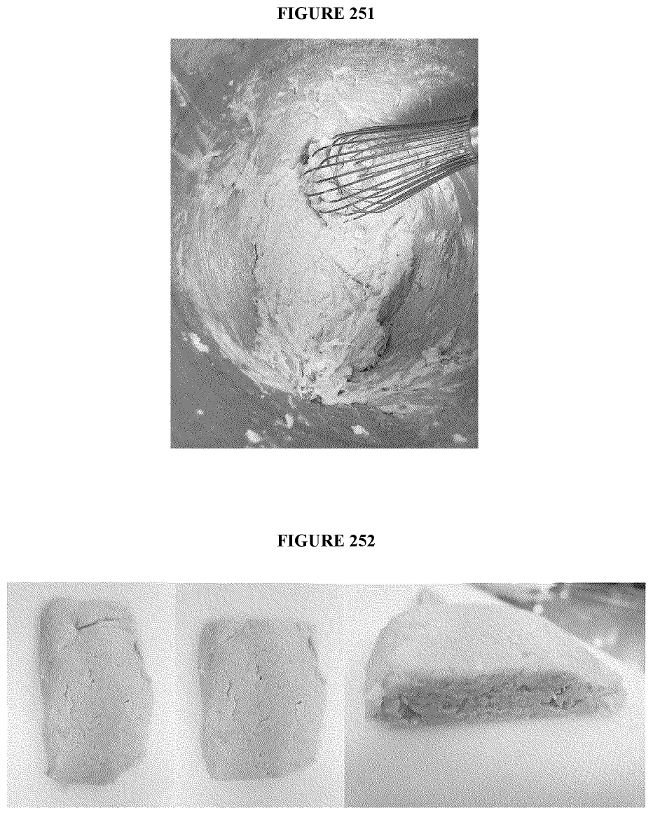

FIG. 251 shows Ingredient mixing and product fabrication-Fish A and Fish B.

FIG. 252 shows Fish A after Sous Vide treatment.

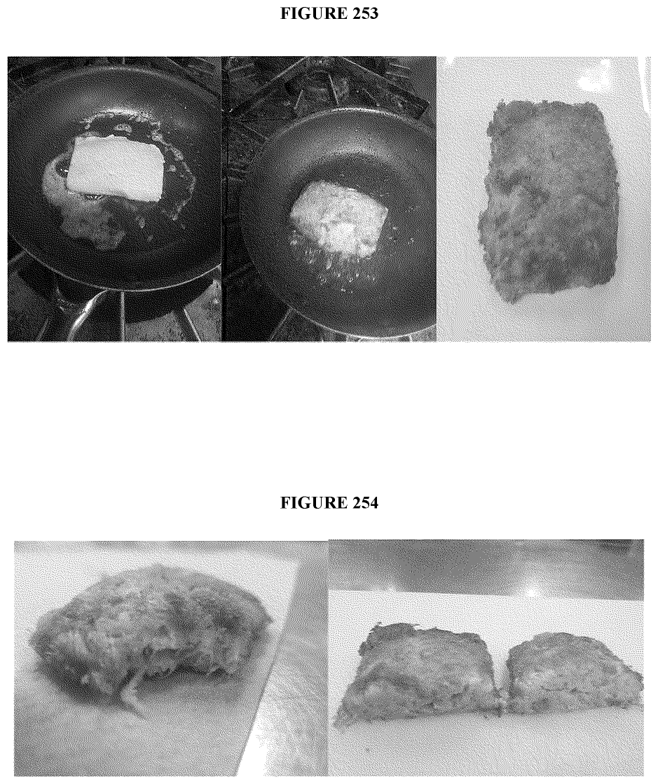

FIG. 253 shows pan-cooking and cooked Fish A.

FIG. 254 shows pan-cooked Fish A-Cross-section.

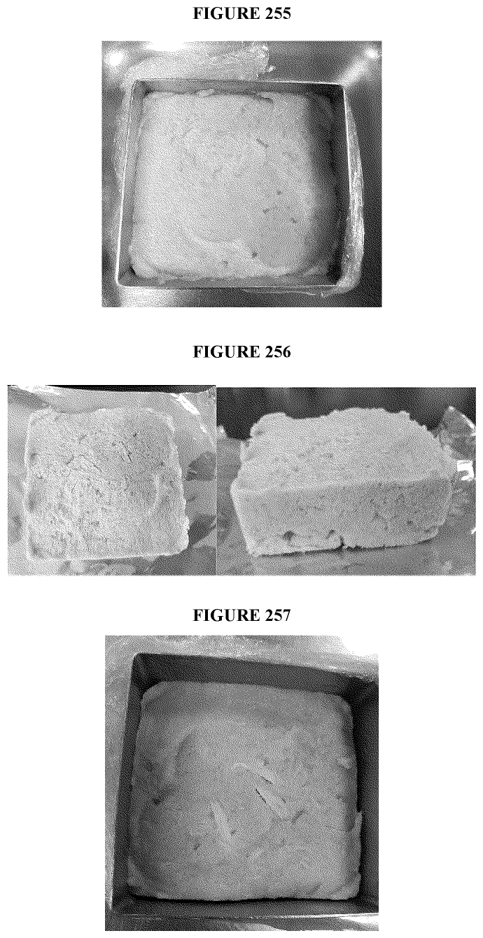

FIG. 255 shows Fish B placed in the inox mold.

FIG. 256 shows lyophilized Fish B.

FIG. 257 shows cross-linked Fish B.

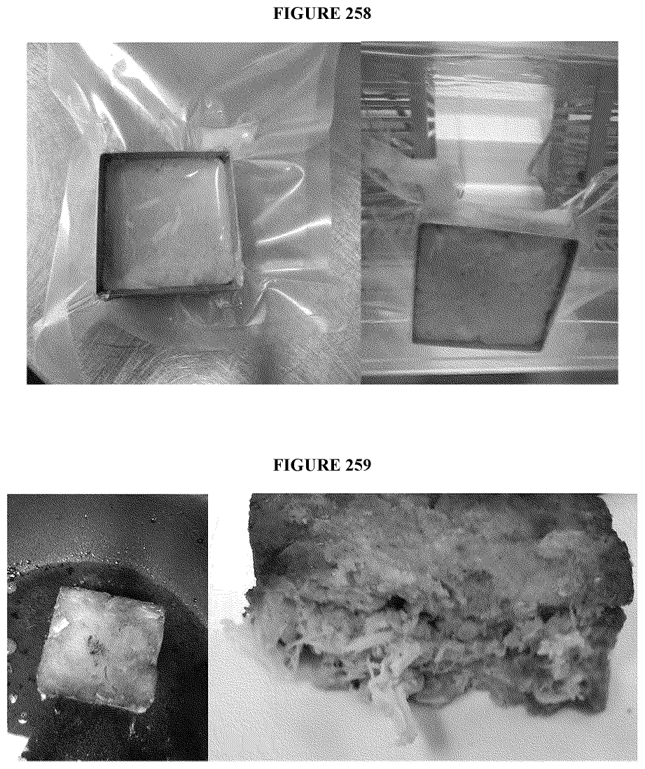

FIG. 258 shows Fish B Vacuum sealed before the Sous Vide (left) and during the Sous Vide (right).

FIG. 259 shows pan-cooking and cross-section of pan-cooked Fish B.

The utilization of decellularized palm heart allows to reproduce vegan formulation with fibre-like texture such as fish fillet and fish products. The proximate composition of fish fillet and fish product can vary for Moisture from 66.30 to 82.30, for protein from 8.20 to 25.90, for fat from 0.1 to 21.0, and for ash from 0.96 to 2.85 (Reddy et al., 2012; Atanasoff, et al., 2013; Venugopal & Shahidi, 1996). In order to mimic fish fillet or fish product, decellularized palm heart was utilized to reproduce the fibres, the mercerized apple was utilized as the scaffold whereas an isolated vegetable protein (pea protein) was added in the formulation instead of animal protein, and sunflower oil substituting animal oil. The fish muscle is composed of sarcoplasmic proteins such as myoglobin, hemoglobin, globulins, albumins, and various enzymes, considered the most water soluble ones. In addition, other types of proteins constituting the fish muscle are stromal proteins, collagen and elastin, the least soluble fractions (Venugopal & Shahidi, 1996). The mercerized apple was incorporated into the formulation in order to provide a better texture to the final product. For the fish fillet project, one formulation was developed and subdivided into two treatments in which different approaches were performed to test the effect on the final texture. However, both treatments demonstrated similar texture. The utilization of the Lyophilization for the treatment B didn't demonstrate necessary. The texture of the two different treatments were similar to fish products but still not to a fish fillet. Thus, the formulation needs an ingredient to increase elasticity and hardness of the formulation such as the utilization of other types of vegetable proteins or Konjac Flour.

Canning is a widespread technique consisting of a combination of processes such as immersion in acid brine, heat treatment, exhaustion, and hermetic sealing which promotes food preservation and shelf life improvement. However, canning can affect the mechanical properties. Stevanato et al. (2020) demonstrated a decrease in the total fibre and cellulose contents. Also showed a decrease in the mechanical properties for the palm heart decreasing the hardness and elasticity (Stevanato et al., 2020).

### Example 27-Continuous Feed Cross-Linking

A current challenge is to increase the scalability of the products to a commercially viable level. Here we present an alternative method for a continuous feed crosslinking, which involves extruding the material into a crosslinker bath to crosslink “on the go”. As the material exits the extruder, it can be crosslinked in that shape. The screen. mold, or perforated plate which the material passes through dictates the shape of the crosslinked material. The bulk material behind the crosslinked portions remain in the fluid, gel, or sol phase until they are pushed into the crosslinker. As such, the bulk material can be stored in syringes, or other configurations that can be delivered to the shape determiner and crosslinking bath such as pumps and platens. This bulk processing allows for high throughput material manufacturing. It is complementary to the inverse methods of molding and crosslinking around spacers.

Potential applications include, but are not limited to: packaging materials, insulations, sealants (vascular, pleural, gastro, muscular, fascia), nucleus pulposus, tissue fillers, wound repair, meniscus, designer tissues, neural scaffolds.

### Methodology

Mercerized decellularized apple (MerAA) mixed with low methoxyl pectin.
Formulation: 7.5 g of MerAA+4.5 mL of water+3 mL of 5% (m/v) pectin.
Production: Mix in luer lock connected syringes the extrude into a CaCl₂) (0.1 M) crosslinking bath via needle delivery or transfer to a platen and perforated plate extruder (as shown below).

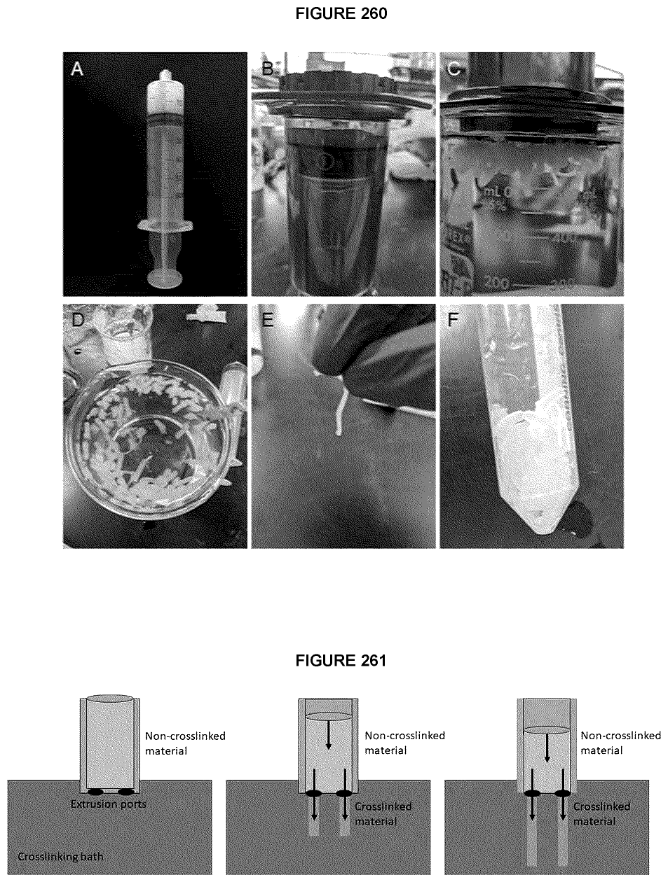

FIG. 260 shows high throughput continuous crosslinking from injectable composite materials. A: injectable pectin and MerAA mixture. B: hydrogel material loaded into a platen extruded with a perforated plate. C: extrusion into the crosslinking bath. D: the resultant crosslinked hydrogels with predefined shapes. E: The physical properties can be tuned; here the material can be handled easily. F: collection and preparation for lyophilization if desired.

FIG. 261 shows schematic of representation of continuous feed crosslinking.

### Example 28-Subcutaneous Implantation of Foam Biomaterial

To test the biocompatibility of the aerogel materials, a study was conducted where the scaffolds were subcutaneously implanted under the skin of Sprague Dawley rats and resected after 4 and 12 weeks. The scaffolds were examined for cell penetrance as well as inflammation.

### Methodology

### Key Chemicals and Solutions

1. Mercerized apple paste made from decellularized McIntosh apples, neutralized
2. 5% Alginate
3. Calcium Chloride

### Implantation

1. Rats are given subcutaneous injections of saline 0.9% and buprenorphine (0.05 mg/kg) prior to surgical implantation.
2. Rats are anesthetized using Isoflurane.
3. Apply ophthalmic liquid gel to protect eyes from drying
4. The rats are shaved from hips to shoulder, on both sides of the back
5. The skin is washed and sterilized with aseptic solutions. Four 1-2 cm incisions are performed (two on the upper back, and two on the lower back on either side of the spinal column)
6. Incisions are made through the epidermis, dermis, and subcutaneous fat layers to the underlying muscle.
7. Biomaterials are implanted into each incision (1 implant per incision).
8. The incisions are sutured and transdermal bupivacaine 2% is applied to the suture sites.
9. Additionally, buprenorphine is then administered subcutaneously 4-6 hours following the first injection.
10. Rats were allowed to recover, then implants were resected after 4 and 12 weeks respectively

### Resection Procedure

1. Transfer the animal to the euthanasia box.
2. When doing two rats at once (two boxes connected) set the initial flow rate of the CO2 to 6 and then increase to 12 when the rats become unconscious.
3. Monitor the breathing pattern of the animal. Wait at least 5 minutes.
4. Ensure the breathing has stopped for 1 minute.
5. Turn off the CO2.
6. Remove the rat from the euthanasia box and place the rat on its backside.
7. Locate the xiphoid and make an incision in the skin.
8. Pierce the diaphragm. The heart should be visible.
9. Cut the heart and ensure blood starts to pool.
10. Turn the animal onto its stomach to expose the back.
11. Cut the skin from the hip, along the centre of the spine, to the shoulder. Peel down the skin and cut away the connective tissue. The flap of skin should contain the implant/injected material.
12. Take a photograph of the material in the skin flap with a ruler for scale.
13. Specimens were collected after 4 and 12 weeks and placed in a 50 mL falcon tube filled with 4% PFA for 72 hours, followed by 70% ethanol, then stored at 4 C.
14. Once in ethanol, the samples were delivered for paraffin embedding, sectioning and staining with Hematoxylin and Eosin, and Masson Trichrome at various levels.
15. Serial sections were cut and stained with either hematoxylin-eosin (H&E) or Masson's trichrome (MT).

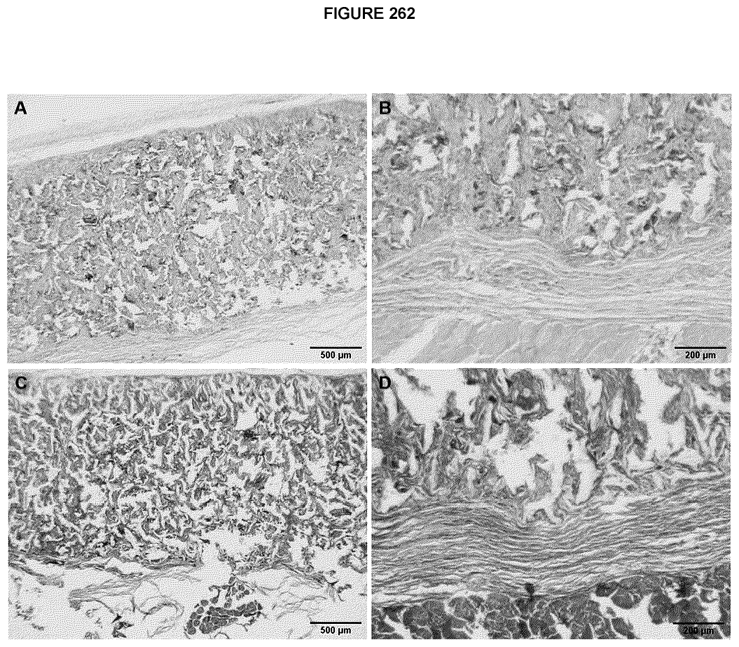

FIG. 262 shows directionally frozen scaffolds—HE (A,B) and MT (C, D) 4× and 10× excised after 4 weeks of subcutaneous implantation.

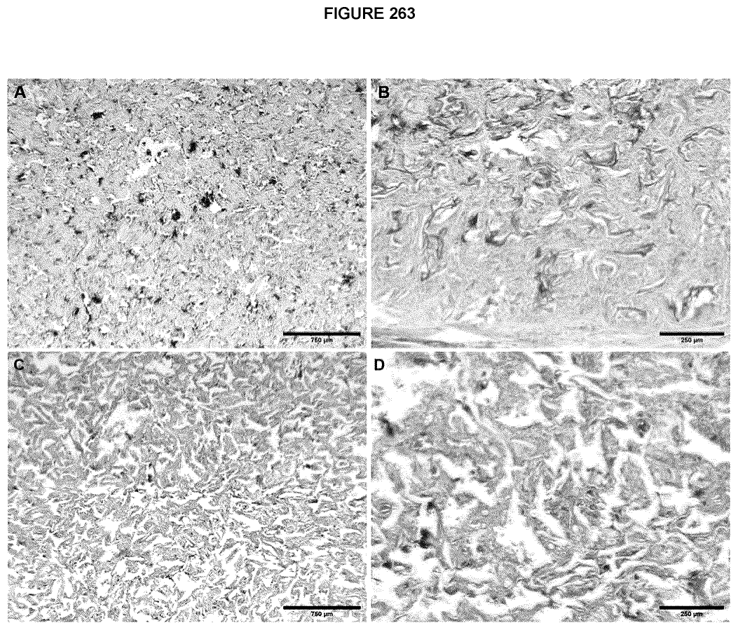

FIG. 263 shows directionally frozen scaffolds—HE (A,B) and MT (C, D) 4× and 10× excised after 12 weeks of subcutaneous implantation.

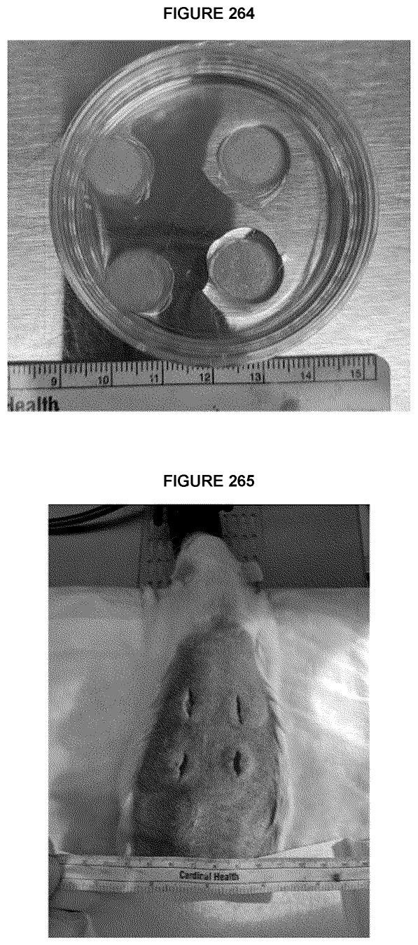

FIG. 264 shows aerogel material prior to surgical subcutaneous implantation in 0.9% sterile saline solution

FIG. 265 shows Sprague Dawley Rat with aerogel materials implanted subcutaneously each into their own site prior to suturing.

The results show significant cellular penetrance into the scaffold material, both around and into the centre of the implanted material. More cells can also be seen in the implants resected after 12 weeks, compared to 4. Additionally, no significant inflammation was noted thus suggesting graft acceptance.

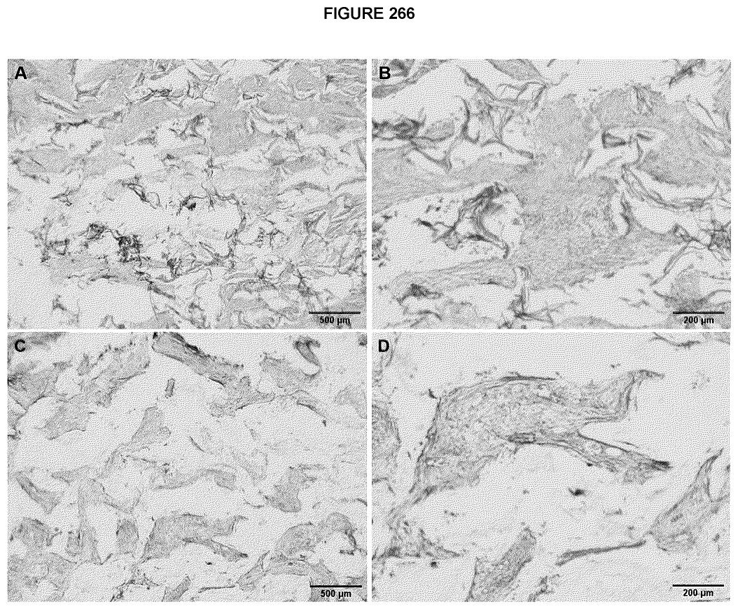

FIG. 266 shows non-directionally frozen aerogel scaffolds—HE (A,B) and MT (C, D) 4× and 10× excised after 4 weeks of subcutaneous implantation.

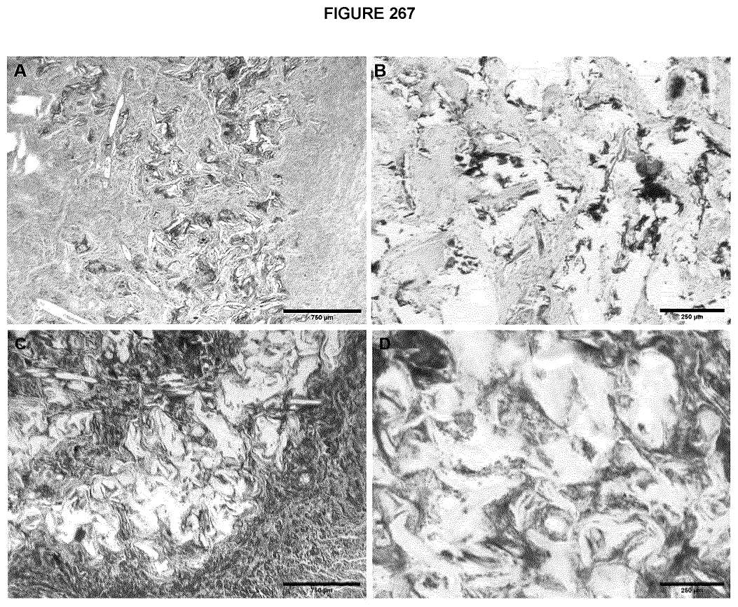

FIG. 267 shows non-directionally frozen aerogel scaffolds—HE (A,B) and MT (C, D) 4× and 10× excised after 12 weeks of subcutaneous implantation.

The non-directionally frozen scaffolds were sectioned into 5 μm thick sections and stained with H&E and MT. Similarly to the directionally frozen scaffolds, staining revealed that the scaffolds remained at their implant sites throughout the duration of the study and revealed vascularization into the native tissue as early as 4 weeks. The fibrin sealant appears to have degraded and collagen deposition is also present.

In contrast to the directionally-frozen scaffolds, there is significantly more open space not occupied by scaffold or cells. Therefore the amount of cellular infiltration, although more is evident after 12 weeks compared to 4, is still significantly less than directionally frozen scaffolds. This suggests that the cellulose scaffold offers a significantly better support structure for cellular infiltration and migration into the tissue.

### Example 29-Spinal Cord Implantation

Given the highly porous nature and structural linearity of the aerogels, it is contemplated that the aerogels described herein would be suitable for spinal cord injury repair. Small scale transection injury studies assessing the effectiveness and biocompatibility of the scaffolds were performed. Here, directionally frozen aerogel scaffolds were implanted into the transected spinal cord of the rat. This was done to determine whether the material would provide a suitable support structure for axonal regeneration. The biomaterials were implanted in rats for 4 and 12 weeks. A complete spinal cord transection was induced between T9-T10 in Sprague Dawley rats and allowed to dieback for 10 minutes. The distance from the dieback was measured and an appropriately sized aerogel scaffold was cut to size then implanted between the cut ends.

### Methodology

Scaffolds were directionally frozen as previously described with the Pelletier apparatus, followed by lyophilization and crosslinking in calcium chloride solution. After sterilization, 4 mm pieces were punched out from the larger sample producing cylindrical samples. The scaffolds were brought to the surgical theatre in 1× sterile PBS solution, and washed in 0.9% saline solution prior to surgical implantation.

### Key Chemicals and Solutions

Mercerized apple paste made from decellularized McIntosh apples, neutralized
5% Alginate
Calcium Chloride

### Transection

1. Using the dissecting microscope, locate the layer surrounding the spinal cord.
2. Using fine nose tweezers, pinch only the dura surrounding the spinal cord and lift it up slightly.
3. With the other hand, using microscissors, make a small incision in the dura cutting up vertically to expose the desired portion of the spinal cord.
4. Once exposed, use a spinal cord hook to lift the desired region of the spinal cord
5. Using microscissors held perpendicular to the cord, make a transverse cut to the cord, and release instruments. Stop any bleeding with a small piece of sterile gel foam, keeping track of how many pieces were used so that the same amount can be removed.
6. Allow the transected cord to dieback for 10 minutes prior to measuring the distance between ends.
7. Measure the distance between cut ends and cut scaffold to desired length.

### Implantation

1. Prepare the biomaterial of the same size for implantation (maintaining sterile conditions).
2. Place the biomaterial in sterile saline solution and bring adjacent to surgical field
3. Place the biomaterial between transected ends of the spinal cord ensuring no extraneous pieces remain
4. Once the biomaterial has been properly inserted, seal it to the spinal cord ends using prepared commercially available Fibrin Tisseel sealing agent (Cat #1503152, Baxter).
5. Apply fibrin sealant by assembling the duploject system and ejecting the combined sealant solutions directly to the site of the wound.
6. Allow sealant to cross-link for 3-4 mins then begin closure of the incision site.

### Perfusion and Resection

1. Set up perfusion apparatus and prepare the pump by flushing the lines and ensuring no air is trapped
2. turn on the hood and the cold water on the surgical table
3. Adjust panels of the table creating a trench to allow the rat to lay on its back without movement
4. stop the pump then place a hemostatic clamp on the in-tubing
5. Transfer the closed in-tubing to the chilled 0.9% Heparinized saline 25 U.I/mL tank
6. Release the hemostatic clamp only when the tubing is submerged below the saline water line to ensure no air bubbles are in the line.
7. Attach a 10 gauge needle to the out-tube of the perfusion pump
8. Flush the system for 4 minutes to ensure 0.9% Heparinized saline is in the tubing
9. Using the morning weight, draw the necessary volume of euthanyl (750 mg/kg) in (Catalog #)
a 10 CC syringe with a 23 gauge needle
10. Grasp the animal using the “burrito technique” by placing the animal in the center of a huck towel, then folding each side over the animal, then grasping the animal gently by the outside of the towel, ensuring that the spine is supported.
11. Inject euthanyl into the animal's right side closest to midline at the level of the umbilicus (See FIG. 3).
12. Place the animal back into its cage to monitor. The animal should begin succumbing to euthanyl in approximately 20 minutes and become unresponsive to a toe pinch.
13. Place the animal into the necropsy hood on its back between the two panels.
14. Perform a final toe pinch to confirm lack of response.
15. Quickly, pull the skin of the abdomen upwards with a hemostat and using scissors, cut through the abdomen to the sternum to expose the diaphragm.
16. Cut the diaphragm and the ribs to the shoulder joint of the forelimbs.
17. Lift the sternum and place it behind the forearms of the rat.
18. Cut through the pericardial membrane to expose the heart.
19. Insert a 10 gauge needle into the base of the left ventricle until the end of the bevel of the needle, then clamp the needle with a hemostat
20. Turn on the pump.
21. Cut the right atrium of the heart with surgical scissors.
22. Continue the perfusion for 12.5 minutes with the chilled heparinized saline.
23 Record the time the liver changes color from a deep red to brown
24 Turn off the pump and place a hemostat clamp onto the in-tube
25 Lift the closed tube from the saline beaker to the PFA reservoir. Do not remove the hemostat until the tube is passed the water line of the PFA
26. Turn on the pump for 13 minutes (the extra half minute is for the remaining saline in the tubing).
27 The animal will begin twitching from the PFA solution. After approximately 13 minutes, the body should be stiff.
28. Remove the 10 gauge needle from the animal and flush the system with water.
29 Take the animal from the vented hood to the necropsy table.

### Resection

1. The spinal column and the skull of the animal are cut away from the remaining tissue.
2. The remaining tissue connective and muscular tissue are removed from the remaining spinal column and skull
3. Starting from the rostral end, the bone snappers are inserted into the vertebral foramen and the spinal column snipped to the posterior process.
4. The dorsal surface of the spinal column is progressively lifted up revealing the next caudal vertebra to cut.
5. A laminectomy is performed until reaching the initial injury site T8-T9.
6. The spinal columns are completely cut away
7. The spinal cord is carefully lifted from the remaining column and the peripheral nerves cut away
8. the entire cord is then placed in a 50 mL falcon tube filled with 4% PFA for 72 hours, followed by 70% ethanol, then stored at 4 C
9. Once in ethanol, the samples were delivered for paraffin embedding, sectioning and staining with Hematoxylin and Eosin, and Masson Trichrome at various levels.

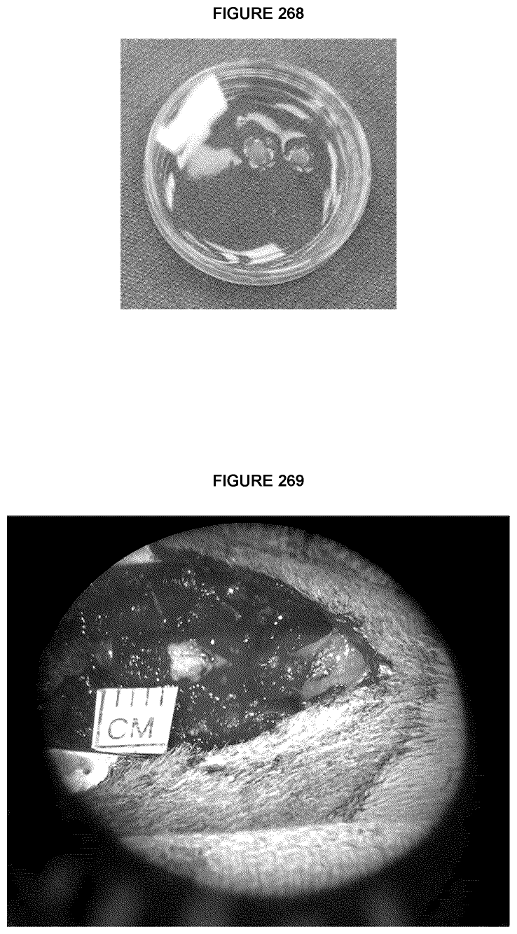

FIG. 268 shows directionally frozen scaffolds prior to implantation in sterile 0.9% Saline solution.

FIG. 269 shows directionally frozen scaffold implanted into spinal cord of Sprague Dawley Rat.

### Example 30-Bone Regeneration

In this example, the aerogel scaffolds was also examined for its ability to serve as a support material for the regeneration of bone tissue, supporting recalcification of surrounding tissues in a rat critical-size bilateral defect model. Here, a trephine was used to generate two 5 mm diameter holes in the cranium of the Sprague Dawley Rat. Once the bone defects were excised, the aerogel formulations were placed within the defect. Overlying skin was sutured and the rat left to recover for a period of 4 to 8 weeks. Specimens were collected at each time point and computational tomography (CT scanning) was performed.

### Methodology

### Key Chemicals and Solutions

Mercerized apple paste made from decellularized McIntosh apples, neutralized
5% alginate
Calcium Chloride

### Implantation

1. The rat is prepared for anesthesia and isoflurane is administered until unconsciousness is observed
2. The rat is then transferred to the preparation area, saline administered via syringe and tear gel applied over the eyes to reduce corneal dryness.
3. The top of the head is shaved from the bridge of the snout between the eyes to the caudal end of the skull, then the fur vacuumed off.
4. The rat is then transferred to the surgical area and secured to the stereotactic equipment.
5. The skin is washed with water and sterilized with chlorhexidine.
6. The biomaterials are photographed in sterile saline next to a ruler.
7. A trephine is secured to the drill and placed next to the surgical area
8. Once the researcher dons a sterile gown and gloves, an incision is made with a scalpel down the periosteum over the scalp from the nasal bone to just caudal to the middle sagittal crest.
9. Using 5.5 mm alm retractors the skin is exposed to the underlying bone.
10. The periosteum is divided down the sagittal midline and dissected.
11. The bone is cleaned with a sterile cotton swab,
12. The left parietal bone is scored with a 5 mm trephine under constant irrigation of sterile normal saline, under 1500 rpm
13. Moving circumferentially around the defect margin with the elevator blade, the defect is completed by applying gentle pressure
14. The blade of the elevator is used to slide under to remove the bone
15. The right bone is similarly removed
16. A non-sterile researcher brings the biomaterials to the surgical area and places them where instructed
17. Carefully, each biomaterial is placed in the defect of each parietal bone.
18. The biomaterials are photographed next to a ruler.
19. The alm retractors are removed and the incision is closed using interrupted sutures.
20 Bupivacaine is applied to the sutures and the rat is transferred to the recovery station.

### Resection

After the rats were allowed to recover for the desired amount of time (e.g. 8 weeks), specimens were collected and scanned with computational tomography (CT). Histology was then also performed.
1. The animal is transferred to the CO2 euthanasia box and the correct flow rate is set
2. After at least 5 minutes, and the rat has been determined to stop breathing for at least one minute, it is removed from the box.
3. Vital signs are examined and a thoracotomy followed by exsanguination is performed
4. Turning the rat on its stomach, the skin above the cranium is lifted and cut off with scissors to expose the implants
5. Using a scalpel, the muscles on either side of the cranium are cut away
6. Then, using a drillbit, the front of the calvarium is severed from the rest of the skull
7. The calvarium is then lifted using tweezers, cutting away tissue from underneath.
8. Once removed, a small notch is made on the bottom left of the calvarium to indicate directionality of the sample, and a picture taken of the implants within the trephinated area
9. The calvarium is then placed into a tube with formalin solution for 72 hours, followed by 70% ethanol, then stored at 4 C.
10. Once in ethanol, the samples were delivered for CT scanning. Each sample was rotated 180° and imaged every 0.7°.

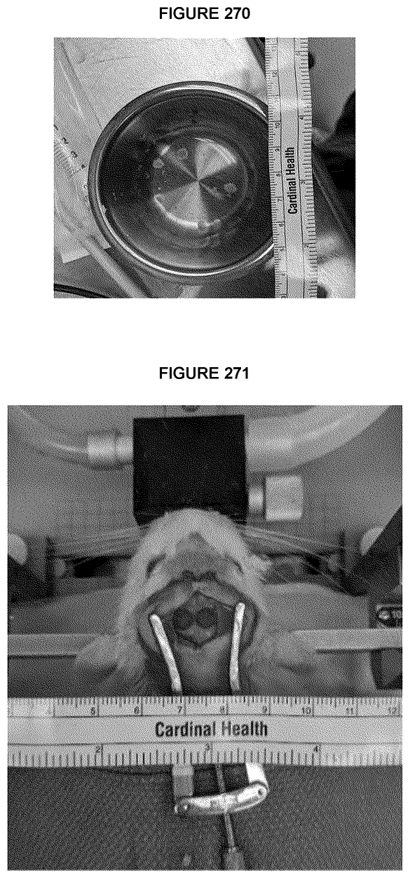

FIG. 270 shows aerogel biomaterials prior to surgical implantation into calvarial defect.

FIG. 271 shows Sprague Dawley Rat with implanted aerogel materials crosslinked with alginate and calcium chloride.

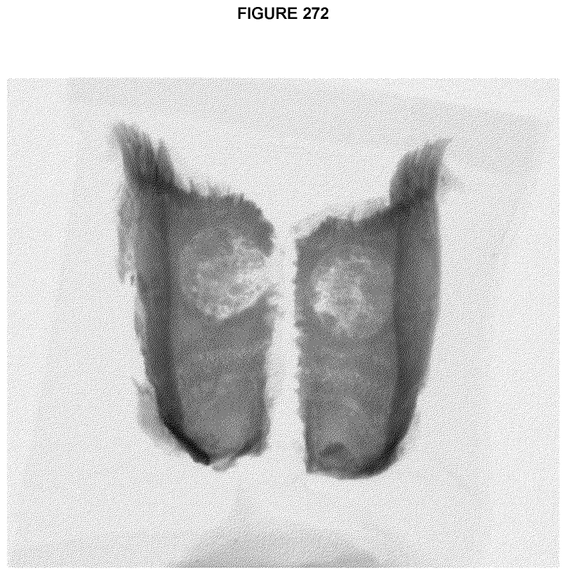

FIG. 272 shows CT scan of resected cranium with calvarial defects in a Sprague Dawley Rat resected after implantation of aerogel material 8 weeks prior.

One or more illustrative embodiments have been described by way of example. It will be understood to persons skilled in the art that a number of variations and modifications can be made without departing from the scope of the invention as defined in the claims.

What is claimed is:

1. An aerogel or foam comprising:
single structural cells, groups of structural cells, or both, derived from a plant or fungal tissue, the single structural cells or groups of structural cells having a decellularized 3-dimensional structure lacking cellular materials and nucleic acids of plant or fungal tissue;
the single structural cells, groups of structural cells, or both, being distributed within a carrier, the carrier derived from a dehydrated, lyophilized, or freeze-dried hydrogel.

2. The aerogel or foam of claim 1, wherein the aerogel or foam is rehydrated.

3. The aerogel or foam of claim 2, wherein the plant or fungal tissue from which the single structural cells or groups of structural cells are derived comprises decellularized plant or fungal tissue.

4. The aerogel or foam of claim 3, wherein the plant or fungal tissue is decellularized using SDS and optionally CaCl₂.

5. The aerogel or foam of claim 4, wherein the single structural cells, groups of structural cells, or both, are derived from the plant or fungal tissue by.

6. The aerogel or foam of claim 5, wherein the maceration or mercerization comprises treatment of the plant or fungal tissue using sodium hydroxide and hydrogen peroxide or sodium bicarbonate and hydrogen peroxide with heating.

7. (canceled)

8. The aerogel or foam of claim 6, having a particle size distribution of the single structural cells with an average feret diameter within a range of about 1 μm to about 1000 μm, such as about 100 to about 500 μm, for example about 100 to about 300 μm.

9. The aerogel or foam of claim 8, wherein the hydrogel comprises alginate, pectin, gelatin, methylcellulose, carboxymethylcellulose, hydroxypropylcellulose, hydroxypropyl methyl cellulose, hydroxyethyl methyl cellulose, agar, pluronic acid, triblock PEO-PPO-PEO copolymers of poly(ethylene oxide) (PEO) and poly(propylene oxide) (PPO), dissolved or regenerated plant cellulose, dissolved cellulose-based hydrogel, hyaluronic acid, vegetable protein (e.g. pea protein), food-grade coloring (e.g. beet root), extracellular matrix proteins (e.g. collagen, gelatin, or fibronectin, or any combinations thereof), monoacrylated poly(ethylene glycol), poly(ethylene glycol) diacrylate, poly(ethylene glycol) diacrylate (PEGDA)-co-PEGMA, poly(vinyl alcohol), poly(vinylpyrrolidone), poly(lactic-co-glycolic acid), chitosan, chitin, xanthan gum, elastin, fibrin, fibrinogen, cellulose derivatives, carrageenan, or microcrystalline cellulose, or any combinations thereof; wherein the hydrogel is optionally cross-linked.

10. The aerogel or foam of claim 9, comprising templated or aligned microchannels created by directional freezing; non-directional freezing; by molding using molds having microscale and/or macroscale features (such as channels); by punching, pressing, stamping, or otherwise forming geometric patterns, depressions, holes, channels, grooves, ridges, wells, or other structural features within and/or onto at least one surface (for example using needles); or any combinations thereof.

11. The aerogel or foam of claim 10, wherein the plant tissue comprises apple tissue, pear tissue or heart of palm tissue.

12. The aerogel or foam of claim 11, comprising about 5% to about 95% m/m, such as about 10-50% m/m (or more), single structural cells, groups of structural cells, or both, when the aerogel or foam is in hydrated form.

13. The aerogel or foam of claim 12, wherein the hydrogel comprises alginate, pectin, or both, and a) wherein the aerogel or foam is rehydrated with a CaCl₂ solution when the aerogel or foam is not crosslined; or b) wherein the aerogel or foam is rehydrated with water, an aqueous solution, a buffer, a cell buffer, an alcohol, any aqueous or non-aqueous solution when the aerogel of foam is crosslinked.

14. The aerogel or foam of claim 13, wherein the aerogel or foam comprises one or more animal cells and has a bulk modulus within a range of about 0.1 to about 500 kPa, such as about 1 to about 200 kPa.

15. (canceled)

16. The aerogel or foam of claim 4, wherein at least some cellulose and/or cellulose derivative(s) of the aerogel or foam is cross-linked by physical cross-linking (e.g. using glycine) and/or chemical cross-linking (e.g. using citric acid in the presence of heat); wherein at least some cellulose and/or cellulose derivative(s) of the aerogel or foam is functionalized with a linker (e.g. succinic acid) to which one or more functional moieties are optionally attached (e.g. amine-containing groups, wherein cross-linking may further optionally be achieved with one or more protein cross-linkers such as formalin, formaldehyde, glutaraldehyde, and/or transglutaminase); or any combinations thereof.

17. The aerogel of foam of claim 1, wherein the aerogel or foam comprises single structural cells, groups of structural cells, or both, derived from a decellularized plant or fungal tissue by maceration or mercerization of the decellularized plant or fungal tissue, the single structural cells or groups of structural cells having a decellularized 3-dimensional structure lacking cellular materials and nucleic acids of plant or fungal tissue, and lacking one or more base-soluble lignin components of the plant or fungal tissue.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. A method for growing or repairing bone tissue or for treating spinal cord injury in a subject in need thereof, comprising:
implanting an aerogel or foam as defined in claim 1 at an affected site of the subject in need thereof;
such that the aerogel or foam promotes bone tissue generation or repair.

53. (canceled)

54. (canceled)

55. (canceled)

56. The method for repairing spinal cord injury of claim 52, further comprising:
implanting an aerogel or foam as defined in claim 1 at an affected site, wherein the aerogel or foam comprises templated or aligned microchannels;
such that the aerogel or foam promotes spinal cord repair by aligning growth of nerve cells along the templated or aligned microchannels.

57. A food product comprising the aerogel or foam as defined in claim 1.

58. The food product of claim 57, further comprising a dye or coloring agent.

59. The food product of claim 57, comprising two or more aerogel or foam subunits glued together, wherein the glues comprises agar.

60. (canceled)

61. The food product of claim 57, wherein the aerogel or foam comprises templated or aligned microchannels optionally formed by directional or non-directional freezing, and wherein the aerogel or foam comprises muscle cells, fibroblasts, myofibroblasts, neurons, dorsal root ganglion cells, neuronal structures such as axons, neural precursor cells, neural stem cells, glial cells, endogenous stem cells, neutrophils, mesenchymal stem cells, satellite cells, myoblasts, myotubes, muscle progenitor cells, adipocytes, preadipocytes, chondrocytes, osteoblasts, osteoclasts, pre-osteoblasts, tendon progenitor cells, tenocytes, periodontal ligament stem cells, or endothelial cells, or any combinations thereof, aligned along the templated or aligned microchannels; preferably wherein the aerogel or foam comprises templated or aligned microchannels optionally formed by directional or non-directional freezing, and wherein the aerogel or foam comprises muscle cells, fat cells, connective tissue cells (e.g. fibroblasts), cartilage, bone, epithelial, or endothelial cells, or any combinations thereof, aligned along the templated or aligned microchannels.

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. A cellulose-based hydrogel comprising cellulose derived from decellularized plant or fungal tissue via dissolution with dimethylacetamide and lithium chloride followed by regeneration with ethanol.

81. (canceled)

82. (canceled)

83. (canceled)

84. The cellulose-based hydrogel of claim 80, comprising cellulose derived from decellularized plant or fungal tissue via dissolution with: dimethylacetamide and lithium chloride, LiClO₄, xanthate, EDA/KSCN, H₃PO₄, NaOH/urea, ZnCl₂, TBAF/DMSO, NMMO, an ionic liquid (IL) (such as 1-butylpyridinium chloride and aluminum chloride, alkyl imidazolium in association with nitrate, preferably a room temperature ionic liquid), or any combinations thereof.

85. (canceled)

86. (canceled)

87. (canceled)

88. A food product comprising the aerogel or foam as defined in claim 1, wherein the food product is a meat mimic and comprises a plurality of lines providing the appearance of fatty white lines found in tuna, salmon, or another fish-type meat.

89. The food product of claim 88, wherein the food product is a mimic of tuna, salmon, or another fish meat.

90. The food product of claim 88, wherein the food product contains one or more dyes or colorants providing the color of tuna, salmon, or another fish meat.

91. The food product of claim 88, wherein the plurality of lines are formed in cuts or channels formed in the aerogel or foam, wherein the plurality of lines comprise titanium dioxide or beetroot, optionally combined with agar or sodium alginate as a binding agent.

92. (canceled)

93. The food product of claim 91, wherein the titanium dioxide, optionally combined with agar or sodium alginate as a binding agent, is applied into cuts or channels formed in the aerogel or foam to provide the appearance of the fatty white lines found in tuna, salmon, or another fish-type meat.

94. (canceled)

95. (canceled)

96. (canceled)

97. (canceled)

98. (canceled)

99. The aerogel, foam or structural cell as defined in claim 1 in the form of a non-resorbable dermal filler.

100. The dermal filler of claim 99, wherein the filler comprises single structural cells, groups of structural cells, or both, derived from a plant or fungal tissue, the single structural cells or groups of structural cells having a decellularized 3-dimensional structure lacking cellular materials and nucleic acids of plant or fungal tissue, the single structural cells, groups of structural cells, or both, being derived from the plant or fungal tissue by maceration or mercerization.

101. The dermal filler of claim 100, wherein the dermal filler further comprises a carrier fluid or gel.

102. The dermal filler of claim 101, wherein the carrier fluid or gel comprises water, an aqueous solution such as a saline solution or a hydrogel such as a collagen, hyaluronic acid, methylcellulose, and/or dissolved plant-derived decellularized cellulose-based hydrogel.

103. (canceled)

104. The dermal filler of claim 102, further comprising an anesthetic agent such as lidocaine, benzocaine, tetracaine, polocaine, epinephrine, or any combinations thereof.

105. (canceled)

106. The dermal filler of claim 104, wherein the dermal filler comprises PBS (saline), hyaluronic acid (cross-linked or non-crosslinked), alginate, collagen, pluronic acid (e.g. pluronic F 127), agar, agarose, or fibrin, calcium hydroxylapatite, Poly-L-lactic acid, autologous fat, silicone, dextran, methylcellulose, or any combinations thereof.

107. The dermal filler of claim 106, wherein the dermal filler comprises, at stock concentrations, of at least one of: 2% lidocaine gel; a triple anesthetic gel comprising 20% benzocaine, 6% lidocaine, and 4% tetracaine (BLTgel); 3% Polocaine; or a mixture of 2% lidocaine with epinephrine.

108. The dermal filler of claim 107, wherein the structural cells have a size, diameter, or feret diameter distribution within a range from at least about 20 μm to about 1000 μm and an average projected particle area within a range of about 30,000 to about 75,000 μm².

109. (canceled)

110. (canceled)

111. The dermal filler of 108, wherein the structural cells have a particle size, diameter, or feret diameter distribution having a peak about 200-300 μm or a mean particle size, diameter, or feret diameter within a range of about 200 μm to about 300 μm.

112. (canceled)

113. (canceled)

114. The dermal filler of claim 111, wherein the dermal filler is sterilized.

115. The dermal filler of claim 114, wherein the sterilization is by gamma sterilization.

116. The dermal filler of claim 114, wherein the dermal filler is formulated for subdermal injection, deep dermal injection, subcutaneous injection (e.g. subcutaneous fat injection), or any combinations thereof and is provided in a syringe or an injection device.

117. (canceled)

118. (canceled)

119. (canceled)

120. (canceled)

121. (canceled)

122. (canceled)

123. (canceled)

124. (canceled)

125. (canceled)

126. The dermal filler of claim 106, wherein the dermal filler comprises a final concentration of 0.3% to 2% lidocaine gel.
